(19) United States
(12) Patent Application Publication
(10) Pub. No.: US 2025/0003951 A1
(43) Pub. Date: Jan. 2, 2025

(54) METHOD AND COMPOSITION

(71) Applicant: Oxford University Innovation Limited, Oxford (GB)

(72) Inventors: Connor SWEENEY, Oxford (Oxfordshire) (GB); Paresh VYAS, Oxford (Oxfordshire) (GB)

(21) Appl. No.: 18/555,910

(22) PCT Filed: Apr. 21, 2022
(86) PCT No.: PCT/GB2022/050996

§ 371 Date: Oct. 18, 2023

(30) Foreign Application Priority Data
Apr. 21, 2021 (GB) 2105673.4
Mar. 14, 2022 (GB) 2203492.0

(51) Int. Cl. G01N 33/50

(52) U.S. Cl. CPC G01N 33/5011

(57) ABSTRACT

The invention relates to a method of selecting an immunotherapeutic agent for treating a disease in an individual, and to treatment of the disease by administering the immunotherapeutic agent or the cognate peptide antigen for the immunotherapeutic agent. The invention also relates to an antigen binding molecule that binds to the peptide antigen.

### CROSS-REFERENCE TO RELATED APPLICATIONS

This application is the National Stage of International Application No. PCT/GB2022/050996, filed Apr. 21, 2022, which claims priority to GB 2105673.4, filed Apr. 21, 2021 and GB 2203492.0, filed Mar. 14, 2022, which are entirely incorporated herein by reference.

### FIELD OF THE INVENTION

The invention relates to a method of selecting an immunotherapeutic agent for treating a disease in an individual, and to treatment of the disease by administering the immunotherapeutic agent or the cognate peptide antigen for the immunotherapeutic agent. The invention also relates to an antigen binding molecule that binds to the peptide antigen.

### BACKGROUND TO THE INVENTION

Allogeneic stem/immune cell transplantation (allo-SCT) is the most established, commonly used cancer cellular immunotherapy. Allo-SCT has been in routine clinical use since the 1960s. Over 20,000 allo-SCTs are performed worldwide annually, mainly for the highest-risk blood cancers such as acute myeloid leukaemia (AML).

Allo-SCT involves transfer of blood stem and immune cells from a healthy person to a patient. Allo-SCT is a very toxic treatment that is generally restricted to patients less than 65 years old. Even then, allo-SCT has a 10 to 40% procedure-related mortality rate, and around 20 to 40% of patients die of disease relapse. Only 30-70% of patients are alive, and cured, after allo-SCT.

While many aspects of allo-SCT are poorly understood, the curative effect of allo-SCT is known to rely on alloreactivity of donor T cells against cancer cells. In the case of AML, this alloreactivity is known as Graft-versus-Leukaemia (GvL). It is desirable to understand the mechanistic basis of graft-versus-disease responses, such as GvL, so that the likelihood of a successful outcome can be maximized, and the safety of allo-SCT improved.

A key problem in understanding the mechanistic basis of graft-versus-disease (e.g. GvL) responses is identifying how donor T cells mediating curative responses target tumour cells for recognition. T cells use their T cell receptors (TCRs) to scan target cells for peptide antigens complexed to MHC molecules on their surface. However, the antigen-MHC complexes on recipient tumour cells recognised by donor T cells have yet to be characterised. Such characterisation is crucial to understanding curative graft-versus-disease immune responses, and distinguishing the antigen-MHC complexes that contribute to such responses from those that elicit pathogenic Graft-versus-Host Disease (GvHD).

### SUMMARY OF THE INVENTION

The present invention relates to identifying the antigenic basis of beneficial graft-versus-disease and pathogenic graft-versus-host disease (GvHD) immune responses. The identification of antigens involved in graft-versus-disease immune responses allows the selection of immunotherapeutic agent(s) specific for antigens presented on diseased cells. The identification of antigens involved in GvHD immune responses allows the selection of immunotherapeutic agent(s) that do not target these antigens, thereby mitigating against GvHD.

In more detail, the present inventors have identified that target cells in an individual having a disease present peptide antigens on their surface that are capable of binding to a MHC molecule, and encoded by a germline variation in the diseased individual relative to an individual that does not have the disease. The present inventors have also identified antigen binding molecules that bind to the peptide antigens and may be used to effect immunotherapy against the disease. The present inventors have also identified that certain of the peptide antigens are also present on the surface of normal, non-diseased cells, and that targeting these peptide antigens may cause pathogenic graft-versus-host disease responses.

Accordingly, the present invention provides a method of selecting an immunotherapeutic agent for treating a disease in an individual, the method comprising: (a) identifying a peptide antigen that is (i) encoded by a germline variation in the individual, (ii) capable of binding to a MHC molecule and (iii) present on the surface of a target cell in the individual and optionally absent on the surface of non-target cells in the individual; and (b) selecting an immunotherapeutic agent that comprises an antigen binding molecule that binds to the peptide antigen.

The present invention also provides:
a method of treating a disease in an individual, the method comprising administering to the individual an immunotherapeutic agent that comprises an antigen binding molecule that binds to a peptide antigen that is (i) encoded by a germline variation in the individual, (ii) capable of binding to a MHC molecule and (iii) present on the surface of a target cell in the individual and optionally absent on the surface of non-target cells in the individual;
a method of treating a disease in an individual, the method comprising administering a composition comprising a peptide antigen to the individual and thereby inducing an immune response specific for the peptide antigen, wherein the peptide antigen is (i) encoded by a germline variation in the individual, (ii) capable of binding to a MHC molecule and (iii) present on the surface of a target cell in the individual and optionally absent on the surface of non-target cells in the individual;
an immunotherapeutic agent for use in a method of treating a disease in an individual, wherein the method comprises administering the therapeutic agent to the individual, and the immunotherapeutic agent comprises an antigen binding molecule that binds to a peptide antigen that is (i) encoded by a germline variation in the individual, (ii) capable of binding to a MHC molecule and (iii) present on the surface of a target cell in the individual and optionally absent on the surface of non-target cells in the individual;
a composition comprising a peptide antigen for use in a method of treating a disease in an individual, the method comprising administering the peptide antigen to the individual and thereby inducing an immune response specific for the peptide antigen, wherein the peptide antigen is (i) encoded by a germline variation in the individual, (ii) capable of binding to a MHC molecule and (iii) present on the surface of a target cell in the individual and optionally absent on the surface of non-target cells in the individual;
an antigen binding molecule that binds to a peptide antigen that is (i) encoded by a germline variation in the individual, (ii) capable of binding to a MHC molecule and (iii) present on the surface of a target cell in the individual and optionally absent on the surface of non-target cells in the individual;
a peptide antigen that is (i) encoded by a germline variation in an individual having a disease, (ii) capable of binding to a MHC molecule and (iii) present on the surface of a target cell in the individual and optionally absent on the surface of non-target cells in the individual;—a polynucleotide encoding the antigen binding molecule or peptide antigen of the invention; and
a vector comprising the polynucleotide of the invention.

### BRIEF DESCRIPTION OF THE FIGURES

FIG. 1: Mismatched exome variants between patients and their stem cell transplant donors in 15 patient donor pairs. a) There are a greater number of germline mismatched variants in unrelated compared with sibling transplants. b) 90.2% variants are single nucleotide variants (SNVs) and the commonest result on the coding sequence is missense (91.06%).

FIG. 2: Predicted HLA-binding peptides in 15 patient donor pairs. On the x-axis individual patients. Y-axis number of predicted HLA-binding epitopes with germline variants. a) Number of predicted patient-donor mismatched HLA Class I-binding epitopes per patient. High affinity binders (rank affinity<0.5%) and low affinity binders (rank affinity 1.5-2%). b) Number of patient-donor mismatched HLA Class II-binding peptides per patient. High affinity binders (IC50<150 nM) and low affinity binders (IC50150-500 nM).

FIG. 3: Characterisation of GvL T cell responses. a) IFNγ ELISpot results following stimulation of post-transplant PB with GvL peptide+/−antibodies blocking HLA pan-class I (W6/32), pan-class II (Tü39), HLA-DR (L243), -DQ (Tü169) and -DP (B7/21). Negative control without peptide (DMSO). b) Flow cytometry with intracellular cytokine staining showing % T cells positive for IFNγ, TNFα, CD107a after treatment with peptide vs no treatment.

FIG. 4: Outline of AMADEUS Trial and sample collection. a) Outline of trial. b) Sample collection. Arrowheads indicate when samples are taken per trial protocol. (AML, acute myeloid leukaemia; MDS, myelodysplasia; MRD, minimal residual disease; allo-SCT, allogeneic stem cell transplant)

FIG. 5: PADI4 expression in normal tissues and in cancers. a) Graph showing RNAseq data for PADI4 in 37 normal human tissues (data obtained from Human Protein Atlas (v18.1); Uhlén et al. Science 2015)). TPM=transcripts per million reads. The PADI4 SNP has an allele frequency of 4.27%, and is present in ˜8% of the population, in either homozygous or heterozygous form. The graph shows that in normal tissues, PADI4 expression is highest in haematopoietic tissues (bone marrow and spleen). b) Graph showing RNAseq data for PADI4 in 21 human tumours (data obtained from Human Protein Atlas (v18.1); Uhlén et al. Science 2017)). c) Graph showing RNAseq data for PADI4 in 15 primary AML (acute myeloid leukaemia) samples.

FIG. 6: Identifying Antigen-Specific T Cell Receptors. The following protocol was used to determine the sequence of TCRs recognising peptide 1 (SEQ ID NO: 1) or peptide 2 (SEQ ID NO: 2): 1. Culture post-transplant blood with peptide (identified from IFNγ ELISpot screen) to enrich for antigen-specific T cells. 2. After peptide stimulation, FACS sort activated (IFNγ+) T cells and control (IFNγ−) T cells. 3. Single cell RNA sequencing with V (D) J enrichment. In peptide-activated (i.e. CD4+ IFNγ+) T cells, two clones are expanded. The two expanded clones (CD4+ IFNγ+) represent putative antigen-specific T cells. The large black bands (labelled 56.9% in the left panel and 11.0% in the right panel represents the sum of clonotypes with <0.1% frequency).

FIG. 7: Cytokines/activation markers in putative antigen-specific T cells. Single cell RNA sequencing of FACS sort activated (IFNγ+) T cells and control (IFNγ−) T cells shows that putative antigen-specific T cells express multiple cytokines (IFNγ, TNFα) and activation markers (FASLG, IL2RA, TNFRSF9, GZMB) in response to peptide stimulation.

FIG. 8: Identifying Antigen-Specific T Cell Receptors. The protocol set out in Example 5 was used to determine the sequence of TCRs recognising OX289 peptide 52. After peptide stimulation and IFNγ catch, there were two expanded clones (27.3% and 10.7%). These clones were present at lower frequency in the control (IFNγ−) repertoire.

FIG. 9: Identifying Antigen-Specific T Cell Receptors. The protocol set out in Example 5 was used to determine the sequence of TCRs recognising OX289 peptide 40. After peptide stimulation and IFNγ catch, there was one very expanded clone (85.2%) This clone was not present in the control (IFNγ−) repertoire. (Note-negative control sorted as IFNγ-population from Peptide 52 culture).

FIG. 10: Identifying Antigen-Specific T Cell Receptors. The protocol set out in Example 5 was used to determine the sequence of TCRs recognising OX289 overlapping peptides 73/74. After peptide stimulation and IFNγ catch, there were two large clones, and one smaller clone. These clones were not present in the control (IFNγ−) repertoire. (Note-negative control sorted as IFNγ-population from Peptide 52 culture).

FIG. 11: Identifying Antigen-Specific T Cell Receptors. The protocol set out in Example 5 was used to determine the sequence of TCRs recognising OX289 peptide 221. After peptide stimulation and IFNγ catch, there were two large clones. These clones were not present in the control (IFNγ−) repertoire. (Note-negative control sorted as IFNγ-population from Peptide 52 culture).

FIG. 12: Identifying Antigen-Specific T Cell Receptors. The protocol set out in Example 5 was used to determine the sequence of TCRs recognising OX289 overlappying peptides 75/205. After peptide stimulation and IFNγ catch, there were three expanded clones. These clones were not present in the control (IFNγ−) repertoire. (Note-negative control sorted as IFNγ-population from Peptide 52 culture).

FIG. 13: Identifying Antigen-Specific T Cell Receptors. The protocol set out in Example 5 was used to determine the sequence of TCRs recognising OX802 overlapping peptides 321/322. After peptide stimulation and IFNγ catch, there were two large clone. Clone 1 was present in the control (IFNγ−) repertoire, but clone 2 was absent.

FIG. 14: Identifying Antigen-Specific T Cell Receptors. The protocol set out in Example 5 was used to determine the sequence of TCRs recognising OX628 peptides 80/81. After peptide stimulation and IFNγ catch, there were two expanded clones (55.1% and 13.5%). These clones were present at lower frequency in the control (IFNγ−) repertoire.

FIG. 15: Identifying Antigen-Specific T Cell Receptors. The protocol set out in Example 5 was used to determine the sequence of TCRs recognising OX747 overlapping peptides 157/158. After peptide stimulation and IFNγ catch, multiple clones were expanded at modest size. All were present at lower frequency in the control (IFNγ−) repertoire.

FIG. 16: Identifying Antigen-Specific T Cell Receptors. The protocol set out in Example 5 was used to determine the sequence of TCRs recognising OX885 overlapping peptides 559/560. After peptide stimulation and IFNγ catch, there was one very expanded clone (96.5%). This clones was also present at high frequency in the control (IFNγ−) repertoire. (Note-Negative control population sorted from the same culture as Activated.)

FIG. 17: Identifying Antigen-Specific T Cell Receptors. The protocol set out in Example 5 was used to determine the sequence of TCRs recognising OX885 overlapping peptides OX628-308/309. After peptide stimulation and IFNγ catch, there was one large clone (76.1%). This clones was present at low frequency in the control (IFNγ−) repertoire. (Note-negative control sorted as IFNγ-population from Peptide 559/560 culture).

FIG. 18: Identifying Antigen-Specific T Cell Receptors. The protocol set out in Example 5 was used to determine the sequence of TCRs recognising OX993 overlapping peptides 413/414. Four clones were expanded after peptide stimulation and IFNγ catch, compared to the control (IFNγ−) repertoire.

FIG. 19: Identifying Antigen-Specific T Cell Receptors. The protocol set out in Example 5 was used to determine the sequence of TCRs recognising OX1149 peptide 190. Three clones were expanded after peptide stimulation and IFNγ catch, compared to the control (IFNγ−) repertoire.

FIG. 20: Identifying Antigen-Specific T Cell Receptors. The protocol set out in Example 5 was used to determine the sequence of TCRs recognising OX1149 peptide 290. After peptide stimulation and IFNγ catch, there were three expanded clones. These clones were not present in the control (IFNγ−) repertoire. (Note-negative control sorted as IFNγ-population from Peptide 190 culture).

FIG. 21: Identifying Antigen-Specific T Cell Receptors. The protocol set out in Example 5 was used to determine the sequence of TCRs recognising OX289 peptide 284. The dominant clone in the negative control (IFNγ−) repertoire was larger in the activated repertoire (96.0% vs 65.1%).

FIG. 22: Identifying Antigen-Specific T Cell Receptors. The protocol set out in Example 5 was used to determine the sequence of TCRs recognising OX289 peptide 9. The dominant clone in the negative control (IFNγ−) repertoire was larger in the activated repertoire (91.1% vs 65.1%). (Note-negative control sorted as IFNγ-population from Peptide 284 culture, not Peptide 9 culture).

FIG. 23: Identifying Antigen-Specific T Cell Receptors. The protocol set out in Example 5 was used to determine the sequence of TCRs recognising OX289 peptide 67. The two largest clones in the activated repertoire are not present in the control repertoire. (Note-negative control sorted as IFNγ-population from Peptide 284 culture).

### DETAILED DESCRIPTION OF THE INVENTION

The present inventors have, for the first time, identified peptide antigens targeted by graft-versus-disease (such as graft-versus-cancer or graft-versus-leukemia, GvL) and graft-versus-host disease immune responses. The inventors have also identified cognate antigen binding molecules, such as T cell receptors (TCRs), that recognise the peptide antigens targeted by graft-versus-disease responses and can therefore be used to provide immunotherapy against the disease.

In more detail, the present inventors have demonstrated that the graft-versus-disease and graft-versus-host disease immune responses are primarily specific for peptide antigens that are encoded by a germline variation in the diseased individual and present on the surface of cells targeted by allogeneic hemopoietic stem cell transplantation.

With knowledge of the antigenic basis of the graft-versus-disease immune response, it is possible to select donor-patient pairings for allo-SCT that maximise the likelihood of a successful treatment outcome. It is also possible to provide an immunotherapeutic agent that comprises an antigen binding molecule (such as a T cell receptor, T cell receptor fragments, chimeric antigen receptor (CAR), antibody, antibody fragment, or bi-specific T cell engager (BiTE)) that mimics the antigen specificity of donor T cells capable of mounting an effective graft-versus-disease immune response. In this way, the graft-versus-disease immune response usually associated with allo-SCT may be harnessed for use in off-the-shelf or autologous approaches to treatment. It is also possible to prime immune cells present in a patient to mount an response against disease-associated target cells by administering a relevant peptide antigen as a vaccine. Any of these approaches may be used in combination.

Knowledge of the antigenic basis of the graft-versus-host immune response may also be used to select donor-patient pairings for allo-SCT. In particular, donor cells that recognise peptide antigens present on non-target cells (such as normal cells in healthy tissue) can be avoided. Immunotherapeutic agents that comprise an antigen binding molecule that mimics the antigen specificity of such donor cells can likewise be avoided, as can the priming of immune cells present in the patient and specific for the graft-versus-host peptide antigen.

To identify the antigenic basis of the graft-versus-disease and graft-versus-host disease immune response, the inventors compared the sequence of patient and donor genomic DNA to identify germline coding variants mismatched between the patient and donor. In the exemplified embodiment, the patient is an AML patient and the donor is a donor without AML. However, the type of antigen identified as the basis of the graft-versus-AML response (namely, a MHC-restricted peptide antigen that is encoded by a germline variation in the patient and present on the surface of target cells) may also be expressed by target cells associated with other diseases, such as other types of cancer. In addition, as the graft-versus-AML response targets a germline variant (rather than, for example, a disease-specific neo-epitope or tumour associated antigen), the peptide antigen may be expressed by different types of target cells in different individuals, such as target cells derived from different tissues. For example, the same peptide antigen may be expressed on the surface of an AML cell in one individual, and on the surface of a solid tumour cell in another individual.

Accordingly, the approach employed by the inventors has wide-reaching impact on the treatment of diseases such as cancer. The approach allows an effective immunotherapeutic agent or peptide antigen vaccine to be selected for different individuals having different types of disease, for example different types of cancer. Furthermore, a single immunotherapeutic agent having a single antigen specificity may be useful in treating different diseases in different individuals each comprising a particular germline variation, depending on the type of target cell upon whose surface the peptide antigen is expressed. This may especially be the case for germline variation-encoded peptides that are expressed by target cells from two or more patients, as these may represent so-called “public” antigens, which frequently occur in the population (e.g. have a frequency of 0.05 or greater).

The various aspects of the invention are described in detail below.

### Method of Selecting an Immunotherapeutic Agent

The invention provides a method of selecting an immunotherapeutic agent for treating a disease in an individual, the method comprising (a) identifying a peptide antigen that is (i) encoded by a germline variation in the individual, (ii) capable of binding to a MHC molecule and (iii) present on the surface of a target cell in the individual and optionally absent on the surface of non-target cells in the individual; and (b) selecting an immunotherapeutic agent that comprises an antigen binding molecule that binds to the peptide antigen. Preferably, the antigen binding molecule binds to the peptide antigen when the peptide antigen is bound to the MHC molecule. That is, the antigen binding molecule may bind to a peptide-MHC complex comprising the peptide antigen.

Identification of the peptide antigen and selection of an immunotherapeutic agent that comprises an antigen binding molecule that binds to the peptide antigen maximises the likelihood that administration of the immunotherapeutic agent to the individual will result in successful treatment of the disease. In essence, the antigen binding ability of the antigen binding molecule comprised in the immunotherapeutic agent can be matched to the antigen expression profile of the target cell, increasing the probability that the target cell is bound by the antigen binding molecule to effect treatment of the disease.

Absence of the peptide antigen on the surface of non-target cells minimizes the risk of graft-versus-host disease resulting from administration of the immunotherapeutic agent to the individual. In this case, the immunotherapeutic agent selectively binds to target cells, but does not bind to non-target cells. Target cells and non-target cells are described in detail below.

### Medicaments and Methods of Treatment

The invention provides a method of treating a disease in an individual, the method comprising administering to the individual an immunotherapeutic agent that comprises an antigen binding molecule that binds to a peptide antigen that is (i) encoded by a germline variation in the individual, (ii) capable of binding to a MHC molecule and (iii) present on the surface of a target cell in the individual and optionally absent on the surface of non-target cells in the individual. The invention also provides an immunotherapeutic agent for use in a method of treating a disease in an individual, wherein the method comprises administering the immunotherapeutic agent to the individual, and the immunotherapeutic agent comprises an antigen binding molecule that binds to a peptide antigen that is (i) encoded by a germline variation in the individual, (ii) capable of binding to a MHC molecule and (iii) present on the surface of a target cell in the individual and optionally absent on the surface of non-target cells in the individual.

The peptide antigen is present on the surface of target cells, because it is bound to cell surface MHC molecules. Following administration of the immunotherapeutic agent, the antigen binding molecule specifically binds to target cells via the peptide antigen. Binding may effect treatment of the disease in a variety of ways, depending on the nature of antigen binding molecule and/or immunotherapeutic agent. For example, the immunotherapeutic agent may comprise a T cell expressing a T cell receptor (TCR) as the antigen binding molecule. Binding of the T cell receptor to the peptide antigen-MHC complex may trigger activation of the T cell and result in, for example, cytolysis of the target cell and/or the production of cytokines (such as interferon gamma (IFNγ) and/or tumour necrosis factor alpha (TNFα)) that are detrimental to the target cell. The immunotherapeutic agent may, for example, comprise an antigen binding molecule that comprises an antibody or antibody fragment. Binding of the antibody or antibody fragment to the peptide antigen may decorate the target cell with an opsonin, marking the target cell for phagocytosis or destruction via the classical complement pathway. The antibody/antibody fragment may be bound to a chemotherapeutic agent, and so binding of the antibody or antibody fragment to the peptide antigen may deliver the chemotherapeutic agent to the target cell. The immunotherapeutic agent may, for example, comprise an antigen binding molecule comprising a bi-specific T cell engager (BiTE). The BiTE may simultaneously bind to (i) the peptide antigen and (ii) a molecule (e.g. CD3) expressed on the surface of a T cell, to form a link between the target cell and the T cell that is independent of the antigen specificity of the T cell. Binding of the surface molecule on the T cell may activate the T cell and lead, for example, to cytolytic activity against the target cell.

Absence of the peptide antigen on the surface of non-target cells minimizes the risk of graft-versus-host disease resulting from administration of the immunotherapeutic agent to the individual. In this case, the immunotherapeutic agent selectively binds to target cells, but does not bind to non-target cells. Target cells and non-target cells are described in detail below.

The step of administering the immunotherapeutic agent to the individual may, for example, comprise administering two or more immunotherapeutic agents to the individual, wherein each immunotherapeutic agent comprises an antigen binding molecule that binds to a peptide antigen that is (i) encoded by a germline variation in the individual, (ii) capable of binding to a MHC molecule, and (iii) present on the surface of a target cell in the individual and optionally absent on the surface of non-target cells in the individual. For instance, the individual may be administered with three or more, four or more, five or more six or more, seven or more, eight or more, nine or more, or ten or more immunotherapeutic agents each comprising an antigen binding molecule that binds to a peptide antigen that is (i) encoded by a germline variation in the individual, (ii) capable of binding to a MHC molecule, and (iii) present on the surface of a target cell in the individual and optionally absent on the surface of non-target cells in the individual. Each immunotherapeutic agent administered to the individual may, for example, comprise an antigen binding molecule that binds to a different peptide antigen. Each different peptide antigen may be encoded by a different germline variation in the individual.

The step of administering the immunotherapeutic agent to the individual may, for example, comprise administering a single immunotherapeutic agent to the individual, wherein the single immunotherapeutic agent comprises two or more antigen binding molecules that each bind to a peptide antigen that is (i) encoded by a germline variation in the individual, (ii) capable of binding to a MHC molecule, and (iii) present on the surface of a target cell in the individual and optionally absent on the surface of non-target cells in the individual. For instance, the individual may be administered with a single immunotherapeutic agent comprising three or more, four or more, five or more six or more, seven or more, eight or more, nine or more, or ten or more antigen binding molecules that each bind to a peptide antigen that is (i) encoded by a germline variation in the individual, (ii) capable of binding to a MHC molecule, and (iii) present on the surface of a target cell in the individual and optionally absent on the surface of non-target cells in the individual. Each antigen binding molecule comprised in the single immunotherapeutic agent may, for example, bind to a different peptide antigen. Each different peptide antigen may be encoded by a different germline variation in the individual.

In another aspect, the invention provides a method of treating a disease in an individual, the method comprising administering a composition comprising a peptide antigen to the individual and thereby inducing an immune response specific for the peptide antigen, wherein the peptide antigen is (i) encoded by a germline variation in the individual, (ii) capable of binding to a MHC molecule, and (iii) present on the surface of a target cell in the individual and optionally absent on the surface of non-target cells in the individual. The invention also provides a composition comprising a peptide antigen for use in a method of treating a disease in an individual, the method comprising administering the peptide antigen to the individual and thereby inducing an immune response specific for the peptide antigen, wherein the peptide antigen is (i) encoded by a germline variation in the individual, (ii) capable of binding to a MHC molecule, and (iii) present on the surface of a target cell in the individual and optionally absent on the surface of non-target cells in the individual.

Following administration of the peptide antigen, components of the individual's own immune system may be primed to mount an immune response against target cells. For instance, administration of the peptide antigen may stimulate expansion of T cells and/or B cells specific for the peptide antigen. Administration of the peptide antigen may stimulate activation of T cells and/or B cells specific for the peptide antigen. In this way, the T cell and/or B cell responses directed against the target cell may be induced. Absence of the peptide antigen on the surface of non-target cells minimizes the risk of graft-versus-host disease resulting from the primed immune response.

The step of administering the peptide antigen to the individual may, for example, comprise administering two or more peptide antigens to the individual, wherein each peptide antigen is (i) encoded by a germline variation in the individual, (ii) capable of binding to a MHC molecule, and (iii) present on the surface of a target cell in the individual and optionally absent on the surface of non-target cells in the individual. For instance, the individual may be administered with three or more, four or more, five or more six or more, seven or more, eight or more, nine or more, or ten or more peptide antigens each (i) encoded by a germline variation in the individual, (ii) capable of binding to a MHC molecule, and (iii) present on the surface of a target cell in the individual and optionally absent on the surface of non-target cells in the individual. Each peptide antigen administered to the individual may, for example, be encoded by a different germline variation in the individual. The peptide antigens may be comprised in the same composition.

The method of treating disease in an individual may further comprise administering to the individual immune cells specific for the peptide antigen. In other words, the individual may be administered with (i) the peptide antigen, and (ii) immune cells specific for the peptide antigen. Following administration of the peptide antigen, the cells specific for the peptide antigen may be primed to mount an immune response against target cells. This may facilitate a therapeutic effect in the event that components of the individual's own immune system are tolerant to the peptide antigen. The peptide antigen and immune cells specific for the peptide antigen may be comprised in the same composition. Absence of the peptide antigen on the surface of non-target cells minimizes the risk of graft-versus-host disease resulting from the primed immune response.

If the individual is administered with a plurality of peptide antigens, as described above, the method may comprise administering to the individual immune cells specific for each of the peptide antigens. The administered immune cells may, for example, comprise separate immune cells each specific for one of the peptide antigens. The administered immune cells may, for example, comprise one or more immune cells each specific for a plurality of the peptide antigens. For example, if the individual is administered with peptide antigen X and peptide antigen Y, the method may comprise administering (a) immune cells specific for peptide antigen X and immune cells specific for peptide antigen Y or (b) immune cells individually specific for both peptide antigen X and peptide antigen Y. In any case, the peptide antigens and immune cells specific for the peptide antigens may be comprised in the same composition.

Alternatively, the peptide antigen and the immune cells specific for the peptide antigen may be comprised in different compositions. In this case, the composition comprising the peptide antigen and the composition comprising the immune cells specific for the peptide antigen may be administered to the individual together. That is, the composition comprising the peptide antigen and the composition comprising the immune cells specific for the peptide antigen may be administered to the individual at substantially the same time. The composition comprising the peptide antigen and the composition comprising the immune cells specific for the peptide antigen may be administered to the individual separately. That is, the composition comprising the peptide antigen and the composition comprising the immune cells specific for the peptide antigen may be administered to the individual at different times. For example, the method may comprise administering the individual with (i) the composition comprising the peptide antigen and, subsequently, (ii) the composition comprising immune cells specific for the peptide antigen. Preferably, the method comprises administering the individual with (i) the composition comprising the immune cells specific for the peptide antigen and, subsequently, (ii) the composition comprising the peptide antigen.

The immune cells specific for the peptide antigen may, for example, comprise T cells, B cells, NK cells, NKT cells, macrophages and/or monocytes. Preferably, the cells specific for the peptide antigen comprise T cells and/or B cells. More preferably, the immune cells specific for the peptide antigen comprise T cells. The immune cells specific for the peptide antigen may exist in nature. For example, the immune cells specific for the peptide antigen may be obtained from a donor. The donor may, for example, be donor whose genome does not comprise the germline variation. Preferably, the donor does not have the disease. The immune cells specific for the peptide antigen may, for example, be engineered cells. The engineered immune cells may be autologous or allogeneic with respect to the individual. The immune cells may be engineered to comprise a receptor conferring specificity for the peptide antigen. For example, the immune cells may comprise a T cell receptor or a chimeric antigen receptor (CAR) specific for the peptide antigen.

The immune response induced by administration of the peptide antigen may be an adaptive immune response. For example, the immune response may comprise a T cell response, a B cell response and/or a NKT cell response. Preferably, the immune response comprises a T cell response. The T cell response may comprise a CD4+ T cell response. The T cell response may comprise a CD8+ T cell response. Preferably, the MHC molecule to which the peptide antigen is capable of binding is a MHC class II molecule, and T cell response comprises a CD4+ T cell response. Preferably, the MHC molecule to which the peptide antigen is capable of binding is a MHC class I molecule, and T cell response comprises a CD8+ T cell response.

The CD4+ T cells and/or CD8+ T cells involved in the T cell response may have cytolytic effector function. The CD4+ T cells and/or CD8+ T cells may express markers of cytolytic effector function. For example, the CD4+ T cells and/or CD8+ T cells may express one or more of perforin, granzyme A, granzyme B, Fas, FasL and TRAIL. The CD4+ T cells and/or CD8+ T cells involved in the T cell response may express CD107a, a degranulation marker.

The CD4+ T cells and/or CD8+ T cells involved in the T cell response may secrete one or more cytokines. For example, the CD4+ T cells and/or CD8+ T cells may secrete interferon gamma (IFNγ). The CD4+ T cells and/or CD8+ T cells may secrete tumour necrosis factor alpha (TNFα).

The immune response induced by administration of the peptide antigen may be an innate immune response. For example, the immune response may comprise a NK cell response, a macrophage response, a dendritic cell response, or a monocyte response.

The method may further comprise administering to the individual an adjuvant. The adjuvant may, for example, comprise one or more of incomplete Freund's adjuvant (IFA), montanide, an aluminium adjuvant (alum), a microparticle, a nanoparticle, a Toll-like receptor (TLR) agonist, a cytokine, or an antibody. The aluminium adjuvant may comprise aluminium hydroxide and/or aluminium phosphate. The microparticle or nanoparticle may, for example, comprise a liposome, a synthetic polymer (such as polystyrene, poly(lactide-co-glycolide) PLG, poly(lactic acid) PLA and/or PLGA), a natural polymer (such as gelatin, collagen and/or chitosan), and/or a carbon nanotube. The TLR agonist may comprise, for example, Pam₃CSK₄. Poly-ICLC, MPLA, Imiquimod, and/or CpG. The cytokine may comprise, for example, IL-2, GM-CSF, IFN or CDN. The antibody may, for example, comprise an anti-PD1 antibody or an anti-CTLA-4 antibody. Preferably, the adjuvant is administered together with the peptide antigen. That is, the adjuvant and the peptide antigen may be administered to the individual at substantially the same time. For example, the adjuvant and the peptide antigen may be comprised in the same composition. In other words, the composition comprising the peptide antigen may further comprise the adjuvant.

### Disease

The disease may be any disease, such as a disease that results in or from the presence of an undesirable cell in the individual. An undesirable cell may, for example, be involved in the aetiopathogenesis of the disease. An undesirable cell may, for example, cause harm in the individual. Targeting the undesirable cell with the immunotherapeutic agent may impair the function of the undesirable of the cell or kill the undesirable cell, thereby treating the disease. The undesirable cell may express MHC class II. The undesirable cell may express MHC class I. The undesirable cell may be a target cell.

The disease may, for example, be cancer. In this case, the undesirable cell may be a cancer cell. The cancer may, for example, be anal cancer, bile duct cancer (cholangiocarcinoma), bladder cancer, blood cancer, bone cancer, bowel cancer, brain tumours, breast cancer, colorectal cancer, cervical cancer, endocrine tumours, Ewing's sarcoma, eye cancer (such as ocular melanoma), fallopian tube cancer, gall bladder cancer, head and/or neck cancer, Kaposi's sarcoma, kidney cancer, larynx cancer, leukaemia, liver cancer, lung cancer, lymph node cancer, lymphoma, melanoma, mesothelioma, myeloma, neuroendocrine tumours, ovarian cancer, oesophageal cancer, pancreatic cancer, penis cancer, primary peritoneal cancer, prostate cancer, Pseudomyxoma peritonei, skin cancer, small bowel cancer, soft tissue sarcoma, spinal cord tumours, stomach cancer, testicular cancer, thymus cancer, thyroid cancer, trachea cancer, unknown primary cancer, vagina cancer, vulva cancer or endometrial cancer. The leukaemia is preferably acute lymphoblastic leukaemia, acute myeloid leukaemia (AML), chronic lymphocytic leukaemia or chronic myeloid leukaemia. The lymphoma may be Hodgkin lymphoma or non-Hodgkin lymphoma. The cancer may, for example, beacute myeloid leukaemia (AML). The cancer cell may, for example, be an AML cell.

The disease may, for example, be an autoimmune disease. In this case, the undesirable cell may be an immune cell. The immune cell may, for example, be a lymphocyte, such as a T cell or a B cell. The lymphocyte may comprise an antigen receptor that is specific for a self-antigen. The autoimmune disease may, for example, be alopecia areata, autoimmune encephalomyelitis, autoimmune hemolytic anemia, autoimmune hepatitis, dermatomyositis, diabetes (type 1), autoimmune juvenile idiopathic arthritis, celiac disease, glomerulonephritis, Graves' disease, Guillain-Barré syndrome, idiopathic thrombocytopenia purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, uveitis or vitiligo.

The disease may, for example, be an allergic disease. In this case, the undesirable cell may be an immune cell. The immune cell may, for example, be a lymphocyte, such as a T cell or a B cell. The lymphocyte may comprise an antigen receptor that is specific for an allergen. The allergic disease may, for example, be atopic dermatitis, allergic airway inflammation or perennial allergic rhinitis.

The disease may, for example, be a fibrosing disease. A fibrosing disease is a disease in which inflammation and/or tissue damage leads to fibrosis. In this case, the undesirable cell may be a fibroblast. The undesirable cell may, for example, be a lymphocyte, such as a T cell or a B cell. The fibrosing disease may, for example, be pulmonary fibrosis (such as cystic fibrosis or idiopathic pulmonary fibrosis), myocardial fibrosis (such as interstitial fibrosis or replacement fibrosis), cirrhosis, bridging fibrosis of the liver, glial scar, arterial stiffness, arthrofibrosis, Crohn's disease, Dupuytran's contracture, keloid, mediastinal fibrosis, myelofibrosis, Peyronie's disease, nephrogenic systemic fibrosis, retroperitoneal fibrosis, scleroderma, systemic sclerosis, or adhesive capsulitis

### Individual

Preferably, the individual is a human individual. Alternatively, the individual may be a non-human mammal. For instance, the individual could be a pet mammal (such as a cat, a dog, a horse, a rabbit or a guinea pig), a commercially farmed mammal (such as an ox, a sheep, a goat or a pig), or a laboratory mammal, (such as a mouse or a rat). The individual may be an infant, a juvenile or an adult. The individual may be known to have the disease, or suspected to have the disease. The terms “individual” and “patient” may be used interchangeably.

### Peptide Antigen

The methods, medicaments and medical uses described above involve a peptide antigen that is (i) encoded by a germline variation in the individual, (ii) capable of binding to a MHC molecule and (iii) present on the surface of a target cell in the individual and optionally absent on the surface of non-target cells in the individual. The invention further provides such peptide antigen. That is, the invention further provides a peptide antigen (such as an isolated peptide antigen) that is (i) encoded by a germline variation in an individual having a disease, (ii) capable of binding to a MHC molecule, and (iii) present on the surface of a target cell in the individual and optionally absent on the surface of non-target cells in the individual. Any of the aspects described above in connection with the methods, medicaments and medical uses of the invention may also apply to the peptide antigen of the invention.

The term “peptide” relates to a short chain of amino acids, linked by peptide (—CO—NH—) bonds. The peptide antigen may, for example, comprise or consist of 10 to 20 amino acids, such as 11 to 19, 12 to 18, 13 to 17, or 14 to 16 amino acids. Preferably, the peptide antigen comprises or consists of 13 to 17 amino acids. The peptide antigen may, for example, comprise 10, 11, 12, 13, 14, 15, 16, 17, 18, 19 or 20 amino acids. The peptide antigen may consist of 10, 11, 12, 13, 14, 15, 16, 17, 18, 19 or 20 amino acids.

The peptide antigen may comprise or consist of an epitope. The epitope may, for example, be a CD4+ T cell epitope, a B cell epitope, or an antibody epitope. A CD4+ T cell epitope is a peptide that is capable of (i) presentation by a MHC molecule and (ii) binding to a T cell receptor (TCR) present on a CD4+ T cell. Preferably, binding of the TCR results in activation of the CD4+ T cell. CD4+ T cell activation may lead to increased proliferation, cytokine production and/or cytotoxic effects. A B cell epitope is a peptide that is capable of binding to a B cell receptor (BCR) present on a B cell. Preferably, binding of the BCR results in activation and/or maturation of the B cell. B cell activation may lead to increased proliferation, and/or antibody production. An antibody epitope is a peptide that is capable of binding to the paratope of an antibody.

Various peptide antigens are identified in the Examples, and include:

(SEQ ID NO: 1)
LTISLLDTFNLELPEAVVFQ
(SEQ ID NO: 2)
ISLLDTFNLELPEAVVFQDS
(SEQ ID NO: 3)
SLLDTFNLELPEAVVF
(SEQ ID NO: 4)
LDTFNLELPEAVVFQ
(SEQ ID NO: 380)
AASCEPLASVLRAKLTSRSS
(SEQ ID NO: 381)
GAGPDPLRLHGHLPVRTSCP
(SEQ ID NO: 382)
TQRSVLLCKVVGARGVGKSA
(SEQ ID NO: 383)
KVVGARGVGKSAFLQAFLGR
(SEQ ID NO: 384)
GQKSPRFRRVSCFLRLGRST
(SEQ ID NO: 385)
RFRRVSCFLRLGRSTLLELE
(SEQ ID NO: 386)
ALAFLLLISIAANLSLLLSR
(SEQ ID NO: 387)
EVQDCLKQLMMSLLQLYRFS
(SEQ ID NO: 388)
GYSPSLHILAIGTRSGAIKL
(SEQ ID NO: 389)
VATFPVYTMVAIPIVCKD
(SEQ ID NO: 390)
PPLYRQRYQFIKNLVDQHEP
(SEQ ID NO: 391)
PLYRQRYQFIKNLVDQHEPK
(SEQ ID NO: 392)
VSRPELLRESISAFLVPMPT
(SEQ ID NO: 393)
TDRALQNKSISAFLVPMPTP
(SEQ ID NO: 394)
NPLSPYLNVDPRYLVQDT
(SEQ ID NO: 395)
EPPVDICLSKAISSSLKGFL
(SEQ ID NO: 396)
GETGMFSLSTIRGHQYATY
(SEQ ID NO: 397)
MNYVSKRLPFAARLNTPMGP
(SEQ ID NO: 398)
LGSLGLIFALTLNRHKYPLN
(SEQ ID NO: 399)
LGLIFALTLNRHKYPLNLYL
(SEQ ID NO: 400)
APISLSSFFNVSTLEREVTD
(SEQ ID NO: 401)
LELGAGTGLASIIAATMART
(SEQ ID NO: 402)
AGTGLASIIAATMARTVYCT
(SEQ ID NO: 403)
VPREYVRALNATKLERVFAK
(SEQ ID NO: 404)
LHRDKALLKRLLKGMQKKRP
(SEQ ID NO: 405)
KALLKRLLKGMQKKRPSDVQ
(SEQ ID NO: 406)
ITVQTVYVQHLITFLDRPIQ
(SEQ ID NO: 407)
QTVYVQHLITFLDRPIQMCC
(SEQ ID NO: 408)
PGLISMFSSSQELGAALAQL
(SEQ ID NO: 409)
WRVRIALALKGIDYETVPIN
(SEQ ID NO: 410)
VRIALALKGIDYETVPINLI
(SEQ ID NO: 411)
DRAEKFNRGIRKLGITPEGQ
(SEQ ID NO: 412)
EKFNRGIRKLGITPEGQSYL

The peptide antigen may comprise or consist of SEQ ID NO: 3. The peptide antigen may, for example, consist of 10 to 20 (such as 11 to 19, 12 to 18, 13 to 17, or 14 to 16) amino acids and comprise SEQ ID NO: 3. For example, the peptide antigen may comprise or consist of SEQ ID NO: 1. The peptide antigen may comprise or consist of SEQ ID NO: 4. The peptide antigen may, for example, consist of 10 to 20 (such as 11 to 19, 12 to 18, 13 to 17, or 14 to 16) amino acids and comprise SEQ ID NO: 4. For example, the peptide antigen may comprise or consist of SEQ ID NO: 2. The peptide antigen may comprise any one of SEQ ID NOs: 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411 and 412. The peptide antigen may consist of any one of SEQ ID NOs: 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411 and 412.

### Target Cell

The peptide antigen is present on the surface of a target cell in the individual. The target cell may express MHC class II. The target cell may express MHC class I. The target cell may be an undesirable cell. The target cell may be involved in the aetiopathogenesis of the disease. The target cell may cause harm in the individual.

The target cell may, for example, be a cancer cell. The cancer may, for example, be an anal cancer cell, a bile duct cancer (cholangiocarcinoma) cell, a bladder cancer cell, a blood cancer cell, a bone cancer cell, a bowel cancer cell, a brain tumour cell, a breast cancer cell, a colorectal cancer cell, a cervical cancer cell, an endocrine tumour cell, an eye cancer (such as ocular melanoma) cell, a fallopian tube cancer cell, a gall bladder cancer cell, a head and/or neck cancer cell, a Kaposi's sarcoma cell, a kidney cancer cell, a larynx cancer cell, a leukaemia cell, a liver cancer cell, a lung cancer cell, a lymph node cancer cell, a lymphoma cell, a melanoma cell, a mesothelioma cell, a myeloma cell, a neuroendocrine tumour cell, an ovarian cancer cell, an oesophageal cancer cell, a pancreatic cancer cell, a penis cancer cell, a primary peritoneal cancer cell, a prostate cancer cell, a Pseudomyxoma peritonei cell, a skin cancer cell, a small bowel cancer cell, a soft tissue sarcoma cell, a spinal cord tumour cell, a stomach cancer cell, a testicular cancer cell, a thymus cancer cell, a thyroid cancer cell, a trachea cancer cell, an unknown primary cancer cell, a vagina cancer cell, a vulva cancer cell or an endometrial cancer cell. The leukaemia may be acute lymphoblastic leukaemia, acute myeloid leukaemia (AML), chronic lymphocytic leukaemia or chronic myeloid leukaemia. The lymphoma may be Hodgkin lymphoma or non-Hodgkin lymphoma. Preferably, the cancer cell is an AML cell.

The target cell may, for example, be an immune cell. For instance, the immune cell may be a lymphocyte, such as a T cell or a B cell. The lymphocyte may comprise an antigen receptor that is specific for a self-antigen. Specificity for the self-antigen may, for example, cause alopecia areata, autoimmune encephalomyelitis, autoimmune hemolytic anemia, autoimmune hepatitis, dermatomyositis, diabetes (type 1), autoimmune juvenile idiopathic arthritis, celiac disease, glomerulonephritis, Graves' disease, Guillain-Barré syndrome, idiopathic thrombocytopenia purpura, myasthenia gravis, autoimmune myocarditis, multiple sclerosis, pemphigus/pemphigoid, pernicious anemia, polyarteritis nodosa, polymyositis, primary biliary cirrhosis, psoriasis, rheumatoid arthritis, scleroderma/systemic sclerosis, Sjögren's syndrome, systemic lupus erythematosus, autoimmune thyroiditis, uveitis or vitiligo. The lymphocyte may comprise an antigen receptor that is specific for an allergen. Specificity for the allergen may, for example, cause atopic dermatitis, allergic airway inflammation or perennial allergic rhinitis.

The target cell may, for example, be a fibroblast.

The peptide antigen is present on the surface of the target cell. The peptide antigen may be considered to be present on the surface of the target cell if any method for determining surface presence shows that the target cell presents a detectable level of the peptide antigen on its surface. Methods for determining surface presence are known in the art. Surface expression may, for example, be determined by immunoprecipitation of MHC molecules from the target cell, and mass spectrometry of eluted MHC-bound peptides. Surface presence may, for example, be determined by flow cytometry. For instance, flow cytometry may be used to measure the mean fluorescent intensity (MFI) of the peptide antigen. MFI measures intensity, time average energy flux measured in watts per square metre.

### Non-Target Cell

As set out above, the peptide antigen is present on the surface of a target cell in an individual having a disease. In any of the aspects described herein, the peptide antigen is preferably absent on the surface of non-target cells in the individual.

Target cells are described in detail above. A non-target cell may be any endogenous cell in the individual that is not a target cell. In other words, a non-target cell may be any endogenous cell that (a) is not an undesirable cell, (b) is not involved in the aetiopathogenesis of the disease, and/or (c) does not cause harm in the individual. Accordingly, a non-target cell may be a desirable cell. A non-target cell may be a non-pathogenic cell. A non-target cell may be a harmless cell. In other words, a non-target cell may be a healthy or normal cell. A non-target cell may, for example, be a non-cancer cell such as a non-AML cell.

The non-target cell may express MHC class II. The non-target cell may express MHC class I.

The term “absent on the surface of non-target cells” may be used interchangeably with the term “undetectable on the surface of non-target cells”. Accordingly, the peptide antigen may be considered to be absent on the surface of a non-target cell if any method for determining surface presence shows that the peptide antigen is undetectable on the surface of the non-target cell. Methods for determining surface presence (and thus surface absence) are known in the art. Surface expression may, for example, be determined by immunoprecipitation of MHC molecules from the target cell, and mass spectrometry of eluted MHC-bound peptides. Surface presence may, for example, be determined by flow cytometry. For instance, flow cytometry may be used to measure the mean fluorescent intensity (MFI) of the peptide antigen. MFI measures intensity, time average energy flux measured in watts per square metre.

The peptide antigen may be absent on the surface of a particular type of non-target cells. For example, the peptide antigen may be absent on the surface of non-target cells in a particular tissue. The peptide antigen may be absent on the surface of non-target cells in a particular organ. For instance, the peptide antigen may be absent on the surface of non target cells in the anus, bile duct, bladder, blood, bone, bowel, brain, breast, colon, rectum, cervix, endocrine organs, eye, fallopian tube, gall bladder, head, neck, kidney, larynx, liver, lung, lymph node, muscle, peripheral nerves, ovary, oesophagus, pancreas, penis, peritoneum, prostate, skin, spinal cord, stomach, testicles, thymus, thyroid, trachea, vagina, vulva and/or endometrium, alone or in any combination.

The peptide antigen may be absent on the surface of any number or proportion of non-target cells in the individual. For instance, the peptide antigen may be absent on the surface of all non target cells. The peptide antigen may be absent on the surface of 70% or more of non-target cells in the individual. For example, the peptide antigen may be absent on the surface of 75% or more, 80% or more, 85% or more, 90% or more, 95% or more, 96% or more, 97% or more, 98% or more, or 99% or more of non-target cells in the individual. In other words, the peptide antigen may be present on no more than 1%, no more than 2%, no more than 3%, no more than 5%, no more than 10%, no more than 15%, no more than 20%, no more than 25%, or no more than 30% of non-target cells in the individual. The peptide antigen may be absent on the surface of any proportion or number of non-target cells in a particular tissue. The peptide antigen may be absent on the surface of any proportion or number of non-target cells in a particular organ. For instance, the peptide antigen may be absent on the surface of any proportion or number of non target cells in the anus, bile duct, bladder, blood, bone, bowel, brain, breast, colon, rectum, cervix, endocrine organs, eye, fallopian tube, gall bladder, head, neck, kidney, larynx, liver, lung, lymph node, muscle, peripheral nerves, ovary, oesophagus, pancreas, penis, peritoneum, prostate, skin, spinal cord, stomach, testicles, thymus, thyroid, trachea, vagina, vulva and/or endometrium, alone or in any combination.

### Germline Variation

The peptide antigen is encoded by a germline variation in the individual. A germline variation is a change in the genetic structure that is inherited from a parent, and capable of being passed to offspring. Accordingly, the peptide antigen is not encoded by a somatic mutation specific to the target cell. In other words, the peptide antigen is not a neoantigen and does not comprise a neo-epitope. The peptide antigen is not a tumour-specific antigen (TSA).

For this reason, the peptide antigen may be present on the surface different types of target cells in different individuals, such as target cells derived from different tissues. For example, the peptide antigen may be present on the surface of an AML cell in one individual, and on the surface of a solid tumour cell in another individual. A single immunotherapeutic agent may therefore be used to treat different diseases in different individuals each comprising the germline variation, depending on the type of target cell upon whose surface the peptide antigen is expressed.

Preferably, the peptide antigen binds to an antigen binding domain that is expressed by an immune cell from a donor whose genome does not comprise the germline variation. Preferably, the donor does not have the disease. The donor may be an actual or prospective donor. The antigen binding domain may, for example, be a T cell receptor, a B cell receptor, an antibody, or an antibody fragment. The antibody fragment may be a fragment antigen-binding (Fab) fragment, a F (ab′)2 fragment, a single chain variable fragment (scFv), a scFv-Fc. The immune cell may be a T cell, a B cell, or a plasma cell, for example. Preferably, the immune cell is a T cell. The immune cell may recognise the peptide antigen as “foreign” or “non-self”. The peptide antigen may be immunogenic with respect to the donor. The peptide antigen may be a minor histocompatibility antigen.

In other words, the germline variation may be a variation relative to the genome of the donor or prospective donor. The germline variation may give rise to polymorphism between the individual and the donor. Polymorphism may, for example, exist between a protein or peptide in the individual, and a corresponding protein or peptide in the donor. The germline variation may render the peptide antigen immunogenic with respect to the donor. The germline variation may encode a minor histocompatibility antigen.

The germline variation may comprise one or more nucleotide substitutions, insertions and/or deletions relative to the donor genome. For example, the germline variation may comprise two or more, three or more, four or more, or five or more nucleotide substitutions relative to the donor genome. The germline variation may comprise two or more, three or more, four or more, or five or more nucleotide insertions relative to the donor genome. The germline variation may comprise two or more, three or more, four or more, or five or more nucleotide deletions relative to the donor genome. The amino acid substitution may, for example, be a conservative amino acid substitution. The amino acid substitution may, for example, be a non-conservative amino acid substitution.

Conservative substitutions replace amino acids with other amino acids of similar chemical structure, similar chemical properties or similar side-chain volume. The amino acids introduced may have similar polarity, hydrophilicity, hydrophobicity, basicity, acidity, neutrality or charge to the amino acids they replace. Alternatively, the conservative substitution may introduce another amino acid that is aromatic or aliphatic in the place of a pre-existing aromatic or aliphatic amino acid. Conservative amino acid changes are well-known in the art and may be selected in accordance with the properties of the 20 main amino acids as defined in Table 1 below. Where amino acids have similar polarity, this can also be determined by reference to the hydropathy scale for amino acid side chains in Table 2.

TABLE 1
Chemical properties of amino acids

| | |
|---|---|
| Ala | aliphatic, hydrophobic, neutral |
| Cys | polar, hydrophobic, neutral |
| Asp | polar, hydrophilic, charged (−) |
| Glu | polar, hydrophilic, charged (−) |
| Phe | aromatic, hydrophobic, neutral |
| Gly | aliphatic, neutral |
| His | aromatic, polar, hydrophilic, |
| | charged (+) |
| Ile | aliphatic, hydrophobic, neutral |
| Lys | polar, hydrophilic, charged(+) |
| Leu | aliphatic, hydrophobic, neutral |
| Met | hydrophobic, neutral |
| Asn | polar, hydrophilic, neutral |
| Pro | hydrophobic, neutral |
| Gln | polar, hydrophilic, neutral |
| Arg | polar, hydrophilic, charged (+) |
| Ser | polar, hydrophilic, neutral |
| Thr | polar, hydrophilic, neutral |
| Val | aliphatic, hydrophobic, neutral |
| Trp | aromatic, hydrophobic, neutral |
| Tyr | aromatic, polar, hydrophobic |

TABLE 2
Hydropathy scale

| Side Chain | Hydropathy |
|---|---|
| Ile | 4.5 |
| Val | 4.2 |
| Leu | 3.8 |
| Phe | 2.8 |
| Cys | 2.5 |
| Met | 1.9 |
| Ala | 1.8 |
| Gly | −0.4 |
| Thr | −0.7 |
| Ser | −0.8 |
| Trp | −0.9 |
| Tyr | −1.3 |
| Pro | −1.6 |
| His | −3.2 |
| Glu | −3.5 |
| Gln | −3.5 |
| Asp | −3.5 |
| Asn | −3.5 |
| Lys | −3.9 |
| Arg | −4.5 |

### MHC Binding

The peptide antigen is capable of binding to a MHC molecule.

The MHC molecule may be a MHC class I molecule. The MHC class I molecule may be a human MHC class I molecule. That is, the MHC class I molecule may be a HLA class I molecule.

The peptide antigen may be capable of binding to one or more HLA class I molecules. For example, the peptide antigen may be capable of binding to one or more of (i) a HLA-A molecule, (ii) a HLA-B molecule, and (iii) a HLA-C molecule. For instance, the peptide antigen may be capable of binding to (i); (ii); (iii); (i) and (ii); (i) and (iii); (ii) and (iii); or (i), (ii) and (iii).

The MHC molecule may preferably be a MHC class II molecule. The expression of MHC class II molecule is traditionally limited to professional antigen-presenting cells. However, upregulation of MHC class II expression has been demonstrated for some, target cells associated with certain diseases in a patient, such as AML. The MHC class II molecule may be a human MHC class II molecule. That is, the MHC class II molecule may be a HLA class II molecule.

The peptide antigen may be capable of binding to one or more HLA class II molecules. For example, the peptide antigen may be capable of binding to one or more of (i) a HLA-DP molecule, (ii) a HLA-DM molecule, (iii) a HLA-DOA molecule, (iv) a HLA-DOB molecule, (v) a HLA-DQ molecule, and (vi) a HLA-DR molecule. For instance, the peptide antigen may be capable of binding to (i); (ii); (iii); (iv); (v); (vi); (i) and (ii); (i) and (iii); (i) and (iv); (i) and (v); (i) and (vi); (ii) and (iii); (ii) and (iv); (ii) and (v); (ii) and (vi); (iii) and (iv); (iii) and (v); (iii) and (vi); (iv) and (v); (iv) and (vi); (v) and (vi); (i), (ii) and (iii); (i), (ii) and (iv); (i), (ii) and (v); (i), (ii) and (vi); (i), (iii) and (iv); (i), (iii) and (v); (i), (iii) and (vi); (i), (iv) and (v); (i), (iv) and (vi); (i), (v) and (vi); (ii), (iii) and (iv); (ii), (iii) and (v); (ii), (iii) and (vi); (ii), (iv) and (v); (ii), (iv) and (vi); (ii), (v) and (vi); (iii), (iv) and (v); (iii), (iv) and (vi); (iii), (v), (vi); (iv), (v) and (vi); (i), (ii), (iii) and (iv); (i), (ii), (iii) and (v); (i), (ii), (iii) and (vi); (i), (ii), (iv) and (v); (i), (ii), (iv) and (vi); (i), (ii), (v) and (vi); (i), (iii), (iv) and (v); (i), (iii), (iv) and (vi); (i), (iii), (v) and (vi); (i), (iv), (v) and (vi); (ii), (iii), (iv) and (v); (ii), (iii), (iv) and (vi); (ii), (iii), (v) and (vi); (ii), (iv), (v) and (vi); (iii), (iv), (v) and (vi); (i), (ii), (iii), (iv) and (v); (i), (ii), (iii), (iv) and (vi); (i), (ii), (iii), (v) and (vi); (i), (ii), (iv), (v) and (vi); (i), (iii), (iv), (v) and (vi); (ii), (iii), (iv), (v) and (vi); (i), (ii), (iii), (iv), (v) and (vi).

Methods for determining MHC (or HLA) binding ability are known in the art. For example, in silico analysis may be used to predict the affinity with which a peptide antigen binds to a MHC (or HLA) molecule. Mass spectrometry may be used to identify a peptide eluted from a MHC (or HLA) molecule immunoprecipitated from a target cell. Various biochemical assays for determining peptide-MHC (or peptide-HLA) binding are also available.

### Antigen Binding Molecule

The invention provides an antigen binding molecule that binds to a peptide antigen that is (i) encoded by a germline variation in an individual having a disease, (ii) capable of binding to a MHC molecule, and (iii) present on the surface of a target cell in the individual and optionally absent on the surface of non-target cells in the individual. Such an antigen binding molecule is comprised in the immunotherapeutic agent selected or administered in the methods and medical uses described herein.

The antigen binding molecule may, for example, be a T cell receptor (TCR), a chimeric antigen receptor (CAR), an antibody, antibody fragment or bi-specific T cell engager (BiTE). The antibody fragment may comprise a fragment antigen-binding (Fab) fragment, a F (ab′)2 fragment, a single chain variable fragment (scFv), a scFv-Fc, or a single domain antibody. Preferably, the antigen binding molecule is a TCR or a CAR.

The antigen binding molecule binds to the peptide antigen. The antigen binding molecule may, for example, bind to the peptide antigen when it is bound to a MHC molecule. That is, the antigen binding molecule may bind to a MHC-peptide antigen complex. Preferably, the binding between the peptide antigen and the antigen binding molecule is non-covalent. The binding may be mediated by, for example, electrostatic interaction, hydrogen bonds, van der Waals forces and/or hydrophobic interactions. Methods for detecting binding between a peptide and an antigen binding molecule are well-known in the art in include, for example, enzyme-linked immunosorbent assay (ELISA), enzyme-linked immune absorbent spot (ELISpot) assay, and flow cytometry. Methods for functional testing of an antigen binding molecule are also known in the art. For example, cells may be engineered to express the antigen binding molecule. For instance, when the antigen binding molecule is a TCR, a lentiviral vector may be used to insert the alpha and beta chains of the TCR into T cells, such as human T cells. Then, the peptide antigen is added to a culture containing the cells that express the antigen binding molecule. Cell activation (such as T cell activation) may be measured by well-known techniques, such as (1) an ELISpot for IFNγ secretion, or (2) flow cytometry following immunostaining for intracellular cytokines and/or surface markes of activation. Technique (1) may, for example, be performed after 18 hours of peptide simulation. Technique (2) may, for example, be performed after 6 to 8 hours of peptide simulation.

The antigen binding molecule may comprise an antigen binding domain that is expressed by an immune cell from a donor whose genome does not comprise the germline variation. The immune cell may, for example, be a T cell or NKT cell. The antigen binding domain may, for example, comprise all or part of a TCR alpha chain, such as a TCR alpha chain disclosed herein. The antigen binding domain may comprise all or part of a TCR beta chain, such as a TCR beta chain disclosed herein. The antigen binding domain may comprise all or part of a TCR alpha chain (such as a TCR alpha chain disclosed herein) and all or part of a TCR beta chain (such as a TCR beta chain disclosed herein).

The antigen binding domain may comprise one or more of the complementarity determining regions (CDRs) from the alpha chain of a TCR. For example, the antigen binding domain may comprise two or three of the complementarity determining regions (CDRs) from the alpha chain of a TCR. The antigen binding domain may comprise one or more of the complementarity determining regions (CDRs) from the beta chain of a TCR. For example, the antigen binding domain may comprise two or three of the CDRs from the beta chain of a TCR. The antigen binding domain may comprise one or more of the CDRs from the alpha chain and one or more of the CDRs from the beta chain of a TCR. For example, the antigen binding domain may comprise (i) one of the CDRs from the alpha chain and one of the CDRs from the beta chain, (ii) one of the CDRs from the alpha chain and two of the CDRs from the beta chain, (iii) one of the CDRs from the alpha chain and three of the CDRs from the beta chain, (iv) two of the CDRs from the alpha chain and one of the CDRs from the beta chain, (v) two of the CDRs from the alpha chain and two of the CDRs from the beta chain, (vi) two of the CDRs from the alpha chain and three of the CDRs from the beta chain, (vii) three of the CDRs from the alpha chain and one of the CDRs from the beta chain, (viii) three of the CDRs from the alpha chain and two of the CDRs from the beta chain, or (ix) three of the CDRs from the alpha chain and three of the CDRs from the beta chain. The antigen binding domain may comprise one or more of (a) CDR1 from the alpha chain; (b) CDR2 from the alpha chain; (c) CDR3 from the alpha chain; (d) CDR1 from the beta chain; (e) CDR2 from the beta chain; and (f) CDR3 from the beta chain. For example, the antigen binding domain may comprise: (a); (b); (c); (d); (e); (f); (a) and (b); (a) and (c); (a) and (d); (a) and (e); (a) and (f); (b) and (c); (b) and (d); (b) and (e); (b) and (f); (c) and (d); (c) and (e); (c) and (f); (d) and (e); (d) and (f); (e) and (f); (a), (b), (c); (a), (b), (d); (a), (b) and (e); (a), (b) and (f); (a), (c) and (d); (a), (c) and (e); (a), (c) and (f); (a), (d) and (e); (a), (d) and (f); (a), (e) and (f); (b), (c) and (d); (b), (c) and (e); (b), (c) and (f); (b), (d) and (e); (b), (d) and (f); (b), (e) and (f); (c), (d) and (e); (c), (d) and (f); (c), (e) and (f); (d), (e) and (f); (a), (b), (c) and (d); (a), (b), (c) and (e); (a), (b), (c) and (f); (a), (b), (d) and (e); (a), (b), (d) and (f); (a), (b), (e) and (f); (a), (c), (d) and (e); (a), (c), (d) and (f); (a), (c), (e) and (f); (a), (d), (e) and (f); (b), (c), (d) and (e); (b), (c), (d) and (f); (b), (c), (e), (f); (b), (d), (e) and (f); (c), (d), (e) and (f); (a), (b), (c), (d) and (e); (a), (b), (c), (d) and (f); (a), (b), (c), (e) and (f); (a), (b), (d), (e) and (f); (a), (c), (d), (e) and (f); (b), (c), (d), (e) and (f); (a), (b), (c), (d), (e) and (f). Preferably, the antigen binding domain comprises CDR3 from the alpha chain of a TCR and CDR3 from the beta chain of the TCR. In any case, the TCR may for example be any TCR disclosed herein.

Exemplary antigen binding domains are set out in the Examples (see Example 1, and Table 5 in Example 5). The antigen binding domain may, for example, comprise a CDR3 from any one or more of the exemplified antigen binding domains. The antigen binding domain may, for example, comprise a CDR3 from an exemplified TCR alpha chain, and a CDR3 from an exemplified TCR beta chain. The antigen binding domain may, for example, comprise CDR1, CDR2 and CDR3 from an exemplified TCR alpha chain, and CDR1, CDR2 and CDR3 from an exemplified TCR beta chain.

The antigen binding domain may, for example, comprise any one or more of the exemplified antigen binding domains. The antigen binding domain may, for example, comprise an exemplified TCR alpha chain and an exemplified TCR beta chain.

The antigen binding domain may, for example, comprise (a) a CDR3 from one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or (b) a CDR3 from one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292. The antigen binding molecule may, for example, comprises (a) CDR1, CDR2 and CDR3 from one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or (b) CDR1, CDR2 and CDR3 from one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292. The antigen binding molecule may, for example, comprises (a) one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or (b) one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292.

The antigen binding domain may, for example, comprise CDR3 from one or more of SEQ ID NOs: 6, 8, 10, 12, 14, 16, 18, 20 and 22. For example, the antigen binding domain may comprise (a) CDR3 from each of SEQ ID NOs: 6 and 8; (b) CDR3 from each of SEQ ID NOs: 10 and 14, or 12 and 14; (c) CDR3 from each of SEQ ID NOs: 16 and 18; (d) CDR3 from each of SEQ ID NOs: 10 and 14; or (e) CDR3 from each of SEQ ID NOs: 20 and 22.

The antigen binding domain may, for example, comprise CDR1, CDR2 and CDR3 from one or more of SEQ ID NOs: 6, 8, 10, 12, 14, 16, 18, 20 and 22. For example, the antigen binding domain may comprise (a) CDR1, CDR2 and CDR3 from each of SEQ ID NOs: 6 and 8; (b) CDR1, CDR2 and CDR3 from each of SEQ ID NOs: 10 and 14, or 12 and 14; (c) CDR1, CDR2 and CDR3 from each of SEQ ID NOs: 16 and 18; (d) CDR1, CDR2 and CDR3 from each of SEQ ID NOs: 10 and 14; or (e) CDR1, CDR2 and CDR3 from each of SEQ ID NOs: 20 and 22.

The antigen binding domain may, for example, comprise a CDR3 encoded within one or more nucleic acid sequences comprising or consisting of SEQ ID NOs: 5, 7, 9, 11, 13, 15, 17, 19, 21 and 23 to 31. For example, the antigen binding domain may comprise a CDR3 encoded within (a) each of a nucleic acid sequence comprising or consisting of SEQ ID NO: 5 and a nucleic acid sequence comprising or consisting of SEQ ID NO: 7; (b) each of a nucleic acid sequence comprising or consisting of SEQ ID NO: 9, a nucleic acid sequence comprising or consisting of SEQ ID NO: 11, and a nucleic acid sequence comprising or consisting of SEQ ID NO: 13; (c) each of a nucleic acid sequence comprising or consisting of SEQ ID NO: 15 and a nucleic acid sequence comprising or consisting of SEQ ID NO: 17; (d) each of a nucleic acid sequence comprising or consisting of SEQ ID NO: 9 and a nucleic acid sequence comprising or consisting of SEQ ID NO: 13; or (e) each of a nucleic acid sequence comprising or consisting of SEQ ID NO: 19 and a nucleic acid sequence comprising or consisting of SEQ ID NO: 21. The antigen binding domain may, for example, comprise a CDR3 encoded within (a) each of a nucleic acid sequence comprising or consisting of SEQ ID NO: 23 and a nucleic acid sequence comprising or consisting of SEQ ID NO: 24; (b) each of a nucleic acid sequence comprising or consisting of SEQ ID NO: 25 and a nucleic acid sequence comprising or consisting of SEQ ID NO: 27, or each of a nucleic acid sequence comprising or consisting of SEQ ID NO: 26 and a nucleic acid sequence comprising or consisting of SEQ ID NO: 27; (c) each of a nucleic acid sequence comprising or consisting of SEQ ID NO: 28 and a nucleic acid sequence comprising or consisting of SEQ ID NO: 29; (d) each of a nucleic acid sequence comprising or consisting of SEQ ID NO: 25 and a nucleic acid sequence comprising or consisting of SEQ ID NO: 27; or (e) each of a nucleic acid sequence comprising or consisting of SEQ ID NO: 30 and a nucleic acid sequence comprising or consisting of SEQ ID NO: 31.

The antigen binding domain may, for example, comprise a CDR1, CDR2 and CDR3 encoded within one or more nucleic acid sequences comprising or consisting of SEQ ID NOs: 5, 7, 9, 11, 13, 15, 17, 19, 21 and 23 to 31. For example, the antigen binding domain may comprise a CDR1, CDR2 and CDR3 encoded within (a) each of a nucleic acid sequence comprising or consisting of SEQ ID NO: 5 and a nucleic acid sequence comprising or consisting of SEQ ID NO: 7; (b) each of a nucleic acid sequence comprising or consisting of SEQ ID NO: 9, a nucleic acid sequence comprising or consisting of SEQ ID NO: 11, and a nucleic acid sequence comprising or consisting of SEQ ID NO: 13; (c) each of a nucleic acid sequence comprising or consisting of SEQ ID NO: 15 and a nucleic acid sequence comprising or consisting of SEQ ID NO: 17; (d) each of a nucleic acid sequence comprising or consisting of SEQ ID NO: 9 and a nucleic acid sequence comprising or consisting of SEQ ID NO: 13; or (e) each of a nucleic acid sequence comprising or consisting of SEQ ID NO: 19 and a nucleic acid sequence comprising or consisting of SEQ ID NO: 21. The antigen binding domain may, for example, comprise a CDR1, CDR2 and CDR3 encoded within (a) each of a nucleic acid sequence comprising or consisting of SEQ ID NO: 23 and a nucleic acid sequence comprising or consisting of SEQ ID NO: 24; (b) each of a nucleic acid sequence comprising or consisting of SEQ ID NO: 25 and a nucleic acid sequence comprising or consisting of SEQ ID NO: 27, or each of a nucleic acid sequence comprising or consisting of SEQ ID NO: 26 and a nucleic acid sequence comprising or consisting of SEQ ID NO: 27; (c) each of a nucleic acid sequence comprising or consisting of SEQ ID NO: 28 and a nucleic acid sequence comprising or consisting of SEQ ID NO: 29; (d) each of a nucleic acid sequence comprising or consisting of SEQ ID NO: 25 and a nucleic acid sequence comprising or consisting of SEQ ID NO: 27; or (e) each of a nucleic acid sequence comprising or consisting of SEQ ID NO: 30 and a nucleic acid sequence comprising or consisting of SEQ ID NO: 31.

The antigen binding domain may, for example, comprise one or more of SEQ ID NOs: 6, 8, 10, 12, 14, 16, 18, 20 and 22. For example, the antigen binding domain may comprise (a) SEQ ID NOs: 6 and 8; (b) SEQ ID NOs: 10 and 14 or 12 and 14; (c) SEQ ID NOs: 16 and 18; (d) SEQ ID NOs: 10 and 14; or (e) SEQ ID NOs: 20 and 22. The antigen binding domain may, for example, be encoded by one or more nucleic acid sequences comprising or consisting of SEQ ID NOs: 5, 7, 9, 11, 13, 15, 17, 19, 21 and 23 to 31. For example, the antigen binding domain may be encoded by (a) a nucleic acid sequence comprising or consisting of SEQ ID NO: 5 and a nucleic acid sequence comprising or consisting of SEQ ID NO: 7; (b) a nucleic acid sequence comprising or consisting of SEQ ID NO: 9, a nucleic acid sequence comprising or consisting of SEQ ID NO: 11, and a nucleic acid sequence comprising or consisting of SEQ ID NO: 13; (c) a nucleic acid sequence comprising or consisting of SEQ ID NO: 15 and a nucleic acid sequence comprising or consisting of SEQ ID NO: 17; (d) a nucleic acid sequence comprising or consisting of SEQ ID NO: 9 and a nucleic acid sequence comprising or consisting of SEQ ID NO: 13; or (e) a nucleic acid sequence comprising or consisting of SEQ ID NO: 19 and a nucleic acid sequence comprising or consisting of SEQ ID NO: 21. Preferably, the antigen binding domain is encoded by (a) a nucleic acid sequence comprising or consisting of SEQ ID NO: 23 and a nucleic acid sequence comprising or consisting of SEQ ID NO: 24; (b) a nucleic acid sequence comprising or consisting of SEQ ID NO: 25 and a nucleic acid sequence comprising or consisting of SEQ ID NO: 27, or a nucleic acid sequence comprising or consisting of SEQ ID NO: 26 and a nucleic acid sequence comprising or consisting of SEQ ID NO: 27; (c) a nucleic acid sequence comprising or consisting of SEQ ID NO: 28 and a nucleic acid sequence comprising or consisting of SEQ ID NO: 29; (d) a nucleic acid sequence comprising or consisting of SEQ ID NO: 25 and a nucleic acid sequence comprising or consisting of SEQ ID NO: 27; or (e) a nucleic acid sequence comprising or consisting of SEQ ID NO: 30 and a nucleic acid sequence comprising or consisting of SEQ ID NO: 31.

The antigen binding domain may, for example, comprise a CDR3 from one or more of SEQ ID NOs: 206, 207, 208, 209, 210, 211, 210, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 289, 259, 260, 261, 262, 263, 264, 265, 266, 267, 268, 269, 27, 271, 272, 273, 274, 275, 276, 277, 278, 279, 280, 281, 282, 283, 284, 285, 286, 287, 288, 289, 290, 291 and 292. For example, the antigen binding domain may comprise a CDR3 from two of SEQ ID NOs: 206, 207, 208, 209, 210, 211, 210, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 289, 259, 260, 261, 262, 263, 264, 265, 266, 267, 268, 269, 27, 271, 272, 273, 274, 275, 276, 277, 278, 279, 280, 281, 282, 283, 284, 285, 286, 287, 288, 289, 290, 291 and 292, in any combination. The antigen binding domain may, for example, comprise a CDR3 from each of SEQ ID NOs: 206 and 207. The antigen binding domain may, for example, comprise a CDR3 from each of SEQ ID Nos 208 and 209, or from each of 208 and 210. The antigen binding domain may, for example, comprise a CDR3 from each of SEQ ID Nos 211 and 212. The antigen binding domain may, for example, comprise a CDR3 from each of SEQ ID Nos 213 and 214. The antigen binding domain may, for example, comprise a CDR3 from each of SEQ ID Nos: 215 and 216, or from each of SEQ ID Nos: 215 and 217. The antigen binding domain may, for example, comprise a CDR3 from each of SEQ ID Nos 218 and 219, or from each of SEQ ID Nos: 218 and 220. The antigen binding domain may, for example, comprise a CDR3 from each of SEQ ID Nos 221 and 223, or from each of SEQ ID Nos: 222 and 223. The antigen binding domain may, for example, comprise a CDR3 from each of SEQ ID Nos 224 and 225. The antigen binding domain may, for example, comprise a CDR3 from each of SEQ ID Nos 226 and 227. The antigen binding domain may, for example, comprise a CDR3 from each of SEQ ID Nos 228 and 229. The antigen binding domain may, for example, comprise a CDR3 from each of SEQ ID Nos 230 and 231. The antigen binding domain may, for example, comprise SEQ ID Nos 232 and 234, or 233 and 234. The antigen binding domain may, for example, comprise a CDR3 from each of SEQ ID Nos 235 and 236. The antigen binding domain may, for example, comprise a CDR3 from each of SEQ ID Nos 237 and 238. The antigen binding domain may, for example, comprise a CDR3 from each of SEQ ID Nos 239 and 240. The antigen binding domain may, for example, comprise a CDR3 from each of SEQ ID Nos 241 and 242. The antigen binding domain may, for example, comprise a CDR3 from each of SEQ ID Nos 243 and 245, or from each of SEQ ID Nos: 244 and 245. The antigen binding domain may, for example, comprise a CDR3 from each of SEQ ID Nos 246 and 247. The antigen binding domain may, for example, comprise a CDR3 from each of SEQ ID Nos 248 and 249. The antigen binding domain may, for example, comprise a CDR3 from each of SEQ ID Nos 250 and 251. The antigen binding domain may, for example, comprise a CDR3 from each of SEQ ID Nos 252 and 253. The antigen binding domain may, for example, comprise a CDR3 from each of SEQ ID Nos 254 and 255. The antigen binding domain may, for example, comprise a CDR3 from each of SEQ ID Nos 256 and 257. The antigen binding domain may, for example, comprise a CDR3 from each of SEQ ID Nos 258 and 259. The antigen binding domain may, for example, comprise a CDR3 from each of SEQ ID Nos 260 and 262, or from each of SEQ ID Nos: 261 and 262. The antigen binding domain may, for example, comprise a CDR3 from each of SEQ ID Nos 263 and 264, or from each of SEQ ID NOs: 263 and 265. The antigen binding domain may, for example, comprise a CDR3 from each of SEQ ID Nos 266 and 267. The antigen binding domain may, for example, comprise a CDR3 from each of SEQ ID Nos 268 and 269. The antigen binding domain may, for example, comprise a CDR3 from each of SEQ ID Nos 270 and 271. The antigen binding domain may, for example, comprise a CDR3 from each of SEQ ID Nos 272 and 273. The antigen binding domain may, for example, comprise a CDR3 from each of SEQ ID Nos 274 and 275. The antigen binding domain may, for example, comprise a CDR3 from each of SEQ ID Nos 276 and 277. The antigen binding domain may, for example, comprise a CDR3 from each of SEQ ID Nos 278 and 279. The antigen binding domain may, for example, comprise a CDR3 from each of SEQ ID Nos 280 and 281. The antigen binding domain may, for example, comprise a CDR3 from each of SEQ ID Nos 282 and 284, or 283 and 284. The antigen binding domain may, for example, comprise a CDR3 from each of SEQ ID Nos 285 and 286. The antigen binding domain may, for example, comprise a CDR3 from each of SEQ ID Nos 287 and 289, or from each of SEQ ID Nos: 288 and 289. The antigen binding domain may, for example, comprise a CDR3 from each of SEQ ID Nos 290 and 291, or from each of SEQ ID Nos: 290 and 292.

The antigen binding domain may, for example, comprise a CDR1, CDR2 and CDR3 from one or more of SEQ ID NOs: 206, 207, 208, 209, 210, 211, 210, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 289, 259, 260, 261, 262, 263, 264, 265, 266, 267, 268, 269, 27, 271, 272, 273, 274, 275, 276, 277, 278, 279, 280, 281, 282, 283, 284, 285, 286, 287, 288, 289, 290, 291 and 292. For example, the antigen binding domain may comprise a CDR1, CDR2 and CDR3 from two of SEQ ID NOs: 206, 207, 208, 209, 210, 211, 210, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 289, 259, 260, 261, 262, 263, 264, 265, 266, 267, 268, 269, 27, 271, 272, 273, 274, 275, 276, 277, 278, 279, 280, 281, 282, 283, 284, 285, 286, 287, 288, 289, 290, 291 and 292, in any combination. The antigen binding domain may, for example, comprise a CDR1, CDR2 and CDR3 from each of SEQ ID NOs: 206 and 207. The antigen binding domain may, for example, comprise a CDR1, CDR2 and CDR3 from each of SEQ ID Nos 208 and 209, or from each of 208 and 210. The antigen binding domain may, for example, comprise a CDR1, CDR2 and CDR3 from each of SEQ ID Nos 211 and 212. The antigen binding domain may, for example, comprise a CDR1, CDR2 and CDR3 from each of SEQ ID Nos 213 and 214. The antigen binding domain may, for example, comprise a CDR1, CDR2 and CDR3 from each of SEQ ID Nos: 215 and 216, or from each of SEQ ID Nos: 215 and 217. The antigen binding domain may, for example, comprise a CDR1, CDR2 and CDR3 from each of SEQ ID Nos 218 and 219, or from each of SEQ ID Nos: 218 and 220. The antigen binding domain may, for example, comprise a CDR1, CDR2 and CDR3 from each of SEQ ID Nos 221 and 223, or from each of SEQ ID Nos: 222 and 223. The antigen binding domain may, for example, comprise a CDR1, CDR2 and CDR3 from each of SEQ ID Nos 224 and 225. The antigen binding domain may, for example, comprise a CDR1, CDR2 and CDR3 from each of SEQ ID Nos 226 and 227. The antigen binding domain may, for example, comprise a CDR1, CDR2 and CDR3 from each of SEQ ID Nos 228 and 229. The antigen binding domain may, for example, comprise a CDR1, CDR2 and CDR3 from each of SEQ ID Nos 230 and 231. The antigen binding domain may, for example, comprise SEQ ID Nos 232 and 234, or 233 and 234. The antigen binding domain may, for example, comprise a CDR1, CDR2 and CDR3 from each of SEQ ID Nos 235 and 236. The antigen binding domain may, for example, comprise a CDR1, CDR2 and CDR3 from each of SEQ ID Nos 237 and 238. The antigen binding domain may, for example, comprise a CDR1, CDR2 and CDR3 from each of SEQ ID Nos 239 and 240. The antigen binding domain may, for example, comprise a CDR1, CDR2 and CDR3 from each of SEQ ID Nos 241 and 242. The antigen binding domain may, for example, comprise a CDR1, CDR2 and CDR3 from each of SEQ ID Nos 243 and 245, or from each of SEQ ID Nos: 244 and 245. The antigen binding domain may, for example, comprise a CDR1, CDR2 and CDR3 from each of SEQ ID Nos 246 and 247. The antigen binding domain may, for example, comprise a CDR1, CDR2 and CDR3 from each of SEQ ID Nos 248 and 249. The antigen binding domain may, for example, comprise a CDR1, CDR2 and CDR3 from each of SEQ ID Nos 250 and 251. The antigen binding domain may, for example, comprise a CDR1, CDR2 and CDR3 from each of SEQ ID Nos 252 and 253. The antigen binding domain may, for example, comprise a CDR1, CDR2 and CDR3 from each of SEQ ID Nos 254 and 255. The antigen binding domain may, for example, comprise a CDR1, CDR2 and CDR3 from each of SEQ ID Nos 256 and 257. The antigen binding domain may, for example, comprise a CDR1, CDR2 and CDR3 from each of SEQ ID Nos 258 and 259. The antigen binding domain may, for example, comprise a CDR1, CDR2 and CDR3 from each of SEQ ID Nos 260 and 262, or from each of SEQ ID Nos: 261 and 262. The antigen binding domain may, for example, comprise a CDR1, CDR2 and CDR3 from each of SEQ ID Nos 263 and 264, or from each of SEQ ID NOs: 263 and 265. The antigen binding domain may, for example, comprise a CDR1, CDR2 and CDR3 from each of SEQ ID Nos 266 and 267. The antigen binding domain may, for example, comprise a CDR1, CDR2 and CDR3 from each of SEQ ID Nos 268 and 269. The antigen binding domain may, for example, comprise a CDR1, CDR2 and CDR3 from each of SEQ ID Nos 270 and 271. The antigen binding domain may, for example, comprise a CDR1, CDR2 and CDR3 from each of SEQ ID Nos 272 and 273. The antigen binding domain may, for example, comprise a CDR1, CDR2 and CDR3 from each of SEQ ID Nos 274 and 275. The antigen binding domain may, for example, comprise a CDR1, CDR2 and CDR3 from each of SEQ ID Nos 276 and 277. The antigen binding domain may, for example, comprise a CDR1, CDR2 and CDR3 from each of SEQ ID Nos 278 and 279. The antigen binding domain may, for example, comprise a CDR1, CDR2 and CDR3 from each of SEQ ID Nos 280 and 281. The antigen binding domain may, for example, comprise a CDR1, CDR2 and CDR3 from each of SEQ ID Nos 282 and 284, or 283 and 284. The antigen binding domain may, for example, comprise a CDR1, CDR2 and CDR3 from each of SEQ ID Nos 285 and 286. The antigen binding domain may, for example, comprise a CDR1, CDR2 and CDR3 from each of SEQ ID Nos 287 and 289, or from each of SEQ ID Nos: 288 and 289. The antigen binding domain may, for example, comprise a CDR1, CDR2 and CDR3 from each of SEQ ID Nos 290 and 291, or from each of SEQ ID Nos: 290 and 292.

The antigen binding domain may, for example, comprise a CDR3 encoded within one or more (such as two more more) nucleic acid sequences comprising or consisting of SEQ ID NOs: 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378 or 379. The antigen binding domain may, for example, comprise a CDR3 encoded within each of a nucleic acid sequence comprising or consisting of SEQ ID NO: 293 and a nucleic acid sequence comprising or consisting of SEQ ID NO: 294. The antigen binding domain may, for example, comprise a CDR3 encoded within each of a nucleic acid sequence comprising or consisting of 295 and a nucleic acid sequence comprising or consisting of SEQ ID NO: 296, or a within each of nucleic acid sequence comprising or consisting of 295 and a nucleic acid sequence comprising or consisting of SEQ ID NO: 297. The antigen binding domain may, for example, comprise a CDR3 encoded within each of a nucleic acid sequence comprising or consisting of 298 and a nucleic acid sequence comprising or consisting of SEQ ID NO: 299. The antigen binding domain may, for example, comprise a CDR3 encoded within each of a nucleic acid sequence comprising or consisting of 300 and a nucleic acid sequence comprising or consisting of SEQ ID NO: 301. The antigen binding domain may, for example, comprise a CDR3 encoded within each of a nucleic acid sequence comprising or consisting of 302 and a nucleic acid sequence comprising or consisting of SEQ ID NO: 303, or within each of a nucleic acid sequence comprising or consisting of 302 and a nucleic acid sequence comprising or consisting of SEQ ID NO: 304, The antigen binding domain may, for example, comprise a CDR3 encoded within each of a nucleic acid sequence comprising or consisting of SEQ ID NO: 305 and a nucleic acid sequence comprising or consisting of SEQ ID NO: 306, or within each of a nucleic acid sequence comprising or consisting of SEQ ID NO: 305 a nucleic acid sequence comprising or consisting of SEQ ID NO: 307. The antigen binding domain may, for example, comprise a CDR3 encoded within each of a nucleic acid sequence comprising or consisting of SEQ ID NO: 308 and a nucleic acid sequence comprising or consisting of SEQ ID NO: 309, or within each of a nucleic acid sequence comprising or consisting of SEQ ID NO: 308 and a nucleic acid sequence comprising or consisting of SEQ ID NO: 310. The antigen binding domain may, for example, comprise a CDR3 encoded within each of a nucleic acid sequence comprising or consisting of 311 and a nucleic acid sequence comprising or consisting of SEQ ID NO: 312. The antigen binding domain may, for example, comprise a CDR3 encoded within each of a nucleic acid sequence comprising or consisting of SEQ ID NO: 313 and a nucleic acid sequence comprising or consisting of SEQ ID NO: 314. The antigen binding domain may, for example, comprise a CDR3 encoded within each of a nucleic acid sequence comprising or consisting of SEQ ID NO: 315 and a nucleic acid sequence comprising or consisting of SEQ ID NO: 316. The antigen binding domain may, for example, comprise a CDR3 encoded within each of a nucleic acid sequence comprising or consisting of SEQ ID NO: 317 and a nucleic acid sequence comprising or consisting of SEQ ID NO: 318. The antigen binding domain may, for example, comprise a CDR3 encoded within each of a nucleic acid sequence comprising or consisting of SEQ ID NO: 319 and a nucleic acid sequence comprising or consisting of SEQ ID NO: 321, or within each of a nucleic acid sequence comprising or consisting of SEQ ID NO: 320 and a nucleic acid sequence comprising or consisting of SEQ ID NO: 321, The antigen binding domain may, for example, comprise a CDR3 encoded within each of a nucleic acid sequence comprising or consisting of SEQ ID NO: 322 and a nucleic acid sequence comprising or consisting of SEQ ID NO: 323. The antigen binding domain may, for example, comprise a CDR3 encoded within each of a nucleic acid sequence comprising or consisting of SEQ ID NO: 324 and a nucleic acid sequence comprising or consisting of SEQ ID NO: 325. The antigen binding domain may, for example, comprise a CDR3 encoded within each of a nucleic acid sequence comprising or consisting of SEQ ID NO: 326 and a nucleic acid sequence comprising or consisting of SEQ ID NO: 327. The antigen binding domain may, for example, comprise a CDR3 encoded within each of a nucleic acid sequence comprising or consisting of SEQ ID NO: 328 and a nucleic acid sequence comprising or consisting of SEQ ID NO: 329. The antigen binding domain may, for example, comprise a CDR3 encoded within each of a nucleic acid sequence comprising or consisting of SEQ ID NO: 330 and a nucleic acid sequence comprising or consisting of SEQ ID NO: 332, or within each of a nucleic acid sequence comprising or consisting of SEQ ID NO: 331 and a nucleic acid sequence comprising or consisting of SEQ ID NO: 332. The antigen binding domain may, for example, comprise a CDR3 encoded within each of a nucleic acid sequence comprising or consisting of SEQ ID NO: 333 and a nucleic acid sequence comprising or consisting of SEQ ID NO: 334. The antigen binding domain may, for example, comprise a CDR3 encoded within each of a nucleic acid sequence comprising or consisting of SEQ ID NO: 335 and a nucleic acid sequence comprising or consisting of SEQ ID NO: 336. The antigen binding domain may, for example, comprise a CDR3 encoded within each of a nucleic acid sequence comprising or consisting of SEQ ID NO: 337 and a nucleic acid sequence comprising or consisting of SEQ ID NO: 338. The antigen binding domain may, for example, comprise a CDR3 encoded within each of a nucleic acid sequence comprising or consisting of SEQ ID NO 339 and a nucleic acid sequence comprising or consisting of SEQ ID NO: 340. The antigen binding domain may, for example, comprise a CDR3 encoded within each of a nucleic acid sequence comprising or consisting of SEQ ID NO: 341 and a nucleic acid sequence comprising or consisting of SEQ ID NO: 342. The antigen binding domain may, for example, comprise a CDR3 encoded within each of a nucleic acid sequence comprising or consisting of SEQ ID NO: 343 and a nucleic acid sequence comprising or consisting of SEQ ID NO: 344. The antigen binding domain may, for example, comprise a CDR3 encoded within each of a nucleic acid sequence comprising or consisting of SEQ ID NO: 345 and a nucleic acid sequence comprising or consisting of SEQ ID NO: 346. The antigen binding domain may, for example, comprise a CDR3 encoded within each of a nucleic acid sequence comprising or consisting of SEQ ID NO: 347 and a nucleic acid sequence comprising or consisting of SEQ ID NO: 349, or within each of a nucleic acid sequence comprising or consisting of SEQ ID NO: 348 and a nucleic acid sequence comprising or consisting of SEQ ID NO: 349. The antigen binding domain may, for example, comprise a CDR3 encoded within each of a nucleic acid sequence comprising or consisting of a nucleic acid sequence comprising or consisting of SEQ ID NO: 350 and a nucleic acid sequence comprising or consisting of SEQ ID NO: 351, or within each of a nucleic acid sequence comprising or consisting of SEQ ID NO: 350 and a nucleic acid sequence comprising or consisting of SEQ ID NO: 352. The antigen binding domain may, for example, comprise a CDR3 encoded within each of a nucleic acid sequence comprising or consisting of SEQ ID NO: 353 and a nucleic acid sequence comprising or consisting of SEQ ID NO: 354. The antigen binding domain may, for example, comprise a CDR3 encoded within each of a nucleic acid sequence comprising or consisting of SEQ ID NO: 355 and a nucleic acid sequence comprising or consisting of SEQ ID NO: 356. The antigen binding domain may, for example, comprise a CDR3 encoded within each of a nucleic acid sequence comprising or consisting of SEQ ID NO: 357 and a nucleic acid sequence comprising or consisting of SEQ ID NO: 358. The antigen binding domain may, for example, comprise a CDR3 encoded within each of a nucleic acid sequence comprising or consisting of SEQ ID NO: 359 and a nucleic acid sequence comprising or consisting of SEQ ID NO: 360. The antigen binding domain may, for example, comprise a CDR3 encoded within each of a nucleic acid sequence comprising or consisting of SEQ ID NO: 361 and a nucleic acid sequence comprising or consisting of SEQ ID NO: 362. The antigen binding domain may, for example, comprise a CDR3 encoded within each of a nucleic acid sequence comprising or consisting of SEQ ID NO: 363 and a nucleic acid sequence comprising or consisting of SEQ ID NO: 364. The antigen binding domain may, for example, comprise a CDR3 encoded within each of a nucleic acid sequence comprising or consisting of SEQ ID NO: 365 and a nucleic acid sequence comprising or consisting of SEQ ID NO: 366. The antigen binding domain may, for example, comprise a CDR3 encoded within each of a nucleic acid sequence comprising or consisting of SEQ ID NO:367 and a nucleic acid sequence comprising or consisting of SEQ ID NO: 368. The antigen binding domain may, for example, comprise a CDR3 encoded within each of a nucleic acid sequence comprising or consisting of SEQ ID NO: 369 and a nucleic acid sequence comprising or consisting of SEQ ID NO: 371, or within each of a nucleic acid sequence comprising or consisting of SEQ ID NO: 370 and a nucleic acid sequence comprising or consisting of SEQ ID NO: 371, The antigen binding domain may, for example, comprise a CDR3 encoded within each of a nucleic acid sequence comprising or consisting of SEQ ID NO: 372 and a nucleic acid sequence comprising or consisting of SEQ ID NO: 373. The antigen binding domain may, for example, comprise a CDR3 encoded within each of a nucleic acid sequence comprising or consisting of SEQ ID NO: 374 and a nucleic acid sequence comprising or consisting of SEQ ID NO: 376, or within each of a nucleic acid sequence comprising or consisting of SEQ ID NO: 375 and a nucleic acid sequence comprising or consisting of SEQ ID NO: 376. The antigen binding domain may, for example, comprise a CDR3 encoded within each of a nucleic acid sequence comprising or consisting of SEQ ID NO: 377 and a nucleic acid sequence comprising or consisting of SEQ ID NO: 378, or within each of a nucleic acid sequence comprising or consisting of SEQ ID NO: 377 and a nucleic acid sequence comprising or consisting of SEQ ID NO: 379.

The antigen binding domain may, for example, comprise a CDR1, CDR2 and CDR3 encoded within one or more (such as two or more) nucleic acid sequences comprising or consisting of SEQ ID NOs: 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378 or 379. The antigen binding domain may, for example, comprise a CDR1, CDR2 and CDR3 encoded within each of a nucleic acid sequence comprising or consisting of SEQ ID NO: 293 and a nucleic acid sequence comprising or consisting of SEQ ID NO: 294. The antigen binding domain may, for example, comprise a CDR1, CDR2 and CDR3 encoded within each of a nucleic acid sequence comprising or consisting of 295 and a nucleic acid sequence comprising or consisting of SEQ ID NO: 296, or a within each of nucleic acid sequence comprising or consisting of 295 and a nucleic acid sequence comprising or consisting of SEQ ID NO: 297. The antigen binding domain may, for example, comprise a CDR1, CDR2 and CDR3 encoded within each of a nucleic acid sequence comprising or consisting of 298 and a nucleic acid sequence comprising or consisting of SEQ ID NO: 299. The antigen binding domain may, for example, comprise a CDR1, CDR2 and CDR3 encoded within each of a nucleic acid sequence comprising or consisting of 300 and a nucleic acid sequence comprising or consisting of SEQ ID NO: 301. The antigen binding domain may, for example, comprise a CDR1, CDR2 and CDR3 encoded within each of a nucleic acid sequence comprising or consisting of 302 and a nucleic acid sequence comprising or consisting of SEQ ID NO: 303, or within each of a nucleic acid sequence comprising or consisting of 302 and a nucleic acid sequence comprising or consisting of SEQ ID NO:304, The antigen binding domain may, for example, comprise a CDR1, CDR2 and CDR3 encoded within each of a nucleic acid sequence comprising or consisting of SEQ ID NO: 305 and a nucleic acid sequence comprising or consisting of SEQ ID NO: 306, or within each of a nucleic acid sequence comprising or consisting of SEQ ID NO: 305 a nucleic acid sequence comprising or consisting of SEQ ID NO: 307. The antigen binding domain may, for example, comprise a CDR1, CDR2 and CDR3 encoded within each of a nucleic acid sequence comprising or consisting of SEQ ID NO: 308 and a nucleic acid sequence comprising or consisting of SEQ ID NO: 309, or within each of a nucleic acid sequence comprising or consisting of SEQ ID NO: 308 and a nucleic acid sequence comprising or consisting of SEQ ID NO: 310. The antigen binding domain may, for example, comprise a CDR1, CDR2 and CDR3 encoded within each of a nucleic acid sequence comprising or consisting of 311 and a nucleic acid sequence comprising or consisting of SEQ ID NO: 312. The antigen binding domain may, for example, comprise a CDR1, CDR2 and CDR3 encoded within each of a nucleic acid sequence comprising or consisting of SEQ ID NO: 313 and a nucleic acid sequence comprising or consisting of SEQ ID NO: 314. The antigen binding domain may, for example, comprise a CDR1, CDR2 and CDR3 encoded within each of a nucleic acid sequence comprising or consisting of SEQ ID NO: 315 and a nucleic acid sequence comprising or consisting of SEQ ID NO: 316. The antigen binding domain may, for example, comprise a CDR1, CDR2 and CDR3 encoded within each of a nucleic acid sequence comprising or consisting of SEQ ID NO: 317 and a nucleic acid sequence comprising or consisting of SEQ ID NO: 318. The antigen binding domain may, for example, comprise a CDR1, CDR2 and CDR3 encoded within each of a nucleic acid sequence comprising or consisting of SEQ ID NO: 319 and a nucleic acid sequence comprising or consisting of SEQ ID NO: 321, or within each of a nucleic acid sequence comprising or consisting of SEQ ID NO: 320 and a nucleic acid sequence comprising or consisting of SEQ ID NO: 321, The antigen binding domain may, for example, comprise a CDR1, CDR2 and CDR3 encoded within each of a nucleic acid sequence comprising or consisting of SEQ ID NO: 322 and a nucleic acid sequence comprising or consisting of SEQ ID NO: 323. The antigen binding domain may, for example, comprise a CDR1, CDR2 and CDR3 encoded within each of a nucleic acid sequence comprising or consisting of SEQ ID NO: 324 and a nucleic acid sequence comprising or consisting of SEQ ID NO: 325. The antigen binding domain may, for example, comprise a CDR1, CDR2 and CDR3 encoded within each of a nucleic acid sequence comprising or consisting of SEQ ID NO: 326 and a nucleic acid sequence comprising or consisting of SEQ ID NO: 327. The antigen binding domain may, for example, comprise a CDR1, CDR2 and CDR3 encoded within each of a nucleic acid sequence comprising or consisting of SEQ ID NO: 328 and a nucleic acid sequence comprising or consisting of SEQ ID NO: 329. The antigen binding domain may, for example, comprise a CDR1, CDR2 and CDR3 encoded within each of a nucleic acid sequence comprising or consisting of SEQ ID NO: 330 and a nucleic acid sequence comprising or consisting of SEQ ID NO: 332, or within each of a nucleic acid sequence comprising or consisting of SEQ ID NO: 331 and a nucleic acid sequence comprising or consisting of SEQ ID NO: 332. The antigen binding domain may, for example, comprise a CDR1, CDR2 and CDR3 encoded within each of a nucleic acid sequence comprising or consisting of SEQ ID NO: 333 and a nucleic acid sequence comprising or consisting of SEQ ID NO: 334. The antigen binding domain may, for example, comprise a CDR1, CDR2 and CDR3 encoded within each of a nucleic acid sequence comprising or consisting of SEQ ID NO: 335 and a nucleic acid sequence comprising or consisting of SEQ ID NO: 336. The antigen binding domain may, for example, comprise a CDR1, CDR2 and CDR3 encoded within each of a nucleic acid sequence comprising or consisting of SEQ ID NO: 337 and a nucleic acid sequence comprising or consisting of SEQ ID NO: 338. The antigen binding domain may, for example, comprise a CDR1, CDR2 and CDR3 encoded within each of a nucleic acid sequence comprising or consisting of SEQ ID NO 339 and a nucleic acid sequence comprising or consisting of SEQ ID NO: 340. The antigen binding domain may, for example, comprise a CDR1, CDR2 and CDR3 encoded within each of a nucleic acid sequence comprising or consisting of SEQ ID NO: 341 and a nucleic acid sequence comprising or consisting of SEQ ID NO: 342. The antigen binding domain may, for example, comprise a CDR1, CDR2 and CDR3 encoded within each of a nucleic acid sequence comprising or consisting of SEQ ID NO: 343 and a nucleic acid sequence comprising or consisting of SEQ ID NO: 344. The antigen binding domain may, for example, comprise a CDR1, CDR2 and CDR3 encoded within each of a nucleic acid sequence comprising or consisting of SEQ ID NO: 345 and a nucleic acid sequence comprising or consisting of SEQ ID NO: 346. The antigen binding domain may, for example, comprise a CDR1, CDR2 and CDR3 encoded within each of a nucleic acid sequence comprising or consisting of SEQ ID NO: 347 and a nucleic acid sequence comprising or consisting of SEQ ID NO: 349, or within each of a nucleic acid sequence comprising or consisting of SEQ ID NO: 348 and a nucleic acid sequence comprising or consisting of SEQ ID NO: 349. The antigen binding domain may, for example, comprise a CDR1, CDR2 and CDR3 encoded within each of a nucleic acid sequence comprising or consisting of a nucleic acid sequence comprising or consisting of SEQ ID NO: 350 and a nucleic acid sequence comprising or consisting of SEQ ID NO: 351, or within each of a nucleic acid sequence comprising or consisting of SEQ ID NO: 350 and a nucleic acid sequence comprising or consisting of SEQ ID NO: 352. The antigen binding domain may, for example, comprise a CDR1, CDR2 and CDR3 encoded within each of a nucleic acid sequence comprising or consisting of SEQ ID NO: 353 and a nucleic acid sequence comprising or consisting of SEQ ID NO: 354. The antigen binding domain may, for example, comprise a CDR1, CDR2 and CDR3 encoded within each of a nucleic acid sequence comprising or consisting of SEQ ID NO: 355 and a nucleic acid sequence comprising or consisting of SEQ ID NO: 356. The antigen binding domain may, for example, comprise a CDR1, CDR2 and CDR3 encoded within each of a nucleic acid sequence comprising or consisting of SEQ ID NO: 357 and a nucleic acid sequence comprising or consisting of SEQ ID NO: 358. The antigen binding domain may, for example, comprise a CDR1, CDR2 and CDR3 encoded within each of a nucleic acid sequence comprising or consisting of SEQ ID NO: 359 and a nucleic acid sequence comprising or consisting of SEQ ID NO: 360. The antigen binding domain may, for example, comprise a CDR1, CDR2 and CDR3 encoded within each of a nucleic acid sequence comprising or consisting of SEQ ID NO: 361 and a nucleic acid sequence comprising or consisting of SEQ ID NO: 362. The antigen binding domain may, for example, comprise a CDR1, CDR2 and CDR3 encoded within each of a nucleic acid sequence comprising or consisting of SEQ ID NO: 363 and a nucleic acid sequence comprising or consisting of SEQ ID NO: 364. The antigen binding domain may, for example, comprise a CDR1, CDR2 and CDR3 encoded within each of a nucleic acid sequence comprising or consisting of SEQ ID NO: 365 and a nucleic acid sequence comprising or consisting of SEQ ID NO: 366. The antigen binding domain may, for example, comprise a CDR1, CDR2 and CDR3 encoded within each of a nucleic acid sequence comprising or consisting of SEQ ID NO:367 and a nucleic acid sequence comprising or consisting of SEQ ID NO: 368. The antigen binding domain may, for example, comprise a CDR1, CDR2 and CDR3 encoded within each of a nucleic acid sequence comprising or consisting of SEQ ID NO: 369 and a nucleic acid sequence comprising or consisting of SEQ ID NO: 371, or within each of a nucleic acid sequence comprising or consisting of SEQ ID NO: 370 and a nucleic acid sequence comprising or consisting of SEQ ID NO: 371, The antigen binding domain may, for example, comprise a CDR1, CDR2 and CDR3 encoded within each of a nucleic acid sequence comprising or consisting of SEQ ID NO: 372 and a nucleic acid sequence comprising or consisting of SEQ ID NO: 373. The antigen binding domain may, for example, comprise a CDR1, CDR2 and CDR3 encoded within each of a nucleic acid sequence comprising or consisting of SEQ ID NO: 374 and a nucleic acid sequence comprising or consisting of SEQ ID NO: 376, or within each of a nucleic acid sequence comprising or consisting of SEQ ID NO: 375 and a nucleic acid sequence comprising or consisting of SEQ ID NO: 376. The antigen binding domain may, for example, comprise a CDR1, CDR2 and CDR3 encoded within each of a nucleic acid sequence comprising or consisting of SEQ ID NO: 377 and a nucleic acid sequence comprising or consisting of SEQ ID NO: 378, or within each of a nucleic acid sequence comprising or consisting of SEQ ID NO: 377 and a nucleic acid sequence comprising or consisting of SEQ ID NO: 379.

The antigen binding domain may, for example, comprise one or more of SEQ ID NOs: 206, 207, 208, 209, 210, 211, 210, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 289, 259, 260, 261, 262, 263, 264, 265, 266, 267, 268, 269, 27, 271, 272, 273, 274, 275, 276, 277, 278, 279, 280, 281, 282, 283, 284, 285, 286, 287, 288, 289, 290, 291 and 292. For example, the antigen binding domain may comprise two of SEQ ID NOs: 206, 207, 208, 209, 210, 211, 210, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 289, 259, 260, 261, 262, 263, 264, 265, 266, 267, 268, 269, 27, 271, 272, 273, 274, 275, 276, 277, 278, 279, 280, 281, 282, 283, 284, 285, 286, 287, 288, 289, 290, 291 and 292, in any combination. The antigen binding domain may, for example, comprise SEQ ID NOs: 206 and 207. The antigen binding domain may, for example, comprise SEQ ID Nos 208 and 209, or 208 and 210. The antigen binding domain may, for example, comprise SEQ ID Nos 211 and 212. The antigen binding domain may, for example, comprise SEQ ID Nos 213 and 214. The antigen binding domain may, for example, comprise SEQ ID Nos 215 and 216, or 215 and 217. The antigen binding domain may, for example, comprise SEQ ID Nos 218 and 219, or 218 and 220. The antigen binding domain may, for example, comprise SEQ ID Nos 221 and 223, or 222 and 223. The antigen binding domain may, for example, comprise SEQ ID Nos 224 and 225. The antigen binding domain may, for example, comprise SEQ ID Nos 226 and 227. The antigen binding domain may, for example, comprise SEQ ID Nos 228 and 229. The antigen binding domain may, for example, comprise SEQ ID Nos 230 and 231. The antigen binding domain may, for example, comprise SEQ ID Nos 232 and 234, or 233 and 234. The antigen binding domain may, for example, comprise SEQ ID Nos 235 and 236. The antigen binding domain may, for example, comprise SEQ ID Nos 237 and 238. The antigen binding domain may, for example, comprise SEQ ID Nos 239 and 240. The antigen binding domain may, for example, comprise SEQ ID Nos 241 and 242. The antigen binding domain may, for example, comprise SEQ ID Nos 243 and 245, or 244 and 245. The antigen binding domain may, for example, comprise SEQ ID Nos 246 and 247. The antigen binding domain may, for example, comprise SEQ ID Nos 248 and 249. The antigen binding domain may, for example, comprise SEQ ID Nos 250 and 251. The antigen binding domain may, for example, comprise SEQ ID Nos 252 and 253. The antigen binding domain may, for example, comprise SEQ ID Nos 254 and 255. The antigen binding domain may, for example, comprise SEQ ID Nos 256 and 257. The antigen binding domain may, for example, comprise SEQ ID Nos 258 and 259. The antigen binding domain may, for example, comprise SEQ ID Nos 260 and 262, or 261 and 262. The antigen binding domain may, for example, comprise SEQ ID Nos 263 and 264, or 263 and 265. The antigen binding domain may, for example, comprise SEQ ID Nos 266 and 267. The antigen binding domain may, for example, comprise SEQ ID Nos 268 and 269. The antigen binding domain may, for example, comprise SEQ ID Nos 270 and 271. The antigen binding domain may, for example, comprise SEQ ID Nos 272 and 273. The antigen binding domain may, for example, comprise SEQ ID Nos 274 and 275. The antigen binding domain may, for example, comprise SEQ ID Nos 276 and 277. The antigen binding domain may, for example, comprise SEQ ID Nos 278 and 279. The antigen binding domain may, for example, comprise SEQ ID Nos 280 and 281. The antigen binding domain may, for example, comprise SEQ ID Nos 282 and 284, or 283 and 284. The antigen binding domain may, for example, comprise SEQ ID Nos 285 and 286. The antigen binding domain may, for example, comprise SEQ ID Nos 287 and 289, or 288 and 289. The antigen binding domain may, for example, comprise SEQ ID Nos 290 and 291, or 290 and 292.

The antigen binding domain may, for example, be encoded by one or more nucleic acid sequences comprising or consisting of SEQ ID NOs: 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378 or 379. The antigen binding domain may, for example, be encoded by a nucleic acid sequence comprising or consisting of SEQ ID NO: 293 and a nucleic acid sequence comprising or consisting of SEQ ID NO: 294. The antigen binding domain may, for example, be encoded by a nucleic acid sequence comprising or consisting of 295 and a nucleic acid sequence comprising or consisting of SEQ ID NO: 296, or a nucleic acid sequence comprising or consisting of 295 and a nucleic acid sequence comprising or consisting of SEQ ID NO: 297. The antigen binding domain may, for example, be encoded by a nucleic acid sequence comprising or consisting of 298 and a nucleic acid sequence comprising or consisting of SEQ ID NO: 299. The antigen binding domain may, for example, be encoded by a nucleic acid sequence comprising or consisting of 300 and a nucleic acid sequence comprising or consisting of SEQ ID NO: 301. The antigen binding domain may, for example, be encoded by a nucleic acid sequence comprising or consisting of 302 and a nucleic acid sequence comprising or consisting of SEQ ID NO: 303, or a nucleic acid sequence comprising or consisting of 302 and a nucleic acid sequence comprising or consisting of SEQ ID NO:304, The antigen binding domain may, for example, be encoded by a nucleic acid sequence comprising or consisting of SEQ ID NO: 305 and a nucleic acid sequence comprising or consisting of SEQ ID NO: 306, or a nucleic acid sequence comprising or consisting of SEQ ID NO: 305 a nucleic acid sequence comprising or consisting of SEQ ID NO: 307. The antigen binding domain may, for example, be encoded by a nucleic acid sequence comprising or consisting of SEQ ID NO: 308 and a nucleic acid sequence comprising or consisting of SEQ ID NO: 309, or a nucleic acid sequence comprising or consisting of SEQ ID NO: 308 and a nucleic acid sequence comprising or consisting of SEQ ID NO: 310. The antigen binding domain may, for example, be encoded by a nucleic acid sequence comprising or consisting of 311 and a nucleic acid sequence comprising or consisting of SEQ ID NO: 312. The antigen binding domain may, for example, be encoded by a nucleic acid sequence comprising or consisting of SEQ ID NO: 313 and a nucleic acid sequence comprising or consisting of SEQ ID NO: 314. The antigen binding domain may, for example, be encoded by a nucleic acid sequence comprising or consisting of SEQ ID NO: 315 and a nucleic acid sequence comprising or consisting of SEQ ID NO: 316. The antigen binding domain may, for example, be encoded by a nucleic acid sequence comprising or consisting of SEQ ID NO: 317 and a nucleic acid sequence comprising or consisting of SEQ ID NO: 318. The antigen binding domain may, for example, be encoded by a nucleic acid sequence comprising or consisting of SEQ ID NO: 319 and a nucleic acid sequence comprising or consisting of SEQ ID NO: 321, a nucleic acid sequence comprising or consisting of SEQ ID NO: 320 and a nucleic acid sequence comprising or consisting of SEQ ID NO: 321, The antigen binding domain may, for example, be encoded by a nucleic acid sequence comprising or consisting of SEQ ID NO: 322 and a nucleic acid sequence comprising or consisting of SEQ ID NO: 323. The antigen binding domain may, for example, be encoded by a nucleic acid sequence comprising or consisting of SEQ ID NO: 324 and a nucleic acid sequence comprising or consisting of SEQ ID NO: 325. The antigen binding domain may, for example, be encoded by a nucleic acid sequence comprising or consisting of SEQ ID NO: 326 and a nucleic acid sequence comprising or consisting of SEQ ID NO: 327. The antigen binding domain may, for example, be encoded by a nucleic acid sequence comprising or consisting of SEQ ID NO: 328 and a nucleic acid sequence comprising or consisting of SEQ ID NO: 329. The antigen binding domain may, for example, be encoded by a nucleic acid sequence comprising or consisting of SEQ ID NO: 330 and a nucleic acid sequence comprising or consisting of SEQ ID NO: 332, or a nucleic acid sequence comprising or consisting of SEQ ID NO: 331 and a nucleic acid sequence comprising or consisting of SEQ ID NO: 332. The antigen binding domain may, for example, be encoded by a nucleic acid sequence comprising or consisting of SEQ ID NO: 333 and a nucleic acid sequence comprising or consisting of SEQ ID NO: 334. The antigen binding domain may, for example, be encoded by a nucleic acid sequence comprising or consisting of SEQ ID NO: 335 and a nucleic acid sequence comprising or consisting of SEQ ID NO: 336. The antigen binding domain may, for example, be encoded by a nucleic acid sequence comprising or consisting of SEQ ID NO: 337 and a nucleic acid sequence comprising or consisting of SEQ ID NO: 338. The antigen binding domain may, for example, be encoded by a nucleic acid sequence comprising or consisting of SEQ ID NO 339 and a nucleic acid sequence comprising or consisting of SEQ ID NO: 340. The antigen binding domain may, for example, be encoded by a nucleic acid sequence comprising or consisting of SEQ ID NO: 341 and a nucleic acid sequence comprising or consisting of SEQ ID NO: 342. The antigen binding domain may, for example, be encoded by a nucleic acid sequence comprising or consisting of SEQ ID NO: 343 and a nucleic acid sequence comprising or consisting of SEQ ID NO: 344. The antigen binding domain may, for example, be encoded by a nucleic acid sequence comprising or consisting of SEQ ID NO: 345 and a nucleic acid sequence comprising or consisting of SEQ ID NO: 346. The antigen binding domain may, for example, be encoded by a nucleic acid sequence comprising or consisting of SEQ ID NO: 347 and a nucleic acid sequence comprising or consisting of SEQ ID NO: 349, or a nucleic acid sequence comprising or consisting of SEQ ID NO: 348 and a nucleic acid sequence comprising or consisting of SEQ ID NO: 349. The antigen binding domain may, for example, be encoded by a nucleic acid sequence comprising or consisting of a nucleic acid sequence comprising or consisting of SEQ ID NO: 350 and a nucleic acid sequence comprising or consisting of SEQ ID NO: 351, or a nucleic acid sequence comprising or consisting of SEQ ID NO: 350 and a nucleic acid sequence comprising or consisting of SEQ ID NO: 352. The antigen binding domain may, for example, be encoded by a nucleic acid sequence comprising or consisting of SEQ ID NO: 353 and a nucleic acid sequence comprising or consisting of SEQ ID NO: 354. The antigen binding domain may, for example, be encoded by a nucleic acid sequence comprising or consisting of SEQ ID NO: 355 and a nucleic acid sequence comprising or consisting of SEQ ID NO: 356. The antigen binding domain may, for example, be encoded by a nucleic acid sequence comprising or consisting of SEQ ID NO: 357 and a nucleic acid sequence comprising or consisting of SEQ ID NO: 358. The antigen binding domain may, for example, be encoded by a nucleic acid sequence comprising or consisting of SEQ ID NO: 359 and a nucleic acid sequence comprising or consisting of SEQ ID NO: 360. The antigen binding domain may, for example, be encoded by a nucleic acid sequence comprising or consisting of SEQ ID NO: 361 and a nucleic acid sequence comprising or consisting of SEQ ID NO: 362. The antigen binding domain may, for example, be encoded by a nucleic acid sequence comprising or consisting of SEQ ID NO: 363 and a nucleic acid sequence comprising or consisting of SEQ ID NO: 364. The antigen binding domain may, for example, be encoded by a nucleic acid sequence comprising or consisting of SEQ ID NO: 365 and a nucleic acid sequence comprising or consisting of SEQ ID NO: 366. The antigen binding domain may, for example, be encoded by a nucleic acid sequence comprising or consisting of SEQ ID NO:367 and a nucleic acid sequence comprising or consisting of SEQ ID NO: 368. The antigen binding domain may, for example, be encoded by a nucleic acid sequence comprising or consisting of SEQ ID NO: 369 and a nucleic acid sequence comprising or consisting of SEQ ID NO: 371, or a nucleic acid sequence comprising or consisting of SEQ ID NO: 370 and a nucleic acid sequence comprising or consisting of SEQ ID NO: 371, The antigen binding domain may, for example, be encoded by a nucleic acid sequence comprising or consisting of SEQ ID NO: 372 and a nucleic acid sequence comprising or consisting of SEQ ID NO: 373. The antigen binding domain may, for example, be encoded by a nucleic acid sequence comprising or consisting of SEQ ID NO: 374 and a nucleic acid sequence comprising or consisting of SEQ ID NO: 376, or a nucleic acid sequence comprising or consisting of SEQ ID NO: 375 and a nucleic acid sequence comprising or consisting of SEQ ID NO: 376. The antigen binding domain may, for example, be encoded by a nucleic acid sequence comprising or consisting of SEQ ID NO: 377 and a nucleic acid sequence comprising or consisting of SEQ ID NO: 378, or a nucleic acid sequence comprising or consisting of SEQ ID NO: 377 and a nucleic acid sequence comprising or consisting of SEQ ID NO: 379.

The immune cell may, for example, be a B cell or a plasma cell. The antigen binding domain may, for example, comprise all or part of a light chain from an antibody or a B cell receptor (BCR). The antigen binding domain may comprise all or part of a heavy chain of an antibody or a BCR. The antigen binding domain may comprise all or part of a light chain and all or part of a heavy chain of an antibody or a BCR. The antigen binding domain may comprise the three CDRs from the light chain of an antibody or a BCR. The antigen binding domain may comprise the three CDRs from the heavy chain of an antibody or a BCR. The antigen binding domain may comprise the three CDRs from the light chain and the three CDRs from the heavy chain of an antibody or a BCR. The antigen binding domain may comprise the variable region of an antibody or BCR. The antigen binding domain may comprise a Fab or (Fab′)₂ from an antibody or BCR.

The antigen binding molecule may comprise a variant of an antigen binding domain that is expressed by an immune cell from a donor whose genome does not comprise the germline variation. Antigen binding domains are described in detail in the preceding paragraphs. Over the entire length of the amino acid sequence of the antigen binding domain, a variant will preferably have at least 50% identity to that sequence. More preferably, the variant will have at least 55%, at least 60%, at least 65%, at least 70%, at least 75%, at least 80%, at least 85%, at least 90%, at least 95%, at least 97% or at least 99% identity to the amino acid sequence of the antigen binding domain. The variant retains the antigen binding ability of the antigen binding domain.

The antigen binding molecule may comprise or consist of an antigen binding molecule that is expressed by an immune cell from a donor whose genome does not comprise the germline variation. The immune cell may, for example, be a T cell or NKT cell. The antigen binding molecule may be a TCR. The immune cell may be a B cell or a plasma cell. The antigen binding molecule may be an antibody or a BCR.

The antigen binding molecule may comprise a variant of an antigen binding molecule that is expressed by an immune cell from a donor whose genome does not comprise the germline variation. Antigen binding molecules are described in the preceding paragraph. Over the entire length of the amino acid sequence of the antigen binding molecule, a variant will preferably have at least 50% identity to that sequence. More preferably, the variant will have at least 55%, at least 60%, at least 65%, at least 70%, at least 75%, at least 80%, at least 85%, at least 90%, at least 95%, at least 97% or at least 99% identity to the amino acid sequence of the antigen binding molecule. The variant retains the antigen binding ability of the antigen binding molecule.

The variant may comprise the CDRs of the antigen binding molecule expressed by an immune cell from a donor whose genome does not comprise the germline variation. For example, the variant may comprise the three CDRs from the alpha chain of a TCR expressed by the immune cell. The variant may comprise the three CDRs from the beta chain of a TCR expressed by the immune cell. The variant may comprise three CDRs from the alpha chain and the three CDRs from the beta chain of a TCR expressed by the immune cell. The variant may comprise the three CDRs from the light chain of an antibody or a BCR expressed by the immune cell. The variant may comprise the three CDRs from the heavy chain of an antibody or a BCR expressed by the immune cell. The variant may comprise the three CDRs from the light chain and the three CDRs from the heavy chain of an antibody or a BCR expressed by the immune cell.

### Immunotherapeutic Agent

The immunotherapeutic agent comprises an antigen binding molecule that binds to a peptide antigen that is (i) encoded by a germline variation in the individual, (ii) capable of binding to a MHC molecule, and (iii) present on the surface of a target cell in the individual and optionally absent on the surface of non-target cells in the individual. Antigen binding molecules and peptide antigens are described in detail above.

The immunotherapeutic agent may further comprise an immune cell that expresses the antigen binding molecule on its surface. The immune cell may be autologous with respect to the individual. The immune cell may be allogeneic with respect to the individual. The immune cell may be derived from a donor whose genome does not comprise the germline variation. Preferably, the donor does not have the disease. The immune cell may, for example, be a T cell, a B cell, a NKT cell, a NK cell, a monocyte or a macrophage. Preferably, the immune cell is a T cell. The T cell may be a CD4+ T cell or a CD8+ T cell. Preferably, the T cell is CD4+ T cell.

The antigen binding molecule expressed on the surface of the immune cell may be derived from the donor. For instance, the immune cell may be a donor-derived T cell or NKT cell. The antigen binding molecule expressed on the surface of the donor-derived immune cell may be a donor-derived TCR. In this way, the donor-derived immune cell may naturally have specificity for the peptide antigen. The immunotherapeutic agent may be administered to the individual to provide an allogeneic immune cell transplant. Following administration, peptide antigen on target cells may bind to the antigen binding molecule, leading to immune cell activation and an immune response against target cells expressing the peptide antigen. Accordingly, the disease in the individual may be treated.

The antigen binding molecule expressed on the surface of the immune cell may be a T cell receptor (TCR) or a chimeric antigen receptor (CAR). Expression of a TCR or CAR specific for the peptide antigen may confer specificity for the peptide antigen onto an immune cell that is autologous or allogeneic with respect to the individual, and that otherwise would not recognise the peptide antigen. In this way immune cells specific for the peptide antigen can be engineered. Preferably, the immune cell is a T cell. Preferably, the T cell is a CD4+ T cell. Following administration of engineered immune cells to the individual, peptide antigen on target cells may bind to the CAR, leading to immune cell activation and an immune response against target cells expressing the peptide antigen. Accordingly, the disease in the individual may be treated.

The immunotherapeutic agent may be administered by any route. Suitable routes include, but are not limited to, the intravenous, intramuscular, intraperitoneal, subcutaneous, intradermal, and transdermal routes.

The immunotherapeutic agent is administered in a manner compatible with the dosage formulation and in such amount will be therapeutically effective. The quantity to be administered depends on the subject to be treated, the disease to be treated, and the capacity of the individual's immune system. Precise amounts of the immunotherapeutic agent required to be administered may depend on the judgement of the practitioner and may be peculiar to each individual.

The immunotherapeutic agent may comprise a pharmaceutically acceptable carrier or diluent. Thus, the immunotherapeutic agent may be provided as a pharmaceutical composition. Typically, such compositions are prepared as liquid suspensions of antigen binding molecules and/or cells. The antigen binding molecules and/or cells may be mixed with an excipient which is pharmaceutically acceptable and compatible with the active ingredient. Suitable excipients are, for example, water, saline, dextrose, glycerol, of the like and combinations thereof. The pharmaceutical composition may be formulated using any suitable method. Formulation of antigen binding molecules and/or cells with standard pharmaceutically acceptable carriers and/or excipients may be carried out using routine methods in the pharmaceutical art. The exact nature of a formulation will depend upon several factors including the cells to be administered and the desired route of administration. Suitable types of formulation are fully described in Remington's Pharmaceutical Sciences, 19th Edition, Mack Publishing Company, Eastern Pennsylvania, USA. In addition, if desired, the pharmaceutical compositions may contain minor amounts of auxiliary substances such as wetting or emulsifying agents, and/or pH buffering agents.

### Polynucleotide

The invention further provides a polynucleotide encoding the antigen binding molecule or peptide antigen of the invention. That is, the invention provides a polynucleotide encoding an antigen binding molecule that is (i) encoded by a germline variation in an individual having a disease, (ii) capable of binding to a MHC molecule, and (iii) present on the surface of a target cell in the individual and optionally absent on the surface of non-target cells in the individual. The invention also provides a polynucleotide encoding a peptide antigen that is (i) encoded by a germline variation in an individual having a disease, (ii) capable of binding to a MHC molecule, and (iii) present on the surface of a target cell in the individual and optionally absent on the surface of non-target cells in the individual. Any of the aspects describe above in connection with the antigen binding molecule or the peptide of the invention may also apply to the polynuleotide of the invention.

The polynucleotide may comprise RNA. The polynucleotide may comprise DNA. The polynucleotide may comprise RNA and DNA.

The polynucleotide may, for example, comprise one or more of SEQ ID NOs: 5, 7, 9, 11, 13, 15, 17, 19, 21, 23, 24, 25, 26, 27, 28, 29, 30, 31, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378 or 379.

### Vector

The invention provides a vector comprising the polynucleotide of the invention. That is, the invention provides a vector comprising a polynucleotide encoding an antigen binding molecule that binds to a peptide antigen that is (i) encoded by a germline variation in the individual, (ii) capable of binding to a MHC molecule, and (iii) present on the surface of a target cell in the individual and optionally absent on the surface of non-target cells in the individual. The invention also provides a vector comprising a polynucleotide encoding a peptide antigen that is (i) encoded by a germline variation in an individual having a disease, (ii) capable of binding to a MHC molecule, and (iii) present on the surface of a target cell in the individual and optionally absent on the surface of non-target cells in the individual.

The vector may be a viral vector. The viral vector may, for example, be a lentivirus, a retrovirus, an adenovirus, an adeno-associated virus (AAV), a vaccinia virus or a herpes simplex virus. Methods for producing and purifying such vectors are known in the art. The retrovirus may be a gamma-retrovirus. The lentivirus may be a modified HIV virus suitable for use in delivering genes. The lentivirus may be a SIV, FIV, or equine infectious anemia virus (EQIA) based vector. The viral vector may comprise a targeting molecule to ensure efficient transduction with the nucleic acid sequence or nucleic acid construct. The targeting molecule will typically be provided wholly or partly on the surface of the viral vector in order for the molecule to be able to target the virus to cells. The viral vector is preferably replication deficient.

The vector may be a non-viral vector. Preferably, the non-viral vector is a DNA plasmid, a naked nucleic acid, a nucleic acid complexed with a delivery vehicle, or an artificial virion. The non-viral vector may be a human artificial chromosome, as described in e.g. Kazuki et al., Mol. Ther. 19 (9): 1591-1601 (2011), and Kouprina et al., Expert Opinion on Drug Delivery 11 (4): 517-535 (2014). When the non-viral vector is a nucleic acid complexed with a delivery vehicle, the delivery vehicle may be a liposome, virosome, or immunoliposome. Integration of a plasmid vector may be facilitated by a transposase such as sleeping beauty or PiggyBAC.

### EXAMPLES

### Example 1—Identifying the Antigenic Basis of GvL

Given the low mutational burden in AML, HLA-bound GvL antigens are likely to result from mismatched expressed germline variants between patient and donor (i.e. minor histocompatibility antigens) rather than neoantigens. It remains unclear whether donor GvL responses are driven by public antigens (those with a frequency>0.05 in the population) or low frequency, private antigens.

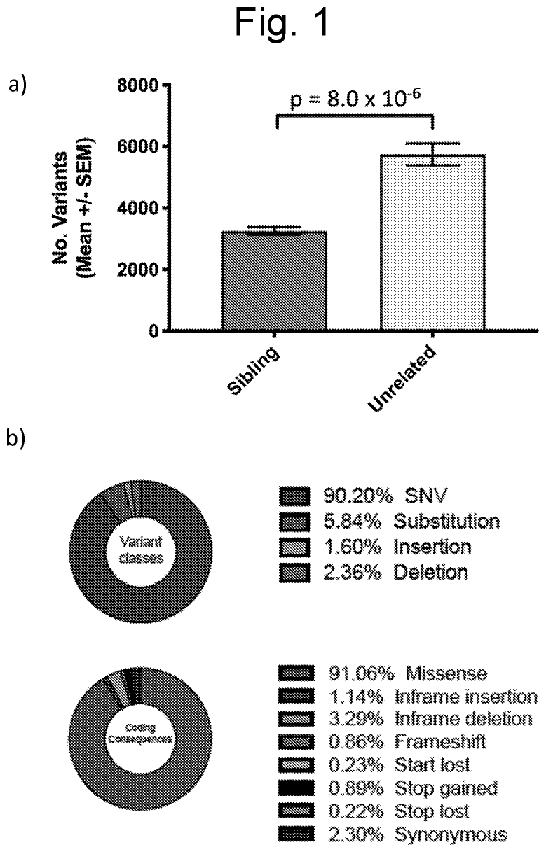

We implemented an unbiased approach to identify GvL antigens in bone marrow samples from 15 AML patients treated with allo-SCT (12/15 have been cured; 3/15 died from relapsed disease). We first performed whole exome sequencing (WES) of patient and donor DNA to identify coding variants mismatched between patient and donor, and then RNA-Seq to identify all variants mismatched between patient AML cells and donor that were expressed on patient AML cells. As expected, more germline mismatched variants were identified in unrelated compared with sibling donor transplants (5744 vs 3253 variants, FIGS. 1a and b).

Of all variants, 98.7% were germline and only 1.3% were AML-specific somatic mutations. A mean of 28.6% variants were detectable in RNA-Seq reads. Of all variants, 47.8% are present in 2 or more patients. Variants present in multiple patients are more likely to have importance for clinical translation. From these variants, putative GvL antigens were identified using two methods:
1. In silico prediction of HLA-binding affinity, using exome-identified variants and RNA-Seq expression data from AML.
2. Mass spectrometry of peptides presented by HLA class I and II.

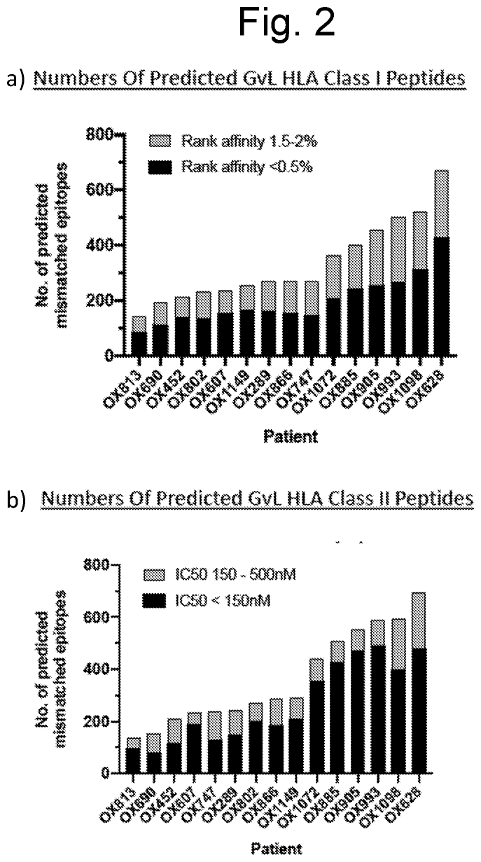

First, we identified putative GvL antigens by integrating WES-identified variants, RNA-Seq expression data and patient-specific HLA alleles. Then we performed in silico HLA class I and II-binding prediction using NetMHCpan 4.0 and NetMHCIIpan 3.0, utilising artificial neural networks trained on HLA-binding data. For HLA class I and II, between 150 and 700 mismatched HLA-binding epitopes were predicted for each patient (FIG. 2). An average of 2864 genes contained potential immunogenic variants/patient. 56% of these genes are expressed in AML blasts by RNA-Seq.

In our second approach, we immunoprecipitated HLA Class I and II from AML blasts, eluted HLA-bound peptides and analysed them by mass spectrometry. Patient-specific proteome databases were produced using the exome-identified variants. Peptide sequences were then identified from the mass spectra with reference to these databases. Immunopeptidome analysis revealed 2.1 patient-donor mismatched peptides per patient (compared to the 150-700 mismatched peptides from computational approaches). This was unsurprising as immunopeptidome analysis identifies only a subset of highest affinity, well ionising HLA-bound peptides. Nevertheless, it did confirm some predicted mis-matched peptides.

### Characterising the GvL T Cell Response

For any individual patient, it is not known whether GvL responses are mediated by a small number of dominant T cell clones or whether a large number of clones contribute to the anti-AML response. To begin to address this, we established a generic workflow. As a proof of principle, we studied one patient in detail. We screened a library of 335 mismatched peptides identified by in silico prediction and mass spectrometry for their ability to activate donor-derived T cells taken from the patient post-allo-SCT, using sensitive cultured IFNγ ELISpot assays. This patient had strong T cell responses to two overlapping peptides (SEQ ID NOs: 3 and 4, see below), targeting the same germline mis-matched variant.

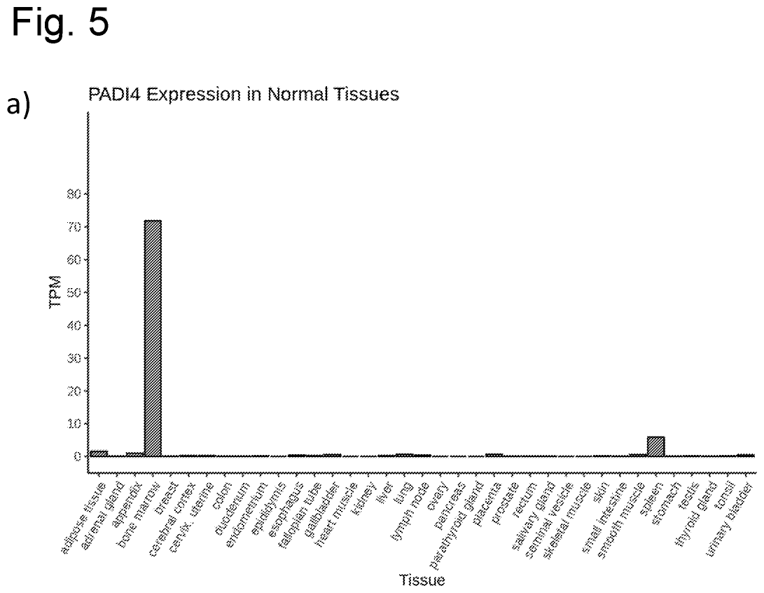

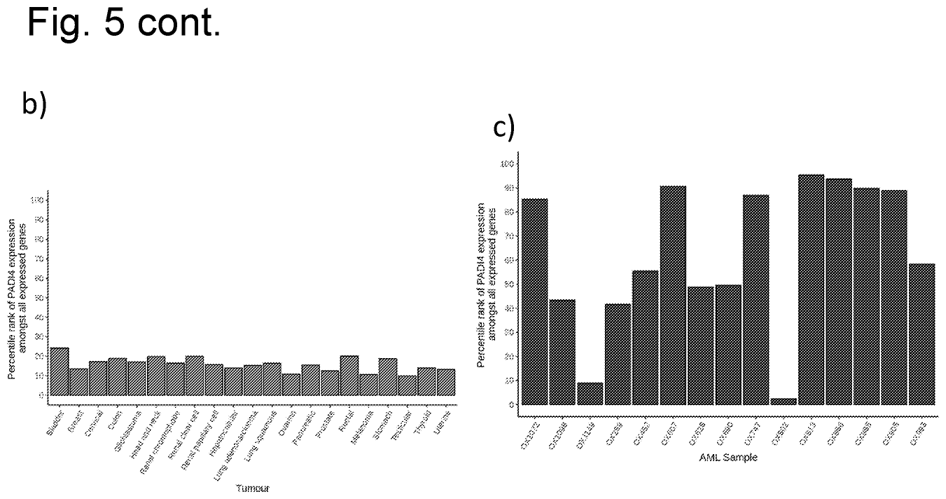

The single nucleotide polymorphism (SNP) producing the immunogenic peptides is a C→T substitution in PADI4 (peptidyl arginine deiminase 4), which results in a serine(S)→phenylalanine (F) amino acid substitution. (dbSNP ID=rs1748020). Expression of PADI14 in normal human tissue, in human tumours, and in primary AML samples is shown in FIG. 5. In the initial IFNγ ELISpot screen, 2 peptides overlapping this PADI4 SNP elicited T cell responses:

Peptide 1
(SEQ ID NO: 1)
LTISLLDTFNLELPEAVVFQ
Peptide 2
(SEQ ID NO: 2)
ISLLDTFNLELPEAVVFQDS

Subsequent ELISpot experiments were used to identify nested peptides within these longer sequences that produce the strongest T cell responses:

Nested peptide 1
(SEQ ID NO: 3)
SLLDTFNLELPEAVVF
Nested peptide 2
(SEQ ID NO: 4)
LDTFNLELPEAVVFQ

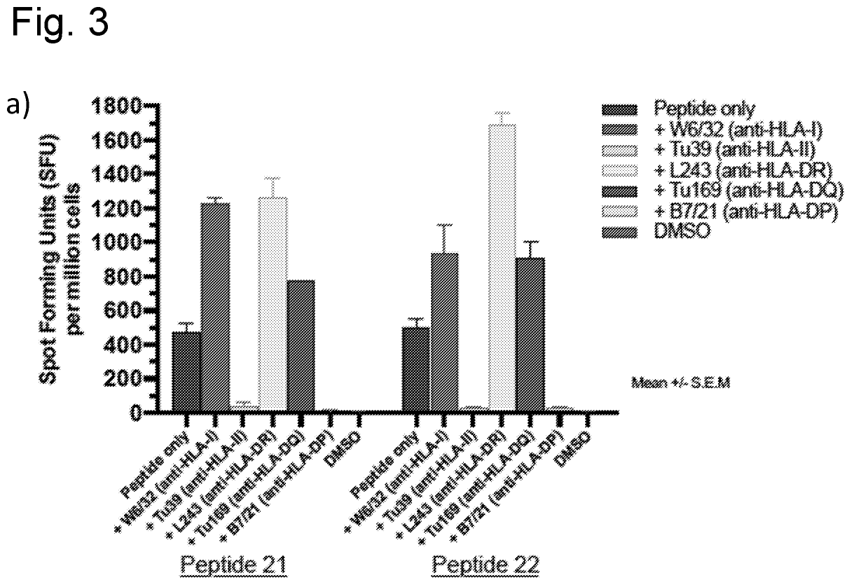

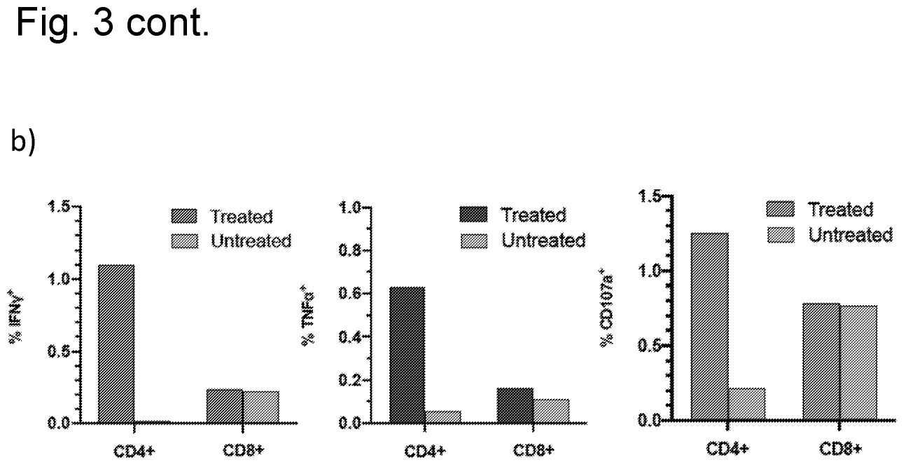

Using IFNγ ELISpots with HLA-blocking antibodies we demonstrated that allo-AML responses were HLA-DP restricted (FIG. 3a). Supporting this observation, immunophenotyping of peptide-responsive T cells identified by production of cytokines (IFNγ, TNFα) and expression of the degranulation marker (CD107a) revealed this response was mediated by CD4+, but not CD8+, T cells (FIG. 3b). All of SEQ ID Nos: 1 to 4 were demonstrated to elicit HLA-DP restricted T cell responses by IFNγ ELISpot using HLA-blocking antibodies.

In line with our observations, importance of HLA class II is supported by recent evidence of downregulation of HLA class II expression on AML blasts post-allo-SCT relapse in one third of patients, i.e. it is a common mechanism of immune evasion. Notably this germline polymorphism is present in 8% of the Caucasian population and the DP allele in ˜60% of the Caucasian population, suggesting this may be a widely applicable target GvL antigen.

The following protocol was used to determine the sequence of TCRs recognising peptide 1 (SEQ ID NO: 1) or peptide 2 (SEQ ID NO: 2):
1. Culture post-transplant blood with peptide (identified from IFNγ ELISpot screen) to enrich for antigen-specific T cells.
2. After peptide stimulation, FACS sort activated (IFNγ+) T cells and control (IFNγ−) T cells.
3. Single cell RNA sequencing with V (D) J enrichment.

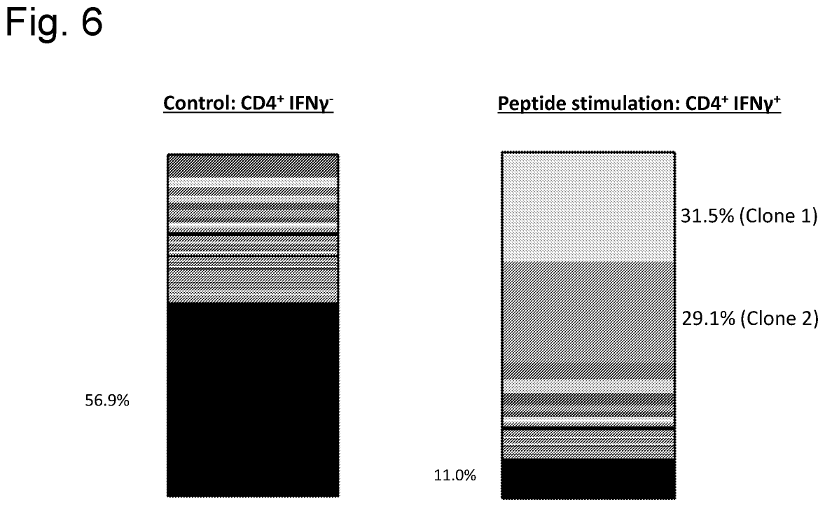

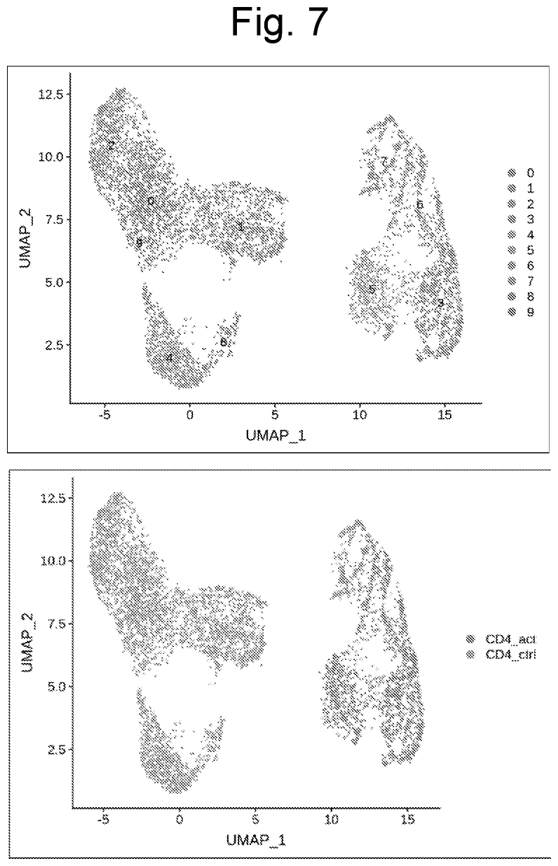

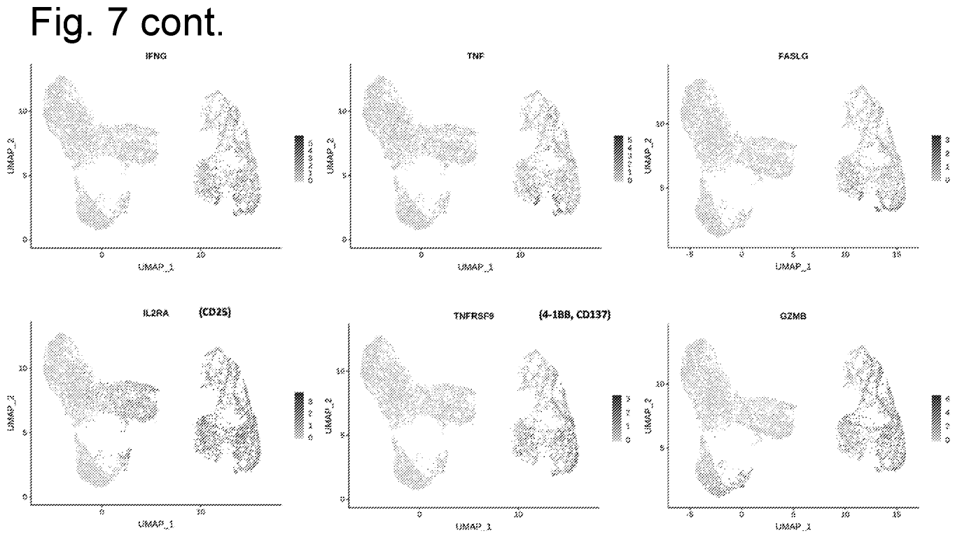

As demonstrated in FIG. 6, single cell sequencing identified two expanded clones (clone 1 and clone 2) within the CD4+ IFN γ+ population. The two expanded clones represent putative antigen-specific T cells. As shown in FIG. 7, putative antigen-specific T cells express multiple cytokines (IFNγ, TNFα) and activation markers (FASLG, IL2RA, TNFRSF9, GZMB) in response to peptide stimulation.

Single cell RNA sequencing was further used to determine the sequence of TCRs recognising nested peptide 1 (SEQ ID NO: 3) or nested peptide 2 (SEQ ID NO: 4). SEQ ID NOs: 5 to 22 provide the sequences of the alpha and beta chains from the five most frequent T cell clones in an experiment that enriched for antigen-specific T cells. Clone 2 contains two alpha sequences and one beta. Clone 4 has one of these alpha chains and the same beta, demonstrating that clones 2 and 4 are very likely to recognise the same epitope.

```
Clone 1 - TCRα
Nucleotide sequence
(SEQ ID NO: 5)
CAGAAGCCTCACACAGCCCAGTAACTTTGCTAGTACCTCTTGAGTGCAAGGTG
GAGAATTAAGATCTGGATTTGAGACGGAGCACGGAACATTTCACTCAGGGGAA

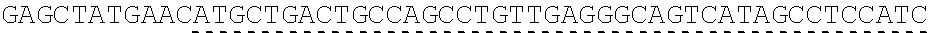

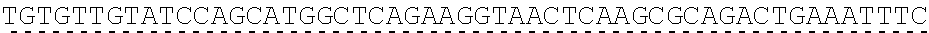

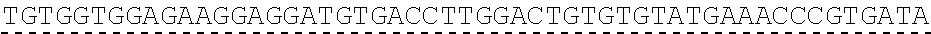

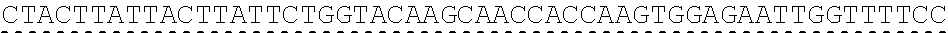

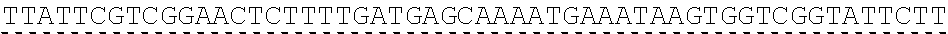

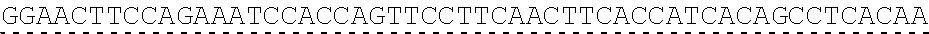

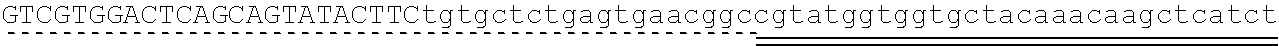

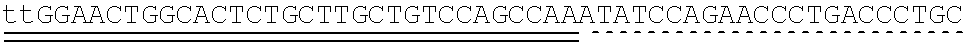

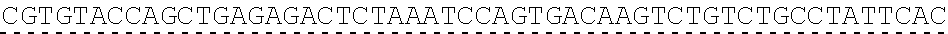

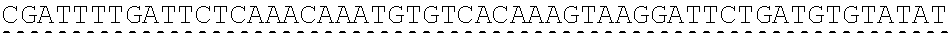

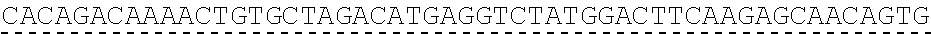

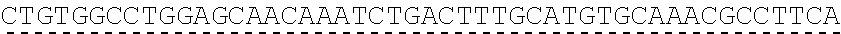












(Consensus sequence ends before completion of TRAC)
Amino acid sequence
(SEQ ID NO: 6)

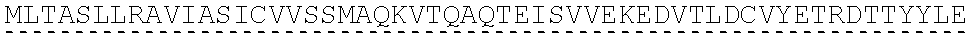

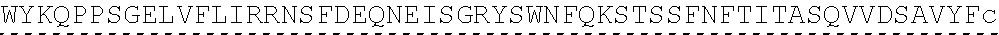

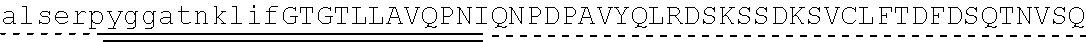

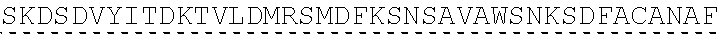




(Consensus sequence ends before completion of TRAC)
not underline = 5′ UTR

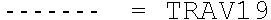

        = TRAJ32

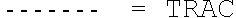

lower case = CDR3 (17-aa length)
Clone 1 - TCRβ
Nucleotide consensus sequence
(SEQ ID NO: 7)
AAAATGCCCCTCCTTTCCTCCACAGGACCAGATGCCTGAGCTAGGAAAGGCCT

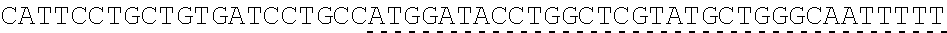

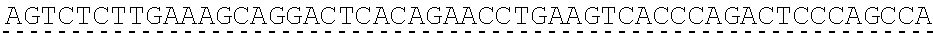

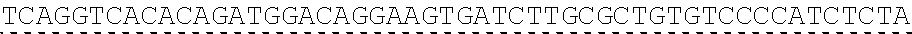

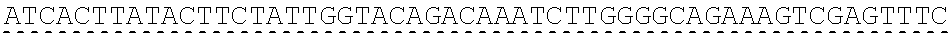

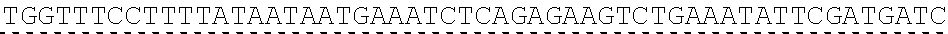

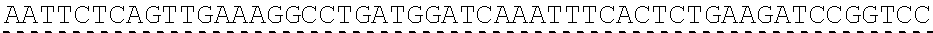

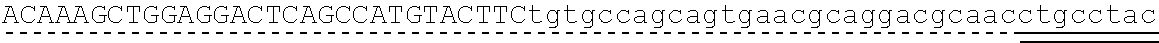

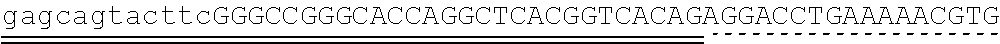

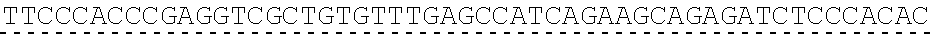

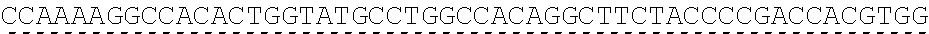

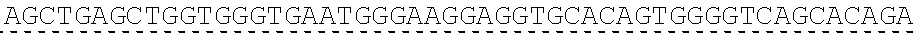

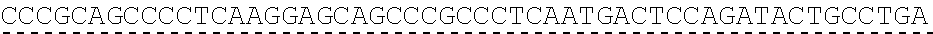

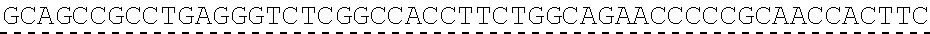

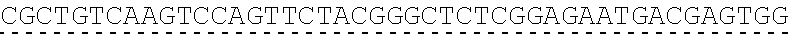














(Consensus sequence ends before completion of TRBC2)
Amino acid consensus sequence (SEQ ID NO: 8)

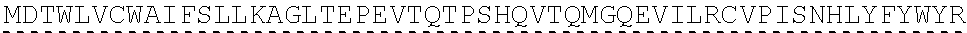

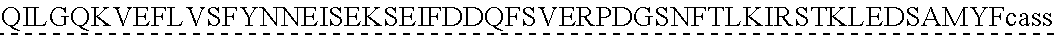

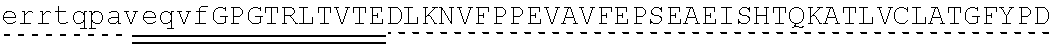

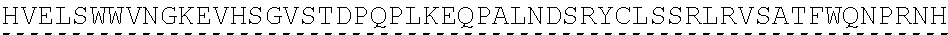

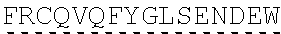





(Consensus sequence ends before completion of TRBC2)
not underlined = 5′ UTR

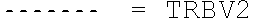

          = TRBJ2-7

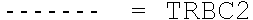

lower case = CDR3 (16-aa length)
Clone 2 - TCRα (1)
Nucleotide consensus sequence
(SEQ ID NO: 9)
AAAGCAGATTCTTTTTATGATTTTTAAAGTAGAAATATCCATTCTAGGTGCATT

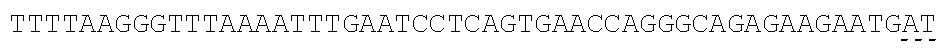

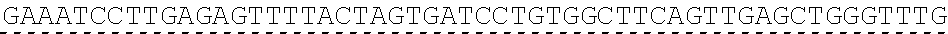

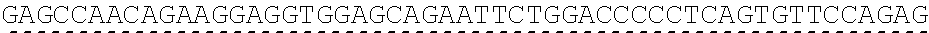

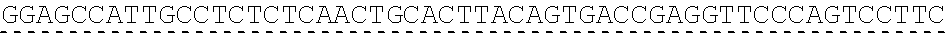

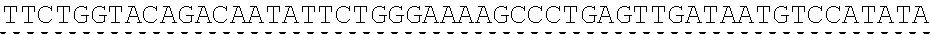

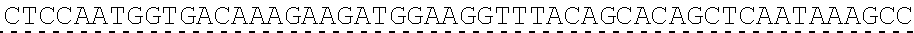

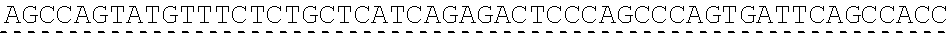

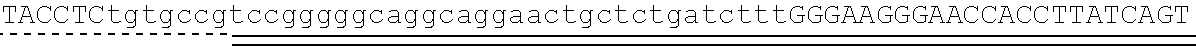

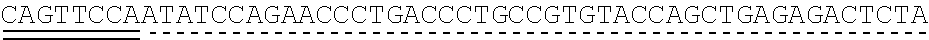

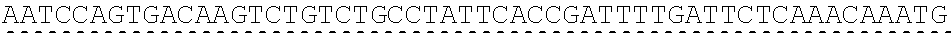

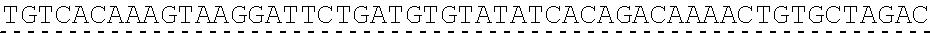

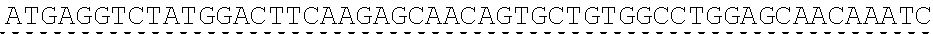

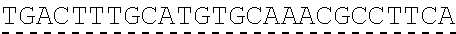













(Consensus sequence ends before completion of TRAC)
Amino acid consensus sequence (SEQ ID NO: 10)

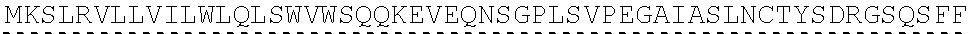

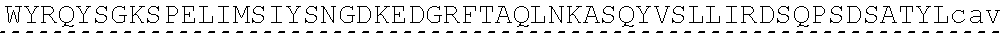

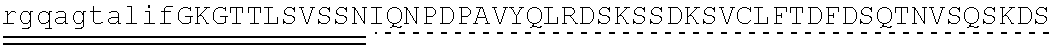

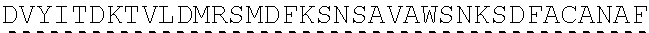




(Consensus sequence ends before completion of TRAC)
Not underlined = 5′ UTR

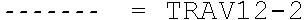

          = TRAJ15

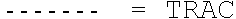

Lower case = CDR3 (13-aa length)
Clone 2 - TCRα (2)
Nucleotide consensus sequence
(SEQ ID NO: 11)
CAGAAGCCTCACACAGCCCAGTAACTTTGCTAGTACCTCTTGAGTGCAAGGTG
GAGAATTAAGATCTGGATTTGAGACGGAGCACGGAACATTTCACTCAGGGGAA

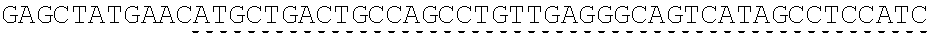

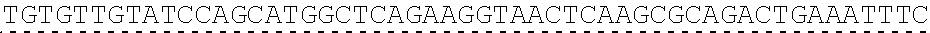

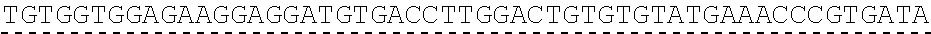

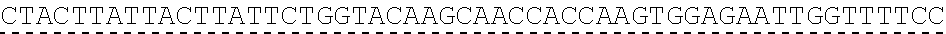

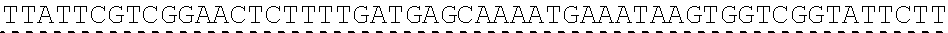

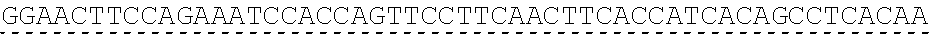

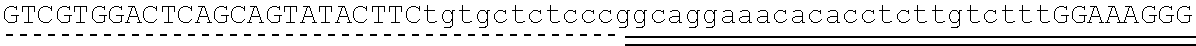

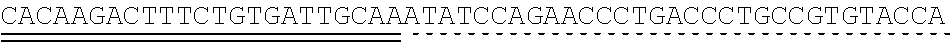

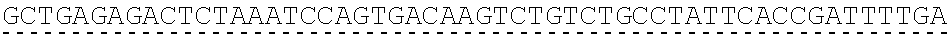

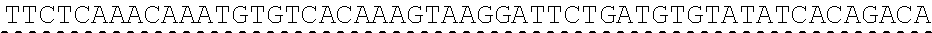

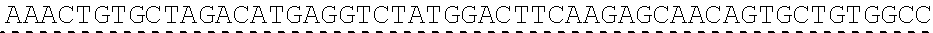

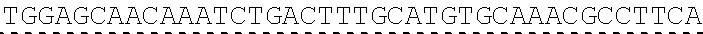












(Consensus sequence ends before completion of TRAC)
Amino acid consensus sequence
(SEQ ID NO: 12)

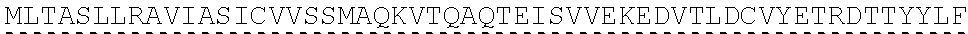

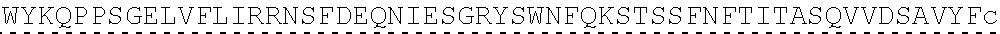

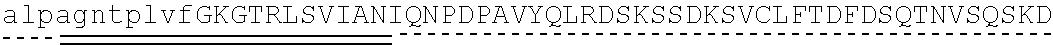

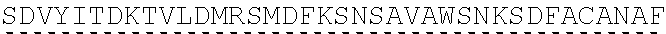




(Consensus sequence ends before completion of TRAC)
Not underlined = 5′ UTR

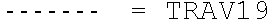

          = TRAJ29

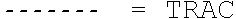

Lower case = CDR3 (12-aa length)
Clone 2 - TCRβ
Nucleotide consensus sequence
(SEQ ID NO: 13)
CTCAGAGGACCAGTATCCCTCACAGGGTGACACCTGACCAGCTCTGTCCCACC
TGGCCATGGGCTCCAGGTACCTCTGATGGGAAGACCTTTGTCTCTTGGGAACA
AGTGAATCCTTGGCACAGGCCCAGTGGATTCTGCTGTGCAGAACAGAGAGCAG
TGGACCTCAGGAGGCCTGCAAGGGGAGGACATAGGACAGTGACATCACAGTA
TGCCCCTCCCACCAGGAAAAGCAAGGCTGAGAATTTAGCTCTTTCCCAGGAGG

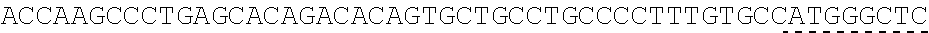

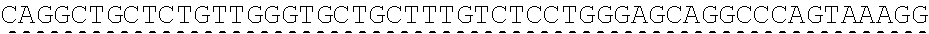

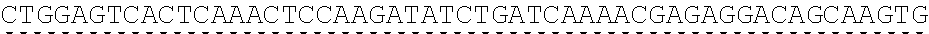

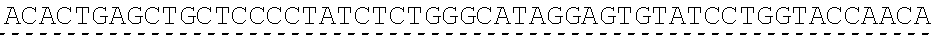

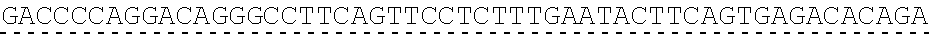

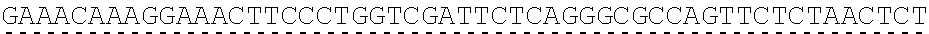

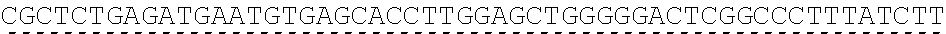

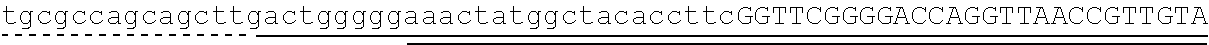

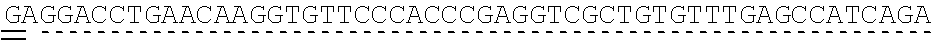

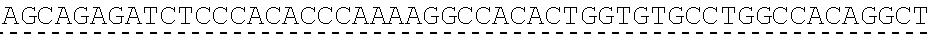

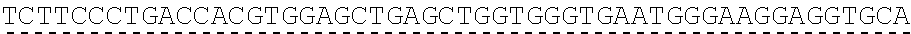

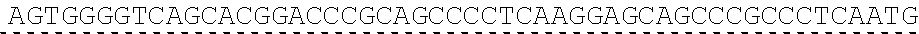

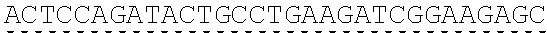













(Consensus sequence ends before completion of TRBC1)
Amino acid consensus sequence
(SEQ ID NO: 14)

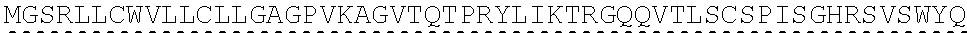

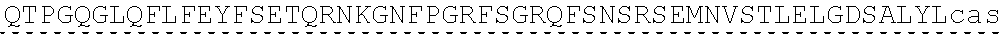

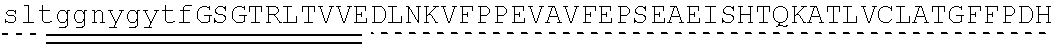

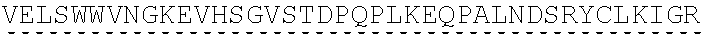




(Consensus sequence ends before completion of TRBC1)
Not underlined = 5′ UTR

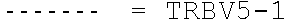

_____ = TRBD1
          = TRBJ1-2

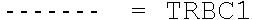

Lower case = CDR3 (14-aa length)
Clone 3 - TCRα
Nucleotide consensus sequence
(SEQ ID NO: 15)
CCAAAACAAGAGACTTGCCTAGCCCAACCTTCCTCACGCTCGCTATTCTCAAG
ACCTGGGTTCCAGCCACTTTCCTACTGGCCCCGAGGAGAATTTCCAAAGAGAC

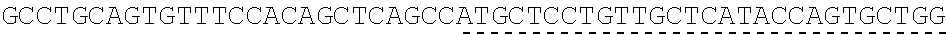

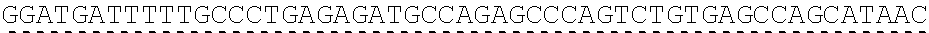

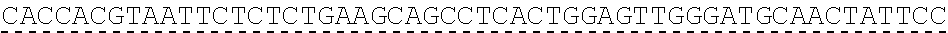

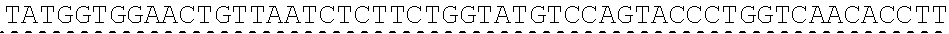

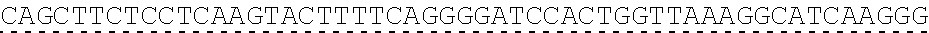

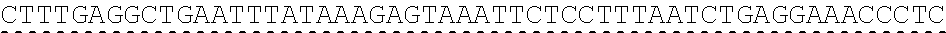

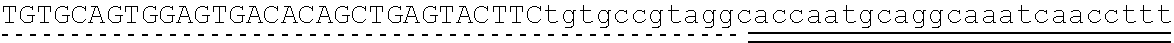

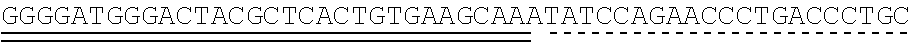

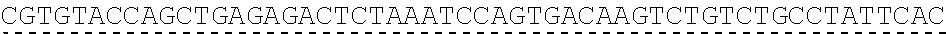

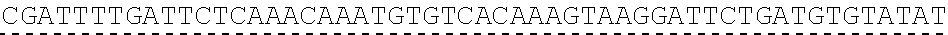

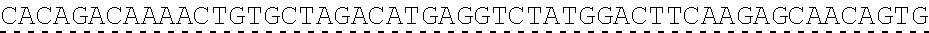

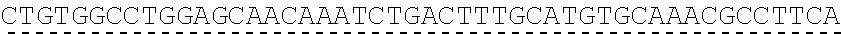












(Consensus sequence ends before completion of TRAC)
Amino acid consensus sequence
(SEQ ID NO: 16)

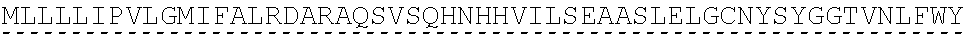

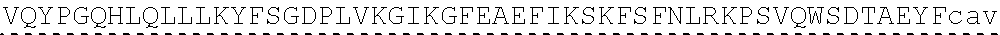

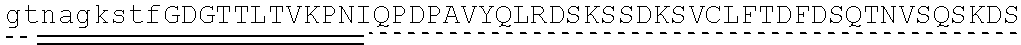

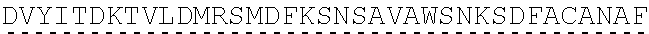




(Consensus sequence ends before completion of TRAC)
Not underlined = 5′ UTR

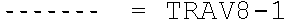

          = TRAJ27

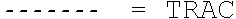

Lower case = CDR3 (12-aa length)
Clone 3 - TCRβ
Nucleotide consensus sequence
(SEQ ID NO: 17)

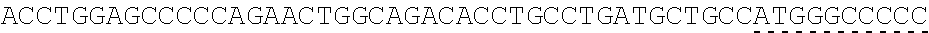

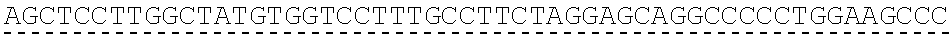

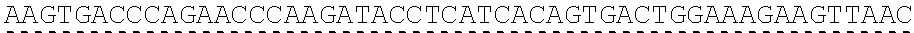

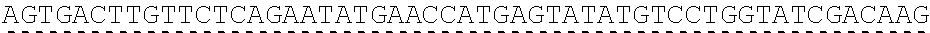

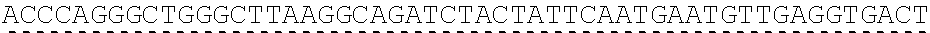

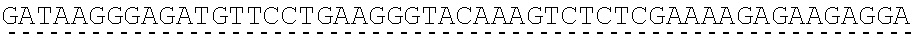

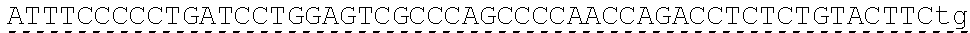

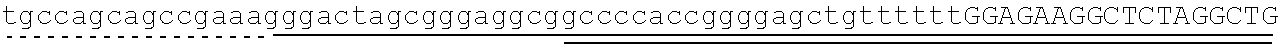

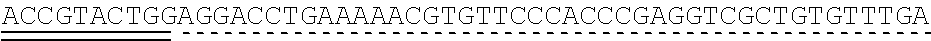

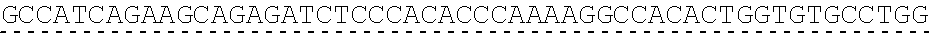

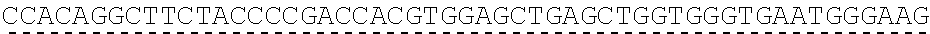

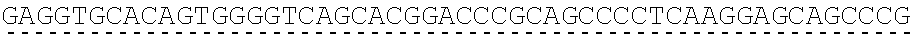

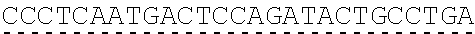













(Consensus sequence ends before completion of TRBC2)
Amino acid consensus sequence
(SEQ ID NO: 18)

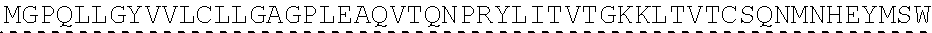

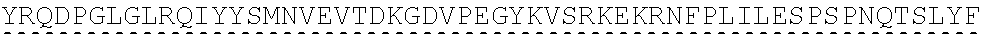

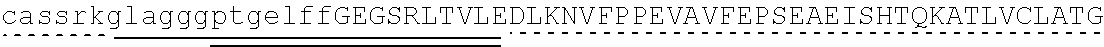

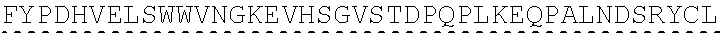




(Consensus sequence ends before completion of TRBC2)
Not underlined = 5′ UTR

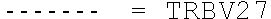

____ = TRBD2
          = TRBJ2-2

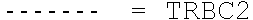

Lower case = CDR3 (19-aa length)
Clone 4
TCRα(1) and TCRβ from Clone 2
Clone 5 - TCRα
Nucleotide consensus sequence
(SEQ ID NO: 19)
AGGAAAAGTTGAGGGGGCTTGACAGACAGAAATTCTAAACTGATGCTTATCTG
TGTGTAAAGAAAGGATTACTGATTCCCAATGAATATATCTTCAGCAATTCTAA
ATTTGGACAAAGTGGGGAAGTGCTTCCTTTGACAGAGACAGCTTTAAGTGAAA
GCACTTGTGAAAGGGCGGGGCCTGCTGAAAGAATTCAGTTGAGGGTGAATTTA
CAGAGTTTCAGCTGGTTGGGAAGACTGGAAGACCACCTGGGCTGTCATTGAGC

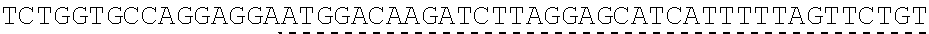

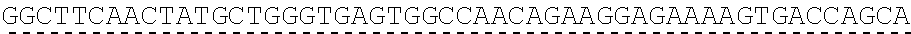

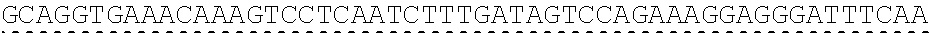

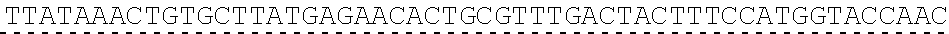

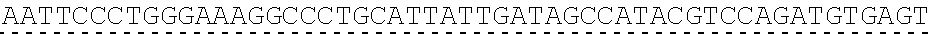

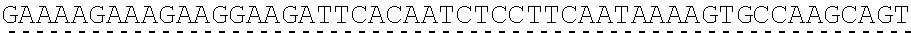

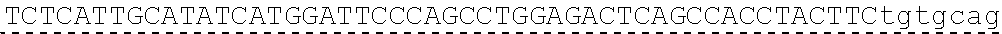

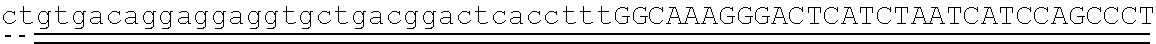

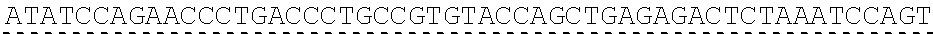

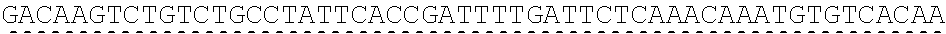

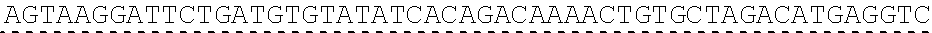

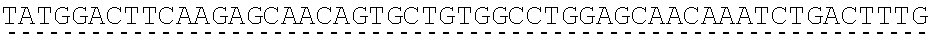

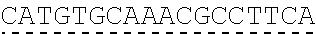













(Consensus sequence ends before completion of TRAC)
(5′ UTR sequence of consensus and reference differ - reference shown)
Amino acid consensus sequence
(SEQ ID NO: 20)

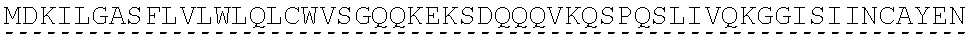

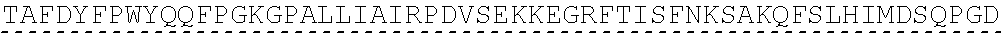

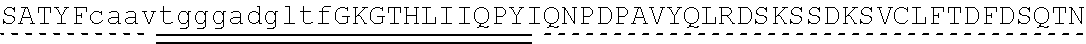

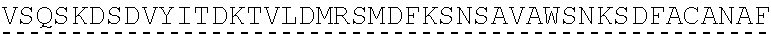




(Consensus sequence ends before completion of TRAC)
Not underlined = 5′ UTR

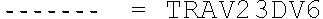

          = TRAJ45

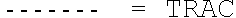

Lower case = CDR3 (14-aa length)
Clone 5 - TCRβ
Nucleotide consensus sequence
(SEQ ID NO: 21)
AGCTGTGAGGTCTGGTTCCCCGACGTGCTGCAGCAAGTGCCTTTGCCCTGCCTG

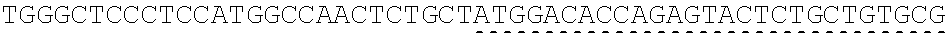

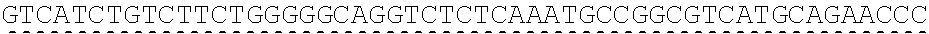

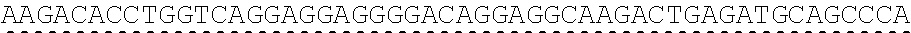

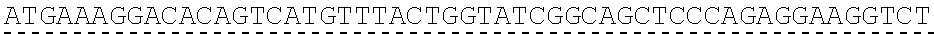

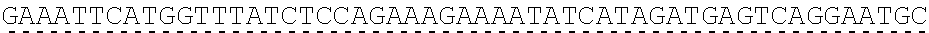

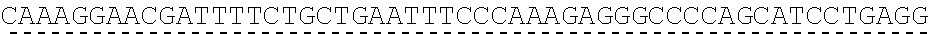

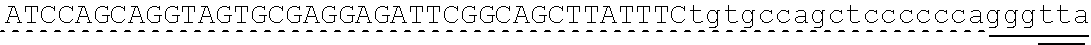

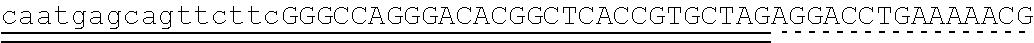

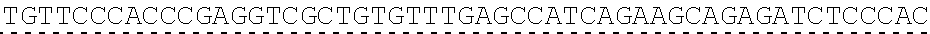

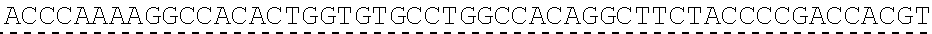

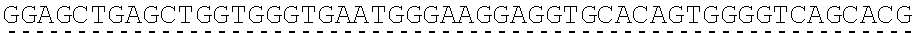

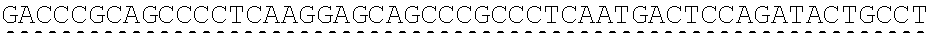

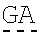













(Consensus sequence ends before completion of TRBC2)
Amino acid consensus sequence
(SEQ ID NO: 22)

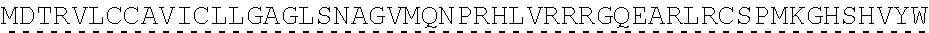

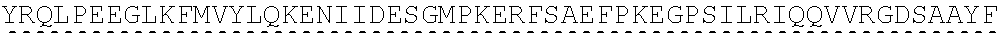

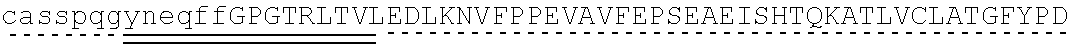

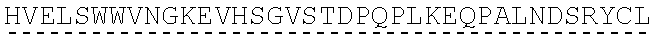




(Consensus sequence ends before completion of TRBC2)
Not underlined = 5′ UTR

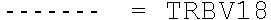

____ = TRBD1
          = TRBJ2-1

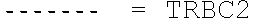

Lower case = CR3 (13-aa length)
```

SEQ ID NOs: 23 to 31 provide human codon optimised versions of SEQ ID NOs: 5, 7, 9, 11, 13, 15, 17, 19 and 21 respectively. These human codon optimised sequences encode the same amino acid sequences as SEQ ID NOs: 5, 7, 9, 11, 13, 15, 17, 19 and 21, but may be more readily expressed in human cells than the original nucleotide sequences. Thus, the human codon optimised sequences may be used in therapeutic applications in preference to the original nucleotide sequences. For each of SEQ ID NOs: 23 to 31:

____ = Restriction site/Murine constant

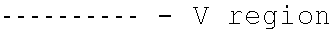

= D region

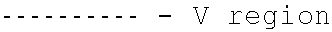

Clone 1 - TCRα
(SEQ ID NO: 23)

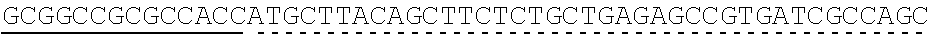

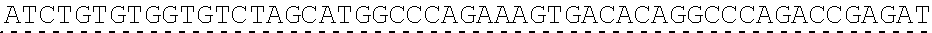

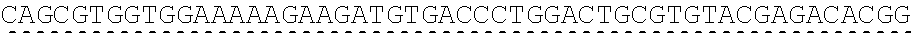

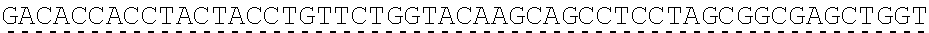

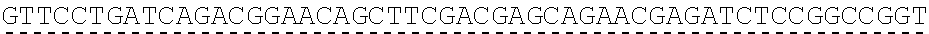

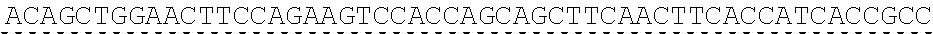

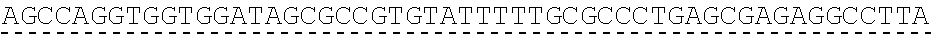

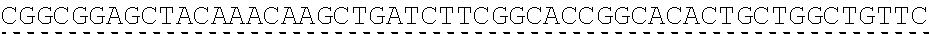

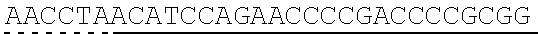

Clone 1 - TCRβ
(SEQ ID NO: 24)

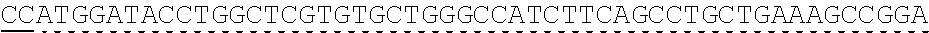

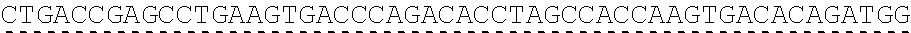

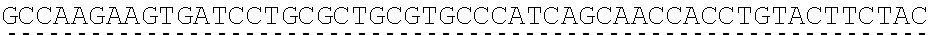

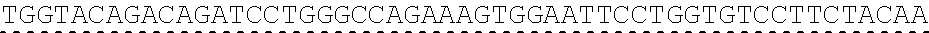

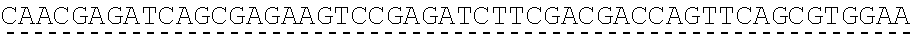

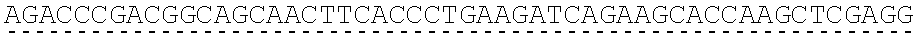

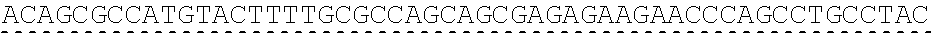

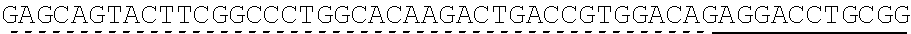

AACGTGACCCCCCCCAAGGTGTCCCTGTTCGAGCCCAGCAAGGCCGAGATCGC
CAACAAGCAGAAAGCCACACTGGTCTGTCTGGCTAGGGGCTTCTTCCCCGACC
ACGTG
Clone 2 - TCRα (1)
(SEQ ID NO: 25)

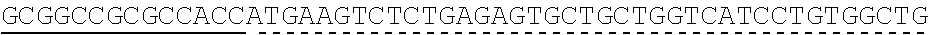

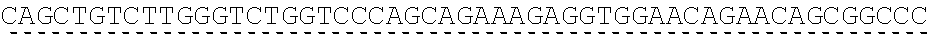

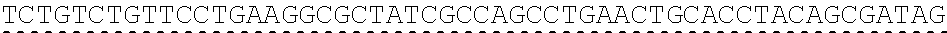

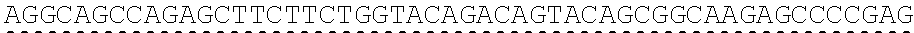

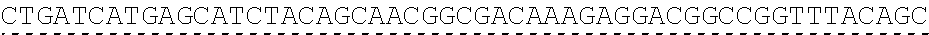

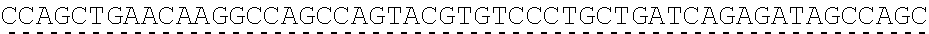

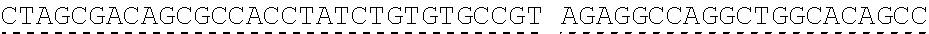

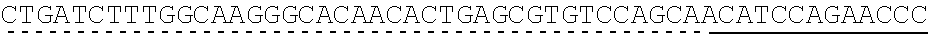

CGACCCCGCGG
Clone 2- TCRα (2)
(SEQ ID NO: 26)

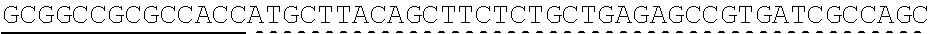

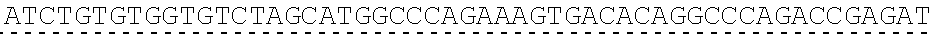

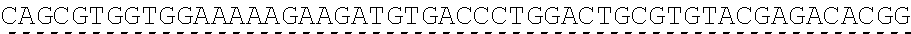

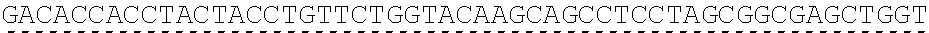

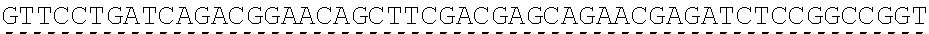

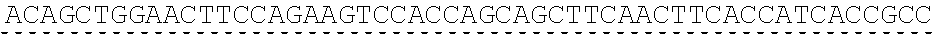

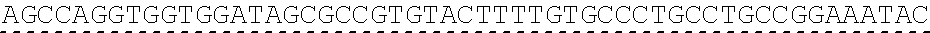

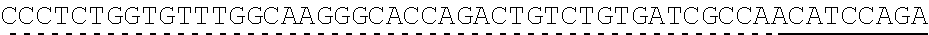

ACCCCGACCCCGCGG
Clone 2 - TCRβ
(SEQ ID NO: 27)

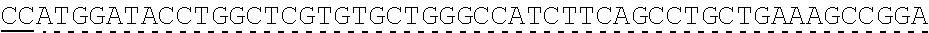

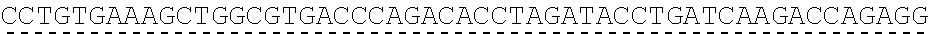

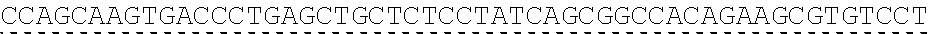

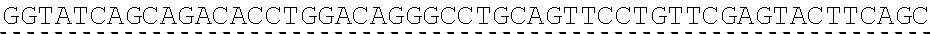

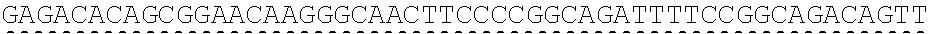

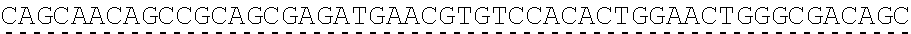

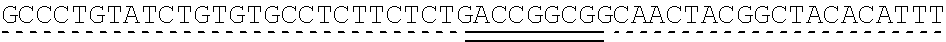

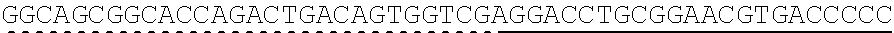

CCCAAGGTGTCCCTGTTCGAGCCCAGCAAGGCCGAGATCGCCAACAAGCAGA
AAGCCACACTGGTCTGTCTGGCTAGGGGCTTCTTCCCCGACCACGTG
Clone 3 - TCRα
(SEQ ID NO: 28)

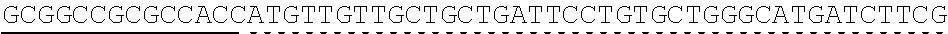

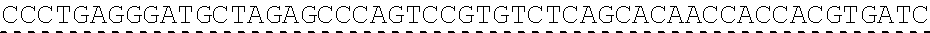

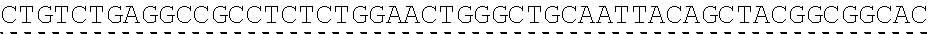

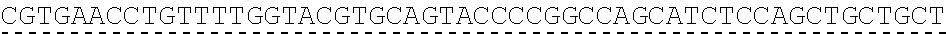

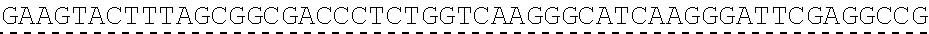

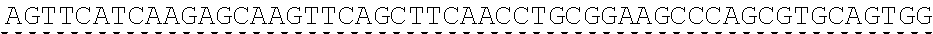

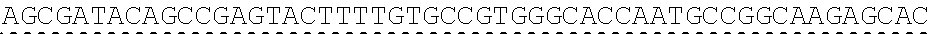

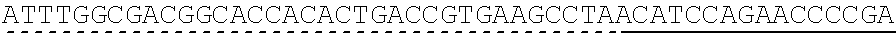

CCCCGCGG
Clone 3 - TCRβ
(SEQ ID NO: 29)

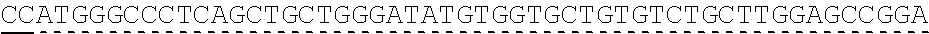

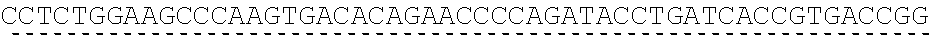

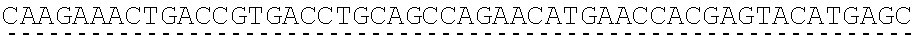

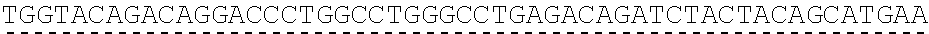

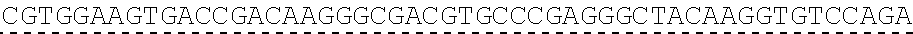

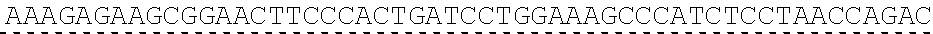

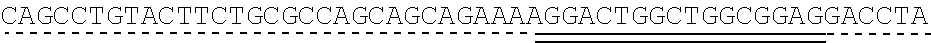

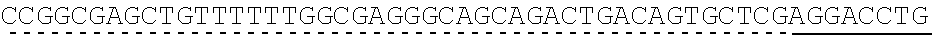

CGGAACGTGACCCCCCCCAAGGTGTCCCTGTTCGAGCCCAGCAAGGCCGAGAT
CGCCAACAAGCAGAAAGCCACACTGGTCTGTCTGGCTAGGGGCTTCTTCCCCG
ACCACGTG
Clone 5 - TCRα
(SEQ ID NO: 30)

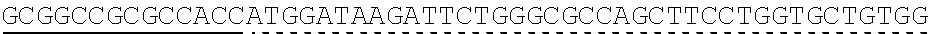

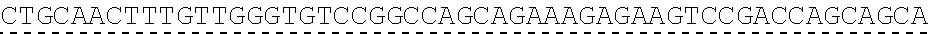

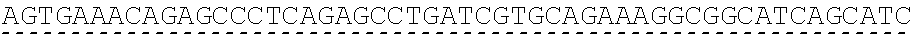

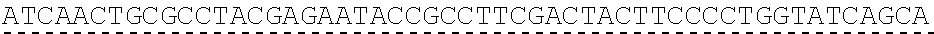

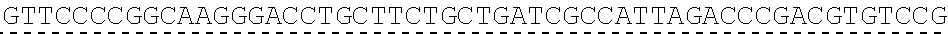

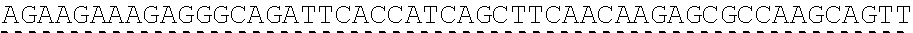

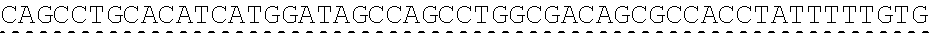

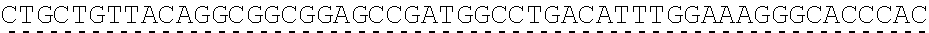

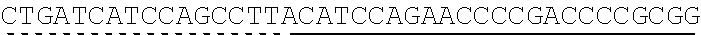

Clone 5 - TCRβ
(SEQ ID NO: 31)

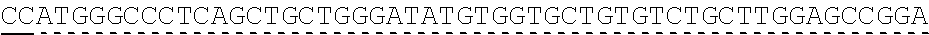

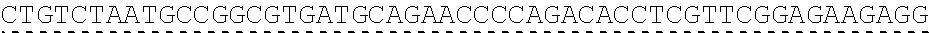

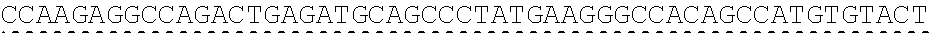

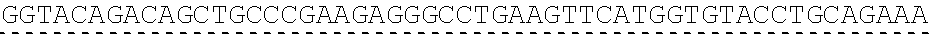

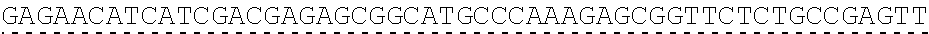

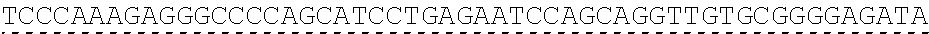

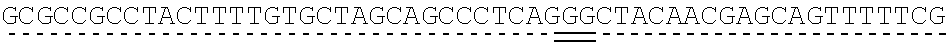

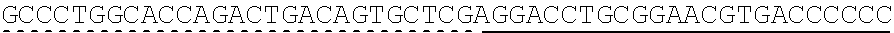

CCAAGGTGTCCCTGTTCGAGCCCAGCAAGGCCGAGATCGCCAACAAGCAGAA
AGCCACACTGGTCTGTCTGGCTAGGGGCTTCTTCCCCGACCACGTG

### Conclusion

The data provides the evidence that this is the first time any group has successful implemented the type of unbiased approach employed here to identify GvL antigens and we have also identified the cognate TCRs recognising the antigen. This opens new field in immunology and development of antigen-binding molecule based therapies, including TCR based therapies, and peptide vaccination with the antigens identified.

### Example 2—Further Identifying the Antigenic Basis of GvL

The generic workflow established in Example 1 was used to identify the antigenic basis of GvL in further patients. In brief, mismatched peptides identified by in silico prediction and mass spectrometry for each patient were screened for their ability to activate donor-derived T cells taken from the patient post-allo-SCT, using IFNγ ELISpot assays. HLA restriction was investigated using IFNγ ELISpots with HLA-blocking antibodies. The nature and frequency of peptide-responsive T cells (i.e. cells producing cytokines (IFNγ, TNFα) and expression of the degranulation marker (CD107a) following peptide contact) were assessed by culturing post-transplant blood with peptide for 12 days and then stimulating with the peptide. Responses were assessed by flow cytometry.

Results are shown in Table 3.

In Table 3, the mismatched peptide sequence expressed by the patient is indicated in column D. The amino acid mismatched between patient and donor is underlined. In many cases, there are 2 peptides covering the same genetic variant that both elicit T cell responses.

The genetic variant leading to the patient-donor mismatch is described in columns F/G/H/I. The dbSNP ID (Column I) refers to an online database of previously described inherited genetic variants.

Column J indicates whether the T cell response is directed against the variant or reference amino acid sequence. The reference amino acid sequence used was NCBI Human Reference Genome Build hg38, which is also referred to as GRCh38 (https://www.ncbi.nlm.nih.gov/assembly/GCF_000001405.26). In most cases, the donor is homozygous for the reference allele and the patient is heterozygous, so the T cell response is directed against the variant sequence. However, particularly for common genetic variants, responses may be directed against the reference sequence. In these cases, the donor is homozygous for the variant allele and the patient is either homozygous reference or heterozygous.

Column K shows the allele frequency of the variant in the population and is taken from the gnomAD database.

Columns L/M/N/O show results from HLA restriction and immunophenotyping experiments.

TABLE 3

| | | | | D | |
|---|---|---|---|---|---|
| | | | | GvL peptide amino | |
| | A | B | C | acid sequence | E |
| | Number | Patient | Peptide(s) | (mismatch underlined) | Donor sequence |
| i | 1 | OX866 | OX866-021 | LTISLLDTFNLELPEAVVFQ | LTISLLDTSNLELPEAVVFQ |
| ii | | | OX866-022 | ISLLDTFNLELPEAVVFQDS | ISLLDTSNLELPEAVVFQDS |
| iii | 2 | OX1149 | OX1149-190 | AASCEPLASVLRAKLTSRSS | AASCEPLASVLRAKPTSRSS |
| iv | 3 | OX1149 | OX1149-290 | GAGPDPLRLHGHLPVRTSCP | GAGPDPLRLRGHLPVRTSCP |
| v | 4 | OX813 | OX813-101 | TQRSVLLCKVVGARGVGKSA | TQRSVLLCKVVGACGVGKSA |
| vi | | | OX813-102 | KVVGARGVGKSAFLQAFLGR | KVVGACGVGKSAFLQAFLGR |
| vii | 5 | OX802 | OX802-321 | GQKSPRFRRVSCFLRLGRST | GQKSPRFRRVTCFLRLGRST |
| viii | | | OX802-322 | RFRRVSCFLRLGRSTLLELE | RFRRVTCFLRLGRSTLLELE |
| ix | 6 | OX289 | OX289-009 | ALAFLLLISIAANLSLLLSR | ALAFLLLISTAANLSLLLSR |
| x | 7 | OX289 | OX289-040 | EVQDCLKQLMMSLLQLYRFS | EVQDCLKQLMMSLLRLYRFS |
| xi | 8 | OX289 | OX289-052 | GYSPSLHILAIGTRSGAIKL | GYSPSLRILAIGTRSGAIKL |
| xii | 9 | OX289 | OX289-067 | VATFPVYTMVAIPIVCKD | VATFPVYTMGAIPIVCKD |
| xiii | 10 | OX289 | OX289-073 | PPLYRQRYQFIKNLVDQHEP | PPLYRQRYQFVKNLVDQHEP |
| xiv | | | OX289-074 | PLYRQRYQFIKNLVDQHEPK | PLYRQRYQFVKNLVDQHEPK |
| xv | 11 | OX289 | OX289-075 | VSRPELLRESISAFLVPMPT | VSRPELLREGISAFLVPMPT |
| xvi | | | OX289-205 | TDRALQNKSISAFLVPMPTP | TDRALQNKGISAFLVPMPTP |
| xvii | 12 | OX289 | OX289-180 | NPLSPYLNVDPRYLVQDT | NPLCPYLNVDPRYLVQDT |
| xviii | 13 | OX289 | OX289-221 | EPPVDICLSKAISSSLKGFL | EPPVDICLSKANSSSLKGFL |
| xix | 14 | OX289 | OX289-284 | GETGMFSLSTIRGHQYATY | GETGMFSLCTIRGHQYATY |
| xx | 15 | OX289 | OX1098-614 | MNYVSKRLPFAARLNTPMGP | MNYVSKSLPFAARLNTPMGP |
| xxi | 16 | OX747 | OX747-157 | LGSLGLIFALTLNRHKYPLN | LGSLGLIFALILNRHKYPLN |
| xxii | | | OX747-158 | LGLIFALTLNRHKYPLNLYL | LGLIFALILNRHKYPLNLYL |
| xxiii | 17 | OX747 | OX747-185 | APISLSSFFNVSTLEREVTD | APISLSSFFSVSTLEREVTD |
| xxiv | 18 | OX747 | OX747-190 | LELGAGTGLASIIAATMART | LELGAGTGLTSIIAATMART |
| xxv | | | OX747-191 | AGTGLASIIAATMARTVYCT | AGTGLTSIIAATMARTVYCT |
| xxvi | 19 | OX747 | OX628-305 | VPREYVRALNATKLERVFAK | VPREYIRALNATKLERVFAK |
| xxvii | 20 | OX993 | OX993-413 | LHRDKALLKRLLKGMQKKRP | LHRDKALLKRLLKGVQKKRP |
| xxviii | | | OX993-414 | KALLKRLLKGMQKKRPSDVQ | KALLKRLLKGVQKKRPSDVQ |
| xxix | 21 | OX628 | OX628-80 | ITVQTVYVQHLITFLDRPIQ | ITVQTVYVQHPITFLDRPIQ |
| xxx | | | OX628-81 | QTVYVQHLITFLDRPIQMCC | QTVYVQHPITFLDRPIQMCC |
| xxxi | 22 | OX628 | OX628-564 | PGLISMFSSSQELGAALAQL | PGLISVFSSSQELGAALAQL |
| xxxii | 23 | OX885 | OX885-559 | WRVRIALALKGIDYETVPIN | WRVRIALALKGIDYKTVPIN |
| xxxiii | | | OX885-560 | VRIALALKGIDYETVPINLI | VRIALALKGIDYKTVPINLI |
| xxxiv | 24 | OX885 | OX628-308 | DRAEKFNRGIRKLGITPEGQ | DRAEKFNRGIRKLGVTPEGQ |
| xxxv | | | OX628-309 | EKFNRGIRKLGITPEGQSYL | EKFNRGIRKLGVTPEGQSYL |

| | | | | | J | |
|---|---|---|---|---|---|---|
| | | | | | T cell response | K |
| | | | | | against reference | Allele |
| | F | G | H | I | or variant amino | frequency |
| | Gene | Chromosome | Position (hg38) | dbSNP ID | acid sequence | (%) |
| i | PADI4 | 1 | 17342114 | rs1748020 | Variant | 4.27 |
| ii | | | | | | |
| iii | RNH1 | 11 | 499120 | rs17585 | Variant | 11.21 |
| iv | SLC26A6 | 3 | 48632014 | rs13324142 | Variant | 9.84 |
| v | RHOT2 | 16 | 672331 | rs3177338 | Reference | 51.51 |
| vi | | | | | | |
| vii | COA6 | 1 | 234373513 | rs10910420 | Reference | 52.52 |
| viii | | | | | | |
| ix | NAGPA | 16 | 5025632 | rs7188856 | Variant | 30.07 |
| x | RNF123 | 3 | 49700521 | rs35620248 | Variant | 5.07 |
| xi | LLGL2 | 17 | 75556104 | rs1671036 | Variant | 50.19 |
| xii | ENDOD1 | 11 | 95129413 | rs3740861 | Variant | 25.36 |
| xiii | HENMT1 | 1 | 108657486 | rs36100901 | Variant | 1.93 |
| xiv | | | | | | |
| xv | ACADS | 12 | 120738280 | rs1799958 | Variant | 26.58 |
| xvi | | | | | | |
| xvii | TIMM23B | 10 | 49945053 | rs148307270 | Variant | 9.11 |
| xviii | LRR1 | 14 | 49607404 | rs17121605 | Variant | 20.48 |
| xix | FTSJ3 | 17 | 63823837 | rs2727288 | Reference | 64.78 |
| xx | LGALS8 | 1 | 236543562 | rs2243525 | Reference | 72.36 |
| xxi | TMBIM4 | 12 | 66152320 | rs8793 | Variant | 43.73 |
| xxii | | | | | | |
| xxiii | DOCK8 | 9 | 334337 | rs10970979 | Reference | 24.54 |
| xxiv | METTL22 | 16 | 8635267 | rs2302607 | Reference | 22.2 |
| xxv | | | | | | |
| xxvi | DCAF13 | 8 | 103420317 | rs3134253 | Variant | 23.64 |
| xxvii | DENND6B | 22 | 50313721 | rs68178377 | Variant | 31.06 |
| xxviii | | | | | | |
| xxix | LITAF | 16 | 11553659 | rs761746674 | Variant | 0.00122 |
| xxx | | | | | | |
| xxxi | PGLS | 19 | 17511703 | rs368232818 | Variant | 0.08 |
| xxxii | GSTZ1 | 14 | 77326864 | rs7975 | Reference | 30.57 |
| xxxiii | | | | | | |
| xxxiv | WASHC4 | 12 | 105152394 | rs1663564 | Variant | 94.67 |
| xxxv | | | | | | |

| | L | |
|---|---|---|
| | HLA | M |
| | restriction | Restricting HLA allele |
| i | HLA-DP | DPA1*01:03-DPB1*04:01 |
| ii | | |
| iii | HLA-DR | DRB1*13:01/DRB1*11:02:01/DRB3*02:02/28/29N/DRB3*01:01/04 |
| iv | HLA-DR | DRB1*13:01/DRB1*11:02:01/DRB3*02:02/28/29N/DRB3*01:01/04 |
| v | Indeterminate | A*68:01:01/A*11:01:01///B*55:01:01/B*15:01:01//C*03:04:01/C*03:03:01 |
| | | ///DRB1*14:54/DRB1*15:01/DRB3*02:02/28/29N/DRB5*01:01:01///DQA1*01: |
| | | 02/06/DQA1*01:01/04/05/07/DQB1*06:02:10/DQB1*05:03:01///DPB1*05:01/ |
| | | DPB1*04:01:01 |
| vi | | |
| vii | Not tested | DRB1*03:01/DRB1*11:01/DRB3*02:02/03/05/06/11/13-29N///DQA§1*05:01/ |
| | | DQA1*05:05/08-11/DQB1*02:01/DQB1*03:01//DPB1*04:01/*121:01 |
| viii | | |
| ix | Class I | A*02:05/A*23:01/05/06/08/10/11-19///B*44:03/13/26/30/32/36-61/ |
| | | B*58:01/04/11/13/15//C*04:01/05/08/10/12/16-33/C*07: |
| | | 01/06/16/18/20/21/24-57 |
| x | Likely HLA-DP | DPB1*04:01:01/DPB1*02:01 |
| xi | Likely HLA-DR | DRB1*03:01/DRB1*07:01:01/DRB3*02:02/05/11/13/14/17-23/DRB4*01:01-05/07 |
| xii | Likely Class | A*02:05/A*23:01/05/06/08/10/11-19///B*44:03/13/26/30/32/36-61/ |
| | I | B*58:01/04/11/13/15///C*04:01/05/08/10/12/16-33/C*07: |
| | | 01/06/16/18/20/21/24-57 |
| xiii | Not tested | DRB1*03:01/DRB1*07:01:01/DRB3*02:02/05/11/13/14/17-23/DRB4*01:01- |
| | | 05/07//DQB1*02:02/DQB1*02:01/02/04//DPB1*04:01:01/DPB1*02:01 |
| xiv | | |
| xv | Not tested | DRB1*03:01/DRB1*07:01:01/DRB3*02:02/05/11/13/14/17-23/DRB4*01:01- |
| | | 05/07//DQB1*02:02/DQB1*02:01/02/04///DPB1*04:01:01/DPB1*02:01 |
| xvi | | |
| xvii | Likely HLA-DR | DRB1*03:01/DRB1*07:01:01/DRB3*02:02/05/11/13/14/17-23/DRB4*01:01-05/07 |
| xviii | Likely Class | DRB1*03:01/DRB1*07:01:01/DRB3*02:02/05/11/13/14/17-23/DRB4*01:01- |
| | II | 05/07///DQB1*02:02/DQB1*02:01/02/04/DPB1*04:01:01/DPB1*02:01 |
| xix | Class | A*02:05/A*23:01/05/06/08/10/11-19/B*44:03/13/26/30/32/36-61/ |
| | | B*58:01/04/11/13/15//C*04:01/05/08/10/12/16-33/C*07: |
| | | 01/06/16/18/20/21/24-57 |
| xx | Indeterminate | A*02:05/A*23:01/05/06/08/10/11-19/B*44:03/13/26/30/32/36- |
| | | 61/B*58:01/04/11/13/15/C*04:01/05/08/10/12/16-33/C*07: |
| | | 01/06/16/18/20/21/24-57///DRB1*03:01/DRB1*07:01:01/DRB3*02:02/ |
| | | 05/11/13/14/17-23/DRB4*01:01-05/07/DQB1*02:02/DQB1*02:01/02/04/ |
| | | DPB1*04:01:01/DPB1*02:01 |
| xxi | Not tested | DRB1*04:04:01/DRB1*13:02:01/DRB3*03:01/DRB4*01:03//DQA1*01:02/06/ |
| | | 08/09/DQA1*03:01-03/DQB1*03:02:01/DQB1*06:04:01/DPB1*10:01/ |
| | | DPB1*03:01:01 |
| xxi | | |
| xxiii | HLA-DR | DRB1*04:04:01/DRB1*13:02:01/DRB3*03:01/DRB4*01:03 |
| xxiv | HLA-DR | DRB1*04:04:01/DRB1*13:02:01/DRB3*03:01/DRB4*01:03 |
| xxv | | |
| xxvi | Indeterminate | A*01:01:01/B*51:01:01/B*40:01/C*03:04:01/C*06:02:01/DRB1*04:04:01/ |
| | | DRB1*13:02:01/DRB3*03:01/DRB4*01:03/DQA1*01:02/06/08/09/DQA1*03:01- |
| | | 03/DQB1*03:02:01/DQB1*06:04:01/DPB1*10:01/DPB1*03:01:01 |
| xxvi | HLA-DR | DRB1*01:01/DRB1*11:01/DRB3*02:02/28/29N |
| xxviii | | |
| xxix | HLA-DR | DRB1*03:01/DRB1*15:01/DRB3*01:01/04/DRB5*01:01:01 |
| xxx | | |
| xxxi | Not tested | A*24:02/A*03:01:01/B*44:02:01/B*08:01/C*07:01/C*05:01/DRB1*03:01/ |
| | | DRB1*15:01/DRB3*01:01/04/DRB5*01:01:01/DQA1*01:02/06 |
| | | /DQA1*05:01/DQB1*02:01:01/DQB1*06:02:01/DPB1*04:01:01 |
| xxxii | Not tested | DRB1*11:04:01/DRB1*04:05:01/DRB3*02:02/28/29N/DRB4*01:03/DQA1*03:01- |
| | | 03/DQA1*05:05/08/09/11/DQB1*03:01:01/DQB1*03:02:01/DPB1*04:01:01/ |
| | | DPB1*04:02:01 |
| xxxii | | |
| xxxiv | Not tested | DRB1*11:04:01/DRB1*04:05:01/DRB3*02:02/28/29N/DRB4*01:03/DQA1*03:01- |
| | | 03/DQA1*05:05/08/09/11/DQB1*03:01:01/DQB1*03:02:01/DPB1*04:01:01/ |
| | | DPB1*04:02:01 |
| xxxv | | |

| | N | O |
|---|---|---|
| | CD4/CD8 | Frequency of response |
| | responses | (% IFN* cells amongst CD4⁺ or CD8⁺ by FACS) |
| i | CD4 | 1.1 |
| ii | | |
| iii | CD4 | 3.62 |
| iv | CD4 | 0.81 |
| v | Indeterminate | Not detected |
| vi | | |
| vii | CD4 | 5.47/3.23 |
| viii | | |
| ix | CD8 | 4.77 |
| x | CD4 | 0.98 |
| xi | CD4 | 1.27 |
| xii | CD8 | 0.55 |
| xiii | CD4 | 0.36/0.24 |
| xiv | | |
| xv | CD4 | 1.33/0.89 |
| xvi | | |
| xvii | CD4 | 0.98 |
| xviii | CD4 | 0.74 |
| xix | CD8 | 2.78 |
| xx | Indeterminate | Not detected |
| xxi | CD4 | 0.325/0.45 |
| xxii | | |
| xxiii | Likely CD4 | Not detected |
| xxiv | Likely CD4 | Not detected |
| xxv | | |
| xxvi | Indeterminate | Not detected |
| xxvii | Likely CD4 | Not tested |
| xxviii | | |
| xxix | CD4 | 2.36/1.12 |
| xxx | | |
| xxxi | Indeterminate | Not detected |
| xxxii | CD4 | 0.83 |
| xxxiii | | |
| xxxiv | CD4 | 1.96/1.06 |
| xxxv | | |

### Example 3—Identifying Putative GvL Antigens in 50 Patient-Donor Pairs by Exome-Seq, RNA-Seq and Peptide Screening by IFNγ ELISpot

To identify common GvL antigens across a larger patient cohort and thus expand the repertoire of putative GvL antigens, the workflow described above (exome sequencing, RNA-Seq and in silico antigen prediction) is applied at scale to bio-banked AMADEUS trial samples. In addition to identifying multiple antigenic targets, the HLA restriction is identified in each case.

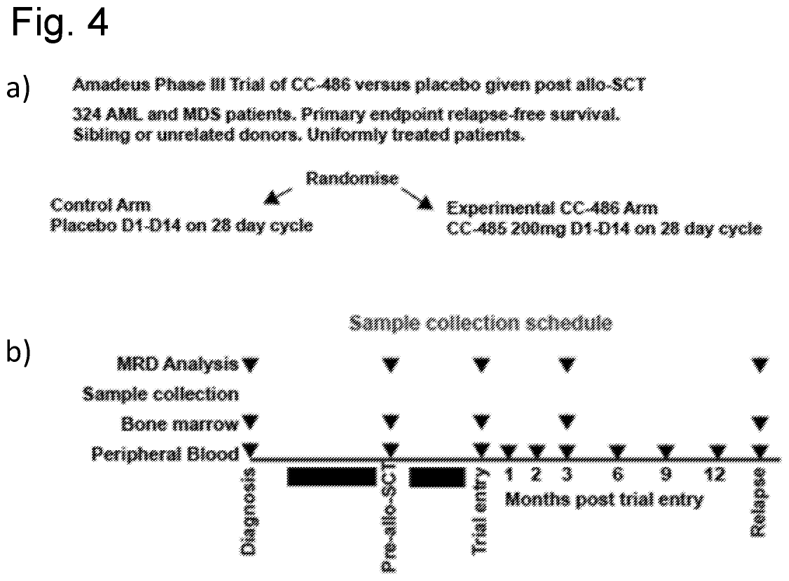

AMADEUS provides a unique opportunity to acquire samples and clinical metadata (FIG. 4). It opened for recruitment 06/2019 and is open in all UK allo-SCT transplant centres. Target recruitment is 324 patients over 3 years (108 patients recruited/year). 49 patients recruited until March 2020 (recruitment ahead of schedule). Recruitment suspended March (COVID) and re-opened in May. Projected number of relapses: year 1-21; year 2-49, year 3-59; year 4-24; year 5-10 patients.

Exome sequencing is performed 50 patient-donor pairs. For patients, AML cells are used for sequencing, with T cells as a germline control. For donors, DNA is available from NHS tissue typing centres. RNA sequencing of AML cells from each patient is also performed. Sequencing data is analysed to identify patient-donor mismatched peptides expressed on AML cells and predicted to bind patient-specific HLA molecules. These peptides are screened for their ability to activate donor-derived T cells from post-transplant blood samples, using ex vivo and sensitive cultured IFNγ ELISpot assays. Component peptides from pools that elicit ELISpot responses are individually tested to identify the peptide(s) recognised and the T cell phenotype (CD4 or CD8) and HLA restriction of the responding T cells is determined by flow cytometry analysis and antibody-blocking approaches (FIG. 3).

Patient selection is based on identifying patients most likely to have mounted a GvL response. Patient selection excludes: (i) patients where GvL is unlikely to have occurred; and (ii) patients who relapse early, within 3 months, after allo-SCT. This is because these patients go into allo-SCT with too much disease and relapse before an adaptive GvL T cell response could control disease. Patient selection includes: (i) patients who have had CC-486 (around 40 patients) and patients who have not as controls (around 10 patients; (ii) patients who go into allo-SCT with low level disease (known as measurable residual disease or MRD) that then slowly clears post allo-SCT, as disease clearance could have occurred through GvL; and (iii) patients who lose detectable MRD and then relapse late (after 12 months), as this may help to elucidate the mechanism of loss of GvL.

GvL antigens are shown to represent germline polymorphisms that could be targeted not only in AML but in multiple cancers. Given that peptide binding to HLA class II is often promiscuous, some peptides bind multiple HLA alleles.

Ultimately, we hope to develop ‘off the shelf’ engineered T cell therapies, combinations of which will be suitable for treating a large proportion of patients, based on their genetic polymorphisms and HLA types.

### Example 4—Detailed Characterisation GvL T Cell Responses and Assessment of the Role CC-486 (Oral Azacitidine) in Augmenting Immune Surveillance and Disease Control

There are multiple mechanisms by which AML could evade GvL including: reduction in HLA presentation; increased immune checkpoint molecule expression by T cells and their ligands on AML blasts; expression of immunosuppressive molecules, for example, indoleamine 2,3-dioxygenase 1 (IDO1) and alteration of the pro- and anti-inflammatory cytokine milieu. Treatment with the DNA methyltransferase inhibitor, Azacitidine (aza) post-transplant has had a beneficial clinical effect but the mechanism is unclear. One the one hand, aza may promote GvL; it upregulates expression of melanoma-associated antigen (MAGE) leading to a CD8+ T cell response. Alternatively, aza may exert a direct anti-leukaemic effect, buying time for a therapeutic GvL response to occur.

To establish how CC-486 works, patients from the Amadeus trial are studied in detail, as follows.

i. Longitudinal Tracking of Antigen-Specific Anti-AML T Cell Responses

Flow-cytometry-based methods are used to identify T cells responsive to antigenic peptides (mapped in IFNγ ELISpot assays) on the basis of activation marker upregulation, cytokine (IFNγ/TNFα) production or degranulation marker (CD107a) upregulation following peptide stimulation, and/or using fluorescent-labelled HLA-peptide tetramers. Multiparameter flow cytometry panels (read out on a Cytek Aurora spectral flow cytometer or BD Symphony, which readily enables simultaneous analysis of at least 30 parameters) are employed, to enable evaluation of naïve, memory, effector, regulatory T cells and maturation/differentiation markers (CD45RA/RO, CCR7, CD27, CD28, CD57); lineage-characteristic transcription factors (Tbet, Eomes, GATA3, RORγt, FoxP3); cytolytic effector function (perforin, granzymes A and B, Fas, FasL, TRAIL) and inhibitory receptors often co-expressed on exhausted T cells (PD-1, CTLA-4, LAG3, TIM3, TIGIT).

NK cells that can have both effector and immunomodulatory roles are also studied. Multiparameter flow cytometry-based analysis of antigen-specific T cell responses is performed on longitudinal, serial samples from each patient to track GvL T cell clonal dynamics and determine whether CC-486 promotes, sustains and amplifies GvL T cell responses, or not. This allows assessment of (i) the time post-transplant when maximal GvL clonal expansion is seen; (ii) correlation between GvL-specific T cell frequency and immunophenotype, amount of disease and clinical outcomes, CC-486 treatment, GvHD and amount of immunosuppression; (iii) GvL-specific T cell status at relapse. In addition to providing correlative data to suggest possible biological mechanisms of disease control by CC-486, it may also provide prognostic biomarker data. Loss of antigen-specific T cell clones is assessed by comparing patients who relapse post-allo-SCT with those who do not, and whether these T cells clones exhibit an exhausted phenotype.

ii. Assess how GvL Response Shapes Post-Transplant T Cell Repertoire

Bulk and single cell (sc) T cell receptor (TCR) sequencing of donor-derived AML-reactive T cells in patient's blood and bone marrow samples is performed. This is combined with whole transcriptome sequencing to get a further indication of the transcriptional state of antigen-specific T cell clones.

In a pilot experiment, we stimulated PBMCs from one patient with overlapping putative GvL peptides identified by IFNγ ELISpot mapping (see FIG. 3), or control peptides, FACS-sorted responding T cells (identified by IFNγ-secretion using a catch reagent) and successfully subjected them to parallel single cell TCR/transcriptome sequencing (10× genomics platform) (Keskin, D. B. et al. Neoantigen vaccine generates intratumoral T cell responses in phase Ib glioblastoma trial. Nature, 2019. 565 (7738): p. 234-239). Analysis of the data has enabled identification of TCRs highly enriched in the peptide-responsive population, and simultaneous interrogation of their gene expression profile. Bulk TCR sequencing was also performed on an unstimulated ex vivo sample from the same patient, to enable determination of the frequency of each peptide-responsive TCR within the in vivo T cell repertoire. This established a workflow in our laboratory that can readily be applied to the larger sample set from the AMADEUS trial.

### Single Cell Sequencing Allows:

(i) identification of paired TCRα and β chains of GvL T cell clones.
(ii) characterisation of GvL T cell phenotype from the transcriptome.
(iii) assessment of the size of antigen-specific T cell clones ex vivo and after stimulation.
   iii. Antigen-Specific T Cell-Mediated AML Cell Killing

Peptide-specific T cell clones are generated using our established single-cell cloning method (Brenna, E. et al. CD4 (+) T Follicular Helper Cells in Human Tonsils and Blood Are Clonally Convergent but Divergent from Non-Tfh CD4 (+) Cells. Cell Rep, 2020. 30 (1): p. 137-152 e) and/or donor-derived CD4 T cells are transduced with lentiviral vectors encoding paired TCRα and β chains to create antigen-specific T cell populations. These are tested using flow cytometry-based methods for in vitro activation (upregulation of CD69, CD25 and CD137), cytokine (IFNγ and TNFα production, degranulation (surface CD107a expression) and cytotoxicity (assessed by loss of fluorescent dye-labelled target cells) following incubation with patient AML cells loaded with relevant peptide (or and irrelevant peptide as a control). Antigens that are most polymorphic and patients with the most common HLA types are chosen. Cases where the T cell clones are the largest are prioritised.

AML patient-derived xenograft models in immunodeficient NSG/NSGW mice are created. In engrafting samples after establishment of AML, survival of mice and % of AML cells are compared after infusion of T cell clones, or patient T cells, transduced with either TCR recognizing a GvL antigen from that AML or recognizing a control peptide. In this way, it is possible to confirm that TCR-transduced T cells kill AML in vivo in patients using.

iv. Quantitate Residual AML Cells in Bone Marrow Samples

Sensitive flow cytometry and next generation sequencing are used to accurately quantitate AML cells that remain after treatment. This is called measurable residual disease analysis (MRD) at 2 time points (pre-trial entry, 3 months post allo-SCT). The sensitivity of this approach is 1 AML cells in 10⁵ cells.

v. Assess HLA Expression in AML Blasts

RNA-Seq performed on AML cells at diagnosis is performed at post-transplant relapse to look for loss/reduction in expression of HLA, as a mechanism of immune evasion. Altered expression of transcriptional and signaling regulators of HLA expression (e.g. CIITA, RFX5, NFYA, NFYB, IL-1A, IL-1B, IRF8), genes regulating peptide processing, and ligands for inhibitory receptors expressed on T cells and immunosuppressive factors is assessed. Cell surface HLA protein expression is also expressed by flow cytometry. This enables the study the role of CC-486 in modulating different modes of GvL failure.

### Example 5

The following protocol was used to determine the sequence of TCRs recognising the alloreactive peptide antigens set out in Table 3 above:
1. Thaw post-transplant PBMCs from the patient from whom the peptide antigen was detected. Culture the cells with peptide, IL-2 and IL-7 for 12 days.
2. Rest cells overnight.
3. Stimulate cells with peptide for 6 hours.
4. Stain cells for FACS using antibodies and IFNγ catch reagent.
5. FACS sorting of activated (IFNγ+) CD4+ or CD8+ T cells and negative control (IFNγ−) populations.
6. 10× Chromium single cell RNA sequencing with V (D) J enrichment.

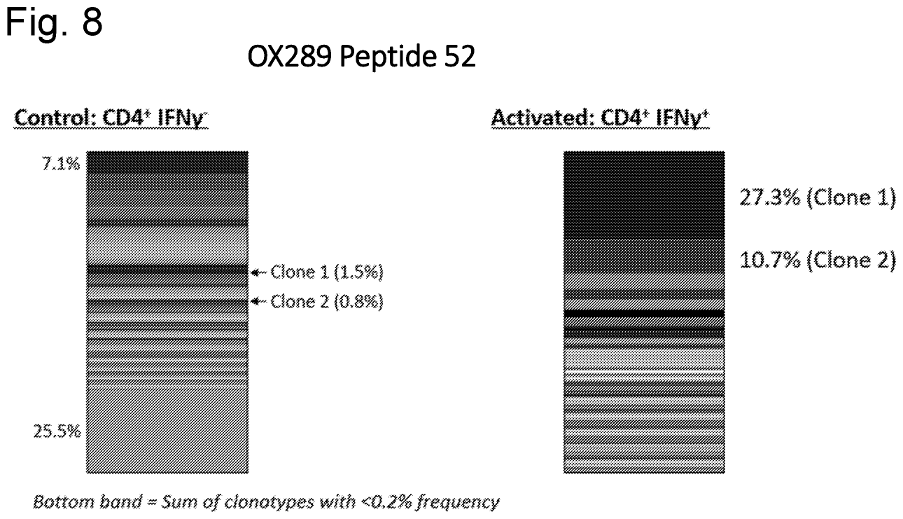

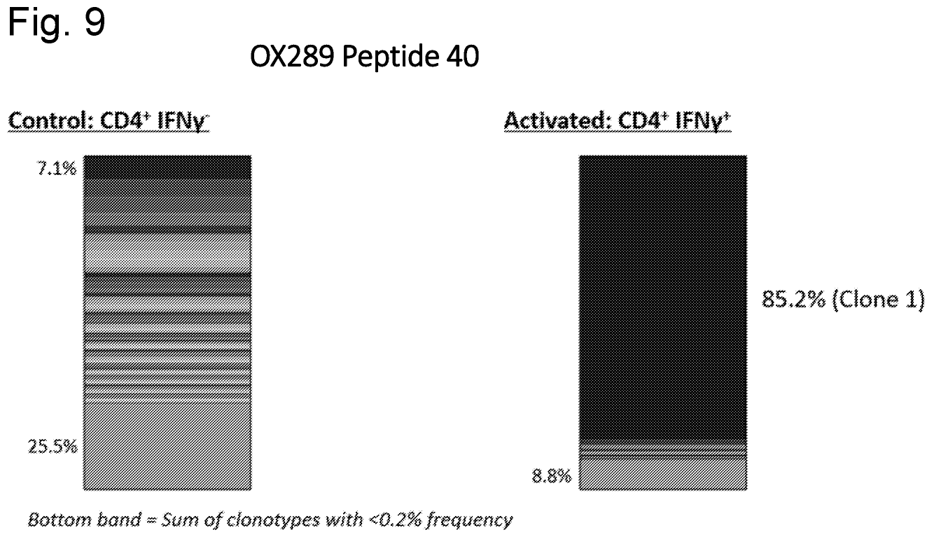

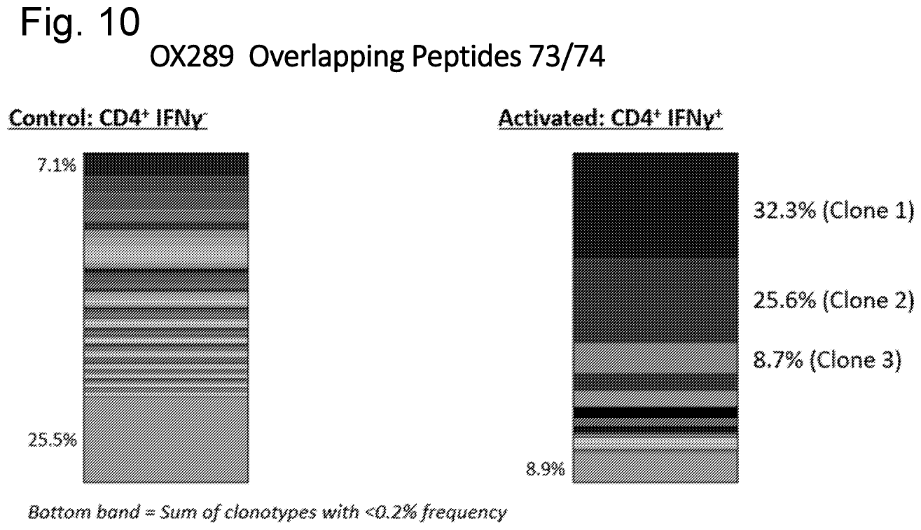

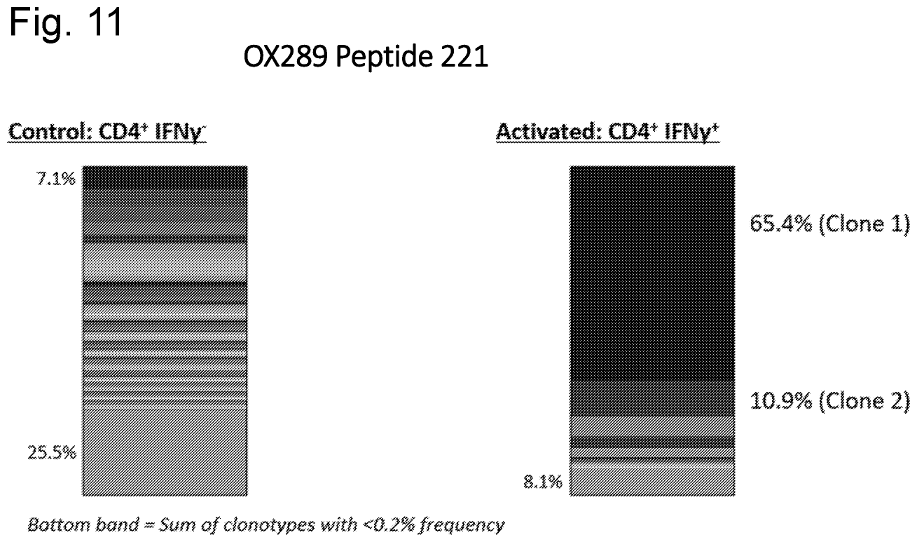

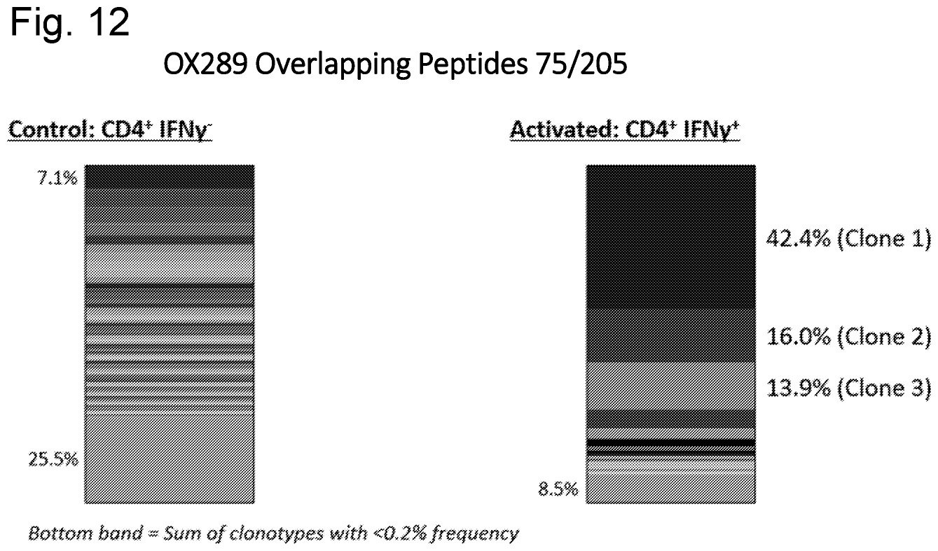

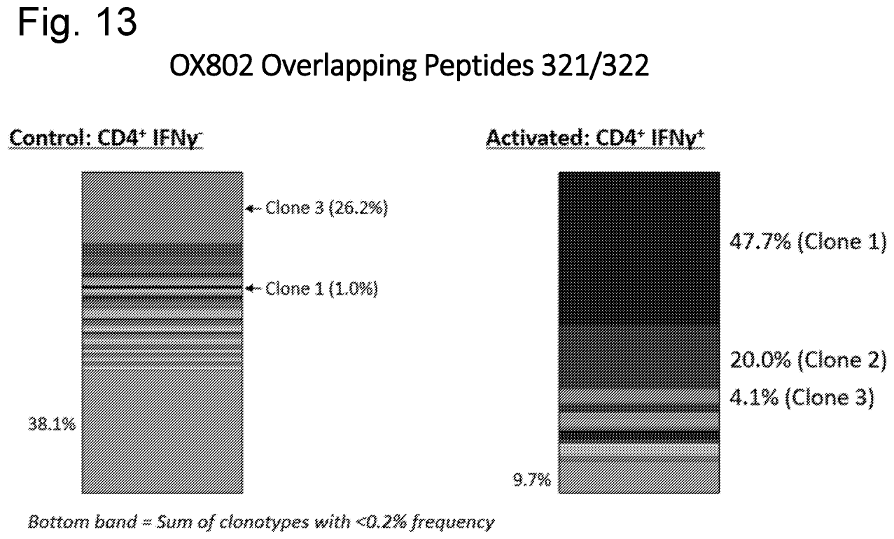

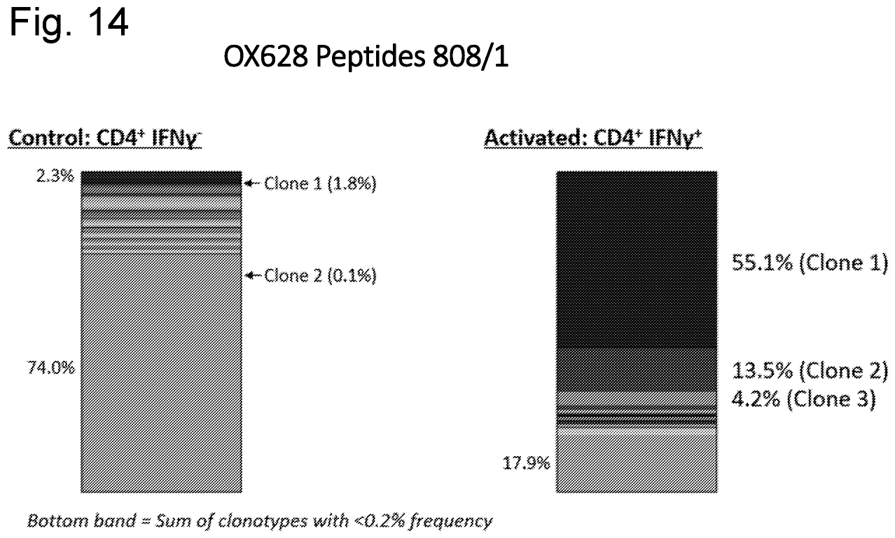

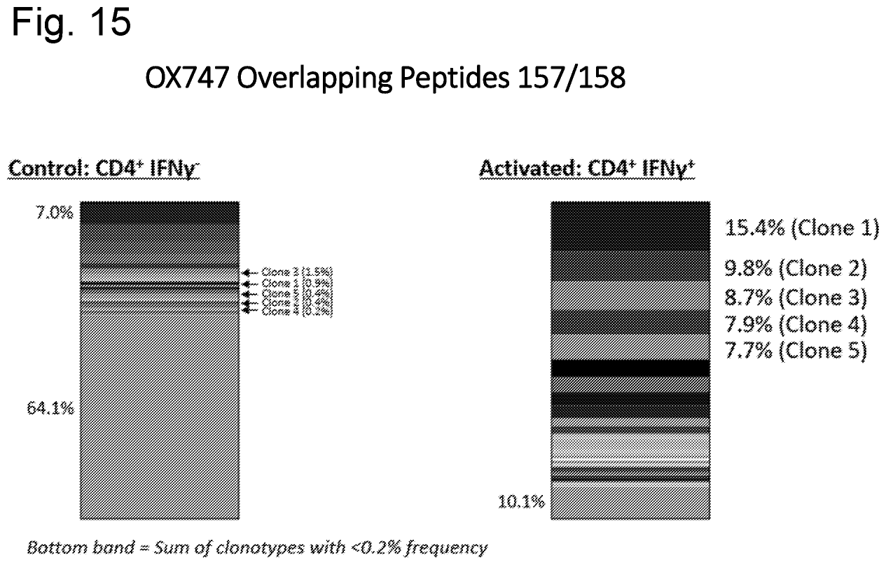

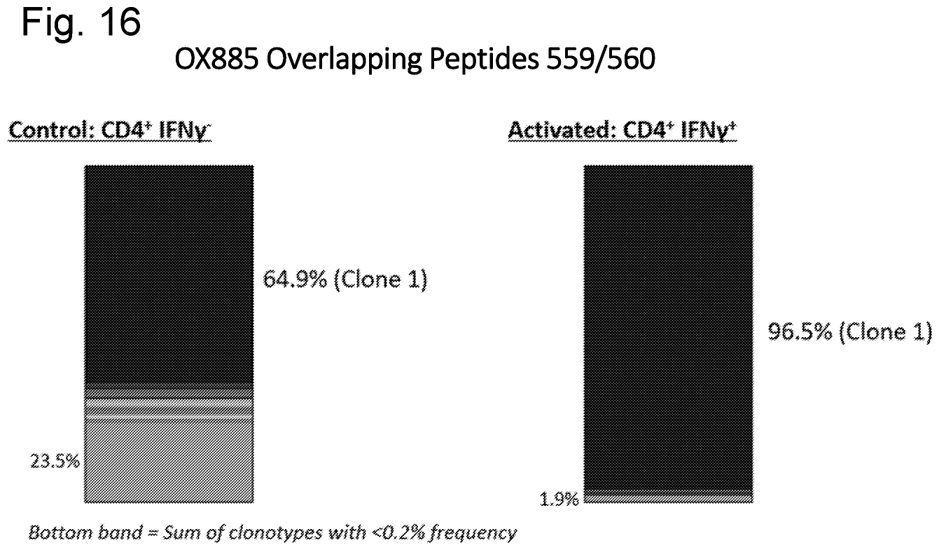

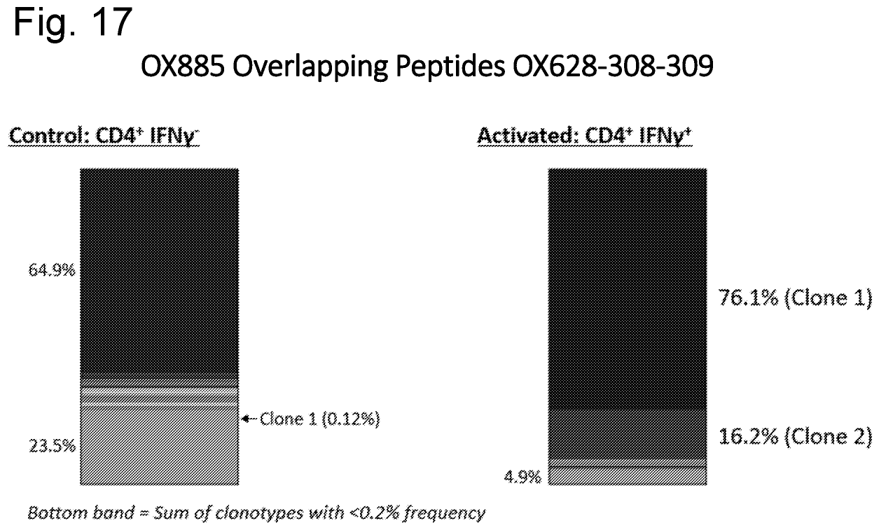

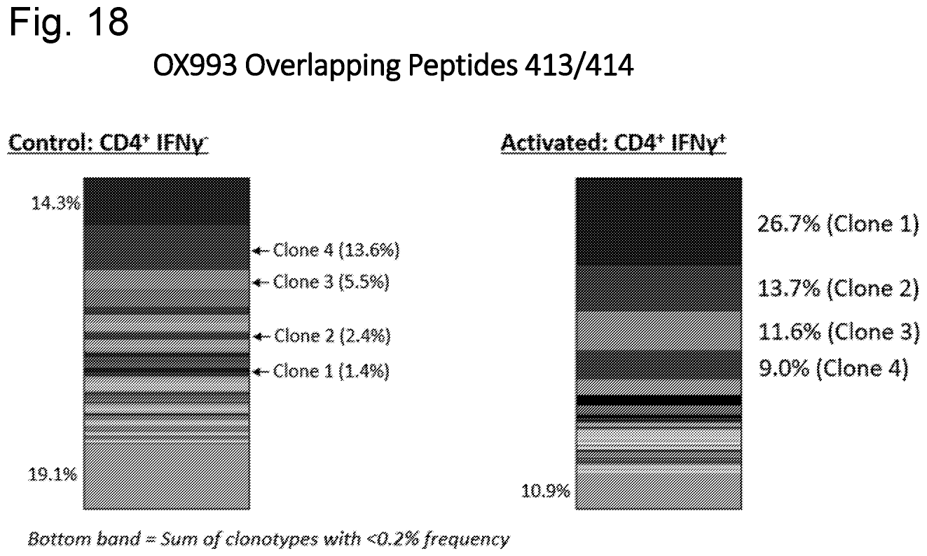

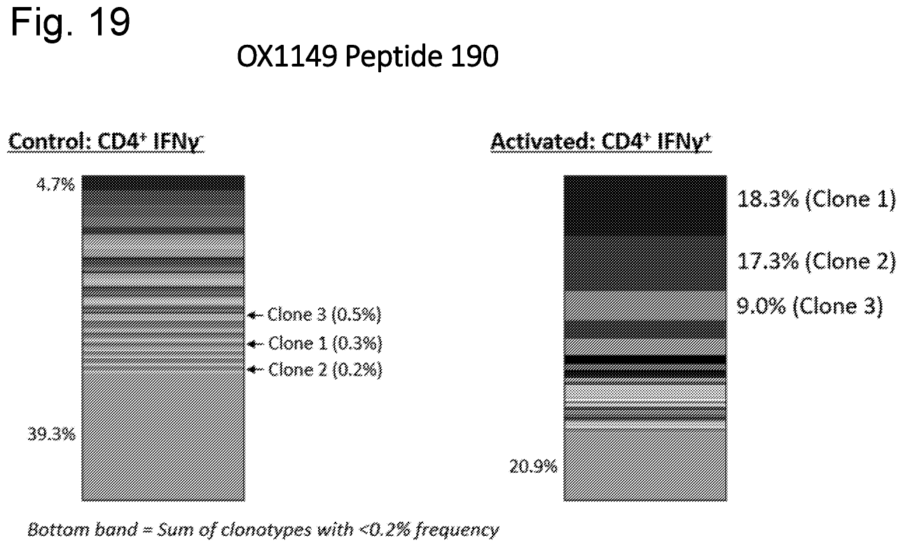

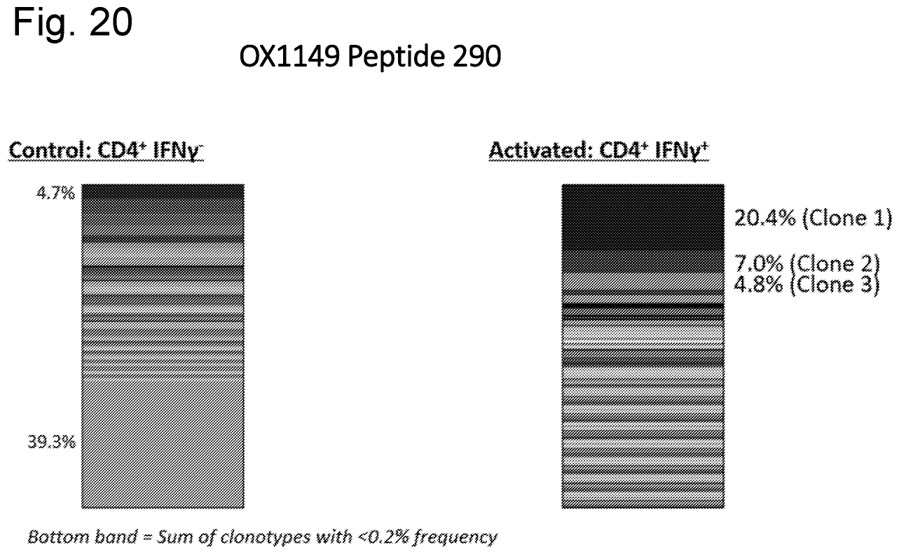

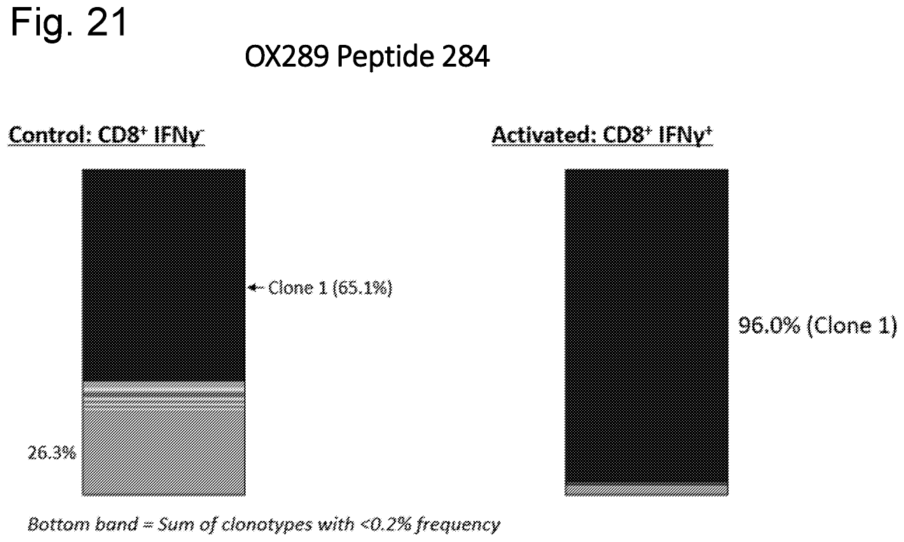

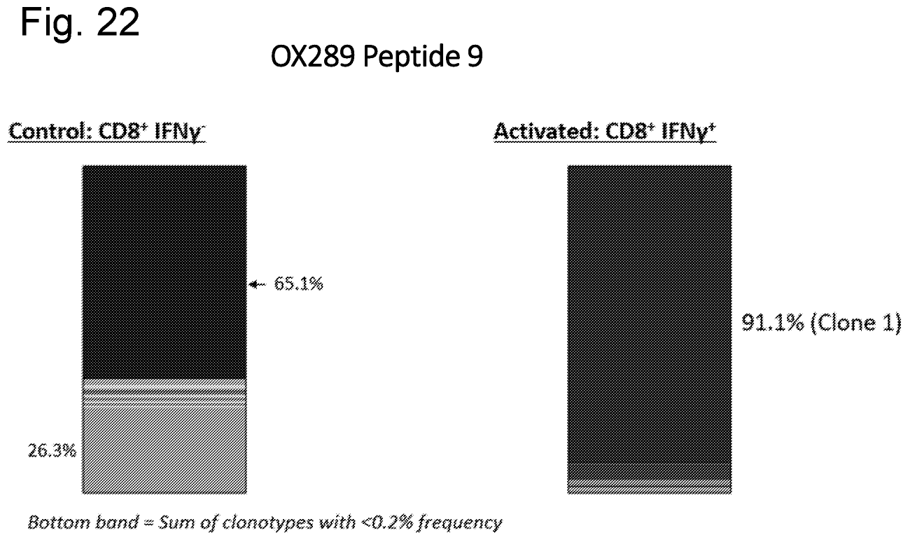

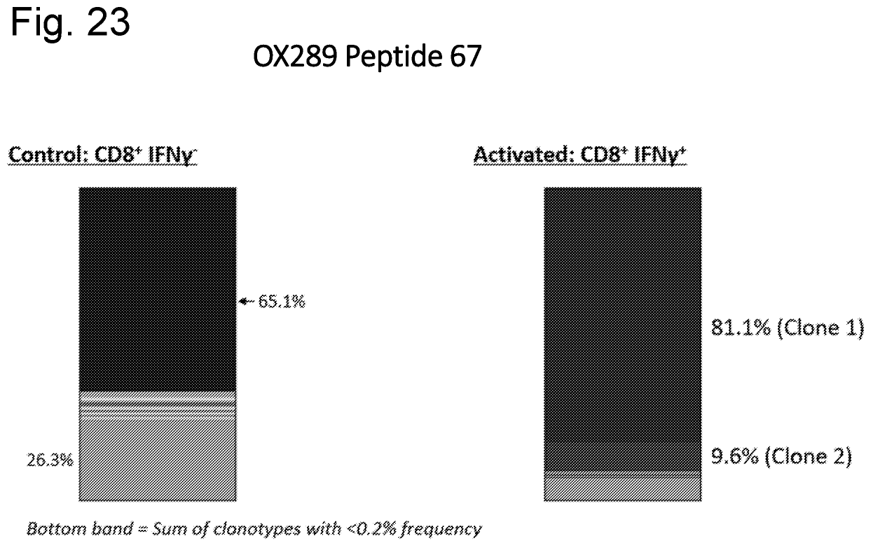

T cells recognizing 18 of the 24 peptides set out in Table 3 (including PADI4) were detectable by FACS. Of these, 17 were detectable using IFNγ catch. Data are shown in Tables 4 and 5 below, and in FIGS. 8 to 23. In Table 5, clones were included that were (a) greater in size than the largest clone in the control repertoire, or (b)>10% of the repertoire, even if not larger than the largest clone in the control repertoire.

TABLE 4
Frequencies of peptide-specific responses

| | | | | | | Freq. of response |
|---|---|---|---|---|---|---|
| | | | Allele | CD4/ | HLA | (% IFNγ⁺ cells amongst |
| Patient | Peptide(s) | Gene | frequency (%) | CD8 responses | restriction | CD4⁺ or CD8⁺ by FACS) |
| OX866 | OX866-21/22 | PADI4 | 4.27 | CD4 | HLA-DP | 1.1 |
| OX1149 | OX1149-190 | RNH1 | 11.21 | CD4 | HLA-DR | 3.62 |
| OX1149 | OX1149-290 | SLC26A6 | 9.84 | CD4 | HLA-DR | 0.81 |
| OX813 | OX813-101/102 | RHOT2 | 51.51 | Not known | Not known | Not detected |
| OX802 | OX802-321/322 | COA6 | 52.52 | CD4 | Not tested | 5.47/3.23 |
| OX289 | OX289-9 | NAGPA | 30.07 | CD8 | Class I | 4.77 |
| OX289 | OX289-040 | RNF123 | 5.07 | CD4 | HLA-DP | 0.98 |
| OX289 | OX289-052 | LLGL2 | 50.19 | CD4 | HLA-DR | 1.27 |
| OX289 | OX289-067 | ENDOD1 | 25.36 | CD8 | Not tested | 0.55 |
| OX289 | OX289-073/074 | HENMT1 | 1.93 | CD4 | Not tested | 0.36/0.24 |
| OX289 | OX289-075/205 | ACADS | 26.58 | CD4 | Not tested | 1.33/0.89 |
| OX289 | OX289-180 | TIMM23B | 9.11 | CD4 | HLA-DR | 0.98 |
| OX289 | OX289-221 | LRR1 | 20.48 | CD4 | HLA-DP | 0.74 |
| OX289 | OX289-284 | FTSJ3 | 64.78 | CD8 | Class I | 2.78 |
| OX289 | OX1098-614 | LGALS8 | 72.36 | Likely CD4 | HLA-DP | Not detected |
| OX747 | OX747-157/158 | TMBIM4 | 43.73 | CD4 | Not tested | 0.325/0.45 |
| OX747 | OX747-185 | DOCK8 | 24.54 | Likely CD4 | HLA-DR | Not detected |
| OX747 | OX747-190/191 | METTL22 | 22.2 | Likely CD4 | HLA-DR | Not detected |
| OX747 | OX628-305 | DCAF13 | 23.64 | Not known | Not known | Not detected |
| OX993 | OX993-413/414 | DENND6B | 31.06 | CD4 | HLA-DR | 0.4 |
| OX628 | OX628-80/81 | LITAF | 0.00122 | CD4 | HLA-DR | 2.36/1.12 |
| OX628 | OX628-564 | PGLS | 0.08 | Not known | Not tested | Not detected |
| OX885 | OX885-559/560 | GSTZ1 | 30.57 | CD4 | Not tested | 0.83 |
| OX885 | OX628-308/309 | WASHC4 | 94.67 | CD4 | Not tested | 1.96/1.06 |

TABLE 5
TCR sequences from expanded T cell clones following peptide stimulation

| | | | | | | | | | | | | | | | Full | Full |
|---|---|---|---|---|---|---|---|---|---|---|---|---|---|---|---|---|
| | | | Clone | | | | | | | | | | | CDR3 | con- | con- |
| | | | size | | | | | | | | | | | nt | sensus | sensus |
| | CD4 | | (percent | | | | | | | | | | | (SEQ | aa | nt |
| | or | | re- | | | | | | CDR1 aa | CDR1 nt | CDR2 aa | CDR2 nt | CDR3 aa | ID | (SEQ | SEQ |
| Peptide | CD8 | Clone | pertoire) | Chain | V gene | D gene | J gene | C gene | (SEQ ID NO) | (SEQ ID NO) | (SEQ ID NO) | (SEQ ID NO) | (SEQ ID NO) | NO) | ID NO) | ID NO) |
| OX289 | CD4 | 1 | 85.2 | TRA | TRAV9-2 | | TRAJ10 | TRAC | 448 | 535 | 622 | 709 | 32 | 119 | 206 | 293 |
| Peptide 40 | | | | TRB | TRBV20-1 | | TRBJ2-2 | TRBC2 | 449 | 536 | 623 | 710 | 33 | 120 | 207 | 294 |
| OX289 | CD4 | 1 | 27.3 | TRA | TRAV8-3 | | TRAJ3 | TRAC | 450 | 537 | 624 | 711 | 34 | 121 | 208 | 295 |
| Peptide 52 | | | | TRB | TRBV2 | TRBD1 | TRBJ2-6 | TRBC2 | 451 | 538 | 625 | 712 | 35 | 122 | 209 | 296 |
| | | | | TRB | TRBV20-1 | | TRBJ2-2 | TRBC2 | 452 | 539 | 626 | 713 | 36 | 123 | 210 | 297 |
| | | 2 | 10.7 | TRA | TRAV36/DV7 | | TRAJ22 | TRAC | 453 | 540 | 627 | 714 | 37 | 124 | 211 | 298 |
| | | | | TRB | TRBV12-3 | | TRBJ2-1 | TRBC2 | 454 | 541 | 628 | 715 | 38 | 125 | 212 | 299 |
| OX289 | CD4 | 1 | 32.3 | TRA | TRAV5 | | TRAJ16 | TRAC | 455 | 542 | 629 | 716 | 39 | 126 | 213 | 300 |
| Peptides | | | | TRB | TRBV27 | | TRBJ2-1 | TRBC2 | 456 | 543 | 630 | 717 | 40 | 127 | 214 | 301 |
| 73/74 | | 2 | 25.6 | TRA | TRAV39 | | TRAJ58 | TRAC | 457 | 544 | 631 | 718 | 41 | 128 | 215 | 302 |
| | | | | TRB | TRBV27 | | TRBJ2-1 | TRBC2 | 458 | 545 | 632 | 719 | 42 | 129 | 216 | 303 |
| | | | | TRB | TRBV27 | TRBD1 | TRBJ1-1 | TRBC1 | 459 | 546 | 633 | 720 | 43 | 130 | 217 | 304 |
| | | 3 | 8.67 | TRA | TRAV8-6 | | TRAJ56 | TRAC | 460 | 547 | 634 | 721 | 44 | 131 | 218 | 305 |
| | | | | TRB | TRBV7-2 | | TRBJ2-1 | TRBC2 | 461 | 548 | 635 | 722 | 45 | 132 | 219 | 306 |
| | | | | TRB | TRBV7-2 | | TRBJ2-1 | TRBC2 | 462 | 549 | 636 | 723 | 46 | 133 | 220 | 307 |
| OX289 | CD4 | 1 | 42.4 | TRA | TRAV9-2 | | TRAJ17 | TRAC | 463 | 550 | 637 | 724 | 47 | 134 | 221 | 308 |
| Peptides | | | | TRA | TRAV23/DV6 | | TRAJ42 | TRAC | 464 | 551 | 638 | 725 | 48 | 135 | 222 | 309 |
| 75/205 | | | | TRB | TRBV20-1 | | TRBJ1-4 | TRBC1 | 465 | 552 | 639 | 726 | 49 | 136 | 223 | 310 |
| | | 2 | 16 | TRA | TRAV12-2 | | TRAJ41 | TRAC | 466 | 553 | 640 | 727 | 50 | 137 | 224 | 311 |
| | | | | TRB | TRBV6-5 | TRBD2 | TRBJ2-5 | \|TRBC2 | 467 | 554 | 641 | 728 | 51 | 138 | 225 | 312 |
| | | 3 | 13.9 | TRA | TRAV9-2 | | TRAJ23 | TRAC | 468 | 555 | 642 | 729 | 52 | 139 | 226 | 313 |
| | | | | TRB | TRBV20-1 | | TRBJ2-1 | TRBC2 | 469 | 556 | 643 | 730 | 53 | 140 | 227 | 314 |
| OX289 | CD4 | 1 | 65.4 | TRA | TRAV4 | | TRAJ23 | TRAC | 470 | 557 | 644 | 731 | 54 | 141 | 228 | 315 |
| Peptide | | | | TRB | TRBV5-1 | TRBD2 | TRBJ2-5 | TRBC2 | 471 | 558 | 645 | 732 | 55 | 142 | 229 | 316 |
| 221 | | 2 | 10.9 | TRA | TRAV26-2 | | TRAJ57 | TRAC | 472 | 559 | 646 | 733 | 56 | 143 | 230 | 317 |
| | | | | TRB | TRBV20-1 | | TRBJ1-5 | TRBC1 | 473 | 560 | 647 | 734 | 57 | 144 | 231 | 318 |
| OX802 | CD4 | 1 | 47.4 | TRA | TRAV1-1 | | TRAJ41 | TRAC | 474 | 561 | 648 | 735 | 58 | 145 | 232 | 319 |
| Peptides | | | | TRA | TRAV2 | | TRAJ4 | TRAC | 475 | 562 | 649 | 736 | 59 | 146 | 233 | 320 |
| 321/322 | | | | TRB | TRBV11-3 | | TRBJ2-7 | TRBC2 | 476 | 563 | 650 | 737 | 60 | 147 | 234 | 321 |
| | | 2 | 20 | TRA | TRAV2 | | TRAJ4 | TRAC | 477 | 564 | 651 | 738 | 61 | 148 | 235 | 322 |
| | | | | TRB | TRBV11-3 | | TRBJ2-7 | TRBC2 | 478 | 565 | 652 | 739 | 62 | 149 | 236 | 323 |
| OX628 | CD4 | 1 | 55.1 | TRA | TRAV35 | | TRAJ54 | TRAC | 479 | 566 | 653 | 740 | 63 | 150 | 237 | 324 |
| Peptides | | | | TRB | TRBV6-6 | | TRBJ1-1 | TRBC1 | 480 | 567 | 654 | 741 | 64 | 151 | 238 | 325 |
| 80/81 | | 2 | 13.5 | TRA | TRAV35 | | TRAJ31 | TRAC | 481 | 568 | 655 | 742 | 65 | 152 | 239 | 326 |
| | | | | TRB | TRBV29-1 | | TRBJ1-1 | TRBC1 | 482 | 569 | 656 | 743 | 66 | 153 | 240 | 327 |
| | | 3 | 4.2 | TRA | TRAV12-2 | | TRAJ52 | TRAC | 483 | 570 | 657 | 744 | 67 | 154 | 241 | 328 |
| | | | | TRB | TRBV3-1 | | TRBJ2-7 | TRBC2 | 484 | 571 | 658 | 745 | 68 | 155 | 242 | 329 |
| OX747 | CD4 | 1 | 15.5 | TRA | TRAV41 | | TRAJ32 | TRAC | 485 | 572 | 659 | 746 | 69 | 156 | 243 | 330 |
| Peptides | | | | TRA | TRAV41 | | TRAJ58 | TRAC | 486 | 573 | 660 | 747 | 70 | 157 | 244 | 331 |
| 157/158 | | | | TRB | TRBV6-5 | | TRBJ1-3 | TRBC1 | 487 | 574 | 661 | 748 | 71 | 158 | 245 | 332 |
| | | 2 | 9.8 | TRA | TRAV19 | | TRAJ34 | TRAC | 488 | 575 | 662 | 749 | 72 | 159 | 246 | 333 |
| | | | | TRB | TRBV5-4 | | TRBJ1-2 | TRBC1 | 489 | 576 | 663 | 750 | 73 | 160 | 247 | 334 |
| | | 3 | 8.7 | TRA | TRAV12-2 | | TRAJ50 | TRAC | 490 | 577 | 664 | 751 | 74 | 161 | 248 | 335 |
| | | | | TRB | TRBV24-1 | | TRBJ1-2 | TRBC1 | 491 | 578 | 665 | 752 | 75 | 162 | 249 | 336 |
| | | 4 | 7.9 | TRA | TRAV19 | | TRAJ30 | TRAC | 492 | 579 | 666 | 753 | 76 | 163 | 250 | 337 |
| | | | | TRB | TRBV18 | TRBD1 | TRBJ1-3 | TRBC1 | 493 | 580 | 667 | 754 | 77 | 164 | 251 | 338 |
| | | 5 | 7.7 | TRA | TRAV35 | | TRAJ58 | TRAC | 494 | 581 | 668 | 755 | 78 | 165 | 252 | 339 |
| | | | | TRB | TRBV19 | TRBD1 | TRBJ1-5 | TRBC1 | 495 | 582 | 669 | 756 | 79 | 166 | 253 | 340 |
| OX885 Peptides 559/560 | CD4 | 1 | 96.5 | TRA | TRAV22 | | TRAJ43 | TRAC | 496 | 583 | 670 | 757 | 80 | 167 | 254 | 341 |
| | | | | TRB | TRBV12-5 | | TRBJ1-5 | TRBC1 | 497 | 584 | 671 | 758 | 81 | 168 | 255 | 342 |
| OX885 | CD4 | 1 | 76.1 | TRA | TRAV26-1 | | TRAJ27 | TRAC | 498 | 585 | 672 | 759 | 82 | 169 | 256 | 343 |
| Peptides | | | | TRB | TRBV27 | | TRBJ2-3 | TRBC2 | 499 | 586 | 673 | 760 | 83 | 170 | 257 | 344 |
| OX628- | | 2 | 16.2 | TRA | TRAV8-6 | | TRAJ10 | TRAC | 500 | 587 | 674 | 761 | 84 | 171 | 258 | 345 |
| 308/309 | | | | TRB | TRBV10-2 | TRBD1 | TRBJ1-5 | TRBC1 | 501 | 588 | 675 | 762 | 85 | 172 | 259 | 346 |
| OX993 | CD4 | 1 | 26.7 | TRA | TRAV13-2 | | TRAJ28 | TRAC | 502 | 589 | 676 | 763 | 86 | 173 | 260 | 347 |
| Peptides | | | | TRA | TRAV13-2 | | TRAJ12 | TRAC | 503 | 590 | 677 | 764 | 87 | 174 | 261 | 348 |
| 413/414 | | | | TRB | TRBV7-9 | | TRBJ1-1 | TRBC1 | 504 | 591 | 678 | 765 | 88 | 175 | 262 | 349 |
| | | 2 | 13.7 | TRA | TRAV8-4 | | TRAJ8 | TRAC | 505 | 592 | 679 | 766 | 89 | 176 | 263 | 350 |
| | | | | TRB | TRBV6-5 | | TRBJ2-2 | TRBC2 | 506 | 593 | 680 | 767 | 90 | 177 | 264 | 351 |
| | | | | TRB | TRBV6-5 | | TRBJ1-6 | TRBC1 | 507 | 594 | 681 | 768 | 91 | 178 | 265 | 352 |
| | | 3 | 11.6 | TRA | TRAV5 | | TRAJ9 | TRAC | 508 | 595 | 682 | 769 | 92 | 179 | 266 | 353 |
| | | | | TRB | TRBV6-5 | | TRBJ2-2 | TRBC2 | 509 | 596 | 683 | 770 | 93 | 180 | 267 | 354 |
| OX1149 | CD4 | 1 | 18.3 | TRA | TRAV17 | | TRAJ17 | TRAC | 510 | 597 | 684 | 771 | 94 | 181 | 268 | 355 |
| Peptide | | | | TRB | TRBV2 | | TRBJ2-2 | TRBC2 | 511 | 598 | 685 | 772 | 95 | 182 | 269 | 356 |
| 190 | | 2 | 17.3 | TRA | TRAV17 | | TRAJ45 | TRAC | 512 | 599 | 686 | 773 | 96 | 183 | 270 | 357 |
| | | | | TRB | TRBV2 | | TRBJ2-7 | \|TRBC2 | 513 | 600 | 687 | 774 | 97 | 184 | 271 | 358 |
| | | 3 | 9 | TRA | TRAV13-1 | | TRAJ53 | TRAC | 514 | 601 | 688 | 775 | 98 | 185 | 272 | 359 |
| | | | | TRB | TRBV12-4 | TRBD1 | TRBJ2-7 | TRBC2 | 515 | 602 | 689 | 776 | 99 | 186 | 273 | 360 |
| | | 4 | 5.7 | TRA | TRAV17 | | TRAJ7 | TRAC | 516 | 603 | 690 | 777 | 100 | 187 | 274 | 361 |
| | | | | TRB | TRBV2 | | TRBJ1-2 | TRBC1 | 517 | 604 | 691 | 778 | 101 | 188 | 275 | 362 |
| | | 5 | 4.8 | TRA | TRAV29/DV5 | | TRAJ52 | TRAC | 518 | 605 | 692 | 779 | 102 | 189 | 276 | 363 |
| | | | | TRB | TRBV30 | | TRBJ2-5 | TRBC2 | 519 | 606 | 693 | 780 | 103 | 190 | 277 | 364 |
| OX1149 | CD4 | 1 | 20.4 | TRA | TRAV13-1 | | TRAJ34 | TRAC | 520 | 607 | 694 | 781 | 104 | 191 | 278 | 365 |
| Peptide | | | | TRB | TRBV5-1 | TRBD1 | TRBJ1-2 | TRBC1 | 521 | 608 | 695 | 782 | 105 | 192 | 279 | 366 |
| 290 | | 2 | 7 | TRA | TRAV10 | | TRAJ36 | TRAC | 522 | 609 | 696 | 783 | 106 | 193 | 280 | 367 |
| | | | | TRB | TRBV5-1 | | TRBJ2-3 | TRBC2 | 523 | 610 | 697 | 784 | 107 | 194 | 281 | 368 |
| | | 3 | 4.8 | TRA | TRAV26-2 | | TRAJ48 | TRAC | 524 | 611 | 698 | 785 | 108 | 195 | 282 | 369 |
| | | | | TRA | TRAV34 | | TRAJ42 | TRAC | 525 | 612 | 699 | 786 | 109 | 196 | 283 | 370 |
| | | | | TRB | TRBV2 | | TRBJ1-1 | RBC1 | 526 | 613 | 700 | 787 | 110 | 197 | 284 | 371 |
| OX289 | CD8 | 1 | 91.1 | TRA | TRAV12-1 | | TRAJ40 | TRAC | 527 | 614 | 701 | 788 | 111 | 198 | 285 | 372 |
| Peptide 9 | | | | TRB | TRBV20-1 | TRBD1 | TRBJ1-2 | TRBC1 | 528 | 615 | 702 | 789 | 112 | 199 | 286 | 373 |
| OX289 | CD8 | 1 | 81.1 | TRA | TRAV27 | | TRAJ30 | TRAC | 529 | 616 | 703 | 790 | 113 | 200 | 287 | 374 |
| Peptide 67 | | | | TRA | TRAV22 | | TRAJ33 | TRAC | 530 | 617 | 704 | 791 | 114 | 201 | 288 | 375 |
| | | | | TRB | TRBV11-2 | | TRBJ2-1 | TRBC2 | 531 | 618 | 705 | 792 | 115 | 202 | 289 | 376 |
| OX289 | CD8 | 1 | 96 | TRA | TRAV12-1 | | TRAJ40 | TRAC | 532 | 619 | 706 | 793 | 116 | 203 | 290 | 377 |
| Peptide 284 | | | | TRB | TRBV20-1 | TRBD1 | TRBJ1-2 | TRBC1 | 533 | 620 | 707 | 794 | 117 | 204 | 291 | 378 |
| | | | | TRB | TRBV20-1 | | TRBJ2-2 | TRBC2 | 534 | 621 | 708 | 795 | 118 | 205 | 292 | 379 |

TABLE OF
SEQUENCES

| SEQ | |
|---|---|
| ID | |
| NO: | Sequence |
| 1 | LTISLLDTFNLELPEAVVFQ |
| 2 | ISLLDTFNLELPEAVVFQDS |
| 3 | SLLDTFNLELPEAVVF |
| 4 | LDTFNLELPEAVVFQ |
| 5 | CAGAAGCCTCACACAGCCCAGTAACTTTGCTAGTACCTCTTGAGTGCAAGGTGGAGAATTAAGATCT |
| | GGATTTGAGACGGAGCACGGAACATTTCACTCAGGGGAAGAGCTATGAACATGCTGACTGCCAGCC |
| | TGTTGAGGGCAGTCATAGCCTCCATCTGTGTTGTATCCAGCATGGCTCAGAAGGTAACTCAAGCGCA |
| | GACTGAAATTTCTGTGGTGGAGAAGGAGGATGTGACCTTGGACTGTGTGTATGAAACCCGTGATACT |
| | ACTTATTACTTATTCTGGTACAAGCAACCACCAAGTGGAGAATTGGTTTTCCTTATTCGTCGGAACTC |
| | TTTTGATGAGCAAAATGAAATAAGTGGTCGGTATTCTTGGAACTTCCAGAAATCCACCAGTTCCTTCA |
| | ACTTCACCATCACAGCCTCACAAGTCGTGGACTCAGCAGTATACTTCtgtgctctgagtgaacggccgtatggtggt |
| | gctacaaacaagctcatctttGGAACTGGCACTCTGCTTGCTGTCCAGCCAAATATCCAGAACCCTGACCCTGC |
| | CGTGTACCAGCTGAGAGACTCTAAATCCAGTGACAAGTCTGTCTGCCTATTCACCGATTTTGATTCTC |
| | AAACAAATGTGTCACAAAGTAAGGATTCTGATGTGTATATCACAGACAAAACTGTGCTAGACATGAGG |
| | TCTATGGACTTCAAGAGCAACAGTGCTGTGGCCTGGAGCAACAAATCTGACTTTGCATGTGCAAACG |
| | CCTTCA |
| 6 | MLTASLLRAVIASICVVSSMAQKVTQAQTEISVVEKEDVTLDCVYETRDTTYYLFWYKQPPSGELVFLIRR |
| | NSFDEQNEISGRYSWNFQKSTSSFNFTITASQVVDSAVYFcalserpyggatnklifGTGTLLAVQPNIQNPDPAV |
| | YQLRDSKSSDKSVCLFTDFDSQTNVSQSKDSDVYITDKTVLDMRSMDFKSNSAVAWSNKSDFACANAF |
| 7 | AAAATGCCCCTCCTTTCCTCCACAGGACCAGATGCCTGAGCTAGGAAAGGCCTCATTCCTGCTGTGA |
| | TCCTGCCATGGATACCTGGCTCGTATGCTGGGCAATTTTTAGTCTCTTGAAAGCAGGACTCACAGAA |
| | CCTGAAGTCACCCAGACTCCCAGCCATCAGGTCACACAGATGGGACAGGAAGTGATCTTGCGCTGT |
| | GTCCCCATCTCTAATCACTTATACTTCTATTGGTACAGACAAATCTTGGGGCAGAAAGTCGAGTTTCT |
| | GGTTTCCTTTTATAATAATGAAATCTCAGAGAAGTCTGAAATATTCGATGATCAATTCTCAGTTGAAAG |
| | GCCTGATGGATCAAATTTCACTCTGAAGATCCGGTCCACAAAGCTGGAGGACTCAGCCATGTACTTCt |
| | gtgccagcagtgaacgcaggacgcaacctgcctacgagcagtacttcGGGCCGGGCACCAGGCTCACGGTCACAGAGGA |
| | CCTGAAAAACGTGTTCCCACCCGAGGTCGCTGTGTTTGAGCCATCAGAAGCAGAGATCTCCCACAC |
| | CCAAAAGGCCACACTGGTATGCCTGGCCACAGGCTTCTACCCCGACCACGTGGAGCTGAGCTGGTG |
| | GGTGAATGGGAAGGAGGTGCACAGTGGGGTCAGCACAGACCCGCAGCCCCTCAAGGAGCAGCCC |
| | GCCCTCAATGACTCCAGATACTGCCTGAGCAGCCGCCTGAGGGTCTCGGCCACCTTCTGGCAGAAC |
| | CCCCGCAACCACTTCCGCTGTCAAGTCCAGTTCTACGGGCTCTCGGAGAATGACGAGTGG |
| | MDTWLVCWAIFSLLKAGLTEPEVTQTPSHQVTQMGQEVILRCVPISNHLYFYWYRQILGQKVEFLVSFYN |
| | NEISEKSEIFDDQFSVERPDGSNFTLKIRSTKLEDSAMYFcasserrtqpayeqyfGPGTRLTVTEDLKNVFPPEV |
| | AVFEPSEAEISHTQKATLVCLATGFYPDHVELSWWVNGKEVHSGVSTDPQPLKEQPALNDSRYCLSSRL |
| | RVSATFWQNPRNHFRCQVQFYGLSENDEW |
| 9 | AAAGCAGATTCTTTTTATGATTTTTAAAGTAGAAATATCCATTCTAGGTGCATTTTTTAAGGGTTTAAAA |
| | TTTGAATCCTCAGTGAACCAGGGCAGAGAAGAATGATGAAATCCTTGAGAGTTTTACTAGTGATCCT |
| | GTGGCTTCAGTTGAGCTGGGTTTGGAGCCAACAGAAGGAGGTGGAGCAGAATTCTGGACCCCTCAG |
| | TGTTCCAGAGGGAGCCATTGCCTCTCTCAACTGCACTTACAGTGACCGAGGTTCCCAGTCCTTCTTC |
| | TGGTACAGACAATATTCTGGGAAAAGCCCTGAGTTGATAATGTCCATATACTCCAATGGTGACAAAGA |
| | AGATGGAAGGTTTACAGCACAGCTCAATAAAGCCAGCCAGTATGTTTCTCTGCTCATCAGAGACTCC |
| | CAGCCCAGTGATTCAGCCACCTACCTCtgtgccgtccgggggcaggcaggaactgctctgatotttGGGAAGGGAACCA |
| | CCTTATCAGTGAGTTCCAATATCCAGAACCCTGACCCTGCCGTGTACCAGCTGAGAGACTCTAAATC |
| | CAGTGACAAGTCTGTCTGCCTATTCACCGATTTTGATTCTCAAACAAATGTGTCACAAAGTAAGGATT |
| | CTGATGTGTATATCACAGACAAAACTGTGCTAGACATGAGGTCTATGGACTTCAAGAGCAACAGTGC |
| | TGTGGCCTGGAGCAACAAATCTGACTTTGCATGTGCAAACGCCTTCA |
| 10 | MKSLRVLLVILWLQLSWVWSQQKEVEQNSGPLSVPEGAIASLNCTYSDRGSQSFFWYRQYSGKSPELIM |
| | SIYSNGDKEDGRFTAQLNKASQYVSLLIRDSQPSDSATYLcavrgqagtalifGKGTTLSVSSNIQNPDPAVYQL |
| | RDSKSSDKSVCLFTDFDSQTNVSQSKDSDVYITDKTVLDMRSMDFKSNSAVAWSNKSDFACANAF |
| 11 | CAGAAGCCTCACACAGCCCAGTAACTTTGCTAGTACCTCTTGAGTGCAAGGTGGAGAATTAAGATCT |
| | GGATTTGAGACGGAGCACGGAACATTTCACTCAGGGGAAGAGCTATGAACATGCTGACTGCCAGCC |
| | TGTTGAGGGCAGTCATAGCCTCCATCTGTGTTGTATCCAGCATGGCTCAGAAGGTAACTCAAGCGCA |
| | GACTGAAATTTCTGTGGTGGAGAAGGAGGATGTGACCTTGGACTGTGTGTATGAAACCCGTGATACT |
| | ACTTATTACTTATTCTGGTACAAGCAACCACCAAGTGGAGAATTGGTTTTCCTTATTCGTCGGAACTC |
| | TTTTGATGAGCAAAATGAAATAAGTGGTCGGTATTCTTGGAACTTCCAGAAATCCACCAGTTCCTTCA |
| | ACTTCACCATCACAGCCTCACAAGTCGTGGACTCAGCAGTATACTTCtgtgctctcccggcaggaaacacacctctt |
| | gtctttGGAAAGGGCACAAGACTTTCTGTGATTGCAAATATCCAGAACCCTGACCCTGCCGTGTACCAG |
| | CTGAGAGACTCTAAATCCAGTGACAAGTCTGTCTGCCTATTCACCGATTTTGATTCTCAAACAAATGT |
| | GTCACAAAGTAAGGATTCTGATGTGTATATCACAGACAAAACTGTGCTAGACATGAGGTCTATGGACT |
| | TCAAGAGCAACAGTGCTGTGGCCTGGAGCAACAAATCTGACTTTGCATGTGCAAACGCCTTCA |
| 12 | MLTASLLRAVIASICVVSSMAQKVTQAQTEISVVEKEDVTLDCVYETRDTTYYLFWYKQPPSGELVFLIRR |
| | NSFDEQNEISGRYSWNFQKSTSSFNFTITASQVVDSAVYFcalpagntplvfGKGTRLSVIANIQNPDPAVYQL |
| | RDSKSSDKSVCLFTDFDSQTNVSQSKDSDVYITDKTVLDMRSMDFKSNSAVAWSNKSDFACANAF |
| 13 | CTCAGAGGACCAGTATCCCTCACAGGGTGACACCTGACCAGCTCTGTCCCACCTGGCCATGGGCTC |
| | CAGGTACCTCTGATGGGAAGACCTTTGTCTCTTGGGAACAAGTGAATCCTTGGCACAGGCCCAGTG |
| | GATTCTGCTGTGCAGAACAGAGAGCAGTGGACCTCAGGAGGCCTGCAAGGGGAGGACATAGGACA |
| | GTGACATCACAGTATGCCCCTCCCACCAGGAAAAGCAAGGCTGAGAATTTAGCTCTTTCCCAGGAG |
| | GACCAAGCCCTGAGCACAGACACAGTGCTGCCTGCCCCTTTGTGCCATGGGCTCCAGGCTGCTCTG |
| | TTGGGTGCTGCTTTGTCTCCTGGGAGCAGGCCCAGTAAAGGCTGGAGTCACTCAAACTCCAAGATAT |
| | CTGATCAAAACGAGAGGACAGCAAGTGACACTGAGCTGCTCCCCTATCTCTGGGCATAGGAGTGTA |
| | TCCTGGTACCAACAGACCCCAGGACAGGGCCTTCAGTTCCTCTTTGAATACTTCAGTGAGACACAGA |
| | GAAACAAAGGAAACTTCCCTGGTCGATTCTCAGGGCGCCAGTTCTCTAACTCTCGCTCTGAGATGAA |
| | TGTGAGCACCTTGGAGCTGGGGGACTCGGCCCTTTATCTTtgcgccagcagcttgactgggggaaactatggctacac |
| | cttcGGTTCGGGGACCAGGTTAACCGTTGTAGAGGACCTGAACAAGGTGTTCCCACCCGAGGTCGCT |
| | GTGTTTGAGCCATCAGAAGCAGAGATCTCCCACACCCAAAAGGCCACACTGGTGTGCCTGGCCACA |
| | GGCTTCTTCCCTGACCACGTGGAGCTGAGCTGGTGGGTGAATGGGAAGGAGGTGCACAGTGGGGT |
| | CAGCACGGACCCGCAGCCCCTCAAGGAGCAGCCCGCCCTCAATGACTCCAGATACTGCCTGAAGAT |
| | CGGAAGAGC |
| 14 | MGSRLLCWVLLCLLGAGPVKAGVTQTPRYLIKTRGQQVTLSCSPISGHRSVSWYQQTPGQGLQFLFEYF |
| | SETQRNKGNFPGRFSGRQFSNSRSEMNVSTLELGDSALYLcassltggnygytfGSGTRLTVVEDLNKVFPPE |
| | VAVFEPSEAEISHTQKATLVCLATGFFPDHVELSWWVNGKEVHSGVSTDPQPLKEQPALNDSRYCLKIG |
| | R |
| 15 | CCAAAACAAGAGACTTGCCTAGCCCAACCTTCCTCACGCTCGCTATTCTCAAGACCTGGGTTCCAGC |
| | CACTTTCCTACTGGCCCCGAGGAGAATTTCCAAAGAGACGCCTGCAGTGTTTCCACAGCTCAGCCAT |
| | GCTCCTGTTGCTCATACCAGTGCTGGGGATGATTTTTGCCCTGAGAGATGCCAGAGCCCAGTCTGT |
| | GAGCCAGCATAACCACCACGTAATTCTCTCTGAAGCAGCCTCACTGGAGTTGGGATGCAACTATTCC |
| | TATGGTGGAACTGTTAATCTCTTCTGGTATGTCCAGTACCCTGGTCAACACCTTCAGCTTCTCCTCAA |
| | GTACTTTTCAGGGGATCCACTGGTTAAAGGCATCAAGGGCTTTGAGGCTGAATTTATAAAGAGTAAAT |
| | TCTCCTTTAATCTGAGGAAACCCTCTGTGCAGTGGAGTGACACAGCTGAGTACTTCtgtgccgtaggcacca |
| | atgcaggcaaatcaacctttGGGGATGGGACTACGCTCACTGTGAAGCCAAATATCCAGAACCCTGACCCTGC |
| | CGTGTACCAGCTGAGAGACTCTAAATCCAGTGACAAGTCTGTCTGCCTATTCACCGATTTTGATTCTC |
| | AAACAAATGTGTCACAAAGTAAGGATTCTGATGTGTATATCACAGACAAAACTGTGCTAGACATGAGG |
| | TCTATGGACTTCAAGAGCAACAGTGCTGTGGCCTGGAGCAACAAATCTGACTTTGCATGTGCAAACG |
| | CCTTCA |
| 16 | MLLLLIPVLGMIFALRDARAQSVSQHNHHVILSEAASLELGCNYSYGGTVNLFWYVQYPGQHLQLLLKYFS |
| | GDPLVKGIKGFEAEFIKSKFSFNLRKPSVQWSDTAEYFcavgtnagkstfGDGTTLTVKPNIQNPDPAVYQLRD |
| | SKSSDKSVCLFTDFDSQTNVSQSKDSDVYITDKTVLDMRSMDFKSNSAVAWSNKSDFACANAF |
| 17 | ACCTGGAGCCCCCAGAACTGGCAGACACCTGCCTGATGCTGCCATGGGCCCCCAGCTCCTTGGCTA |
| | TGTGGTCCTTTGCCTTCTAGGAGCAGGCCCCCTGGAAGCCCAAGTGACCCAGAACCCAAGATACCT |
| | CATCACAGTGACTGGAAAGAAGTTAACAGTGACTTGTTCTCAGAATATGAACCATGAGTATATGTCCT |
| | GGTATCGACAAGACCCAGGGCTGGGCTTAAGGCAGATCTACTATTCAATGAATGTTGAGGTGACTGA |
| | TAAGGGAGATGTTCCTGAAGGGTACAAAGTCTCTCGAAAAGAGAAGAGGAATTTCCCCCTGATCCTG |
| | GAGTCGCCCAGCCCCAACCAGACCTCTCTGTACTTCtgtgccagcagccgaaagggactagcgggaggcggccccac |
| | cggggagctgttttttGGAGAAGGCTCTAGGCTGACCGTACTGGAGGACCTGAAAAACGTGTTCCCACCCGA |
| | GGTCGCTGTGTTTGAGCCATCAGAAGCAGAGATCTCCCACACCCAAAAGGCCACACTGGTGTGCCT |
| | GGCCACAGGCTTCTACCCCGACCACGTGGAGCTGAGCTGGTGGGTGAATGGGAAGGAGGTGCACA |
| | GTGGGGTCAGCACGGACCCGCAGCCCCTCAAGGAGCAGCCCGCCCTCAATGACTCCAGATACTGC |
| | CTGA |
| 18 | MGPQLLGYVVLCLLGAGPLEAQVTQNPRYLITVTGKKLTVTCSQNMNHEYMSWYRQDPGLGLRQIYYS |
| | MNVEVTDKGDVPEGYKVSRKEKRNFPLILESPSPNQTSLYFcassrkglagggptgelffGEGSRLTVLEDLKNVF |
| | PPEVAVFEPSEAEISHTQKATLVCLATGFYPDHVELSWWVNGKEVHSGVSTDPQPLKEQPALNDSRYCL |
| 19 | AGGAAAAGTTGAGGGGGCTTGACAGACAGAAATTCTAAACTGATGCTTATCTGTGTGTAAAGAAAGG |
| | ATTACTGATTCCCAATGAATATATCTTCAGCAATTCTAAATTTGGACAAAGTGGGGAAGTGCTTCCTTT |
| | GACAGAGACAGCTTTAAGTGAAAGCACTTGTGAAAGGGGGGGGCCTGCTGAAAGAATTCAGTTGAG |
| | GGTGAATTTACAGAGTTTCAGCTGGTTGGGAAGACTGGAAGACCACCTGGGCTGTCATTGAGCTCT |
| | GGTGCCAGGAGGAATGGACAAGATCTTAGGAGCATCATTTTTAGTTCTGTGGCTTCAACTATGCTGG |
| | GTGAGTGGCCAACAGAAGGAGAAAAGTGACCAGCAGCAGGTGAAACAAAGTCCTCAATCTTTGATA |
| | GTCCAGAAAGGAGGGATTTCAATTATAAACTGTGCTTATGAGAACACTGCGTTTGACTACTTTCCATG |
| | GTACCAACAATTCCCTGGGAAAGGCCCTGCATTATTGATAGCCATACGTCCAGATGTGAGTGAAAAG |
| | AAAGAAGGAAGATTCACAATCTCCTTCAATAAAAGTGCCAAGCAGTTCTCATTGCATATCATGGATTC |
| | CCAGCCTGGAGACTCAGCCACCTACTTCtgtgcagctgtgacaggaggaggtgctgacggactcacctttGGCAAAGGG |
| | ACTCATCTAATCATCCAGCCCTATATCCAGAACCCTGACCCTGCCGTGTACCAGCTGAGAGACTCTA |
| | AATCCAGTGACAAGTCTGTCTGCCTATTCACCGATTTTGATTCTCAAACAAATGTGTCACAAAGTAAG |
| | GATTCTGATGTGTATATCACAGACAAAACTGTGCTAGACATGAGGTCTATGGACTTCAAGAGCAACA |
| | GTGCTGTGGCCTGGAGCAACAAATCTGACTTTGCATGTGCAAACGCCTTCA |
| 20 | MDKILGASFLVLWLQLCWVSGQQKEKSDQQQVKQSPQSLIVQKGGISIINCAYENTAFDYFPWYQQFPG |
| | KGPALLIAIRPDVSEKKEGRFTISFNKSAKQFSLHIMDSQPGDSATYFcaavtgggadgltfGKGTHLIIQPYIQNP |
| | DPAVYQLRDSKSSDKSVCLFTDFDSQTNVSQSKDSDVYITDKTVLDMRSMDFKSNSAVAWSNKSDFAC |
| | ANAF |
| 21 | AGCTGTGAGGTCTGGTTCCCCGACGTGCTGCAGCAAGTGCCTTTGCCCTGCCTGTGGGCTCCCTCC |
| | ATGGCCAACTCTGCTATGGACACCAGAGTACTCTGCTGTGCGGTCATCTGTCTTCTGGGGGCAGGT |
| | CTCTCAAATGCCGGCGTCATGCAGAACCCAAGACACCTGGTCAGGAGGAGGGGACAGGAGGCAAG |
| | ACTGAGATGCAGCCCAATGAAAGGACACAGTCATGTTTACTGGTATCGGCAGCTCCCAGAGGAAGG |
| | TCTGAAATTCATGGTTTATCTCCAGAAAGAAAATATCATAGATGAGTCAGGAATGCCAAAGGAACGAT |
| | TTTCTGCTGAATTTCCCAAAGAGGGCCCCAGCATCCTGAGGATCCAGCAGGTAGTGCGAGGAGATT |
| | CGGCAGCTTATTTCtgtgccagctccccccagggttacaatgagcagttcttcGGGCCAGGGACACGGCTCACCGTGCT |
| | AGAGGACCTGAAAAACGTGTTCCCACCCGAGGTCGCTGTGTTTGAGCCATCAGAAGCAGAGATCTC |
| | CCACACCCAAAAGGCCACACTGGTGTGCCTGGCCACAGGCTTCTACCCCGACCACGTGGAGCTGA |
| | GCTGGTGGGTGAATGGGAAGGAGGTGCACAGTGGGGTCAGCACGGACCCGCAGCCCCTCAAGGA |
| | GCAGCCCGCCCTCAATGACTCCAGATACTGCCTGA |
| 22 | MDTRVLCCAVICLLGAGLSNAGVMQNPRHLVRRRGQEARLRCSPMKGHSHVYWYRQLPEEGLKFMVY |
| | LQKENIIDESGMPKERFSAEFPKEGPSILRIQQVVRGDSAAYFcasspqgyneqffGPGTRLTVLEDLKNVFPP |
| | EVAVFEPSEAEISHTQKATLVCLATGFYPDHVELSWWVNGKEVHSGVSTDPQPLKEQPALNDSRYCL |
| 23 | GCGGCCGCGCCACCATGCTTACAGCTTCTCTGCTGAGAGCCGTGATCGCCAGCATCTGTGTGGTGT |
| | CTAGCATGGCCCAGAAAGTGACACAGGCCCAGACCGAGATCAGCGTGGTGGAAAAAGAAGATGTGA |
| | CCCTGGACTGCGTGTACGAGACACGGGACACCACCTACTACCTGTTCTGGTACAAGCAGCCTCCTA |
| | GCGGCGAGCTGGTGTTCCTGATCAGACGGAACAGCTTCGACGAGCAGAACGAGATCTCCGGCCGG |
| | TACAGCTGGAACTTCCAGAAGTCCACCAGCAGCTTCAACTTCACCATCACCGCCAGCCAGGTGGTG |
| | GATAGCGCCGTGTATTTTTGCGCCCTGAGCGAGAGGCCTTACGGCGGAGCTACAAACAAGCTGATC |
| | TTCGGCACCGGCACACTGCTGGCTGTTCAACCTA ACATCCAGAACCCCGACCCCGCGG |
| 24 | CCATGGATACCTGGCTCGTGTGCTGGGCCATCTTCAGCCTGCTGAAAGCCGGACTGACCGAGCCTG |
| | AAGTGACCCAGACACCTAGCCACCAAGTGACACAGATGGGCCAAGAAGTGATCCTGCGCTGCGTGC |
| | CCATCAGCAACCACCTGTACTTCTACTGGTACAGACAGATCCTGGGCCAGAAAGTGGAATTCCTGGT |
| | GTCCTTCTACAACAACGAGATCAGCGAGAAGTCCGAGATCTTCGACGACCAGTTCAGCGTGGAAAG |
| | ACCCGACGGCAGCAACTTCACCCTGAAGATCAGAAGCACCAAGCTCGAGGACAGCGCCATGTACTT |
| | TTGCGCCAGCAGCGAGAGAAGAACCCAGCCTGCCTACGAGCAGTACTTCGGCCCTGGCACAAGACT |
| | GACCGTGACAG AGGACCTGCGG |
| | AACGTGACCCCCCCCAAGGTGTCCCTGTTCGAGCCCAGCAAGGCCGAGATCGCCAACAAGCAGAAA |
| | GCCACACTGGTCTGTCTGGCTAGGGGCTTCTTCCCCGACCACGTG |
| 25 | GCGGCCGCGCCACCATGAAGTCTCTGAGAGTGCTGCTGGTCATCCTGTGGCTGCAGCTGTCTTGGG |
| | TCTGGTCCCAGCAGAAAGAGGTGGAACAGAACAGCGGCCCTCTGTCTGTTCCTGAAGGCGCTATCG |
| | CCAGCCTGAACTGCACCTACAGCGATAGAGGCAGCCAGAGCTTCTTCTGGTACAGACAGTACAGCG |
| | GCAAGAGCCCCGAGCTGATCATGAGCATCTACAGCAACGGCGACAAAGAGGACGGCCGGTTTACA |
| | GCCCAGCTGAACAAGGCCAGCCAGTACGTGTCCCTGCTGATCAGAGATAGCCAGCCTAGCGACAGC |
| | GCCACCTATCTGTGTGCCGTTAGAGGCCAGGCTGGCACAGCCCTGATCTTTGGCAAGGGCACAACA |
| | CTGAGCGTGTCCAGCA ACATCCAGAACCC CGACCCCGCGG |
| 26 | GCGGCCGCGCCACCATGCTTACAGCTTCTCTGCTGAGAGCCGTGATCGCCAGCATCTGTGTGGTGT |
| | CTAGCATGGCCCAGAAAGTGACACAGGCCCAGACCGAGATCAGCGTGGTGGAAAAAGAAGATGTGA |
| | CCCTGGACTGCGTGTACGAGACACGGGACACCACCTACTACCTGTTCTGGTACAAGCAGCCTCCTA |
| | GCGGCGAGCTGGTGTTCCTGATCAGACGGAACAGCTTCGACGAGCAGAACGAGATCTCCGGCCGG |
| | TACAGCTGGAACTTCCAGAAGTCCACCAGCAGCTTCAACTTCACCATCACCGCCAGCCAGGTGGTG |
| | GATAGCGCCGTGTACTTTTGTGCCCTGCCTGCCGGAAATACCCCTCTGGTGTTTGGCAAGGGCACC |
| | AGACTGTCTGTGATCGCCA ACATCCAGA ACCCCGACCCCGCGG |
| 27 | CCATGGGCAGCAGACTGCTGTGTTGGGTGCTGCTGTGTCTGCTTGGAGCCGGACCTGTGAAAGCTG |
| | GCGTGACCCAGACACCTAGATACCTGATCAAGACCAGAGGCCAGCAAGTGACCCTGAGCTGCTCTC |
| | CTATCAGCGGCCACAGAAGCGTGTCCTGGTATCAGCAGACACCTGGACAGGGCCTGCAGTTCCTGT |
| | TCGAGTACTTCAGCGAGACACAGCGGAACAAGGGCAACTTCCCCGGCAGATTTTCCGGCAGACAGT |
| | TCAGCAACAGCCGCAGCGAGATGAACGTGTCCACACTGGAACTGGGCGACAGCGCCCTGTATCTGT |
| | GTGCCTCTTCTCTGACCGGCGGCAACTACGGCTACACATTTGGCAGCGGCACCAGACTGACAGTGG |
| | TCG AGGACCTGCGGAACGTGACCCCC |
| | CCCAAGGTGTCCCTGTTCGAGCCCAGCAAGGCCGAGATCGCCAACAAGCAGAAAGCCACACTGGTC |
| | TGTCTGGCTAGGGGCTTCTTCCCCGACCACGTG |
| 28 | GCGGCCGCGCCACCATGTTGTTGCTGCTGATTCCTGTGCTGGGCATGATCTTCGCCCTGAGGGATG |
| | CTAGAGCCCAGTCCGTGTCTCAGCACAACCACCACGTGATCCTGTCTGAGGCCGCCTCTCTGGAAC |
| | TGGGCTGCAATTACAGCTACGGCGGCACCGTGAACCTGTTTTGGTACGTGCAGTACCCCGGCCAGC |
| | ATCTCCAGCTGCTGCTGAAGTACTTTAGCGGCGACCCTCTGGTCAAGGGCATCAAGGGATTCGAGG |
| | CCGAGTTCATCAAGAGCAAGTTCAGCTTCAACCTGCGGAAGCCCAGCGTGCAGTGGAGCGATACAG |
| | CCGAGTACTTTTGTGCCGTGGGCACCAATGCCGGCAAGAGCACATTTGGCGACGGCACCACACTGA |
| | CCGTGAAGCCTA ACATCCAGAACCCCGA CCCCGCGG |
| 29 | CCATGGGCCCTCAGCTGCTGGGATATGTGGTGCTGTGTCTGCTTGGAGCCGGACCTCTGGAAGCCC |
| | AAGTGACACAGAACCCCAGATACCTGATCACCGTGACCGGCAAGAAACTGACCGTGACCTGCAGCC |
| | AGAACATGAACCACGAGTACATGAGCTGGTACAGACAGGACCCTGGCCTGGGCCTGAGACAGATCT |
| | ACTACAGCATGAACGTGGAAGTGACCGACAAGGGCGACGTGCCCGAGGGCTACAAGGTGTCCAGA |
| | AAAGAGAAGCGGAACTTCCCACTGATCCTGGAAAGCCCATCTCCTAACCAGACCAGCCTGTACTTCT |
| | GCGCCAGCAGCAGAAAAGGACTGGCTGGCGGAGGACCTACCGGCGAGCTGTTTTTTGGCGAGGGC |
| | AGCAGACTGACAGTGCTCG AGGACCTG |
| | CGGAACGTGACCCCCCCCAAGGTGTCCCTGTTCGAGCCCAGCAAGGCCGAGATCGCCAACAAGCA |
| | GAAAGCCACACTGGTCTGTCTGGCTAGGGGCTTCTTCCCCGACCACGTG |
| 30 | GCGGCCGCGCCACCATGGATAAGATTCTGGGCGCCAGCTTCCTGGTGCTGTGGCTGCAACTTTGTT |
| | GGGTGTCCGGCCAGCAGAAAGAGAAGTCCGACCAGCAGCAAGTGAAACAGAGCCCTCAGAGCCTG |
| | ATCGTGCAGAAAGGCGGCATCAGCATCATCAACTGCGCCTACGAGAATACCGCCTTCGACTACTTCC |
| | CCTGGTATCAGCAGTTCCCCGGCAAGGGACCTGCTCTGCTGATCGCCATTAGACCCGACGTGTCCG |
| | AGAAGAAAGAGGGCAGATTCACCATCAGCTTCAACAAGAGCGCCAAGCAGTTCAGCCTGCACATCA |
| | TGGATAGCCAGCCTGGCGACAGCGCCACCTATTTTTGTGCTGCTGTTACAGGCGGCGGAGCCGATG |
| | GCCTGACATTTGGAAAGGGCACCCACCTGATCATCCAGCCTT ACATCCAGAACCCCGACCCCGCGG |
| 31 | CCATGGACACCAGAGTGCTGTGCTGCGCCGTGATCTGTCTGCTTGGAGCCGGACTGTCTAATGCCG |
| | GCGTGATGCAGAACCCCAGACACCTCGTTCGGAGAAGAGGCCAAGAGGCCAGACTGAGATGCAGC |
| | CCTATGAAGGGCCACAGCCATGTGTACTGGTACAGACAGCTGCCCGAAGAGGGCCTGAAGTTCATG |
| | GTGTACCTGCAGAAAGAGAACATCATCGACGAGAGCGGCATGCCCAAAGAGCGGTTCTCTGCCGAG |
| | TTTCCCAAAGAGGGCCCCAGCATCCTGAGAATCCAGCAGGTTGTGCGGGGAGATAGCGCCGCCTAC |
| | TTTTGTGCTAGCAGCCCTCAGGGCTACAACGAGCAGTTTTTCGGCCCTGGCACCAGACTGACAGTG |
| | CTCG AGGACCTGCGGAACGTGACCCCCC |
| | CCAAGGTGTCCCTGTTCGAGCCCAGCAAGGCCGAGATCGCCAACAAGCAGAAAGCCACACTGGTCT |
| | GTCTGGCTAGGGGCTTCTTCCCCGACCACGTG |
| 32 | CALIPTGGGNKLTF |
| 33 | CSARLAHSGTNTGELFF |
| 34 | CAVGAYSSASKIIF |
| 35 | CASRDRGSGANVLTF |
| 36 | CSGGWGPNTGELFF |
| 37 | CAVGHFSSRSSGSARQLTF |
| 38 | CASSLKVGVDSSYNEQFF |
| 39 | CAESPSDGQKLLF |
| 40 | CASSLRQGISPEQFF |
| 41 | CAVEETSGSRLTF |
| 42 | CASSLRQGISPEQFF |
| 43 | CASSERQGITEAFF |
| 44 | CAVSGTGANSKLTF |
| 45 | CASSDPISGRGDEQFF |
| 46 | CASSFGGGAWNEQFF |
| 47 | CALRHKAAGNKLTF |
| 48 | CAALFDGGSQGNLIF |
| 49 | CSASGPEKLFF |
| 50 | CASRGLAGETQYF |
| 51 | CAVIPNSGYALNF |
| 52 | CALRGRNQGGKLIF |
| 53 | CSARLSSGGGYEQFF |
| 54 | CLVVPVYNQGGKLIF |
| 55 | CASSSGTSGMGETQYF |
| 56 | CILRDVGGSEKLVF |
| 57 | CSAKGLENQPQHF |
| 58 | CAVLKKGYALNF |
| 59 | CAVAGGYNKLIF |
| 60 | CASSLEAGDSYEQYF |
| 61 | CAVTGGYNKLIF |
| 62 | CASSLEAGDSYEQYF |
| 63 | CAGHQIQGAQKLVF |
| 64 | CASSYSGMNTEAFF |
| 65 | CAGANNNARLMF |
| 66 | CSVTPGGGVNTEAFF |
| 67 | CAVSAGGTSYGKLTF |
| 68 | CASSQVLRGEQYF |
| 69 | CAVSTGGATNKLIF |
| 70 | CAVQEGETSGSRLTF |
| 71 | CASSPGFNGNTIYF |
| 72 | CALSEATYNTDKLIF |
| 73 | CASSSTGTDYGYTF |
| 74 | CAGRQTSYDKVIF |
| 75 | CATSDVTGQGEMRGYTF |
| 76 | CALSEMNRDDKIIF |
| 77 | CASSPTGVSGNTIYF |
| 78 | CAGQQKTSGSRLTF |
| 79 | CASSSPRDRVGQPQHF |
| 80 | CAARRVDNNNDMRF |
| 81 | CASGLAQPQHF |
| 82 | CIVRVAVTNAGKSTF |
| 83 | CASRYYSADTQYF |
| 84 | CAVSGVLTGGGNKLTF |
| 85 | CASSEGTVSNQPQHF |
| 86 | CAEIGSGAGSYQLTF |
| 87 | CAENQGGSSYKLIF |
| 88 | CASSSQSGVDTEAFF |
| 89 | CAVSPPAQKLVF |
| 90 | CASQETGVGGELFF |
| 91 | CASSTTAVVSPLHF |
| 92 | CAEKKEGFKTIF |
| 93 | CASQETGVGGELFF |
| 94 | CATDEAAGNKLTF |
| 95 | CASKRESLATGELFF |
| 96 | CAAMYSGGGADGLTF |
| 97 | CASSTQGQAYEQYF |
| 98 | CAARGGSNYKLTF |
| 99 | CASSTGGEQYF |
| 100 | CATKGGNNRLAF |
| 101 | CASSAWTGETGYTF |
| 102 | CAATRAGGTSYGKLTF |
| 103 | CAWSVLAGVSQYF |
| 104 | CAASIATDKLIF |
| 105 | CASSWRGQGEGYTF |
| 106 | CVVTGGANNLFF |
| 107 | CASSEAGEWTQYF |
| 108 | CILVEGNEKLTF |
| 109 | CGADVQGSQGNLIF |
| 110 | CASRLGGRTTEAFF |
| 111 | CVVDFYTSGTYKYIF |
| 112 | CSARDREAAGYGYTF |
| 113 | CAGLDDKIIF |
| 114 | CAVAGSNYQLIW |
| 115 | CASSDPISGRGDEQFF |
| 116 | CVVNKATSGTYKYIF |
| 117 | CSARDREAAGYGYTF |
| 118 | CSARLAHSGTNTGELFF |
| 119 | TGTGCTCTGATTCCCACGGGAGGAGGAAACAAACTCACCTTT |
| 120 | TGCAGTGCTAGATTGGCCCATAGCGGGACCAACACCGGGGAGCTGTTTTTT |
| 121 | TGTGCTGTGGGTGCGTACAGCAGTGCTTCCAAGATAATCTTT |
| 122 | TGTGCCAGCAGGGACAGGGGATCTGGGGCCAACGTCCTGACTTTC |
| 123 | TGCAGTGGGGGGTGGGGACCTAACACCGGGGAGCTGTTTTTT |
| 124 | TGTGCTGTGGGGCACTTCTCATCTCGGTCCTCTGGTTCTGCAAGGCAACTGACCTTT |
| 125 | TGTGCCAGCAGTTTAAAGGTCGGTGTAGACAGCTCCTACAATGAGCAGTTCTTC |
| 126 | TGTGCAGAGAGCCCTTCAGATGGCCAGAAGCTGCTCTTT |
| 127 | TGTGCCAGCAGTTTGAGACAGGGTATAAGTCCTGAGCAGTTCTTC |
| 128 | TGTGCCGTGGAAGAAACCAGTGGCTCTAGGTTGACCTTT |
| 129 | TGTGCCAGCAGTTTGAGACAGGGTATAAGTCCTGAGCAGTTCTTC |
| 130 | TGTGCCAGCAGTGAGCGACAGGGGATAACTGAAGCTTTCTTT |
| 131 | TGTGCTGTGAGTGGTACTGGAGCCAATAGTAAGCTGACATTT |
| 132 | TGTGCCAGCAGCGACCCCATTAGCGGGAGAGGGGATGAGCAGTTCTTC |
| 133 | TGTGCCAGCAGCTTTGGGGGGGGGGGGTGGAATGAGCAGTTCTTC |
| 134 | TGTGCTCTGAGGCACAAAGCTGCAGGCAACAAGCTAACTTTT |
| 135 | TGTGCAGCGCTATTTGATGGAGGAAGCCAAGGAAATCTCATCTTT |
| 136 | TGCAGTGCTAGCGGCCCTGAAAAACTGTTTTTT |
| 137 | TGTGCCAGCAGGGGACTAGCGGGAGAGACCCAGTACTTC |
| 138 | TGTGCCGTGATCCCGAATTCCGGGTATGCACTCAACTTC |
| 139 | TGTGCTCTGAGGGGCCGGAACCAGGGAGGAAAGCTTATCTTC |
| 140 | TGCAGTGCTAGACTTTCTAGCGGGGGGGGCTATGAGCAGTTCTTC |
| 141 | TGCCTCGTGGTCCCTGTTTATAACCAGGGAGGAAAGCTTATCTTC |
| 142 | TGCGCCAGCAGCTCGGGGACTAGCGGGATGGGAGAGACCCAGTACTTC |
| 143 | TGCATCCTGAGAGACGTGGGCGGATCTGAAAAGCTGGTCTTT |
| 144 | TGCAGTGCCAAAGGCCTCGAAAATCAGCCCCAGCATTTT |
| 145 | TGCGCTGTCCTTAAAAAGGGGTATGCACTCAACTTC |
| 146 | TGTGCTGTGGCTGGTGGCTACAATAAGCTGATTTTT |
| 147 | TGTGCCAGCAGCTTAGAAGCGGGGGACTCCTACGAGCAGTACTTC |
| 148 | TGTGCTGTGACTGGTGGCTACAATAAGCTGATTTTT |
| 149 | TGTGCCAGCAGCTTAGAAGCGGGAGATTCCTACGAGCAGTACTTC |
| 150 | TGTGCTGGGCACCAAATTCAGGGAGCCCAGAAGCTGGTATTT |
| 151 | TGTGCCAGCAGTTACTCGGGGATGAACACTGAAGCTTTCTTT |
| 152 | TGTGCTGGGGCGAATAACAATGCCAGACTCATGTTT |
| 153 | TGCAGCGTCACTCCGGGGGGCGGGGTGAACACTGAAGCTTTCTTT |
| 154 | TGTGCCGTGAGCGCTGGTGGTACTAGCTATGGAAAGCTGACATTT |
| 155 | TGTGCCAGCAGCCAAGTTCTTAGGGGTGAGCAGTACTTC |
| 156 | TGTGCTGTCAGTACTGGTGGTGCTACAAACAAGCTCATCTTT |
| 157 | TGTGCTGTTCAGGAGGGAGAAACCAGTGGCTCTAGGTTGACCTTT |
| 158 | TGTGCCAGCAGTCCAGGGTTTAATGGAAACACCATATATTTT |
| 159 | TGTGCTCTGAGTGAGGCAACATATAACACCGACAAGCTCATCTTT |
| 160 | TGTGCCAGCAGCTCCACCGGGACGGACTATGGCTACACCTTC |
| 161 | TGTGCCGGGAGGCAAACCTCCTACGACAAGGTGATATTT |
| 162 | TGTGCCACCAGTGATGTGACTGGGCAGGGCGAGATGCGTGGCTACACCTTC |
| 163 | TGTGCTCTGAGTGAGATGAACAGAGATGACAAGATCATCTTT |
| 164 | TGTGCCAGCTCACCGACAGGGGTCTCTGGAAACACCATATATTTT |
| 165 | TGTGCTGGGCAGCAAAAAACCAGTGGCTCTAGGTTGACCTTT |
| 166 | TGTGCCAGTAGTTCCCCCCGGGACAGGGTCGGTCAGCCCCAGCATTTT |
| 167 | TGTGCTGCCCGTCGGGTCGACAATAACAATGACATGCGCTTT |
| 168 | TGTGCTAGTGGTTTAGCTCAGCCCCAGCATTTT |
| 169 | TGCATCGTCAGAGTCGCGGTAACCAATGCAGGCAAATCAACCTTT |
| 170 | TGTGCCAGCAGATATTATAGCGCGGATACGCAGTATTTT |
| 171 | TGTGCTGTGAGTGGGGTACTCACGGGAGGAGGAAACAAACTCACCTTT |
| 172 | TGCGCCAGCAGTGAGGGGACAGTTAGCAATCAGCCCCAGCATTTT |
| 173 | TGTGCAGAGATCGGGTCTGGGGCTGGGAGTTACCAACTCACTTTC |
| 174 | TGTGCAGAGAATCAGGGAGGCAGCAGCTATAAATTGATCTTC |
| 175 | TGTGCCAGCAGCTCTCAGTCGGGTGTGGACACTGAAGCTTTCTTT |
| 176 | TGTGCTGTGAGTCCCCCCGCGCAGAAACTTGTATTT |
| 177 | TGTGCCAGCCAAGAGACAGGGGTTGGCGGGGAGCTGTTTTTT |
| 178 | TGTGCCAGCAGTACGACAGCCGTGGTTTCACCCCTCCACTTT |
| 179 | TGTGCAGAGAAGAAGGAAGGCTTCAAAACTATCTTT |
| 180 | TGTGCCAGCCAAGAGACAGGGGTTGGCGGGGAGCTGTTTTTT |
| 181 | TGTGCTACGGACGAGGCTGCAGGCAACAAGCTAACTTTT |
| 182 | TGTGCCAGCAAAAGGGAATCACTAGCCACCGGGGAGCTGTTTTTT |
| 183 | TGTGCTGCCATGTATTCAGGAGGAGGTGCTGACGGACTCACCTTT |
| 184 | TGTGCCAGCAGTACCCAGGGACAGGCCTACGAGCAGTACTTC |
| 185 | TGTGCAGCAAGAGGAGGTAGCAACTATAAACTGACATTT |
| 186 | TGTGCCAGCAGCACAGGGGGCGAGCAGTACTTC |
| 187 | TGTGCTACAAAGGGTGGGAACAACAGACTCGCTTTT |
| 188 | TGTGCCAGCAGTGCCTGGACAGGGGAGACGGGCTACACCTTC |
| 189 | TGTGCAGCAACCCGCGCTGGTGGTACTAGCTATGGAAAGCTGACATTT |
| 190 | TGTGCCTGGAGTGTACTAGCAGGGGTTTCCCAGTACTTC |
| 191 | TGTGCAGCAAGTATAGCCACCGACAAGCTCATCTTT |
| 192 | TGCGCCAGCAGCTGGAGGGGACAGGGGGAAGGCTACACCTTC |
| 193 | TGTGTGGTGACCGGGGGGGCAAACAACCTCTTCTTT |
| 194 | TGCGCCAGCAGTGAGGCTGGGGAGTGGACGCAGTATTTT |
| 195 | TGCATCCTGGTAGAAGGAAATGAGAAATTAACCTTT |
| 196 | TGTGGAGCAGACGTACAAGGAAGCCAAGGAAATCTCATCTTT |
| 197 | TGTGCCAGCAGGCTGGGGGGAAGGACCACTGAAGCTTTCTTT |
| 198 | TGTGTGGTGGATTTTTATACCTCAGGAACCTACAAATACATCTTT |
| 199 | TGCAGTGCTAGAGATCGGGAGGCGGCCGGCTATGGCTACACCTTC |
| 200 | TGTGCAGGGTTAGATGACAAGATCATCTTT |
| 201 | TGTGCTGTGGGGGGTAGCAACTATCAGTTAATCTGG |
| 202 | TGTGCCAGCAGCGACCCCATTAGCGGGAGAGGGGATGAGCAGTTCTTC |
| 203 | TGTGTGGTGAACAAAGCCACCTCAGGAACCTACAAATACATCTTT |
| 204 | TGCAGTGCTAGAGATCGGGAGGCGGCCGGCTATGGCTACACCTTC |
| 205 | TGCAGTGCTAGATTGGCCCATAGCGGGACCAACACCGGGGAGCTGTTTTTT |
| 206 | MNYSPGLVSLILLLLGRTRGNSVTQMEGPVTLSEEAFLTINCTYTATGYPSLFWYVQYPGEGLQLLLKATK |
| | ADDKGSNKGFEATYRKETTSFHLEKGSVQVSDSAVYFCALIPTGGGNKLTFGTGTQLKVELNIQNPDPAV |
| | YQLRD |
| 207 | MLLLLLLLGPGSGLGAVVSQHPSWVICKSGTSVKIECRSLDFQATTMFWYRQFPKQSLMLMATSNEGSK |
| | ATYEQGVEKDKFLINHASLTLSTLTVTSAHPEDSSFYICSARLAHSGTNTGELFFGEGSRLTVLEDLKNVF |
| | PPEVAVFEPS |
| 208 | MLLELIPLLGIHFVLRTARAQSVTQPDIHITVSEGASLELRCNYSYGATPYLFWYVQSPGQGLQLLLKYFSG |
| | DTLVQGIKGFEAEFKRSQSSFNLRKPSVHWSDAAEYFCAVGAYSSASKIIFGSGTRLSIRPNIQNPDPAVY |
| | QLRD |
| 209 | MDTWLVCWAIFSLLKAGLTEPEVTQTPSHQVTQMGQEVILRCVPISNHLYFYWYRQILGQKVEFLVSFYN |
| | NEISEKSEIFDDQFSVERPDGSNFTLKIRSTKLEDSAMYFCASRDRGSGANVLTFGAGSRLTVLEDLKNVF |
| | PPEVAVFEPS |
| 210 | MLLLLLLLGPGSGLGAVVSQHPSWVICKSGTSVKIECRSLDFQATTMFWYRQFPKQSLMLMATSNEGSK |
| | ATYEQGVEKDKFLINHASLTLSTLTVTSAHPEDSSFYICSGGWGPNTGELFFGEGSRLTVLEDLKNVFPPE |
| | VAVFEPS |
| 211 | MMKCPQALLAIFWLLLSWVSSEDKVVQSPLSLVVHEGDTVTLNCSYEVTNFRSLLWYKQEKKAPTFLFML |
| | TSSGIEKKSGRLSSILDKKELSSILNITATQTGDSAIYLCAVGHFSSRSSGSARQLTFGSGTQLTVLPDIQNP |
| | DPAVYQLRD |
| 212 | MDSWTFCCVSLCILVAKHTDAGVIQSPRHEVTEMGQEVTLRCKPISGHNSLFWYRQTMMRGLELLIYFN |
| | NNVPIDDSGMPEDRFSAKMPNASFSTLKIQPSEPRDSAVYFCASSLKVGVDSSYNEQFFGPGTRLTVLE |
| | DLKNVFPPEVAVFEPS |
| 213 | MKTFAGFSFLFLWLQLDCMSRGEDVEQSLFLSVREGDSSVINCTYTDSSSTYLYWYKQEPGAGLQLLTYI |
| | FSNMDMKQDQRLTVLLNKKDKHLSLRIADTQTGDSAIYFCAESPSDGQKLLFARGTMLKVDLNIQNPDPA |
| | VYQLRD |
| 214 | MGPQLLGYVVLCLLGAGPLEAQVTQNPRYLITVTGKKLTVTCSQNMNHEYMSWYRQDPGLGLRQIYYS |
| | MNVEVTDKGDVPEGYKVSRKEKRNFPLILESPSPNQTSLYFCASSLRQGISPEQFFGPGTRLTVLEDLKN |
| | VFPPEVAVFEPS |
| 215 | MKKLLAMILWLQLDRLSGELKVEQNPLFLSMQEGKNYTIYCNYSTTSDRLYWYRQDPGKSLESLFVLLSN |
| | GAVKQEGRLMASLDTKARLSTLHITAAVHDLSATYFCAVEETSGSRLTFGEGTQLTVNPDIQNPDPAVYQ |
| | LRD |
| 216 | MGPQLLGYVVLCLLGAGPLEAQVTQNPRYLITVTGKKLTVTCSQNMNHEYMSWYRQDPGLGLRQIYYS |
| | MNVEVTDKGDVPEGYKVSRKEKRNFPLILESPSPNQTSLYFCASSLRQGISPEQFFGPGTRLTVLEDLKN |
| | VFPPEVAVFEPS |
| 217 | MGPQLLGYVVLCLLGAGPLEAQVTQNPRYLITVTGKKLTVTCSQNMNHEYMSWYRQDPGLGLRQIYYS |
| | MNVEVTDKGDVPEGYKVSRKEKRNFPLILESPSPNQTSLYFCASSERQGITEAFFGQGTRLTVVEDLNKV |
| | FPPEVAVFEPS |
| 218 | MLLLLVPAFQVIFTLGGTRAQSVTQLDSQVPVFEEAPVELRCNYSSSVSVYLFWYVQYPNQGLQLLLKYL |
| | SGSTLVKGINGFEAEFNKSQTSFHLRKPSVHISDTAEYFCAVSGTGANSKLTFGKGITLSVRPDIQNPDPA |
| | VYQLRD |
| 219 | MGTRLLFWVAFCLLGADHTGAGVSQSPSNKVTEKGKDVELRCDPISGHTALYWYRQSLGQGLEFLIYFQ |
| | GNSAPDKSGLPSDRFSAERTGGSVSTLTIQRTQQEDSAVYLCASSDPISGRGDEQFFGPGTRLTVLEDL |
| | KNVFPPEVAVFEPS |
| 220 | MGTRLLFWVAFCLLGADHTGAGVSQSPSNKVTEKGKDVELRCDPISGHTALYWYRQSLGQGLEFLIYFQ |
| | GNSAPDKSGLPSDRFSAERTGGSVSTLTIQRTQQEDSAVYLCASSFGGGAWNEQFFGPGTRLTVLEDLK |
| | NVFPPEVAVFEPS |
| 221 | MNYSPGLVSLILLLLGRTRGNSVTQMEGPVTLSEEAFLTINCTYTATGYPSLFWYVQYPGEGLQLLLKATK |
| | ADDKGSNKGFEATYRKETTSFHLEKGSVQVSDSAVYFCALRHKAAGNKLTFGGGTRVLVKPNIQNPDPA |
| | VYQLRD |
| 222 | MDKILGASFLVLWLQLCWVSGQQKEKSDQQQVKQSPQSLIVQKGGISIINCAYENTAFDYFPWYQQFPG |
| | KGPALLIAIRPDVSEKKEGRFTISFNKSAKQFSLHIMDSQPGDSATYFCAALFDGGSQGNLIFGKGTKLSV |
| | KPNIQNPDPAVYQLRD |
| 223 | MLLLLLLLGPGSGLGAVVSQHPSWVICKSGTSVKIECRSLDFQATTMFWYRQFPKQSLMLMATSNEGSK |
| | ATYEQGVEKDKFLINHASLTLSTLTVTSAHPEDSSFYICSASGPEKLFFGSGTQLSVLEDLNKVFPPEVAV |
| | FEPS |
| 224 | MKSLRVLLVILWLQLSWVWSQQKEVEQNSGPLSVPEGAIASLNCTYSDRGSQSFFWYRQYSGKSPELIM |
| | FIYSNGDKEDGRFTAQLNKASQYVSLLIRDSQPSDSATYLCAVIPNSGYALNFGKGTSLLVTPHIQNPDPA |
| | VYQLRD |
| 225 | MSIGLLCCAALSLLWAGPVNAGVTQTPKFQVLKTGQSMTLQCAQDMNHEYMSWYRQDPGMGLRLIHYS |
| | VGAGITDQGEVPNGYNVSRSTTEDFPLRLLSAAPSQTSVYFCASRGLAGETQYFGPGTRLLVLEDLKNVF |
| | PPEVAVFEPS |
| 226 | MNYSPGLVSLILLLLGRTRGNSVTQMEGPVTLSEEAFLTINCTYTATGYPSLFWYVQYPGEGLQLLLKATK |
| | ADDKGSNKGFEATYRKETTSFHLEKGSVQVSDSAVYFCALRGRNQGGKLIFGQGTELSVKPNIQNPDPA |
| | VYQLRD |
| 227 | MLLLLLLLGPGSGLGAVVSQHPSRVICKSGTSVKIECRSLDFQATTMFWYRQFPKQSLMLMATSNEGSK |
| | ATYEQGVEKDKFLINHASLTLSTLTVTSAHPEDSSFYICSARLSSGGGYEQFFGPGTRLTVLEDLKNVFPP |
| | EVAVFEPS |
| 228 | MRQVARVIVFLTLSTLSLAKTTQPISMDSYEGQEVNITCSHNNIATNDYITWYQQFPSQGPRFIIQGYKTKV |
| | TNEVASLFIPADRKSSTLSLPRVSLSDTAVYYCLVVPVYNQGGKLIFGQGTELSVKPNIQNPDPAVYQLRD |
| 229 | MGSRLLCWVLLCLLGAGPVKAGVTQTPRYLIKTRGQQVTLSCSPISGHRSVSWYQQTPGQGLQFLFEYF |
| | SETQRNKGNFPGRFSGRQFSNSRSEMNVSTLELGDSALYLCASSSGTSGMGETQYFGPGTRLLVLEDL |
| | KNVFPPEVAVFEPS |
| 230 | MKLVTSITVLLSLGIMGDAKTTQPNSMESNEEEPVHLPCNHSTISGTDYIHWYRQLPSQGPEYVIHGLTSN |
| | VNNRMASLAIAEDRKSSTLILHRATLRDAAVYYCILRDVGGSEKLVFGKGTKLTVNPYIQNPDPAVYQLRD |
| 231 | MLLLLLLLGPGSGLGAVVSQHPSWVICKSGTSVKIECRSLDFQATTMFWYRQFPKQSLMLMATSNEGSK |
| | ATYEQGVEKDKFLINHASLTLSTLTVTSAHPEDSSFYICSAKGLENQPQHFGDGTRLSILEDLNKVFPPEV |
| | AVFEPS |
| 232 | MWGAFLLYVSMKMGGTAGQSLEQPSEVTAVEGAIVQINCTYQTSGFYGLSWYQQHDGGAPTFLSYNAL |
| | DGLEETGRFSSFLSRSDSYGYLLLQELQMKDSASYFCAVLKKGYALNFGKGTSLLVTPHIQNPDPAVYQL |
| | RD |
| 233 | MALQSTLGAVWLGLLLNSLWKVAESKDQVFQPSTVASSEGAVVEIFCNHSVSNAYNFFWYLHFPGCAPR |
| | LLVKGSKPSQQGRYNMTYERFSSSLLILQVREADAAVYYCAVAGGYNKLIFGAGTRLAVHPYIQNPDPAV |
| | YQLRD |
| 234 | MGTRLLCWVAFCLLVEELIEAGVVQSPRYKIIEKKQPVAFWCNPISGHNTLYWYLQNLGQGPELLIRYENE |
| | EAVDDSQLPKDRFSAERLKGVDSTLKIQPAELGDSAVYLCASSLEAGDSYEQYFGPGTRLTVTEDLKNVF |
| | PPEVAVFEPS |
| 235 | MALQSTLGAVWLGLLLNSLWKVAESKDQVFQPSTVASSEGAVVEIFCNHSVSNAYNFFWYLHFPGCAPR |
| | LLVKGSKPSQQGRYNMTYERFSSSLLILQVREADAAVYYCAVTGGYNKLIFGAGTRLAVHPYIQNPDPAV |
| | YQLRD |
| 236 | MGTRLLCWVAFCLLVEELIEAGVVQSPRYKIIEKKQPVAFWCNPISGHNTLYWYLQNLGQGPELLIRYENE |
| | EAVDDSQLPKDRFSAERLKGVDSTLKIQPAELGDSAVYLCASSLEAGDSYEQYFGPGTRLTVTEDLKNVF |
| | PPEVAVFEPS |
| 237 | MLLEHLLIILWMQLTWVSGQQLNQSPQSMFIQEGEDVSMNCTSSSIFNTWLWYKQEPGEGPVLLIALYKA |
| | GELTSNGRLTAQFGITRKDSFLNISASIPSDVGIYFCAGHQIQGAQKLVFGQGTRLTINPNIQNPDPAVYQL |
| | RD |
| 238 | MSISLLCCAAFPLLWAGPVNAGVTQTPKFRILKIGQSMTLQCTQDMNHNYMYWYRQDPGMGLKLIYYSV |
| | GAGITDKGEVPNGYNVSRSTTEDFPLRLELAAPSQTSVYFCASSYSGMNTEAFFGQGTRLTVVEDLNKV |
| | FPPEVAVFEPS |
| 239 | MLLEHLLIILWMQLTWVSGQQLNQSPQSMFIQEGEDVSMNCTSSSIFNTWLWYKQDPGEGPVLLIALYKA |
| | GELTSNGRLTAQFGITRKDSFLNISASIPSDVGIYFCAGANNNARLMFGDGTQLVVKPNIQNPDPAVYQLR |
| | D |
| 240 | MLSLLLLLLGLGSVFSAVISQKPSRDICQRGTSLTIQCQVDSQVTMMFWYRQQPGQSLTLIATANQGSEA |
| | TYESGFVIDKFPISRPNLTFSTLTVSNMSPEDSSIYLCSVTPGGGVNTEAFFGQGTRLTVVEDLNKVFPPE |
| | VAVFEPS |
| 241 | MKSLRVLLVILWLQLSWVWSQQKEVEQNSGPLSVPEGAIASLNCTYSDRGSQSFFWYRQYSGKSPELIM |
| | SIYSNGDKEDGRFTAQLNKASQYVSLLIRDSQPSDSATYLCAVSAGGTSYGKLTFGQGTILTVHPNIQNPD |
| | PAVYQLRD |
| 242 | MGCRLLCCVVFCLLQAGPLDTAVSQTPKYLVTQMGNDKSIKCEQNLGHDTMYWYKQDSKKFLKIMFSYN |
| | NKELIINETVPNRFSPKSPDKAHLNLHINSLELGDSAVYFCASSQVLRGEQYFGPGTRLTVTEDLKNVFPP |
| | EVAVFEPS |
| 243 | MVKIRQFLLAILWLQLSCVSAAKNEVEQSPQNLTAQEGEFITINCSYSVGISALHWLQQHPGGGIVSLFML |
| | SSGKKKHGRLIATINIQEKHSSLHITASHPRDSAVYICAVSTGGATNKLIFGTGTLLAVQPNIQNPDPAVYQL |
| | RD |
| 244 | MVKIRQFLLAILWLQLSCVSAAKNEVEQSPQNLTAQEGEFITINCSYSVGISALHWLQQHPGGGIVSLFML |
| | SSGKKKHGRLIATINIQEKHSSLHITASHPRDSAVYICAVQEGETSGSRLTFGEGTQLTVNPDIQNPDPAVY |
| | QLRD |
| 245 | MSIGLLCCAALSLLWAGPVNAGVTQTPKFQVLKTGQSMTLQCAQDMNHEYMSWYRQDPGMGLRLIHYS |
| | VGAGITDQGEVPNGYNVSRSTTEDFPLRLLSAAPSQTSVYFCASSPGFNGNTIYFGEGSWLTVVEDLNK |
| | VFPPEVAVFEPS |
| 246 | MLTASLLRAVIASICVVSSMAQKVTQAQTEISVVEKEDVTLDCVYETRDTTYYLFWYKQPPSGELVFLIRR |
| | NSFDEQNEISGRYSWNFQKSTSSFNFTITASQVVDSAVYFCALSEATYNTDKLIFGTGTRLQVFPNIQNPD |
| | PAVYQLRD |
| 247 | MGPGLLCWALLCLLGAGSVETGVTQSPTHLIKTRGQQVTLRCSSQSGHNTVSWYQQALGQGPQFIFQY |
| | YREEENGRGNFPPRFSGLQFPNYSSELNVNALELDDSALYLCASSSTGTDYGYTFGSGTRLTVVEDLNK |
| | VFPPEVAVFEPS |
| 248 | MKSLRVLLVILWLQLSWVWSQQKEVEQNSGPLSVPEGAIASLNCTYSDRGSQSFFWYRQYSGKSPELIM |
| | FIYSNGDKEDGRFTAQLNKASQYVSLLIRDSQPSDSATYLCAGRQTSYDKVIFGPGTSLSVIPNIQNPDPA |
| | VYQLRD |
| 249 | MASLLFFCGAFYLLGTGSMDADVTQTPRNRITKTGKRIMLECSQTKGHDRMYWYRQDPGLGLRLIYYSF |
| | DVKDINKGEISDGYSVSRQAQAKFSLSLESAIPNQTALYFCATSDVTGQGEMRGYTFGSGTRLTVVEDLN |
| | KVFPPEVAVFEPS |
| 250 | MLTASLLRAVIASICVVSSMAQKVTQAQTEISVVEKEDVTLDCVYETRDTTYYLFWYKQPPSGELVFLIRR |
| | NSFDEQNEISGRYSWNFQKSTSSFNFTITASQVVDSAVYFCALSEMNRDDKIIFGKGTRLHILPNIQNPDP |
| | AVYQLRD |
| 251 | MDTRVLCCAVICLLGAGLSNAGVMQNPRHLVRRRGQEARLRCSPMKGHSHVYWYRQLPEEGLKFMVY |
| | LQKENIIDESGMPKERFSAEFPKEGPSILRIQQVVRGDSAAYFCASSPTGVSGNTIYFGEGSWLTVVEDLN |
| | KVFPPEVAVFEPS |
| 252 | MLLEHLLIILWMQLTWVSGQQLNQSPQSMFIQEGEDVSMNCTSSSIFNTWLWYKQDPGEGPVLLIALYKA |
| | GELTSNGRLTAQFGITRKDSFLNISASIPSDVGIYFCAGQQKTSGSRLTFGEGTQLTVNPDIQNPDPAVYQ |
| | LRD |
| 253 | MSNQVLCCVVLCFLGANTVDGGITQSPKYLFRKEGQNVTLSCEQNLNHDAMYWYRQDPGQGLRLIYYS |
| | QIVNDFQKGDIAEGYSVSREKKESFPLTVTSAQKNPTAFYLCASSSPRDRVGQPQHFGDGTRLSILEDLN |
| | KVFPPEVAVFEPS |
| 254 | MKRILGALLGLLSAQVCCVRGIQVEQSPPDLILQEGANSTLRCNFSDSVNNLQWFHQNPWGQLINLFYIP |
| | SGTKQNGRLSATTVATERYSLLYISSSQTTDSGVYFCAARRVDNNNDMRFGAGTRLTVKPNIQKPDPAV |
| | YQLRD |
| 255 | MATRLLCCVVLCLLGEELIDARVTQTPRHKVTEMGQEVTMRCQPILGHNTVFWYRQTMMQGLELLAYFR |
| | NRAPLDDSGMPKDRFSAEMPDATLATLKIQPSEPRDSAVYFCASGLAQPQHFGDGTRLSILEDLNKVFPP |
| | EVAVFEPS |
| 256 | MRLVARVTVFLTFGTIIDAKTTQPPSMDCAEGRAANLPCNHSTISGNEYVYWYRQIHSQGPQYIIHGLKNN |
| | ETNEMASLIITEDRKSSTLILPHATLRDTAVYYCIVRVAVTNAGKSTFGDGTTLTVKPNIQKPDPAVYQLRD |
| 257 | MGPQLLGYVVLCLLGAGPLEAQVTQNPRYLITVTGKKLTVTCSQNMNHEYMSWYRQDPGLGLRQIYYS |
| | MNVEVTDKGDVPEGYKVSRKEKRNFPLILESPSPNQTSLYFCASRYYSADTQYFGPGTRLTVLEDLKNVF |
| | PPEVAVFEPS |
| 258 | MLLLLVPAFQVIFTLGGTRAQSVTQLDSQVPVFEEAPVELRCNYSSSVSVYLFWYVQYPNQGLQLLLKYL |
| | SGSTLVKGINGFEAEFNKSQTSFHLRKPSVHISDTAEYFCAVSGVLTGGGNKLTFGTGTQLKVELNIQKPD |
| | PAVYQLRD |
| 259 | MGTRLFFYVALCLLWAGHRDAGITQSPRYKITETGRQVTLMCHQTWSHSYMFWYRQDLGHGLRLIYYSA |
| | AADITDKGEVPDGYVVSRSKTENFPLTLESATRSQTSVYFCASSEGTVSNQPQHFGDGTRLSILEDLNKV |
| | FPPEVAVFEPS |
| 260 | MAGIRALFMYLWLQLDWVSRGESVGLHLPTLSVQEGDNSIINCAYSNSASDYFIWYKQESGKGPQFIIDIR |
| | SNMDKRQGQRVTVLLNKTVKHLSLQIAATQPGDSAVYFCAEIGSGAGSYQLTFGKGTKLSVIPNIQNPDP |
| | AVYQLRD |
| 261 | MAGIRALFMYLWLQLDWVSRGESVGLHLPTLSVQEGDNSIINCAYSNSASDYFIWYKQESGKGPQFIIDIR |
| | SNMDKRQGQRVTVLLNKTVKHLSLQIAATQPGDSAVYFCAENQGGSSYKLIFGSGTRLLVRPDIQNPDPA |
| | VYQLRD |
| 262 | MGTSLLCWMALCLLGADHADTGVSQNPRHKITKRGQNVTFRCDPISEHNRLYWYRQTLGQGPEFLTYF |
| | QNEAQLEKSRLLSDRFSAERPKGSFSTLEIQRTEQGDSAMYLCASSSQSGVDTEAFFGQGTRLTVVEDL |
| | NKVFPPEVAVFEPS |
| 263 | MLLLLVPVLEVIFTLGGTRAQSVTQLGSHVSVSEGALVLLRCNYSSSVPPYLFWYVQYPNQGLQLLLKYT |
| | TGATLVKGINGFEAEFKKSETSFHLTKPSAHMSDAAEYFCAVSPPAQKLVFGTGTRLLVSPNIQNPDPAV |
| | YQLRD |
| 264 | MSIGLLCCAALSLLWAGPVNAGVTQTPKFQVLKTGQSMTLQCAQDMNHEYMSWYRQDPGMGLRLIHYS |
| | VGAGITDQGEVPNGYNVSRSTTEDFPLRLLSAAPSQTSVYFCASQETGVGGELFFGEGSRLTVLEDLKN |
| | VFPPEVAVFEPS |
| 265 | MSIGLLCCAALSLLWAGPVNAGVTQTPKFQVLKTGQSMTLQCAQDMNHEYMSWYRQDPGMGLRLIHYS |
| | VGAGITDQGEVPNGYNVSRSTTEDFPLRLLSAAPSQTSVYFCASSTTAVVSPLHFGNGTRLTVTEDLNKV |
| | FPPEVAVFEPS |
| 266 | MKTFAGFSFLFLWLQLDCMSRGEDVEQSLFLSVREGDSSVINCTYTDSSSTYLYWYKQEPGAGLQLLTYI |
| | FSNMDMKQDQRLTVLLNKKDKHLSLRIADTQTGDSAIYFCAEKKEGFKTIFGAGTRLFVKANIQNPDPAVY |
| | QLRD |
| 267 | MSIGLLCCAALSLLWAGPVNAGVTQTPKFQVLKTGQSMTLQCAQDMNHEYMSWYRQDPGMGLRLIHYS |
| | VGAGITDQGEVPNGYNVSRSTTEDFPLRLLSAAPSQTSVYFCASQETGVGGELFFGEGSRLTVLEDLKN |
| | VFPPEVAVFEPS |
| 268 | METLLGVSLVILWLQLARVNSQQGEEDPQALSIQEGENATMNCSYKTSINNLQWYRQNSGRGLVHLILIR |
| | SNEREKHSGRLRVTLDTSKKSSSLLITASRAADTASYFCATDEAAGNKLTFGGGTRVLVKPNIQNPDPAV |
| | YQLRD |
| 269 | MDTWLVCWAIFSLLKAGLTEPEVTQTPSHQVTQMGQEVILRCVPISNHLYFYWYRQILGQKVEFLVSFYN |
| | NEISEKSEIFDDQFSVERPDGSNFTLKIRSTKLEDSAMYFCASKRESLATGELFFGEGSRLTVLEDLKNVF |
| | PPEVAVFEPS |
| 270 | METLLGVSLVILWLQLARVNSQQGEEDPQALSIQEGENATMNCSYKTSINNLQWYRQNSGRGLVHLILIR |
| | SNEREKHSGRLRVTLDTSKKSSSLLITASRAADTASYFCAAMYSGGGADGLTFGKGTHLIIQPYIQNPDPA |
| | VYQLRD |
| 271 | MDTWLVCWAIFSLLKAGLTEPEVTQTPSHQVTQMGQEVILRCVPISNHLYFYWYRQILGQKVEFLVSFYN |
| | NEISEKSEIFDDQFSVERPDGSNFTLKIRSTKLEDSAMYFCASSTQGQAYEQYFGPGTRLTVTEDLKNVF |
| | PPEVAVFEPS |
| 272 | MTSIRAVFIFLWLQLDLVNGENVEQHPSTLSVQEGDSAVIKCTYSDSASNYFPWYKQELGKRPQLIIDIRS |
| | NVGEKKDQRIAVTLNKTAKHFSLHITETQPEDSAVYFCAARGGSNYKLTFGKGTLLTVNPNIQNPDPAVY |
| | QLRD |
| 273 | MGSWTLCCVSLCILVAKHTDAGVIQSPRHEVTEMGQEVTLRCKPISGHDYLFWYRQTMMRGLELLIYFN |
| | NNVPIDDSGMPEDRFSAKMPNASFSTLKIQPSEPRDSAVYFCASSTGGEQYFGPGTRLTVTEDLKNVFP |
| | PEVAVFEPS |
| 274 | METLLGVSLVILWLQLARVNSQQGEEDPQALSIQEGENATMNCSYKTSINNLQWYRQNSGRGLVHLILIR |
| | SNEREKHSGRLRVTLDTSKKSSSLLITASRAADTASYFCATKGGNNRLAFGKGNQVVVIPNIQNPDPAVY |
| | QLRD |
| 275 | MDTWLVCWAIFSLLKAGLTEPEVTQTPSHQVTQMGQEVILRCVPISNHLYFYWYRQILGQKVEFLVSFYN |
| | NEISEKSEIFDDQFSVERPDGSNFTLKIRSTKLEDSAMYFCASSAWTGETGYTFGSGTRLTVVEDLNKVF |
| | PPEVAVFEPS |
| 276 | MAMLLGASVLILWLQPDWVNSQQKNDDQQVKQNSPSLSVQEGRISILNCDYTNSMFDYFLWYKKYPAE |
| | GPTFLISISSIKDKNEDGRFTVFLNKSAKHLSLHIVPSQPGDSAVYFCAATRAGGTSYGKLTFGQGTILTVH |
| | PNIQNPDPAVYQLRD |
| 277 | MLCSLLALLLGTFFGVRSQTIHQWPATLVQPVGSPLSLECTVEGTSNPNLYWYRQAAGRGLQLLFYSVGI |
| | GQISSEVPQNLSASRPQDRQFILSSKKLLLSDSGFYLCAWSVLAGVSQYFGPGTRLLVLEDLKNVFPPEV |
| | AVFEPS |
| 278 | MTSIRAVFIFLWLQLDLVNGENVEQHPSTLSVQEGDSAVIKCTYSDSASNYFPWYKQELGKRPQLIIDIRS |
| | NVGEKKDQRIAVTLNKTAKHFSLHITETQPEDSAVYFCAASIATDKLIFGTGTRLQVFPNIQNPDPAVYQLR |
| | D |
| 279 | MGSRLLCWVLLCLLGAGPVKAGVTQTPRYLIKTRGQQVTLSCSPISGHRSVSWYQQTPGQGLQFLFEYF |
| | SETQRNKGNFPGRFSGRQFSNSRSEMNVSTLELGDSALYLCASSWRGQGEGYTFGSGTRLTVVEDLNK |
| | VFPPEVAVFEPS |
| 280 | MKKHLTTFLVILWLYFYRGNGKNQVEQSPQSLIILEGKNCTLQCNYTVSPFSNLRWYKQDTGRGPVSLTI |
| | MTFSENTKSNGRYTATLDADTKQSSLHITASQLSDSASYICVVTGGANNLFFGTGTRLTVIPYIQNPDPAV |
| | YQLRD |
| 281 | MGSRLLCWVLLCLLGAGPVKAGVTQTPRYLIKTRGQQVTLSCSPISGHRSVSWYQQTPGQGLQFLFEYF |
| | SETQRNKGNFPGRFSGRQFSNSRSEMNVSTLELGDSALYLCASSEAGEWTQYFGPGTRLTVLEDLKNV |
| | FPPEVAVFEPS |
| 282 | MKLVTSITVLLSLGIMGDAKTTQPNSMESNEEEPVHLPCNHSTISGTDYIHWYRQLPSQGPEYVIHGLTSN |
| | VNNRMASLAIAEDRKSSTLILHRATLRDAAVYYCILVEGNEKLTFGTGTRLTIIPNIQNPDPAVYQLRD |
| 283 | METVLQVLLGILGFQAAWVSSQELEQSPQSLIVQEGKNLTINCTSSKTLYGLYWYKQKYGEGLIFLMMLQ |
| | KGGEEKSHEKITAKLDEKKQQSSLHITASQPSHAGIYLCGADVQGSQGNLIFGKGTKLSVKPNIQNPDPAV |
| | YQLRD |
| 284 | MDTWLVCWAIFSLLKAGLTEPEVTQTPSHQVTQMGQEVILRCVPISNHLYFYWYRQILGQKVEFLVSFYN |
| | NEISEKSEIFDDQFSVERPDGSNFTLKIRSTKLEDSAMYFCASRLGGRTTEAFFGQGTRLTVVEDLNKVFP |
| | PEVAVFEPS |
| 285 | MISLRVLLVILWLQLSWVWSQRKEVEQDPGPFNVPEGATVAFNCTYSNSASQSFFWYRQDCRKEPKLL |
| | MSVYSSGNEDGRFTAQLNRASQYISLLIRDSKLSDSATYLCVVDFYTSGTYKYIFGTGTRLKVLANIQNPD |
| | PAVYQLRD |
| 286 | MLLLLLLLGPGSGLGAVVSQHPSRVICKSGTSVKIECRSLDFQATTMFWYRQFPKQSLMLMATSNEGSK |
| | ATYEQGVEKDKFLINHASLTLSTLTVTSAHPEDSSFYICSARDREAAGYGYTFGSGTRLTVVEDLNKVFPP |
| | EVAVFEPS |
| 287 | MVLKFSVSILWIQLAWVSTQLLEQSPQFLSIQEGENLTVYCNSSSVFSSLQWYRQEPGEGPVLLVTVVTG |
| | GEVKKLKRLTFQFGDARKDSSLHITAAQPGDTGLYLCAGLDDKIIFGKGTRLHILPNIQNPDPAVYQLRD |
| 288 | MKRILGALLGLLSAQVCCVRGIQVEQSPPDLILQEGANSTLRCNFSDSVNNLQWFHQNPWGQLINLFYIP |
| | SGTKQNGRLSATTVATERYSLLYISSSQTTDSGVYFCAVAGSNYQLIWGAGTKLIIKPDIQNPDPAVYQLR |
| | D |
| 289 | MGTRLLCWAALCLLGAELTEAGVAQSPRYKIIEKRQSVAFWCNPISGHATLYWYQQILGQGPKLLIQFQN |
| | NGVVDDSQLPKDRFSAERLKGVDSTLKIQPAKLEDSAVYLCASSDPISGRGDEQFFGPGTRLTVLEDLKN |
| | VFPPEVAVFEPS |
| 290 | MISLRVLLVILWLQLSWVWSQRKEVEQDPGPFNVPEGATVAFNCTYSNSASQSFFWYRQDCRKEPKLL |
| | MSVYSSGNEDGRFTAQLNRASQYISLLIRDSKLSDSATYLCVVNKATSGTYKYIFGTGTRLKVLANIQNPD |
| | PAVYQLRD |
| 291 | MLLLLLLLGPGSGLGAVVSQHPSWVICKSGTSVKIECRSLDFQATTMFWYRQFPKQSLMLMATSNEGSK |
| | ATYEQGVEKDKFLINHASLTLSTLTVTSAHPEDSSFYICSARDREAAGYGYTFGSGTRLTVVEDLNKVFPP |
| | EVAVFEPS |
| 292 | MLLLLLLLGPGSGLGAVVSQHPSWVICKSGTSVKIECRSLDFQATTMFWYRQFPKQSLMLMATSNEGSK |
| | ATYEQGVEKDKFLINHASLTLSTLTVTSAHPEDSSFYICSARLAHSGTNTGELFFGEGSRLTVLEDLKNVF |
| | PPEVAVFEPS |
| 293 | GACTGTGATTTCTTCATGTTAAGGATCAAGACCATTATTTGGGTAACACACTAAAGATGAACTATTCTC |
| | CAGGCTTAGTATCTCTGATACTCTTACTGCTTGGAAGAACCCGTGGAAATTCAGTGACCCAGATGGA |
| | AGGGCCAGTGACTCTCTCAGAAGAGGCCTTCCTGACTATAAACTGCACGTACACAGCCACAGGATA |
| | CCCTTCCCTTTTCTGGTATGTCCAATATCCTGGAGAAGGTCTACAGCTCCTCCTGAAAGCCACGAAG |
| | GCTGATGACAAGGGAAGCAACAAAGGTTTTGAAGCCACATACCGTAAAGAAACCACTTCTTTCCACT |
| | TGGAGAAAGGCTCAGTTCAAGTGTCAGACTCAGCGGTGTACTTCTGTGCTCTGATTCCCACGGGAG |
| | GAGGAAACAAACTCACCTTTGGGACAGGCACTCAGCTAAAAGTGGAACTCAATATCCAGAACCCTGA |
| | CCCTGCCGTGTACCAGCTGAGAGACT |
| 294 | GTCATCCCTCCTCGCTGGTGAATGGAGGCAGTGGTCACAACTCTCCCCAGAGAAGGTGGTGTGAGG |
| | CCATCACGGAAGATGCTGCTGCTTCTGCTGCTTCTGGGGCCAGGCTCCGGGCTTGGTGCTGTCGTC |
| | TCTCAACATCCGAGCTGGGTTATCTGTAAGAGTGGAACCTCTGTGAAGATCGAGTGCCGTTCCCTGG |
| | ACTTTCAGGCCACAACTATGTTTTGGTATCGTCAGTTCCCGAAACAGAGTCTCATGCTGATGGCAACT |
| | TCCAATGAGGGCTCCAAGGCCACATACGAGCAAGGCGTCGAGAAGGACAAGTTTCTCATCAACCAT |
| | GCAAGCCTGACCTTGTCCACTCTGACAGTGACCAGTGCCCATCCTGAAGACAGCAGCTTCTACATCT |
| | GCAGTGCTAGATTGGCCCATAGCGGGACCAACACCGGGGAGCTGTTTTTTGGAGAAGGCTCTAGGC |
| | TGACCGTACTGGAGGACCTGAAAAACGTGTTCCCACCCGAGGTCGCTGTGTTTGAGCCATCAGA |
| 295 | GGTTTTGATACATCTCAGAAATTAGGAGAAACACACTAATGAGCTCTCTCTGCTAAACTGGTTCTGCA |
| | CTTGGCCTCCAGAGGGCGATGCTGCACACACTGGAGCTTTTGTTTCTGTTGAAGATCAATCCACTGC |
| | TCAGTTTCTTCTTCCTGCAGCTGGTTGAGTTCTTTCCAGACAAAGACAAGTGACAAGAATTAGAGGTT |
| | TAAAAAGCAACCAGATTCATCTCAGCAGCTTTTGTAGTTTTAAATAAGCAAGGAGTTTCTCCAGCGAA |
| | ACTTCCTCACACCTCTTGGTCTTGGTCTCTTCAGACACTTTCCTTCCTGTTCTCTGGAGATCTTGCAG |
| | AAAAGAGCCTGCAGTGTTTCCCTTGTTCAGCCATGCTCCTGGAGCTTATCCCACTGCTGGGGATACA |
| | TTTTGTCCTGAGAACTGCCAGAGCCCAGTCAGTGACCCAGCCTGACATCCACATCACTGTCTCTGAA |
| | GGAGCCTCACTGGAGTTGAGATGTAACTATTCCTATGGGGCAACACCTTATCTCTTCTGGTATGTCC |
| | AGTCCCCCGGCCAAGGCCTCCAGCTGCTCCTGAAGTACTTTTCAGGAGACACTCTGGTTCAAGGCA |
| | TTAAAGGCTTTGAGGCTGAATTTAAGAGGAGTCAATCTTCCTTCAATCTGAGGAAACCCTCTGTGCAT |
| | TGGAGTGATGCTGCTGAGTACTTCTGTGCTGTGGGTGCGTACAGCAGTGCTTCCAAGATAATCTTTG |
| | GATCAGGGACCAGACTCAGCATCCGGCCAAATATCCAGAACCCTGACCCTGCCGTGTACCAGCTGA |
| | GAGACT |
| 296 | AAAATGCCCCTCCTTTCCTCCACAGGACCAGATGCCTGAGCTAGGAAAGGCCTCATTCCTGCTGTGA |
| | TCCTGCCATGGATACCTGGCTCGTATGCTGGGCAATTTTTAGTCTCTTGAAAGCAGGACTCACAGAA |
| | CCTGAAGTCACCCAGACTCCCAGCCATCAGGTCACACAGATGGGACAGGAAGTGATCTTGCGCTGT |
| | GTCCCCATCTCTAATCACTTATACTTCTATTGGTACAGACAAATCTTGGGGCAGAAAGTCGAGTTTCT |
| | GGTTTCCTTTTATAATAATGAAATCTCAGAGAAGTCTGAAATATTCGATGATCAATTCTCAGTTGAAAG |
| | GCCTGATGGATCAAATTTCACTCTGAAGATCCGGTCCACAAAGCTGGAGGACTCAGCCATGTACTTC |
| | TGTGCCAGCAGGGACAGGGGATCTGGGGCCAACGTCCTGACTTTCGGGGCCGGCAGCAGGCTGAC |
| | CGTGCTGGAGGACCTGAAAAACGTGTTCCCACCCGAGGTCGCTGTGTTTGAGCCATCAGA |
| 297 | GTCATCCCTCCTCGCTGGTGAATGGAGGCAGTGGTCACAACTCTCCCCAGAGAAGGTGGTGTGAGG |
| | CCATCACGGAAGATGCTGCTGCTTCTGCTGCTTCTGGGGCCAGGCTCCGGGCTTGGTGCTGTCGTC |
| | TCTCAACATCCGAGCTGGGTTATCTGTAAGAGTGGAACCTCTGTGAAGATCGAGTGCCGTTCCCTGG |
| | ACTTTCAGGCCACAACTATGTTTTGGTATCGTCAGTTCCCGAAACAGAGTCTCATGCTGATGGCAACT |
| | TCCAATGAGGGCTCCAAGGCCACATACGAGCAAGGCGTCGAGAAGGACAAGTTTCTCATCAACCAT |
| | GCAAGCCTGACCTTGTCCACTCTGACAGTGACCAGTGCCCATCCTGAAGACAGCAGCTTCTACATCT |
| | GCAGTGGGGGGTGGGGACCTAACACCGGGGAGCTGTTTTTTGGAGAAGGCTCTAGGCTGACCGTA |
| | CTGGAGGACCTGAAAAACGTGTTCCCACCCGAGGTCGCTGTGTTTGAGCCATCAGA |
| 298 | GGTTGGCTGAATTGCCAAATTGCTATTTCCCTTTTTCTGAGTGTGTGTCTGTGTCCGTGTGTGTGCAT |
| | GTGTGTGTGCACGCACGCGCACACAGGCAGACGCTAGGGGTAGAGTGTGATGGTTCAAAAAGAAAA |
| | GGAAACGCTTAAAGGGAGTGAATCACGTTTTGCCCAGGAAAACACACTTGATAACTGAAGGATGATG |
| | AAGTGTCCACAGGCTTTACTAGCTATCTTTTGGCTTCTACTGAGCTGGGTGAGCAGTGAAGACAAGG |
| | TGGTACAAAGCCCTCTATCTCTGGTTGTCCACGAGGGAGACACCGTAACTCTCAATTGCAGTTATGA |
| | AGTGACTAACTTTCGAAGCCTACTATGGTACAAGCAGGAAAAGAAAGCTCCCACATTTCTATTTATGC |
| | TAACTTCAAGTGGAATTGAAAAGAAGTCAGGAAGACTAAGTAGCATATTAGATAAGAAAGAACTTTCC |
| | AGCATCCTGAACATCACAGCCACCCAGACCGGAGACTCGGCCATCTACCTCTGTGCTGTGGGGCAC |
| | TTCTCATCTCGGTCCTCTGGTTCTGCAAGGCAACTGACCTTTGGATCTGGGACACAATTGACTGTTTT |
| | ACCTGATATCCAGAACCCTGACCCTGCCGTGTACCAGCTGAGAGACT |
| 299 | ATGCTCACAGAGGGCCTGGTCTAGAATATTCCACATCTGCTCTCACTCTGCCATGGACTCCTGGACC |
| | TTCTGCTGTGTGTCCCTTTGCATCCTGGTAGCGAAGCATACAGATGCTGGAGTTATCCAGTCACCCC |
| | GCCATGAGGTGACAGAGATGGGACAAGAAGTGACTCTGAGATGTAAACCAATTTCAGGCCACAACT |
| | CCCTTTTCTGGTACAGACAGACCATGATGCGGGGACTGGAGTTGCTCATTTACTTTAACAACAACGT |
| | TCCGATAGATGATTCAGGGATGCCCGAGGATCGATTCTCAGCTAAGATGCCTAATGCATCATTCTCC |
| | ACTCTGAAGATCCAGCCCTCAGAACCCAGGGACTCAGCTGTGTACTTCTGTGCCAGCAGTTTAAAGG |
| | TCGGTGTAGACAGCTCCTACAATGAGCAGTTCTTCGGGCCAGGGACACGGCTCACCGTGCTAGAGG |
| | ACCTGAAAAACGTGTTCCCACCCGAGGTCGCTGTGTTTGAGCCATCAGA |
| 300 | GGCCACAAGGTGGCAGAGCTTCTCTGCTCATGTAGAATGTACGAAAGGATCCTTTTGGTTGACTTCT |
| | GTGAGTAGGCTGACGACAGAAGCAGATGCCTCTGTGGACAGTTTAAGAAACCACAGTGCTTTGGAG |
| | GAAAGGAAGAGATACTTGATAATATAGCTCTCTTGGCTGGAGATTGCAGGTCCCAGTGGGGAGAACA |
| | ATGAAGACATTTGCTGGATTTTCGTTCCTGTTTTTGTGGCTGCAGCTGGACTGTATGAGTAGAGGAG |
| | AGGATGTGGAGCAGAGTCTTTTCCTGAGTGTCCGAGAGGGAGACAGCTCCGTTATAAACTGCACTTA |
| | CACAGACAGCTCCTCCACCTACTTATACTGGTATAAGCAAGAACCTGGAGCAGGTCTCCAGTTGCTG |
| | ACGTATATTTTTTCAAATATGGACATGAAACAAGACCAAAGACTCACTGTTCTATTGAATAAAAAGGAT |
| | AAACATCTGTCTCTGCGCATTGCAGACACCCAGACTGGGGACTCAGCTATCTACTTCTGTGCAGAGA |
| | GCCCTTCAGATGGCCAGAAGCTGCTCTTTGCAAGGGGAACCATGTTAAAGGTGGATCTTAATATCCA |
| | GAACCCTGACCCTGCCGTGTACCAGCTGAGAGACT |
| 301 | ACCTGGAGCCCCCAGAACTGGCAGACACCTGCCTGATGCTGCCATGGGCCCCCAGCTCCTTGGCTA |
| | TGTGGTCCTTTGCCTTCTAGGAGCAGGCCCCCTGGAAGCCCAAGTGACCCAGAACCCAAGATACCT |
| | CATCACAGTGACTGGAAAGAAGTTAACAGTGACTTGTTCTCAGAATATGAACCATGAGTATATGTCCT |
| | GGTATCGACAAGACCCAGGGCTGGGCTTAAGGCAGATCTACTATTCAATGAATGTTGAGGTGACTGA |
| | TAAGGGAGATGTTCCTGAAGGGTACAAAGTCTCTCGAAAAGAGAAGAGGAATTTCCCCCTGATCCTG |
| | GAGTCGCCCAGCCCCAACCAGACCTCTCTGTACTTCTGTGCCAGCAGTTTGAGACAGGGTATAAGT |
| | CCTGAGCAGTTCTTCGGGCCAGGGACACGGCTCACCGTGCTAGAGGACCTGAAAAACGTGTTCCCA |
| | CCCGAGGTCGCTGTGTTTGAGCCATCAGA |
| 302 | GGAAATCGTGTTTCTGTAGAGAAAGAAAAACTACCATATTTGGATAGCCCTGGCCAACTTTCAAGGCT |
| | CCTAAATCTGAGTTTTCAGTGAACTGGACAGAAAAAAAAAATGAAGAAGCTACTAGCAATGATTCTGT |
| | GGCTTCAACTAGACCGGTTAAGTGGAGAGCTGAAAGTGGAACAAAACCCTCTGTTCCTGAGCATGCA |
| | GGAGGGAAAAAACTATACCATCTACTGCAATTATTCAACCACTTCAGACAGACTGTATTGGTACAGGC |
| | AGGATCCTGGGAAAAGTCTGGAATCTCTGTTTGTGTTGCTATCAAATGGAGCAGTGAAGCAGGAGG |
| | GACGATTAATGGCCTCACTTGATACCAAAGCCCGTCTCAGCACCCTCCACATCACAGCTGCCGTGCA |
| | TGACCTCTCTGCCACCTACTTCTGTGCCGTGGAAGAAACCAGTGGCTCTAGGTTGACCTTTGGGGAA |
| | GGAACACAGCTCACAGTGAATCCTGATATCCAGAACCCTGACCCTGCCGTGTACCAGCTGAGAGAC |
| | T |
| 303 | ACCTGGAGCCCCCAGAACTGGCAGACACCTGCCTGATGCTGCCATGGGCCCCCAGCTCCTTGGCTA |
| | TGTGGTCCTTTGCCTTCTAGGAGCAGGCCCCCTGGAAGCCCAAGTGACCCAGAACCCAAGATACCT |
| | CATCACAGTGACTGGAAAGAAGTTAACAGTGACTTGTTCTCAGAATATGAACCATGAGTATATGTCCT |
| | GGTATCGACAAGACCCAGGGCTGGGCTTAAGGCAGATCTACTATTCAATGAATGTTGAGGTGACTGA |
| | TAAGGGAGATGTTCCTGAAGGGTACAAAGTCTCTCGAAAAGAGAAGAGGAATTTCCCCCTGATCCTG |
| | GAGTCGCCCAGCCCCAACCAGACCTCTCTGTACTTCTGTGCCAGCAGTTTGAGACAGGGTATAAGT |
| | CCTGAGCAGTTCTTCGGGCCAGGGACACGGCTCACCGTGCTAGAGGACCTGAAAAACGTGTTCCCA |
| | CCCGAGGTCGCTGTGTTTGAGCCATCAGA |
| 304 | ACCTGGAGCCCCCAGAACTGGCAGACACCTGCCTGATGCTGCCATGGGCCCCCAGCTCCTTGGCTA |
| | TGTGGTCCTTTGCCTTCTAGGAGCAGGCCCCCTGGAAGCCCAAGTGACCCAGAACCCAAGATACCT |
| | CATCACAGTGACTGGAAAGAAGTTAACAGTGACTTGTTCTCAGAATATGAACCATGAGTATATGTCCT |
| | GGTATCGACAAGACCCAGGGCTGGGCTTAAGGCAGATCTACTATTCAATGAATGTTGAGGTGACTGA |
| | TAAGGGAGATGTTCCTGAAGGGTACAAAGTCTCTCGAAAAGAGAAGAGGAATTTCCCCCTGATCCTG |
| | GAGTCGCCCAGCCCCAACCAGACCTCTCTGTACTTCTGTGCCAGCAGTGAGCGACAGGGGATAACT |
| | GAAGCTTTCTTTGGACAAGGCACCAGACTCACAGTTGTAGAGGACCTGAACAAGGTGTTCCCACCC |
| | GAGGTCGCTGTGTTTGAGCCATCAGA |
| 305 | GTAGCTCGTTGATATCTGTGTGGATAGGGAGCTGTGACGAGAGCAAGAGGTCAGAACACATCCAGG |
| | CTCCTTAAGGGAAAGCCTCTTTCTGTTTCTGAAACTTTTCAAAGCCAGGGACTTGTCCAATCCAACCT |
| | CCTCACAGTTCCTAGCTCCTGAGGCTCAGCGCCCTTGGCTTCTGTCCGCCCAGCTCAAGGTCCTGC |
| | AGCATTGCCACTGCTCAGCCATGCTCCTGCTGCTCGTCCCAGCGTTCCAGGTGATTTTTACCCTGGG |
| | AGGAACCAGAGCCCAGTCTGTGACCCAGCTTGACAGCCAAGTCCCTGTCTTTGAAGAAGCCCCTGT |
| | GGAGCTGAGGTGCAACTACTCATCGTCTGTTTCAGTGTATCTCTTCTGGTATGTGCAATACCCCAAC |
| | CAAGGACTCCAGCTTCTCCTGAAGTATTTATCAGGATCCACCCTGGTTAAAGGCATCAACGGTTTTG |
| | AGGCTGAATTTAACAAGAGTCAAACTTCCTTCCACTTGAGGAAACCCTCAGTCCATATAAGCGACAC |
| | GGCTGAGTACTTCTGTGCTGTGAGTGGTACTGGAGCCAATAGTAAGCTGACATTTGGAAAAGGAATA |
| | ACTCTGAGTGTTAGACCAGATATCCAGAACCCTGACCCTGCCGTGTACCAGCTGAGAGACT |
| 306 | ACAGTGATCCTGATCTGGTAAAGCTCCCATCCTGCCCTGACCCTGCCATGGGCACCAGGCTCCTCTT |
| | CTGGGTGGCCTTCTGTCTCCTGGGGGCAGATCACACAGGAGCTGGAGTCTCCCAGTCCCCCAGTAA |
| | CAAGGTCACAGAGAAGGGAAAGGATGTAGAGCTCAGGTGTGATCCAATTTCAGGTCATACTGCCCTT |
| | TACTGGTACCGACAGAGCCTGGGGCAGGGCCTGGAGTTTTTAATTTACTTCCAAGGCAACAGTGCA |
| | CCAGACAAATCAGGGCTGCCCAGTGATCGCTTCTCTGCAGAGAGGACTGGGGGATCCGTCTCCACT |
| | CTGACGATCCAGCGCACACAGCAGGAGGACTCGGCCGTGTATCTCTGTGCCAGCAGCGACCCCATT |
| | AGCGGGAGAGGGGATGAGCAGTTCTTCGGGCCAGGGACACGGCTCACCGTGCTAGAGGACCTGAA |
| | AAACGTGTTCCCACCCGAGGTCGCTGTGTTTGAGCCATCAGA |
| 307 | ACAGTGATCCTGATCTGGTAAAGCTCCCATCCTGCCCTGACCCTGCCATGGGCACCAGGCTCCTCTT |
| | CTGGGTGGCCTTCTGTCTCCTGGGGGCAGATCACACAGGAGCTGGAGTCTCCCAGTCCCCCAGTAA |
| | CAAGGTCACAGAGAAGGGAAAGGATGTAGAGCTCAGGTGTGATCCAATTTCAGGTCATACTGCCCTT |
| | TACTGGTACCGACAGAGCCTGGGGCAGGGCCTGGAGTTTTTAATTTACTTCCAAGGCAACAGTGCA |
| | CCAGACAAATCAGGGCTGCCCAGTGATCGCTTCTCTGCAGAGAGGACTGGGGGATCCGTCTCCACT |
| | CTGACGATCCAGCGCACACAGCAGGAGGACTCGGCCGTGTATCTCTGTGCCAGCAGCTTTGGGGG |
| | GGGGGCGTGGAATGAGCAGTTCTTCGGGCCAGGGACACGGCTCACCGTGCTAGAGGACCTGAAAA |
| | ACGTGTTCCCACCCGAGGTCGCTGTGTTTGAGCCATCAGA |
| 308 | GACTGTGATTTCTTCATGTTAAGGATCAAGACCATTATTTGGGTAACACACTAAAGATGAACTATTCTC |
| | CAGGCTTAGTATCTCTGATACTCTTACTGCTTGGAAGAACCCGTGGAAATTCAGTGACCCAGATGGA |
| | AGGGCCAGTGACTCTCTCAGAAGAGGCCTTCCTGACTATAAACTGCACGTACACAGCCACAGGATA |
| | CCCTTCCCTTTTCTGGTATGTCCAATATCCTGGAGAAGGTCTACAGCTCCTCCTGAAAGCCACGAAG |
| | GCTGATGACAAGGGAAGCAACAAAGGTTTTGAAGCCACATACCGTAAAGAAACCACTTCTTTCCACT |
| | TGGAGAAAGGCTCAGTTCAAGTGTCAGACTCAGCGGTGTACTTCTGTGCTCTGAGGCACAAAGCTG |
| | CAGGCAACAAGCTAACTTTTGGAGGAGGAACCAGGGTGCTAGTTAAACCAAATATCCAGAACCCTGA |
| | CCCTGCCGTGTACCAGCTGAGAGACT |
| 309 | CTGGAAGACCACCTGGGCTGTCATTGAGCTCTGGTGCCAGGAGGAATGGACAAGATCTTAGGAGCA |
| | TCATTTTTAGTTCTGTGGCTTCAACTATGCTGGGTGAGTGGCCAACAGAAGGAGAAAAGTGACCAGC |
| | AGCAGGTGAAACAAAGTCCTCAATCTTTGATAGTCCAGAAAGGAGGGATTTCAATTATAAACTGTGCT |
| | TATGAGAACACTGCGTTTGACTACTTTCCATGGTACCAACAATTCCCTGGGAAAGGCCCTGCATTATT |
| | GATAGCCATACGTCCAGATGTGAGTGAAAAGAAAGAAGGAAGATTCACAATCTCCTTCAATAAAAGT |
| | GCCAAGCAGTTCTCATTGCATATCATGGATTCCCAGCCTGGAGACTCAGCCACCTACTTCTGTGCAG |
| | CGCTATTTGATGGAGGAAGCCAAGGAAATCTCATCTTTGGAAAAGGCACTAAACTCTCTGTTAAACCA |
| | AATATCCAGAACCCTGACCCTGCCGTGTACCAGCTGAGAGACT |
| 310 | GTCATCCCTCCTCGCTGGTGAATGGAGGCAGTGGTCACAACTCTCCCCAGAGAAGGTGGTGTGAGG |
| | CCATCACGGAAGATGCTGCTGCTTCTGCTGCTTCTGGGGCCAGGCTCCGGGCTTGGTGCTGTCGTC |
| | TCTCAACATCCGAGCTGGGTTATCTGTAAGAGTGGAACCTCTGTGAAGATCGAGTGCCGTTCCCTGG |
| | ACTTTCAGGCCACAACTATGTTTTGGTATCGTCAGTTCCCGAAACAGAGTCTCATGCTGATGGCAACT |
| | TCCAATGAGGGCTCCAAGGCCACATACGAGCAAGGCGTCGAGAAGGACAAGTTTCTCATCAACCAT |
| | GCAAGCCTGACCTTGTCCACTCTGACAGTGACCAGTGCCCATCCTGAAGACAGCAGCTTCTACATCT |
| | GCAGTGCTAGCGGCCCTGAAAAACTGTTTTTTGGCAGTGGAACCCAGCTCTCTGTCTTGGAGGACCT |
| | GAACAAGGTGTTCCCACCCGAGGTCGCTGTGTTTGAGCCATCAGA |
| 311 | AAAGCAGATTCTTTTTATGATTTTTAAAGTAGAAATATCCATTCTAGGTGCATTTTTTAAGGGTTTAAAA |
| | TTTGAATCCTCAGTGAACCAGGGCAGAGAAGAATGATGAAATCCTTGAGAGTTTTACTAGTGATCCT |
| | GTGGCTTCAGTTGAGCTGGGTTTGGAGCCAACAGAAGGAGGTGGAGCAGAATTCTGGACCCCTCAG |
| | TGTTCCAGAGGGAGCCATTGCCTCTCTCAACTGCACTTACAGTGACCGAGGTTCCCAGTCCTTCTTC |
| | TGGTACAGACAATATTCTGGGAAAAGCCCTGAGTTGATAATGTTCATATACTCCAATGGTGACAAAGA |
| | AGATGGAAGGTTTACAGCACAGCTCAATAAAGCCAGCCAGTATGTTTCTCTGCTCATCAGAGACTCC |
| | CAGCCCAGTGATTCAGCCACCTACCTCTGTGCCGTGATCCCGAATTCCGGGTATGCACTCAACTTCG |
| | GCAAAGGCACCTCGCTGTTGGTCACACCCCATATCCAGAACCCTGACCCTGCCGTGTACCAGCTGA |
| | GAGACT |
| 312 | GAGAGTCCTGCTCCCCTTTCATCAATGCACAGATACAGAAGACCCCTCCGTCATGCAGCATCTGCCA |
| | TGAGCATCGGCCTCCTGTGCTGTGCAGCCTTGTCTCTCCTGTGGGCAGGTCCAGTGAATGCTGGTG |
| | TCACTCAGACCCCAAAATTCCAGGTCCTGAAGACAGGACAGAGCATGACACTGCAGTGTGCCCAGG |
| | ATATGAACCATGAATACATGTCCTGGTATCGACAAGACCCAGGCATGGGGCTGAGGCTGATTCATTA |
| | CTCAGTTGGTGCTGGTATCACTGACCAAGGAGAAGTCCCCAATGGCTACAATGTCTCCAGATCAACC |
| | ACAGAGGATTTCCCGCTCAGGCTGCTGTCGGCTGCTCCCTCCCAGACATCTGTGTACTTCTGTGCCA |
| | GCAGGGGACTAGCGGGAGAGACCCAGTACTTCGGGCCAGGCACGCGGCTCCTGGTGCTCGAGGA |
| | CCTGAAAAACGTGTTCCCACCCGAGGTCGCTGTGTTTGAGCCATCAGA |
| 313 | GACTGTGATTTCTTCATGTTAAGGATCAAGACCATTATTTGGGTAACACACTAAAGATGAACTATTCTC |
| | CAGGCTTAGTATCTCTGATACTCTTACTGCTTGGAAGAACCCGTGGAAATTCAGTGACCCAGATGGA |
| | AGGGCCAGTGACTCTCTCAGAAGAGGCCTTCCTGACTATAAACTGCACGTACACAGCCACAGGATA |
| | CCCTTCCCTTTTCTGGTATGTCCAATATCCTGGAGAAGGTCTACAGCTCCTCCTGAAAGCCACGAAG |
| | GCTGATGACAAGGGAAGCAACAAAGGTTTTGAAGCCACATACCGTAAAGAAACCACTTCTTTCCACT |
| | TGGAGAAAGGCTCAGTTCAAGTGTCAGACTCAGCGGTGTACTTCTGTGCTCTGAGGGGCCGGAACC |
| | AGGGAGGAAAGCTTATCTTCGGACAGGGAACGGAGTTATCTGTGAAACCCAATATCCAGAACCCTGA |
| | CCCTGCCGTGTACCAGCTGAGAGACT |
| 314 | GTCATCCCTCCTCGCTGGTGAATGGAGGCAGTGGTCACAACTCTCCCCAGAGAAGGTGGTGTGAGG |
| | CCATCACGGAAGATGCTGCTGCTTCTGCTGCTTCTGGGGCCAGGCTCCGGGCTTGGTGCTGTCGTC |
| | TCTCAACATCCGAGCAGGGTTATCTGTAAGAGTGGAACCTCTGTGAAGATCGAGTGCCGTTCCCTGG |
| | ACTTTCAGGCCACAACTATGTTTTGGTATCGTCAGTTCCCGAAACAGAGTCTCATGCTGATGGCAACT |
| | TCCAATGAGGGCTCCAAGGCCACATACGAGCAAGGCGTCGAGAAGGACAAGTTTCTCATCAACCAT |
| | GCAAGCCTGACCTTGTCCACTCTGACAGTGACCAGTGCCCATCCTGAAGACAGCAGCTTCTACATCT |
| | GCAGTGCTAGACTTTCTAGCGGGGGGGGCTATGAGCAGTTCTTCGGGCCAGGGACACGGCTCACC |
| | GTGCTAGAGGACCTGAAAAACGTGTTCCCACCCGAGGTCGCTGTGTTTGAGCCATCAGA |
| 315 | CTGGGCTCATTGCAGCTCAGACACAGCAAAAGAGCCTAGAACCTGGGTCCTAGTTTGCACCTAGAAT |
| | ATGAGGCAAGTGGCGAGAGTGATCGTGTTCCTGACCCTGAGTACTTTGAGCCTTGCTAAGACCACC |
| | CAGCCCATCTCCATGGACTCATATGAAGGACAAGAAGTGAACATAACCTGTAGCCACAACAACATTG |
| | CTACAAATGATTATATCACGTGGTACCAACAGTTTCCCAGCCAAGGACCACGATTTATTATTCAAGGA |
| | TACAAGACAAAAGTTACAAACGAAGTGGCCTCCCTGTTTATCCCTGCCGACAGAAAGTCCAGCACTC |
| | TGAGCCTGCCCCGGGTTTCCCTGAGCGACACTGCTGTGTACTACTGCCTCGTGGTCCCTGTTTATAA |
| | CCAGGGAGGAAAGCTTATCTTCGGACAGGGAACGGAGTTATCTGTGAAACCCAATATCCAGAACCCT |
| | GACCCTGCCGTGTACCAGCTGAGAGACT |
| 316 | AGAGGACCAGTATCCCTCACAGGGTGACACCTGACCAGCTCTGTCCCACCTGGCCATGGGCTCCAG |
| | GTACCTCTGATGGGAAGACCTTTGTCTCTTGGGAACAAGTGAATCCTTGGCACAGGCCCAGTGGATT |
| | CTGCTGTGCAGAACAGAGAGCAGTGGACCTCAGGAGGCCTGCAAGGGGAGGACATAGGACAGTGA |
| | CATCACAGTATGCCCCTCCCACCAGGAAAAGCAAGGCTGAGAATTTAGCTCTTTCCCAGGAGGACCA |
| | AGCCCTGAGCACAGACACAGTGCTGCCTGCCCCTTTGTGCCATGGGCTCCAGGCTGCTCTGTTGGG |
| | TGCTGCTTTGTCTCCTGGGAGCAGGCCCAGTAAAGGCTGGAGTCACTCAAACTCCAAGATATCTGAT |
| | CAAAACGAGAGGACAGCAAGTGACACTGAGCTGCTCCCCTATCTCTGGGCATAGGAGTGTATCCTG |
| | GTACCAACAGACCCCAGGACAGGGCCTTCAGTTCCTCTTTGAATACTTCAGTGAGACACAGAGAAAC |
| | AAAGGAAACTTCCCTGGTCGATTCTCAGGGCGCCAGTTCTCTAACTCTCGCTCTGAGATGAATGTGA |
| | GCACCTTGGAGCTGGGGGACTCGGCCCTTTATCTTTGCGCCAGCAGCTCGGGGACTAGCGGGATG |
| | GGAGAGACCCAGTACTTCGGGCCAGGCACGCGGCTCCTGGTGCTCGAGGACCTGAAAAACGTGTT |
| | CCCACCCGAGGTCGCTGTGTTTGAGCCATCAGA |
| 317 | GGTTCTTGTTACATTTGCGTTTGGGTTCTTGGTCATGAAGTCTTTGCCTAAGCCAGTGTCTAGAAGGG |
| | TTTTTCTGATGGTATCTTCTAGAATTTTTATGGTTTTAGGTGTTTTATTTTCTTTTTTTGTTTATGTGGAA |
| | GCTGTTCTATCTTTAGCTGACGATTTCTGGGGAGTGGGGAAATTGAAACCTGCCTGATGTGGGATGT |
| | GCTGTGGCTGCTGCTTTGTTGCTTGGGACCTCCTCTGACCTAGGATCAGACACAGAGTCTGAGTTCT |
| | GGGGCCTGGAACCTCAATGTGCACTTGAACAATGAAGTTGGTGACAAGCATTACTGTACTCCTATCT |
| | TTGGGTATTATGGGTGATGCTAAGACCACACAGCCAAATTCAATGGAGAGTAACGAAGAAGAGCCTG |
| | TTCACTTGCCTTGTAACCACTCCACAATCAGTGGAACTGATTACATACATTGGTATCGACAGCTTCCC |
| | TCCCAGGGTCCAGAGTACGTGATTCATGGTCTTACAAGCAATGTGAACAACAGAATGGCCTCTCTGG |
| | CAATCGCTGAAGACAGAAAGTCCAGTACCTTGATCCTGCACCGTGCTACCTTGAGAGATGCTGCTGT |
| | GTACTACTGCATCCTGAGAGACGTGGGCGGATCTGAAAAGCTGGTCTTTGGAAAGGGAACGAAACT |
| | GACAGTAAACCCATATATCCAGAACCCTGACCCTGCCGTGTACCAGCTGAGAGACT |
| 318 | GTCATCCCTCCTCGCTGGTGAATGGAGGCAGTGGTCACAACTCTCCCCAGAGAAGGTGGTGTGAGG |
| | CCATCACGGAAGATGCTGCTGCTTCTGCTGCTTCTGGGGCCAGGCTCCGGGCTTGGTGCTGTCGTC |
| | TCTCAACATCCGAGCTGGGTTATCTGTAAGAGTGGAACCTCTGTGAAGATCGAGTGCCGTTCCCTGG |
| | ACTTTCAGGCCACAACTATGTTTTGGTATCGTCAGTTCCCGAAACAGAGTCTCATGCTGATGGCAACT |
| | TCCAATGAGGGCTCCAAGGCCACATACGAGCAAGGCGTCGAGAAGGACAAGTTTCTCATCAACCAT |
| | GCAAGCCTGACCTTGTCCACTCTGACAGTGACCAGTGCCCATCCTGAAGACAGCAGCTTCTACATCT |
| | GCAGTGCCAAAGGCCTCGAAAATCAGCCCCAGCATTTTGGTGATGGGACTCGACTCTCCATCCTAG |
| | AGGACCTGAACAAGGTGTTCCCACCCGAGGTCGCTGTGTTTGAGCCATCAGA |
| 319 | CTTCTGCAGACTACAGTGGCTCAGGAACCGGGGATGCAGTGCCAGGCTCATGGTATCCTGCAGCAG |
| | ATGTGGGGAGCTTTCCTTCTCTATGTTTCCATGAAGATGGGAGGCACTGCAGGACAAAGCCTTGAGC |
| | AGCCCTCTGAAGTGACAGCTGTGGAAGGAGCCATTGTCCAGATAAACTGCACGTACCAGACATCTG |
| | GGTTTTATGGGCTGTCCTGGTACCAGCAACATGATGGCGGAGCACCCACATTTCTTTCTTACAATGC |
| | TCTGGATGGTTTGGAGGAGACAGGTCGTTTTTCTTCATTCCTTAGTCGCTCTGATAGTTATGGTTACC |
| | TCCTTCTACAGGAGCTCCAGATGAAAGACTCTGCCTCTTACTTCTGCGCTGTCCTTAAAAAGGGGTA |
| | TGCACTCAACTTCGGCAAAGGCACCTCGCTGTTGGTCACACCCCATATCCAGAACCCTGACCCTGC |
| | CGTGTACCAGCTGAGAGACT |
| 320 | GAGATTCTGGCTGATGATGTCACTGAGACAAAGGAAAAAATGCAAAACAGGTAGTCTTAAAGAAGCA |
| | TTCTGGTGAGACGGGGGGCATTTTGGCCATGGCTTTGCAGAGCACTCTGGGGGCGGTGTGGCTAG |
| | GGCTTCTCCTCAACTCTCTCTGGAAGGTTGCAGAAAGCAAGGACCAAGTGTTTCAGCCTTCCACAGT |
| | GGCATCTTCAGAGGGAGCTGTGGTGGAAATCTTCTGTAATCACTCTGTGTCCAATGCTTACAACTTCT |
| | TCTGGTACCTTCACTTCCCGGGATGTGCACCAAGACTCCTTGTTAAAGGCTCAAAGCCTTCTCAGCA |
| | GGGACGATACAACATGACCTATGAACGGTTCTCTTCATCGCTGCTCATCCTCCAGGTGCGGGAGGC |
| | AGATGCTGCTGTTTACTACTGTGCTGTGGCTGGTGGCTACAATAAGCTGATTTTTGGAGCAGGGACC |
| | AGGCTGGCTGTACACCCATATATCCAGAACCCTGACCCTGCCGTGTACCAGCTGAGAGACT |
| 321 | AGTGACCCTGATCTGGCAAAGCTTCCATCCTGCCCTGACCCTGCCATGGGTACCAGGCTCCTCTGC |
| | TGGGTGGCCTTCTGTCTCCTGGTGGAAGAACTCATAGAAGCTGGAGTGGTTCAGTCTCCCAGATATA |
| | AGATTATAGAGAAAAAACAGCCTGTGGCTTTTTGGTGCAATCCTATTTCTGGCCACAATACCCTTTAC |
| | TGGTACCTGCAGAACTTGGGACAGGGCCCGGAGCTTCTGATTCGATATGAGAATGAGGAAGCAGTA |
| | GACGATTCACAGTTGCCTAAGGATCGATTTTCTGCAGAGAGGCTCAAAGGAGTAGACTCCACTCTCA |
| | AGATCCAGCCTGCAGAGCTTGGGGACTCGGCCGTGTATCTCTGTGCCAGCAGCTTAGAAGCGGGG |
| | GACTCCTACGAGCAGTACTTCGGGCCGGGCACCAGGCTCACGGTCACAGAGGACCTGAAAAACGT |
| | GTTCCCACCCGAGGTCGCTGTGTTTGAGCCATCAGA |
| 322 | GTCACTGACACAAAGGAAAAAATGCAAAACAGGTAGTATTGGAGAAGCATTCTGGTGTGTTGGGGG |
| | GCATTTTGGCCATGGCTTTGCAGAGCACTCTGGGGGCGGTGTGGCTAGGGCTTCTCCTCAACTCTC |
| | TCTGGAAGGTTGCAGAAAGCAAGGACCAAGTGTTTCAGCCTTCCACAGTGGCATCTTCAGAGGGAG |
| | CTGTGGTGGAAATCTTCTGTAATCACTCTGTGTCCAATGCTTACAACTTCTTCTGGTACCTTCACTTC |
| | CCGGGATGTGCACCAAGACTCCTTGTTAAAGGCTCAAAGCCTTCTCAGCAGGGACGATACAACATGA |
| | CCTATGAACGGTTCTCTTCATCGCTGCTCATCCTCCAGGTGCGGGAGGCAGATGCTGCTGTTTACTA |
| | CTGTGCTGTGACTGGTGGCTACAATAAGCTGATTTTTGGAGCAGGGACCAGGCTGGCTGTACACCC |
| | ATATATCCAGAACCCTGACCCTGCCGTGTACCAGCTGAGAGACT |
| 323 | AGTGACCCTGATCTGGCAAAGCTTCCATCCTGCCCTGACCCTGCCATGGGTACCAGGCTCCTCTGC |
| | TGGGTGGCCTTCTGTCTCCTGGTGGAAGAACTCATAGAAGCTGGAGTGGTTCAGTCTCCCAGATATA |
| | AGATTATAGAGAAAAAACAGCCTGTGGCTTTTTGGTGCAATCCTATTTCTGGCCACAATACCCTTTAC |
| | TGGTACCTGCAGAACTTGGGACAGGGCCCGGAGCTTCTGATTCGATATGAGAATGAGGAAGCAGTA |
| | GACGATTCACAGTTGCCTAAGGATCGATTTTCTGCAGAGAGGCTCAAAGGAGTAGACTCCACTCTCA |
| | AGATCCAGCCTGCAGAGCTTGGGGACTCGGCCGTGTATCTCTGTGCCAGCAGCTTAGAAGCGGGA |
| | GATTCCTACGAGCAGTACTTCGGGCCGGGCACCAGGCTCACGGTCACAGAGGACCTGAAAAACGTG |
| | TTCCCACCCGAGGTCGCTGTGTTTGAGCCATCAGA |
| 324 | CTGGAACAGAGATTTAAATTTCGTGGTAGTGTTTTTAGGGGGACTCTAAGCCCAAGAGAGTTTTTTTA |
| | AAAAAAAAAAAAAAAAAACCCATTCAGGAAATAATTCTTTGCTGATAAGGATGCTCCTTGAACATTTAT |
| | TAATAATCTTGTGGATGCAGCTGACATGGGTCAGTGGTCAACAGCTGAATCAGAGTCCTCAATCTAT |
| | GTTTATCCAGGAAGGAGAAGATGTCTCCATGAACTGCACTTCTTCAAGCATATTTAACACCTGGCTAT |
| | GGTACAAGCAGGAACCTGGGGAAGGTCCTGTCCTCTTGATAGCCTTATATAAGGCTGGTGAATTGAC |
| | CTCAAATGGAAGACTGACTGCTCAGTTTGGTATAACCAGAAAGGACAGCTTCCTGAATATCTCAGCA |
| | TCCATACCTAGTGATGTAGGCATCTACTTCTGTGCTGGGCACCAAATTCAGGGAGCCCAGAAGCTGG |
| | TATTTGGCCAAGGAACCAGGCTGACTATCAACCCAAATATCCAGAACCCTGACCCTGCCGTGTACCA |
| | GCTGAGAGACT |
| 325 | ATCAATGCACAGATACAGAAGACCCCTCCGTCCTGGAGCACCTGCCATGAGCATCAGCCTCCTGTG |
| | CTGTGCAGCCTTTCCTCTCCTGTGGGCAGGTCCAGTGAATGCTGGTGTCACTCAGACCCCAAAATTC |
| | CGCATCCTGAAGATAGGACAGAGCATGACACTGCAGTGTACCCAGGATATGAACCATAACTACATGT |
| | ACTGGTATCGACAAGACCCAGGCATGGGGCTGAAGCTGATTTATTATTCAGTTGGTGCTGGTATCAC |
| | TGATAAAGGAGAAGTCCCGAATGGCTACAACGTCTCCAGATCAACCACAGAGGATTTCCCGCTCAG |
| | GCTGGAGTTGGCTGCTCCCTCCCAGACATCTGTGTACTTCTGTGCCAGCAGTTACTCGGGGATGAA |
| | CACTGAAGCTTTCTTTGGACAAGGCACCAGACTCACAGTTGTAGAGGACCTGAACAAGGTGTTCCCA |
| | CCCGAGGTCGCTGTGTTTGAGCCATCAGA |
| 326 | AAGTCTCTCAGCTGGTACACGGCAGGGTCAGGGTTCTGGATATTGGGCTTCACCACCAGCTGAGTT |
| | CCATCTCCAAACATGAGTCTGGCATTGTTATTCGCCCCAGCACAGAAGTAGATGCCTACATCACTAG |
| | GTATGGATGCTGAGATATTCAGGAAGCTGTCCTTTCTGGTTATACCAAACTGAGCAGTCAGTCTTCCA |
| | TTTGAGGTCAATTCACCAGCCTTATATAAGGCTATCAAGAGGACAGGACCTTCCCCAGGGTCCTGCT |
| | TGTACCATAGCCAGGTGTTAAATATGCTTGAAGAAGTGCAGTTCATGGAGACATCTTCTCCTTCCTGG |
| | ATAAACATAGATTGAGGACTCTGATTCAGCTGTTGACCACTGACCCATGTCAGCTGCATCCACAAGA |
| | TTATTAATATGAATTCGGATTGGGTTGGTTTTTTTGTTTTTAGTGTCACTCTAAGCCCAATAGAGTTTTT |
| | TTAAAAAAAAAAAAAAAAAAACCCATTCAGGAAATAATTCTTTGCTGATAAGGATGCTCCTTGAACATT |
| | TATTAATAATCTTGTGGATGCAGCTGACATGGGTCAGTGGTCAACAGCTGAATCAGAGTCCTCAATCT |
| | ATGTTTATCCAGGAAGGAGAAGATGTCTCCATGAACTGCACTTCTTCAAGCATATTTAACACCTGGCT |
| | ATGGTACAAGCAGGACCCTGGGGAAGGTCCTGTCCTCTTGATAGCCTTATATAAGGCTGGTGAATTG |
| | ACCTCAAATGGAAGACTGACTGCTCAGTTTGGTATAACCAGAAAGGACAGCTTCCTGAATATCTCAG |
| | CATCCATACCTAGTGATGTAGGCATCTACTTCTGTGCTGGGGCGAATAACAATGCCAGACTCATGTT |
| | TGGAGATGGAACTCAGCTGGTGGTGAAGCCCAATATCCAGAACCCTGACCCTGCCGTGTACCAGCT |
| | GAGAGACT |
| 327 | GAAGGAAAGATGAACTTGAGTTTCACTTCTTAGTGCCTTTTCTCAGGGGAGAGGCCATCACTTGAAG |
| | ATGCTGAGTCTTCTGCTCCTTCTCCTGGGACTAGGCTCTGTGTTCAGTGCTGTCATCTCTCAAAAGC |
| | CAAGCAGGGATATCTGTCAACGTGGAACCTCCCTGACGATCCAGTGTCAAGTCGATAGCCAAGTCA |
| | CCATGATGTTCTGGTACCGTCAGCAACCTGGACAGAGCCTGACACTGATCGCAACTGCAAATCAGG |
| | GCTCTGAGGCCACATATGAGAGTGGATTTGTCATTGACAAGTTTCCCATCAGCCGCCCAAACCTAAC |
| | ATTCTCAACTCTGACTGTGAGCAACATGAGCCCTGAAGACAGCAGCATATATCTCTGCAGCGTCACT |
| | CCGGGGGGCGGGGTGAACACTGAAGCTTTCTTTGGACAAGGCACCAGACTCACAGTTGTAGAGGAC |
| | CTGAACAAGGTGTTCCCACCCGAGGTCGCTGTGTTTGAGCCATCAGA |
| 328 | AAAGCAGATTCTTTTTATGATTTTTAAAGTAGAAATATCCATTCCAGGTGCATTTTTTAAGGGTTTAAAA |
| | TTTGAATCCTCAGTGAACCAGGGCAGAGAAGAATGATGAAATCCTTGAGAGTTTTACTAGTGATCCT |
| | GTGGCTTCAGTTGAGCTGGGTTTGGAGCCAACAGAAGGAGGTGGAGCAGAATTCTGGACCCCTCAG |
| | TGTTCCAGAGGGAGCCATTGCCTCTCTCAACTGCACTTACAGTGACCGAGGTTCCCAGTCCTTCTTC |
| | TGGTACAGACAATATTCTGGGAAAAGCCCTGAGTTGATAATGTCCATATACTCCAATGGTGACAAAGA |
| | AGATGGAAGGTTTACAGCACAGCTCAATAAAGCCAGCCAGTATGTTTCTCTGCTCATCAGAGACTCC |
| | CAGCCCAGTGATTCAGCCACCTACCTCTGTGCCGTGAGCGCTGGTGGTACTAGCTATGGAAAGCTG |
| | ACATTTGGACAAGGGACCATCTTGACTGTCCATCCAAATATCCAGAACCCTGACCCTGCCGTGTACC |
| | AGCTGAGAGACT |
| 329 | AGACCAGAATCCTGCCCTGGGCCTTGCCTGGTCTGCCTCACTCTGCCATGGGCTGCAGGCTCCTCT |
| | GCTGTGTGGTCTTCTGCCTCCTCCAAGCAGGTCCCTTGGACACAGCTGTTTCCCAGACTCCAAAATA |
| | CCTGGTCACACAGATGGGAAACGACAAGTCCATTAAATGTGAACAAAATCTGGGCCATGATACTATG |
| | TATTGGTATAAACAGGACTCTAAGAAATTTCTGAAGATAATGTTTAGCTACAATAATAAGGAGCTCATT |
| | ATAAATGAAACAGTTCCAAATCGCTTCTCACCTAAATCTCCAGACAAAGCTCACTTAAATCTTCACATC |
| | AATTCCCTGGAGCTTGGTGACTCTGCTGTGTATTTCTGTGCCAGCAGCCAAGTTCTTAGGGGTGAGC |
| | AGTACTTCGGGCCGGGCACCAGGCTCACGGTCACAGAGGACCTGAAAAACGTGTTCCCACCCGAG |
| | GTCGCTGTGTTTGAGCCATCAGA |
| 330 | GGAGAGACAGGGTCTTGCTCTGTTGCCCAGGCTAGAGTGGAGTAACATGATCATATCTCACTGTATC |
| | CTCAAACTCCTGGGCTCAAGTATTTGACCTGCATAATAAATGTCTACGCCTCATGCCACTAGGTGGC |
| | AATGTGGGTGTTATACTGAAAAGATCACAGATGGTTCACTTGGGAAGTAAAACTGTAAATGTTCTTAA |
| | GTGTGCATTTCTGCTGCTTCTGATGGGCTGAAAATCCCCTTTGATTTCTAAAGTAAATGTAGAGACGT |
| | TTTAAAAATAAAGGACTCCTTTGTCCAAGATATATTCCGAAATCCTCCAACAGAGACCTGTGTGAGCT |
| | TCTGCTGCAGTAATAATGGTGAAGATCCGGCAATTTTTGTTGGCTATTTTGTGGCTTCAGCTAAGCTG |
| | TGTAAGTGCCGCCAAAAATGAAGTGGAGCAGAGTCCTCAGAACCTGACTGCCCAGGAAGGAGAATT |
| | TATCACAATCAACTGCAGTTACTCGGTAGGAATAAGTGCCTTACACTGGCTGCAACAGCATCCAGGA |
| | GGAGGCATTGTTTCCTTGTTTATGCTGAGCTCAGGGAAGAAGAAGCATGGAAGATTAATTGCCACAA |
| | TAAACATACAGGAAAAGCACAGCTCCCTGCACATCACAGCCTCCCATCCCAGAGACTCTGCCGTCTA |
| | CATCTGTGCTGTCAGTACTGGTGGTGCTACAAACAAGCTCATCTTTGGAACTGGCACTCTGCTTGCT |
| | GTCCAGCCAAATATCCAGAACCCTGACCCTGCCGTGTACCAGCTGAGAGACT |
| 331 | GGTTGCCCAGGCTAGAGTGGAGTAACATGATCATATCTCACTGTATCCTCAAACTCCTGGGCTCAAG |
| | TATTTGACCTGCATAATAAATGTCTACGCCTCATGCCACTAGGTGGCAATGTGGGTGTTATACTGAAA |
| | AGATCACAGATGGTTCACTTGGGAAGTAAAACTGTAAATGTTCTTAAGTGTGCATTTCTGCTGCTTCT |
| | GATGGGCTGAAAATCCCCTTTGATTTCTAAAGTAAATGTAGAGACGTTTTAAAAATAAAGGACTCCTT |
| | TGTCCAAGATATATTCCGAAATCCTCCAACAGAGACCTGTGTGAGCTTCTGCTGCAGTAATAATGGT |
| | GAAGATCCGGCAATTTTTGTTGGCTATTTTGTGGCTTCAGCTAAGCTGTGTAAGTGCCGCCAAAAAT |
| | GAAGTGGAGCAGAGTCCTCAGAACCTGACTGCCCAGGAAGGAGAATTTATCACAATCAACTGCAGTT |
| | ACTCGGTAGGAATAAGTGCCTTACACTGGCTGCAACAGCATCCAGGAGGAGGCATTGTTTCCTTGTT |
| | TATGCTGAGCTCAGGGAAGAAGAAGCATGGAAGATTAATTGCCACAATAAACATACAGGAAAAGCAC |
| | AGCTCCCTGCACATCACAGCCTCCCATCCCAGAGACTCTGCCGTCTACATCTGTGCTGTTCAGGAG |
| | GGAGAAACCAGTGGCTCTAGGTTGACCTTTGGGGAAGGAACACAGCTCACAGTGAATCCTGATATC |
| | CAGAACCCTGACCCTGCCGTGTACCAGCTGAGAGACT |
| 332 | GAGAGTCCTGCTCCCCTTTCATCAATGCACAGATACAGAAGACCCCTCCGTCATGCAGCCCCTGCCA |
| | TGAGCATCGGCCTCCTGTGCTGTGCAGCCTTGTCTCTCCTGTGGGCAGGTCCAGTGAATGCTGGTG |
| | TCACTCAGACCCCAAAATTCCAGGTCCTGAAGACAGGACAGAGCATGACACTGCAGTGTGCCCAGG |
| | ATATGAACCATGAATACATGTCCTGGTATCGACAAGACCCAGGCATGGGGCTGAGGCTGATTCATTA |
| | CTCAGTTGGTGCTGGTATCACTGACCAAGGAGAAGTCCCCAATGGCTACAATGTCTCCAGATCAACC |
| | ACAGAGGATTTCCCGCTCAGGCTGCTGTCGGCTGCTCCCTCCCAGACATCTGTGTACTTCTGTGCCA |
| | GCAGTCCAGGGTTTAATGGAAACACCATATATTTTGGAGAGGGAAGTTGGCTCACTGTTGTAGAGGA |
| | CCTGAACAAGGTGTTCCCACCCGAGGTCGCTGTGTTTGAGCCATCAGA |
| 333 | GAGAAGCCTCACACAGCCCAGTAACTTTGCTAGTACCTCTTGAGTGCAAGGTGGAGAATTAAGATCT |
| | GGATTTGAGACGGAGCACGGAACATTTCACTCAGGGGAAGAGCTATGAACATGCTGACTGCCAGCC |
| | TGTTGAGGGCAGTCATAGCCTCCATCTGTGTTGTATCCAGCATGGCTCAGAAGGTAACTCAAGCGCA |
| | GACTGAAATTTCTGTGGTGGAGAAGGAGGATGTGACCTTGGACTGTGTGTATGAAACCCGTGATACT |
| | ACTTATTACTTATTCTGGTACAAGCAACCACCAAGTGGAGAATTGGTTTTCCTTATTCGTCGGAACTC |
| | TTTTGATGAGCAAAATGAAATAAGTGGTCGGTATTCTTGGAACTTCCAGAAATCCACCAGTTCCTTCA |
| | ACTTCACCATCACAGCCTCACAAGTCGTGGACTCAGCAGTATACTTCTGTGCTCTGAGTGAGGCAAC |
| | ATATAACACCGACAAGCTCATCTTTGGGACTGGGACCAGATTACAAGTCTTTCCAAATATCCAGAACC |
| | CTGACCCTGCCGTGTACCAGCTGAGAGACT |
| 334 | TTCAGCTCTGCAGGACAGGTAGAGACTCCAGGATCATCCACTGAGCACTGGACATAAGGAAGGCTG |
| | CATGGGGAGGACACAGGACAGTGACATCACAGGATACCCCTCCTATTAGGAAAATCAAGGCCCAGA |
| | ATTCACTCGGCTCTTCCCCAGGAGGACCAAGCCCTGAATCAGGTGCAGTGCTGCCTGCCCCACTGT |
| | GCCATGGGCCCTGGGCTCCTCTGCTGGGCGCTGCTTTGTCTCCTGGGAGCAGGCTCAGTGGAGAC |
| | TGGAGTCACCCAAAGTCCCACACACCTGATCAAAACGAGAGGACAGCAAGTGACTCTGAGATGCTC |
| | TTCTCAGTCTGGGCACAACACTGTGTCCTGGTACCAACAGGCCCTGGGTCAGGGGCCCCAGTTTAT |
| | CTTTCAGTATTATAGGGAGGAAGAGAATGGCAGAGGAAACTTCCCTCCTAGATTCTCAGGTCTCCAG |
| | TTCCCTAATTATAGCTCTGAGCTGAATGTGAACGCCTTGGAGCTGGACGACTCGGCCCTGTATCTCT |
| | GTGCCAGCAGCTCCACCGGGACGGACTATGGCTACACCTTCGGTTCGGGGACCAGGTTAACCGTTG |
| | TAGAGGACCTGAACAAGGTGTTCCCACCCGAGGTCGCTGTGTTTGAGCCATCAGA |
| 335 | AAAGCAGATTCTTTTTATGATTTTTAAAGTAGAAATATCCATTCTAGGTGCATTTTTTAAGGGTTTAAAA |
| | TTTGAATCCTCAGTGAACCAGGGCAGAGAAGAATGATGAAATCCTTGAGAGTTTTACTAGTGATCCT |
| | GTGGCTTCAGTTGAGCTGGGTTTGGAGCCAACAGAAGGAGGTGGAGCAGAATTCTGGACCCCTCAG |
| | TGTTCCAGAGGGAGCCATTGCCTCTCTCAACTGCACTTACAGTGACCGAGGTTCCCAGTCCTTCTTC |
| | TGGTACAGACAATATTCTGGGAAAAGCCCTGAGTTGATAATGTTCATATACTCCAATGGTGACAAAGA |
| | AGATGGAAGGTTTACAGCACAGCTCAATAAAGCCAGCCAGTATGTTTCTCTGCTCATCAGAGACTCC |
| | CAGCCCAGTGATTCAGCCACCTACCTCTGTGCCGGGAGGCAAACCTCCTACGACAAGGTGATATTT |
| | GGGCCAGGGACAAGCTTATCAGTCATTCCAAATATCCAGAACCCTGACCCTGCCGTGTACCAGCTG |
| | AGAGACT |
| 336 | AGAGCTGGAAACACCTCCATCCTGCCTCTTCATGCCATGGCCTCCCTGCTCTTCTTCTGTGGGGCCT |
| | TTTATCTCCTGGGAACAGGGTCCATGGATGCTGATGTTACCCAGACCCCAAGGAATAGGATCACAAA |
| | GACAGGAAAGAGGATTATGCTGGAATGTTCTCAGACTAAGGGTCATGATAGAATGTACTGGTATCGA |
| | CAAGACCCAGGACTGGGCCTACGGTTGATCTATTACTCCTTTGATGTCAAAGATATAAACAAAGGAG |
| | AGATCTCTGATGGATACAGTGTCTCTCGACAGGCACAGGCTAAATTCTCCCTGTCCCTAGAGTCTGC |
| | CATCCCCAACCAGACAGCTCTTTACTTCTGTGCCACCAGTGATGTGACTGGGCAGGGCGAGATGCG |
| | TGGCTACACCTTCGGTTCGGGGACCAGGTTAACCGTTGTAGAGGACCTGAACAAGGTGTTCCCACC |
| | CGAGGTCGCTGTGTTTGAGCCATCAGA |
| 337 | GAGAAGCCTCACACAGCCCAGTAACTTTGCTAGTACCTCTTGAGTGCAAGGTGGAGAATTAAGATCT |
| | GGATTTGAGACGGAGCACGGAACATTTCACTCAGGGGAAGAGCTATGAACATGCTGACTGCCAGCC |
| | TGTTGAGGGCAGTCATAGCCTCCATCTGTGTTGTATCCAGCATGGCTCAGAAGGTAACTCAAGCGCA |
| | GACTGAAATTTCTGTGGTGGAGAAGGAGGATGTGACCTTGGACTGTGTGTATGAAACCCGTGATACT |
| | ACTTATTACTTATTCTGGTACAAGCAACCACCAAGTGGAGAATTGGTTTTCCTTATTCGTCGGAACTC |
| | TTTTGATGAGCAAAATGAAATAAGTGGTCGGTATTCTTGGAACTTCCAGAAATCCACCAGTTCCTTCA |
| | ACTTCACCATCACAGCCTCACAAGTCGTGGACTCAGCAGTATACTTCTGTGCTCTGAGTGAGATGAA |
| | CAGAGATGACAAGATCATCTTTGGAAAAGGGACACGACTTCATATTCTCCCCAATATCCAGAACCCT |
| | GACCCTGCCGTGTACCAGCTGAGAGACT |
| 338 | AGCTGTGAGGTCTGGTTCCCCGACGTGCTGCAGCAAGTGCCTTTGCCCTGCCTGTGGGCTCCCTCC |
| | ATGGCCAACTCTGCTATGGACACCAGAGTACTCTGCTGTGCGGTCATCTGTCTTCTGGGGGCAGGT |
| | CTCTCAAATGCCGGCGTCATGCAGAACCCAAGACACCTGGTCAGGAGGAGGGGACAGGAGGCAAG |
| | ACTGAGATGCAGCCCAATGAAAGGACACAGTCATGTTTACTGGTATCGGCAGCTCCCAGAGGAAGG |
| | TCTGAAATTCATGGTTTATCTCCAGAAAGAAAATATCATAGATGAGTCAGGAATGCCAAAGGAACGAT |
| | TTTCTGCTGAATTTCCCAAAGAGGGCCCCAGCATCCTGAGGATCCAGCAGGTAGTGCGAGGAGATT |
| | CGGCAGCTTATTTCTGTGCCAGCTCACCGACAGGGGTCTCTGGAAACACCATATATTTTGGAGAGGG |
| | AAGTTGGCTCACTGTTGTAGAGGACCTGAACAAGGTGTTCCCACCCGAGGTCGCTGTGTTTGAGCC |
| | ATCAGA |
| 339 | CGGGAAGAGCAGAGATTTAAATTTCTTGGTTGTTTTTAGTGGGACTCTAAGCCCAAGAGAGTTTCTTG |
| | AAAAAAAAAAAAAAAAAAACCCATTCAGGAAATAATTCTTTGCTGATAAGGATGCTCCTTGAACATTTA |
| | TTAATAATCTTGTGGATGCAGCTGACATGGGTCAGTGGTCAACAGCTGAATCAGAGTCCTCAATCTAT |
| | GTTTATCCAGGAAGGAGAAGATGTCTCCATGAACTGCACTTCTTCAAGCATATTTAACACCTGGCTAT |
| | GGTACAAGCAGGACCCTGGGGAAGGTCCTGTCCTCTTGATAGCCTTATATAAGGCTGGTGAATTGAC |
| | CTCAAATGGAAGACTGACTGCTCAGTTTGGTATAACCAGAAAGGACAGCTTCCTGAATATCTCAGCA |
| | TCCATACCTAGTGATGTAGGCATCTACTTCTGTGCTGGGCAGCAAAAAACCAGTGGCTCTAGGTTGA |
| | CCTTTGGGGAAGGAACACAGCTCACAGTGAATCCTGATATCCAGAACCCTGACCCTGCCGTGTACC |
| | AGCTGAGAGACT |
| 340 | GGAGGTGCGAATGACTCTGCTCTCTGTCCTGTCTCCTCATCTGCAAAATTAGGAAGCCTGTCTTGAT |
| | TATCTCCAGGAACCTCCCACCTCTTCATTCCAGCCTCTGACAAACTCTGCACATTAGGCCAGGAGAA |
| | GCCCCCGAGCCAAGTCTCTTTTCTCATTCTCTTCCAACAAGTGCTTGGAGCTCCAAGAAGGCCCCCT |
| | TTGCACTATGAGCAACCAGGTGCTCTGCTGTGTGGTCCTTTGTTTCCTGGGAGCAAACACCGTGGAT |
| | GGTGGAATCACTCAGTCCCCAAAGTACCTGTTCAGAAAGGAAGGACAGAATGTGACCCTGAGTTGT |
| | GAACAGAATTTGAACCACGATGCCATGTACTGGTACCGACAGGACCCAGGGCAAGGGCTGAGATTG |
| | ATCTACTACTCACAGATAGTAAATGACTTTCAGAAAGGAGATATAGCTGAAGGGTACAGCGTCTCTC |
| | GGGAGAAGAAGGAATCCTTTCCTCTCACTGTGACATCGGCCCAAAAGAACCCGACAGCTTTCTATCT |
| | CTGTGCCAGTAGTTCCCCCCGGGACAGGGTCGGTCAGCCCCAGCATTTTGGTGATGGGACTCGACT |
| | CTCCATCCTAGAGGACCTGAACAAGGTGTTCCCACCCGAGGTCGCTGTGTTTGAGCCATCAGA |
| 341 | GGCTTTGTCGGGTGGAGCTGATTGGTTGCAGGAGCAGCAACAGTTCCAGAGCCAAGTCATGACACC |
| | GACCTCCCCAAGGTTTAGTTAAATATATCTTATGGTGAAAATGCCCGGAGCAAGAAGGCAAAGCATC |
| | ATGAAGAGGATATTGGGAGCTCTGCTGGGGCTCTTGAGTGCCCAGGTTTGCTGTGTGAGAGGAATA |
| | CAAGTGGAGCAGAGTCCTCCAGACCTGATTCTCCAGGAGGGAGCCAATTCCACGCTGCGGTGCAAT |
| | TTTTCTGACTCTGTGAACAATTTGCAGTGGTTTCATCAAAACCCTTGGGGACAGCTCATCAACCTGTT |
| | TTACATTCCCTCAGGGACAAAACAGAATGGAAGATTAAGCGCCACGACTGTCGCTACGGAACGCTAC |
| | AGCTTATTGTACATTTCCTCTTCCCAGACCACAGACTCAGGCGTTTATTTCTGTGCTGCCCGTCGGGT |
| | CGACAATAACAATGACATGCGCTTTGGAGCAGGGACCAGACTGACAGTAAAACCAAATATCCAGAAG |
| | CCTGACCCTGCCGTGTACCAGCTGAGAGACT |
| 342 | AGCTCCTGTATTCGTGCCCACAAGGGCCTCATCTAGGTGAAGGCTCCACCTGCCCCACCCTGCCAT |
| | GGCCACCAGGCTCCTCTGCTGTGTGGTTCTTTGTCTCCTGGGAGAAGAGCTTATAGATGCTAGAGTC |
| | ACCCAGACACCAAGGCACAAGGTGACAGAGATGGGACAAGAAGTAACAATGAGATGTCAGCCAATT |
| | TTAGGCCACAATACTGTTTTCTGGTACAGACAGACCATGATGCAAGGACTGGAGTTGCTGGCTTACT |
| | TCCGCAACCGGGCTCCTCTAGATGATTCGGGGATGCCGAAGGATCGATTCTCAGCAGAGATGCCTG |
| | ATGCAACTTTAGCCACTCTGAAGATCCAGCCCTCAGAACCCAGGGACTCAGCTGTGTATTTTTGTGC |
| | TAGTGGTTTAGCTCAGCCCCAGCATTTTGGTGATGGGACTCGACTCTCCATCCTAGAGGACCTGAAC |
| | AAGGTGTTCCCACCCGAGGTCGCTGTGTTTGAGCCATCAGA |
| 343 | AGGAACATGACTAAACCTGGGGAGGAAGACATAGAAAAGCAACTGCTCTATCATTGGCTGACAAGAT |
| | CCTGACAACAACACAATGAGAGCTGCACTCCTTGCAAGTCTTTACTAGTAGCTGTTGCTTCTGTTCTC |
| | TGGAAATTCTTTAAACCTAGAATCAGACACAAAAACTGAACTCTGGGTCCACAATCCTCATTTGTCCT |
| | TGAAGTATGAGGCTGGTGGCAAGAGTAACTGTGTTTCTGACCTTTGGAACTATAATTGATGCTAAGA |
| | CCACCCAGCCCCCCTCCATGGATTGCGCTGAAGGAAGAGCTGCAAACCTGCCTTGTAATCACTCTA |
| | CCATCAGTGGAAATGAGTATGTGTATTGGTATCGACAGATTCACTCCCAGGGGCCACAGTATATCAT |
| | TCATGGTCTAAAAAACAATGAAACCAATGAAATGGCCTCTCTGATCATCACAGAAGACAGAAAGTCCA |
| | GCACCTTGATCCTGCCCCACGCTACGCTGAGAGACACTGCTGTGTACTATTGCATCGTCAGAGTCG |
| | CGGTAACCAATGCAGGCAAATCAACCTTTGGGGATGGGACTACGCTCACTGTGAAGCCAAATATCCA |
| | GAAGCCTGACCCTGCCGTGTACCAGCTGAGAGACT |
| 344 | ACCTGGAGCCCCCAGAACTGGCAGACACCTGCCTGATGCTGCCATGGGCCCCCAGCTCCTTGGCTA |
| | TGTGGTCCTTTGCCTTCTAGGAGCAGGCCCCCTGGAAGCCCAAGTGACCCAGAACCCAAGATACCT |
| | CATCACAGTGACTGGAAAGAAGTTAACAGTGACTTGTTCTCAGAATATGAACCATGAGTATATGTCCT |
| | GGTATCGACAAGACCCAGGGCTGGGCTTAAGGCAGATCTACTATTCAATGAATGTTGAGGTGACTGA |
| | TAAGGGAGATGTTCCTGAAGGGTACAAAGTCTCTCGAAAAGAGAAGAGGAATTTCCCCCTGATCCTG |
| | GAGTCGCCCAGCCCCAACCAGACCTCTCTGTACTTCTGTGCCAGCAGATATTATAGCGCGGATACG |
| | CAGTATTTTGGCCCAGGCACCCGGCTGACAGTGCTCGAGGACCTGAAAAACGTGTTCCCACCCGAG |
| | GTCGCTGTGTTTGAGCCATCAGA |
| 345 | GTAGCTCGTTGATATCTGTGTGGATAGGGAGCTGTGACGAGAGCAAGAGGTCAGAACACATCCAGG |
| | CTCCTTAAGGGAAAGCCTCTTTCTGTTTCTGAAACTTTTCAAAGCCAGGGACTTGTCCAATCCAACCT |
| | CCTCACAGTTCCTAGCTCCTGAGGCTCAGCGCCCTTGGCTTCTGTCCGCCCAGCTCAAGGTCCTGC |
| | AGCATTGCCACTGCTCAGCCATGCTCCTGCTGCTCGTCCCAGCGTTCCAGGTGATTTTTACCCTGGG |
| | AGGAACCAGAGCCCAGTCTGTGACCCAGCTTGACAGCCAAGTCCCTGTCTTTGAAGAAGCCCCTGT |
| | GGAGCTGAGGTGCAACTACTCATCGTCTGTTTCAGTGTATCTCTTCTGGTATGTGCAATACCCCAAC |
| | CAAGGACTCCAGCTTCTCCTGAAGTATTTATCAGGATCCACCCTGGTTAAAGGCATCAACGGTTTTG |
| | AGGCTGAATTTAACAAGAGTCAAACTTCCTTCCACTTGAGGAAACCCTCAGTCCATATAAGCGACAC |
| | GGCTGAGTACTTCTGTGCTGTGAGTGGGGTACTCACGGGAGGAGGAAACAAACTCACCTTTGGGAC |
| | AGGCACTCAGCTAAAAGTGGAACTCAATATCCAGAAGCCTGACCCTGCCGTGTACCAGCTGAGAGA |
| | CT |
| 346 | AATTTGCCCACAGCAGGGCTGGGAGACACAAGATCCTGCCCTGGAGCTGAAATGGGCACCAGGCTC |
| | TTCTTCTATGTGGCCCTTTGTCTGCTGTGGGCAGGACACAGGGATGCTGGAATCACCCAGAGCCCA |
| | AGATACAAGATCACAGAGACAGGAAGGCAGGTGACCTTGATGTGTCACCAGACTTGGAGCCACAGC |
| | TATATGTTCTGGTATCGACAAGACCTGGGACATGGGCTGAGGCTGATCTATTACTCAGCAGCTGCTG |
| | ATATTACAGATAAAGGAGAAGTCCCCGATGGCTATGTTGTCTCCAGATCCAAGACAGAGAATTTCCC |
| | CCTCACTCTGGAGTCAGCTACCCGCTCCCAGACATCTGTGTATTTCTGCGCCAGCAGTGAGGGGAC |
| | AGTTAGCAATCAGCCCCAGCATTTTGGTGATGGGACTCGACTCTCCATCCTAGAGGACCTGAACAAG |
| | GTGTTCCCACCCGAGGTCGCTGTGTTTGAGCCATCAGA |
| 347 | CGATGATGGAAGTAGCTCTTATGGCTGGAGATTGCAGGTTTATGACTGATCCTATTTGGGAAGAACA |
| | ATGATGGCAGGCATTCGAGCTTTATTTATGTACTTGTGGCTGCAGCTGGACTGGGTGAGCAGAGGA |
| | GAGAGTGTGGGGCTGCATCTTCCTACCCTGAGTGTCCAGGAGGGTGACAACTCTATTATCAACTGTG |
| | CTTATTCAAACAGCGCCTCAGACTACTTCATTTGGTACAAGCAAGAATCTGGAAAAGGTCCTCAATTC |
| | ATTATAGACATTCGTTCAAATATGGACAAAAGGCAAGGCCAAAGAGTCACCGTTTTATTGAATAAGAC |
| | AGTGAAACATCTCTCTCTGCAAATTGCAGCTACTCAACCTGGAGACTCAGCTGTCTACTTTTGTGCAG |
| | AGATCGGGTCTGGGGCTGGGAGTTACCAACTCACTTTCGGGAAGGGGACCAAACTCTCGGTCATAC |
| | CAAATATCCAGAACCCTGACCCTGCCGTGTACCAGCTGAGAGACT |
| 348 | CGATGATGGAAGTAGCTCTTATGGCTGGAGATTGCAGGTTTATGACTGATCCTATTTGGGAAGAACA |
| | ATGATGGCAGGCATTCGAGCTTTATTTATGTACTTGTGGCTGCAGCTGGACTGGGTGAGCAGAGGA |
| | GAGAGTGTGGGGCTGCATCTTCCTACCCTGAGTGTCCAGGAGGGTGACAACTCTATTATCAACTGTG |
| | CTTATTCAAACAGCGCCTCAGACTACTTCATTTGGTACAAGCAAGAATCTGGAAAAGGTCCTCAATTC |
| | ATTATAGACATTCGTTCAAATATGGACAAAAGGCAAGGCCAAAGAGTCACCGTTTTATTGAATAAGAC |
| | AGTGAAACATCTCTCTCTGCAAATTGCAGCTACTCAACCTGGAGACTCAGCTGTCTACTTTTGTGCAG |
| | AGAATCAGGGAGGCAGCAGCTATAAATTGATCTTCGGGAGTGGGACCAGACTGCTGGTCAGGCCTG |
| | ATATCCAGAACCCTGACCCTGCCGTGTACCAGCTGAGAGACT |
| 349 | GTGGAAACCCACTTCTGACTTATCACTTGTCATGAATTCTATGCTTCATGGTGTTACACCGTTTATTGT |
| | TTCTGATGAGTGACAGTAATTATTTTCTTTCTTGCTGGTACATAATAAAGTGGTGCACATCAGAGTTG |
| | CTGCCATCTTAGACTTAACTCATCAGTATCAGGTGATCCTGAGGCTCAGTGATGTGACTGTGGGAAC |
| | TGCTCTGTGGCGACAAGGACGTCCCTCATCCTCTGCTCCTGGTGACAGTGACCCTGATCTGGTAAA |
| | GCTCCCATCCTGCCCTGACCCTGCCATGGGCACCAGCCTCCTCTGCTGGATGGCCCTGTGTCTCCT |
| | GGGGGCAGATCACGCAGATACTGGAGTCTCCCAGAACCCCAGACACAAGATCACAAAGAGGGGAC |
| | AGAATGTAACTTTCAGGTGTGATCCAATTTCTGAACACAACCGCCTTTATTGGTACCGACAGACCCTG |
| | GGGCAGGGCCCAGAGTTTCTGACTTACTTCCAGAATGAAGCTCAACTAGAAAAATCAAGGCTGCTCA |
| | GTGATCGGTTCTCTGCAGAGAGGCCTAAGGGATCTTTCTCCACCTTGGAGATCCAGCGCACAGAGC |
| | AGGGGGACTCGGCCATGTATCTCTGTGCCAGCAGCTCTCAGTCGGGTGTGGACACTGAAGCTTTCT |
| | TTGGACAAGGCACCAGACTCACAGTTGTAGAGGACCTGAACAAGGTGTTCCCACCCGAGGTCGCTG |
| | TGTTTGAGCCATCAGA |
| 350 | TTTTGAAACCCTTCAAAGGCAGAGACTTGTCCAGCCTAACCTGCCTGCTGCTCCTAGCTCCTGAGGC |
| | TCAGGGCCCTTGGCTTCTGTCCGCTCTGCTCAGGGCCCTCCAGCGTGGCCACTGCTCAGCCATGCT |
| | CCTGCTGCTCGTCCCAGTGCTCGAGGTGATTTTTACCCTGGGAGGAACCAGAGCCCAGTCGGTGAC |
| | CCAGCTTGGCAGCCACGTCTCTGTCTCTGAGGGAGCCCTGGTTCTGCTGAGGTGCAACTACTCATC |
| | GTCTGTTCCACCATATCTCTTCTGGTATGTGCAATACCCCAACCAAGGACTCCAGCTTCTCCTGAAGT |
| | ACACAACAGGGGCCACCCTGGTTAAAGGCATCAACGGTTTTGAGGCTGAATTTAAGAAGAGTGAAAC |
| | CTCCTTCCACCTGACGAAACCCTCAGCCCATATGAGCGACGCGGCTGAGTACTTCTGTGCTGTGAG |
| | TCCCCCCGCGCAGAAACTTGTATTTGGAACTGGCACCCGACTTCTGGTCAGTCCAAATATCCAGAAC |
| | CCTGACCCTGCCGTGTACCAGCTGAGAGACT |
| 351 | GAGAGTCCTGCTCCCCTTTCATCAATGCACAGATACAGAAGACCCCTCCGTCATGCAGCATCTGCCA |
| | TGAGCATCGGCCTCCTGTGCTGTGCAGCCTTGTCTCTCCTGTGGGCAGGTCCAGTGAATGCTGGTG |
| | TCACTCAGACCCCAAAATTCCAGGTCCTGAAGACAGGACAGAGCATGACACTGCAGTGTGCCCAGG |
| | ATATGAACCATGAATACATGTCCTGGTATCGACAAGACCCAGGCATGGGGCTGAGGCTGATTCATTA |
| | CTCAGTTGGTGCTGGTATCACTGACCAAGGAGAAGTCCCCAATGGCTACAATGTCTCCAGATCAACC |
| | ACAGAGGATTTCCCGCTCAGGCTGCTGTCGGCTGCTCCCTCCCAGACATCTGTGTACTTCTGTGCCA |
| | GCCAAGAGACAGGGGTTGGCGGGGAGCTGTTTTTTGGAGAAGGCTCTAGGCTGACCGTACTGGAG |
| | GACCTGAAAAACGTGTTCCCACCCGAGGTCGCTGTGTTTGAGCCATCAGA |
| 352 | GAGAGTCCTGCTCCCCTTTCATCAATGCACAGATACAGAAGACCCCTCCGTCATGCAGCATCTGCCA |
| | TGAGCATCGGCCTCCTGTGCTGTGCAGCCTTGTCTCTCCTGTGGGCAGGTCCAGTGAATGCTGGTG |
| | TCACTCAGACCCCAAAATTCCAGGTCCTGAAGACAGGACAGAGCATGACACTGCAGTGTGCCCAGG |
| | ATATGAACCATGAATACATGTCCTGGTATCGACAAGACCCAGGCATGGGGCTGAGGCTGATTCATTA |
| | CTCAGTTGGTGCTGGTATCACTGACCAAGGAGAAGTCCCCAATGGCTACAATGTCTCCAGATCAACC |
| | ACAGAGGATTTCCCGCTCAGGCTGCTGTCGGCTGCTCCCTCCCAGACATCTGTGTACTTCTGTGCCA |
| | GCAGTACGACAGCCGTGGTTTCACCCCTCCACTTTGGGAATGGGACCAGGCTCACTGTGACAGAGG |
| | ACCTGAACAAGGTGTTCCCACCCGAGGTCGCTGTGTTTGAGCCATCAGA |
| 353 | AGAATCTCCCACAGATACAGGATAATTGGTTTAGCGTTTGGGGATCTGGCCACAAGGTGGCAGAGCT |
| | TCTCTGCTCATGTAGAATGTACGAAAGGATCCTTTTGGTTGACTTCTGTGAGTAGGCTGACGGCAGA |
| | AGCAGATGCCTCTGTGGACAGTTTAAGAAACCACAGTGCTTTGGAGGAAAGGAAGAGATACTTGATA |
| | ATATAGCTCTCTTGGCTGGAGATTGCAGGTCCCAGTGGGGAGAACAATGAAGACATTTGCTGGATTT |
| | TCGTTCCTGTTTTTGTGGCTGCAGCTGGACTGTATGAGTAGAGGAGAGGATGTGGAGCAGAGTCTTT |
| | TCCTGAGTGTCCGAGAGGGAGACAGCTCCGTTATAAACTGCACTTACACAGACAGCTCCTCCACCTA |
| | CTTATACTGGTATAAGCAAGAACCTGGAGCAGGTCTCCAGTTGCTGACGTATATTTTTTCAAATATGG |
| | ACATGAAACAAGACCAAAGACTCACTGTTCTATTGAATAAAAAGGATAAACATCTGTCTCTGCGCATT |
| | GCAGACACCCAGACTGGGGACTCAGCTATCTACTTCTGTGCAGAGAAGAAGGAAGGCTTCAAAACT |
| | ATCTTTGGAGCAGGAACAAGACTATTTGTTAAAGCAAATATCCAGAACCCTGACCCTGCCGTGTACC |
| | AGCTGAGAGACT |
| 354 | GAGAGTCCTGCTCCCCTTTCATCAATGCACAGATACAGAAGACCCCTCCGTCATGCAGCATCTGCCA |
| | TGAGCATCGGCCTCCTGTGCTGTGCAGCCTTGTCTCTCCTGTGGGCAGGTCCAGTGAATGCTGGTG |
| | TCACTCAGACCCCAAAATTCCAGGTCCTGAAGACAGGACAGAGCATGACACTGCAGTGTGCCCAGG |
| | ATATGAACCATGAATACATGTCCTGGTATCGACAAGACCCAGGCATGGGGCTGAGGCTGATTCATTA |
| | CTCAGTTGGTGCTGGTATCACTGACCAAGGAGAAGTCCCCAATGGCTACAATGTCTCCAGATCAACC |
| | ACAGAGGATTTCCCGCTCAGGCTGCTGTCGGCTGCTCCCTCCCAGACATCTGTGTACTTCTGTGCCA |
| | GCCAAGAGACAGGGGTTGGCGGGGAGCTGTTTTTTGGAGAAGGCTCTAGGCTGACCGTACTGGAG |
| | GACCTGAAAAACGTGTTCCCACCCGAGGTCGCTGTGTTTGAGCCATCAGA |
| 355 | AGGACTGAGCTTGCCTGTGACTGGCTAGGGAGGAACCTGAGACTAGGGGACAGAAAGACTAGGGA |
| | TTCACCCAGTAAAGAGAGCTCATCTGTGACTGAGGAGCCTTGCTCCATTTCAGGTCTTCTGTGATTTC |
| | AATAAGGAAGAAGAATGGAAACTCTCCTGGGAGTGTCTTTGGTGATTCTATGGCTTCAACTGGCTAG |
| | GGTGAACAGTCAACAGGGAGAAGAGGATCCTCAGGCCTTGAGCATCCAGGAGGGTGAAAATGCCAC |
| | CATGAACTGCAGTTACAAAACTAGTATAAACAATTTACAGTGGTATAGACAAAATTCAGGTAGAGGCC |
| | TTGTCCACCTAATTTTAATACGTTCAAATGAAAGAGAGAAACACAGTGGAAGATTAAGAGTCACGCTT |
| | GACACTTCCAAGAAAAGCAGTTCCTTGTTGATCACGGCTTCCCGGGCAGCAGACACTGCTTCTTACT |
| | TCTGTGCTACGGACGAGGCTGCAGGCAACAAGCTAACTTTTGGAGGAGGAACCAGGGTGCTAGTTA |
| | AACCAAATATCCAGAACCCTGACCCTGCCGTGTACCAGCTGAGAGACT |
| 356 | AAAATGCCCCTCCTTTCCTCCACAGGACCAGATGCCTGAGCTAGGAAAGGCCTCATTCCTGCTGTGA |
| | TCCTGCCATGGATACCTGGCTCGTATGCTGGGCAATTTTTAGTCTCTTGAAAGCAGGACTCACAGAA |
| | CCTGAAGTCACCCAGACTCCCAGCCATCAGGTCACACAGATGGGACAGGAAGTGATCTTGCGCTGT |
| | GTCCCCATCTCTAATCACTTATACTTCTATTGGTACAGACAAATCTTGGGGCAGAAAGTCGAGTTTCT |
| | GGTTTCCTTTTATAATAATGAAATCTCAGAGAAGTCTGAAATATTCGATGATCAATTCTCAGTTGAAAG |
| | GCCTGATGGATCAAATTTCACTCTGAAGATCCGGTCCACAAAGCTGGAGGACTCAGCCATGTACTTC |
| | TGTGCCAGCAAAAGGGAATCACTAGCCACCGGGGAGCTGTTTTTTGGAGAAGGCTCTAGGCTGACC |
| | GTACTGGAGGACCTGAAAAACGTGTTCCCACCCGAGGTCGCTGTGTTTGAGCCATCAGA |
| 357 | AGGACTGAGCTTGCCTGTGACTGGCTAGGGAGGAACCTGAGACTAGGGGACAGAAAGACTAGGGA |
| | TTCACCCAGTAAAGAGAGCTCATCTGTGACTGAGGAGCCTTGCTCCATTTCAGGTCTTCTGTGATTTC |
| | AATAAGGAAGAAGAATGGAAACTCTCCTGGGAGTGTCTTTGGTGATTCTATGGCTTCAACTGGCTAG |
| | GGTGAACAGTCAACAGGGAGAAGAGGATCCTCAGGCCTTGAGCATCCAGGAGGGTGAAAATGCCAC |
| | CATGAACTGCAGTTACAAAACTAGTATAAACAATTTACAGTGGTATAGACAAAATTCAGGTAGAGGCC |
| | TTGTCCACCTAATTTTAATACGTTCAAATGAAAGAGAGAAACACAGTGGAAGATTAAGAGTCACGCTT |
| | GACACTTCCAAGAAAAGCAGTTCCTTGTTGATCACGGCTTCCCGGGCAGCAGACACTGCTTCTTACT |
| | TCTGTGCTGCCATGTATTCAGGAGGAGGTGCTGACGGACTCACCTTTGGCAAAGGGACTCATCTAAT |
| | CATCCAGCCCTATATCCAGAACCCTGACCCTGCCGTGTACCAGCTGAGAGACT |
| 358 | AAAATGCCCCTCCTTTCCTCCACAGGACCAGATGCCTGAGCTAGGAAAGGCCTCATTCCTGCTGTGA |
| | TCCTGCCATGGATACCTGGCTCGTATGCTGGGCAATTTTTAGTCTCTTGAAAGCAGGACTCACAGAA |
| | CCTGAAGTCACCCAGACTCCCAGCCATCAGGTCACACAGATGGGACAGGAAGTGATCTTGCGCTGT |
| | GTCCCCATCTCTAATCACTTATACTTCTATTGGTACAGACAAATCTTGGGGCAGAAAGTCGAGTTTCT |
| | GGTTTCCTTTTATAATAATGAAATCTCAGAGAAGTCTGAAATATTCGATGATCAATTCTCAGTTGAAAG |
| | GCCTGATGGATCAAATTTCACTCTGAAGATCCGGTCCACAAAGCTGGAGGACTCAGCCATGTACTTC |
| | TGTGCCAGCAGTACCCAGGGACAGGCCTACGAGCAGTACTTCGGGCCGGGCACCAGGCTCACGGT |
| | CACAGAGGACCTGAAAAACGTGTTCCCACCCGAGGTCGCTGTGTTTGAGCCATCAGA |
| 359 | GATCTTAATTGGGAAGAACAAGGATGACATCCATTCGAGCTGTATTTATATTCCTGTGGCTGCAGCTG |
| | GACTTGGTGAATGGAGAGAATGTGGAGCAGCATCCTTCAACCCTGAGTGTCCAGGAGGGAGACAGC |
| | GCTGTTATCAAGTGTACTTATTCAGACAGTGCCTCAAACTACTTCCCTTGGTATAAGCAAGAACTTGG |
| | AAAAAGACCTCAGCTTATTATAGACATTCGTTCAAATGTGGGCGAAAAGAAAGACCAACGAATTGCTG |
| | TTACATTGAACAAGACAGCCAAACATTTCTCCCTGCACATCACAGAGACCCAACCTGAAGACTCGGC |
| | TGTCTACTTCTGTGCAGCAAGAGGAGGTAGCAACTATAAACTGACATTTGGAAAAGGAACTCTCTTAA |
| | CCGTGAATCCAAATATCCAGAACCCTGACCCTGCCGTGTACCAGCTGAGAGACT |
| 360 | TATGGGGATGTTCACAGAGGGCCTGGTCTGGAATATTCCACATCTGCTCTCACTCTGCCATGGGCTC |
| | CTGGACCCTCTGCTGTGTGTCCCTTTGCATCCTGGTAGCAAAGCACACAGATGCTGGAGTTATCCAG |
| | TCACCCCGGCACGAGGTGACAGAGATGGGACAAGAAGTGACTCTGAGATGTAAACCAATTTCAGGA |
| | CACGACTACCTTTTCTGGTACAGACAGACCATGATGCGGGGACTGGAGTTGCTCATTTACTTTAACA |
| | ACAACGTTCCGATAGATGATTCAGGGATGCCCGAGGATCGATTCTCAGCTAAGATGCCTAATGCATC |
| | ATTCTCCACTCTGAAGATCCAGCCCTCAGAACCCAGGGACTCAGCTGTGTACTTCTGTGCCAGCAGC |
| | ACAGGGGGCGAGCAGTACTTCGGGCCGGGCACCAGGCTCACGGTCACAGAGGACCTGAAAAACGT |
| | GTTCCCACCCGAGGTCGCTGTGTTTGAGCCATCAGA |
| 361 | AGGACTGAGCTTGCCTGTGACTGGCTAGGGAGGAACCTGAGACTAGGGGACAGAAAGACTAGGGA |
| | TTCACCCAGTAAAGAGAGCTCATCTGTGACTGAGGAGCCTTGCTCCATTTCAGGTCTTCTGTGATTTC |
| | AATAAGGAAGAAGAATGGAAACTCTCCTGGGAGTGTCTTTGGTGATTCTATGGCTTCAACTGGCTAG |
| | GGTGAACAGTCAACAGGGAGAAGAGGATCCTCAGGCCTTGAGCATCCAGGAGGGTGAAAATGCCAC |
| | CATGAACTGCAGTTACAAAACTAGTATAAACAATTTACAGTGGTATAGACAAAATTCAGGTAGAGGCC |
| | TTGTCCACCTAATTTTAATACGTTCAAATGAAAGAGAGAAACACAGTGGAAGATTAAGAGTCACGCTT |
| | GACACTTCCAAGAAAAGCAGTTCCTTGTTGATCACGGCTTCCCGGGCAGCAGACACTGCTTCTTACT |
| | TCTGTGCTACAAAGGGTGGGAACAACAGACTCGCTTTTGGGAAGGGGAACCAAGTGGTGGTCATAC |
| | CAAATATCCAGAACCCTGACCCTGCCGTGTACCAGCTGAGAGACT |
| 362 | AAAATGCCCCTCCTTTCCTCCACAGGACCAGATGCCTGAGCTAGGAAAGGCCTCATTCCTGCTGTGA |
| | TCCTGCCATGGATACCTGGCTCGTATGCTGGGCAATTTTTAGTCTCTTGAAAGCAGGACTCACAGAA |
| | CCTGAAGTCACCCAGACTCCCAGCCATCAGGTCACACAGATGGGACAGGAAGTGATCTTGCGCTGT |
| | GTCCCCATCTCTAATCACTTATACTTCTATTGGTACAGACAAATCTTGGGGCAGAAAGTCGAGTTTCT |
| | GGTTTCCTTTTATAATAATGAAATCTCAGAGAAGTCTGAAATATTCGATGATCAATTCTCAGTTGAAAG |
| | GCCTGATGGATCAAATTTCACTCTGAAGATCCGGTCCACAAAGCTGGAGGACTCAGCCATGTACTTC |
| | TGTGCCAGCAGTGCCTGGACAGGGGAGACGGGCTACACCTTCGGTTCGGGGACCAGGTTAACCGT |
| | TGTAGAGGACCTGAACAAGGTGTTCCCACCCGAGGTCGCTGTGTTTGAGCCATCAGA |
| 363 | ATATGTAAAATGAAGGGTCTGTGGAAGGACATGAATAAAGCACAGGAGGTTGAAGTCAGATTTGCAG |
| | CTTTCTAGGCAGGAGACAAGACAATCTGCATCTTCACAGGAGGGATGGCCATGCTCCTGGGGGCAT |
| | CAGTGCTGATTCTGTGGCTTCAGCCAGACTGGGTAAACAGTCAACAGAAGAATGATGACCAGCAAGT |
| | TAAGCAAAATTCACCATCCCTGAGCGTCCAGGAAGGAAGAATTTCTATTCTGAACTGTGACTATACTA |
| | ACAGCATGTTTGATTATTTCCTATGGTACAAAAAATACCCTGCTGAAGGTCCTACATTCCTGATATCTA |
| | TAAGTTCCATTAAGGATAAAAATGAAGATGGAAGATTCACTGTCTTCTTAAACAAAAGTGCCAAGCAC |
| | CTCTCTCTGCACATTGTGCCCTCCCAGCCTGGAGACTCTGCAGTGTACTTCTGTGCAGCAACCCGC |
| | GCTGGTGGTACTAGCTATGGAAAGCTGACATTTGGACAAGGGACCATCTTGACTGTCCATCCAAATA |
| | TCCAGAACCCTGACCCTGCCGTGTACCAGCTGAGAGACT |
| 364 | TTCCTCTGCTCTGGCAGCAGATCTCCCAGAGGGAGCAGCCTGACCACATCACTGGCCCAGAAGAGG |
| | AGGCGTCTGTCCCCCAGACTAGCTGAAGGAAAGGCTGGCTTGGATGATGCTCTGCTCTCTCCTTGC |
| | CCTTCTCCTGGGCACTTTCTTTGGGGTCAGATCTCAGACTATTCATCAATGGCCAGCGACCCTGGTG |
| | CAGCCTGTGGGCAGCCCGCTCTCTCTGGAGTGCACTGTGGAGGGAACATCAAACCCCAACCTATAC |
| | TGGTACCGACAGGCTGCAGGCAGGGGCCTCCAGCTGCTCTTCTACTCCGTTGGTATTGGCCAGATC |
| | AGCTCTGAGGTGCCCCAGAATCTCTCAGCCTCCAGACCCCAGGACCGGCAGTTCATCCTGAGTTCT |
| | AAGAAGCTCCTTCTCAGTGACTCTGGCTTCTATCTCTGTGCCTGGAGTGTACTAGCAGGGGTTTCCC |
| | AGTACTTCGGGCCAGGCACGCGGCTCCTGGTGCTCGAGGACCTGAAAAACGTGTTCCCACCCGAG |
| | GTCGCTGTGTTTGAGCCATCAGA |
| 365 | GATCTTAATTGGGAAGAACAAGGATGACATCCATTCGAGCTGTATTTATATTCCTGTGGCTGCAGCTG |
| | GACTTGGTGAATGGAGAGAATGTGGAGCAGCATCCTTCAACCCTGAGTGTCCAGGAGGGAGACAGC |
| | GCTGTTATCAAGTGTACTTATTCAGACAGTGCCTCAAACTACTTCCCTTGGTATAAGCAAGAACTTGG |
| | AAAAAGACCTCAGCTTATTATAGACATTCGTTCAAATGTGGGCGAAAAGAAAGACCAACGAATTGCTG |
| | TTACATTGAACAAGACAGCCAAACATTTCTCCCTGCACATCACAGAGACCCAACCTGAAGACTCGGC |
| | TGTCTACTTCTGTGCAGCAAGTATAGCCACCGACAAGCTCATCTTTGGGACTGGGACCAGATTACAA |
| | GTCTTTCCAAATATCCAGAACCCTGACCCTGCCGTGTACCAGCTGAGAGACT |
| 366 | AGAGGACCAGTATCCCTCACAGGGTGACACCTGACCAGCTCTGTCCCACCTGGCCATGGGCTCCAG |
| | GTACCTCTGATGGGAAGACCTTTGTCTCTTGGGAACAAGTGAATCCTTGGCACAGGCCCAGTGGATT |
| | CTGCTGTGCAGAACAGAGAGCAGTGGACCTCAGGAGGCCTGCAAGGGGAGGACATAGGACAGTGA |
| | CATCACAGTATGCCCCTCCCACCAGGAAAAGCAAGGCTGAGAATTTAGCTCTTTCCCAGGAGGACCA |
| | AGCCCTGAGCACAGACACAGTGCTGCCTGCCCCTTTGTGCCATGGGCTCCAGGCTGCTCTGTTGGG |
| | TGCTGCTTTGTCTCCTGGGAGCAGGCCCAGTAAAGGCTGGAGTCACTCAAACTCCAAGATATCTGAT |
| | CAAAACGAGAGGACAGCAAGTGACACTGAGCTGCTCCCCTATCTCTGGGCATAGGAGTGTATCCTG |
| | GTACCAACAGACCCCAGGACAGGGCCTTCAGTTCCTCTTTGAATACTTCAGTGAGACACAGAGAAAC |
| | AAAGGAAACTTCCCTGGTCGATTCTCAGGGCGCCAGTTCTCTAACTCTCGCTCTGAGATGAATGTGA |
| | GCACCTTGGAGCTGGGGGACTCGGCCCTTTATCTTTGCGCCAGCAGCTGGAGGGGACAGGGGGAA |
| | GGCTACACCTTCGGTTCGGGGACCAGGTTAACCGTTGTAGAGGACCTGAACAAGGTGTTCCCACCC |
| | GAGGTCGCTGTGTTTGAGCCATCAGA |
| 367 | AGTCAACTTCTGGGAGCAGATCTCTGCAGAATAAAAATGAAAAAGCATCTGACGACCTTCTTGGTGA |
| | TTTTGTGGCTTTATTTTTATAGGGGGAATGGCAAAAACCAAGTGGAGCAGAGTCCTCAGTCCCTGAT |
| | CATCCTGGAGGGAAAGAACTGCACTCTTCAATGCAATTATACAGTGAGCCCCTTCAGCAACTTAAGG |
| | TGGTATAAGCAAGATACTGGGAGAGGTCCTGTTTCCCTGACAATCATGACTTTCAGTGAGAACACAA |
| | AGTCGAACGGAAGATATACAGCAACTCTGGATGCAGACACAAAGCAAAGCTCTCTGCACATCACAGC |
| | CTCCCAGCTCAGCGATTCAGCCTCCTACATCTGTGTGGTGACCGGGGGGGCAAACAACCTCTTCTTT |
| | GGGACTGGAACGAGACTCACCGTTATTCCCTATATCCAGAACCCTGACCCTGCCGTGTACCAGCTG |
| | AGAGACT |
| 368 | CTCAGAGGACCAGTATCCCTCACAGGGTGACACCTGACCAGCTCTGTCCCACCTGGCCATGGGCTC |
| | CAGGTACCTCTGATGGGAAGACCTTTGTCTCTTGGGAACAAGTGAATCCTTGGCACAGGCCCAGTG |
| | GATTCTGCTGTGCAGAACAGAGAGCAGTGGACCTCAGGAGGCCTGCAAGGGGAGGACATAGGACA |
| | GTGACATCACAGTATGCCCCTCCCACCAGGAAAAGCAAGGCTGAGAATTTAGCTCTTTCCCAGGAG |
| | GACCAAGCCCTGAGCACAGACACAGTGCTGCCTGCCCCTTTGTGCCATGGGCTCCAGGCTGCTCTG |
| | TTGGGTGCTGCTTTGTCTCCTGGGAGCAGGCCCAGTAAAGGCTGGAGTCACTCAAACTCCAAGATAT |
| | CTGATCAAAACGAGAGGACAGCAAGTGACACTGAGCTGCTCCCCTATCTCTGGGCATAGGAGTGTA |
| | TCCTGGTACCAACAGACCCCAGGACAGGGCCTTCAGTTCCTCTTTGAATACTTCAGTGAGACACAGA |
| | GAAACAAAGGAAACTTCCCTGGTCGATTCTCAGGGCGCCAGTTCTCTAACTCTCGCTCTGAGATGAA |
| | TGTGAGCACCTTGGAGCTGGGGGACTCGGCCCTTTATCTTTGCGCCAGCAGTGAGGCTGGGGAGT |
| | GGACGCAGTATTTTGGCCCAGGCACCCGGCTGACAGTGCTCGAGGACCTGAAAAACGTGTTCCCAC |
| | CCGAGGTCGCTGTGTTTGAGCCATCAGA |
| 369 | AGGGGGGAAATTGAAACCTGCCTGATGTGGGATGTGCTGTGGCTGCTGCTTTGTTGCTTGGGACCT |
| | CCTCTGACCTAGGATCAGACACAGAGTCTGAGTTCTGGGGCCTGGAACCTCAATGTGCACTTGAACA |
| | ATGAAGTTGGTGACAAGCATTACTGTACTCCTATCTTTGGGTATTATGGGTGATGCTAAGACCACACA |
| | GCCAAATTCAATGGAGAGTAACGAAGAAGAGCCTGTTCACTTGCCTTGTAACCACTCCACAATCAGT |
| | GGAACTGATTACATACATTGGTATCGACAGCTTCCCTCCCAGGGTCCAGAGTACGTGATTCATGGTC |
| | TTACAAGCAATGTGAACAACAGAATGGCCTCTCTGGCAATCGCTGAAGACAGAAAGTCCAGTACCTT |
| | GATCCTGCACCGTGCTACCTTGAGAGATGCTGCTGTGTACTACTGCATCCTGGTAGAAGGAAATGAG |
| | AAATTAACCTTTGGGACTGGAACAAGACTCACCATCATACCCAATATCCAGAACCCTGACCCTGCCG |
| | TGTACCAGCTGAGAGACT |
| 370 | GAATCCAGGTATGGCTTCTGATTGGTGCAATCTCCTGCACCAATGAGCAAAGTAACTTCTGCTGGGG |
| | AAGCTCATTCAGTAAAATCTGATTGAACTGTGTTTTCTAAATAGCTAAGGGATGGAGACTGTTCTGCA |
| | AGTACTCCTAGGGATATTGGGGTTCCAAGCAGCCTGGGTCAGTAGCCAAGAACTGGAGCAGAGTCC |
| | TCAGTCCTTGATCGTCCAAGAGGGAAAGAATCTCACCATAAACTGCACGTCATCAAAGACGTTATAT |
| | GGCTTATACTGGTATAAGCAAAAGTATGGTGAAGGTCTTATCTTCTTGATGATGCTACAGAAAGGTGG |
| | GGAAGAGAAAAGTCATGAAAAGATAACTGCCAAGTTGGATGAGAAAAAGCAGCAAAGTTCCCTGCAT |
| | ATCACAGCCTCCCAGCCCAGCCATGCAGGCATCTACCTCTGTGGAGCAGACGTACAAGGAAGCCAA |
| | GGAAATCTCATCTTTGGAAAAGGCACTAAACTCTCTGTTAAACCAAATATCCAGAACCCTGACCCTGC |
| | CGTGTACCAGCTGAGAGACT |
| 371 | AAAATGCCCCTCCTTTCCTCCACAGGACCAGATGCCTGAGCTAGGAAAGGCCTCATTCCTGCTGTGA |
| | TCCTGCCATGGATACCTGGCTCGTATGCTGGGCAATTTTTAGTCTCTTGAAAGCAGGACTCACAGAA |
| | CCTGAAGTCACCCAGACTCCCAGCCATCAGGTCACACAGATGGGACAGGAAGTGATCTTGCGCTGT |
| | GTCCCCATCTCTAATCACTTATACTTCTATTGGTACAGACAAATCTTGGGGCAGAAAGTCGAGTTTCT |
| | GGTTTCCTTTTATAATAATGAAATCTCAGAGAAGTCTGAAATATTCGATGATCAATTCTCAGTTGAAAG |
| | GCCTGATGGATCAAATTTCACTCTGAAGATCCGGTCCACAAAGCTGGAGGACTCAGCCATGTACTTC |
| | TGTGCCAGCAGGCTGGGGGGAAGGACCACTGAAGCTTTCTTTGGACAAGGCACCAGACTCACAGTT |
| | GTAGAGGACCTGAACAAGGTGTTCCCACCCGAGGTCGCTGTGTTTGAGCCATCAGA |
| 372 | GAGGGCGATAAGAGGAGGGGGTAGTGATATACCAGGGGTTGTGAAAATAACCTCTTTTTTCTAATTG |
| | GTAGGACAGATTCTTTTTATGATTCCTAAAGTGGAAGAAATAAAGTATCTCTGCTATGTTCATTTCTTT |
| | TTGGATTGAAAATTTTAATCCTCAGTGAACCAGGGCAGAAAAGAATGATGATATCCTTGAGAGTTTTA |
| | CTGGTGATCCTGTGGCTTCAGTTAAGCTGGGTTTGGAGCCAACGGAAGGAGGTGGAGCAGGATCCT |
| | GGACCCTTCAATGTTCCAGAGGGAGCCACTGTCGCTTTCAACTGTACTTACAGCAACAGTGCTTCTC |
| | AGTCTTTCTTCTGGTACAGACAGGATTGCAGGAAAGAACCTAAGTTGCTGATGTCCGTATACTCCAG |
| | TGGTAATGAAGATGGAAGGTTTACAGCACAGCTCAATAGAGCCAGCCAGTATATTTCCCTGCTCATC |
| | AGAGACTCCAAGCTCAGTGATTCAGCCACCTACCTCTGTGTGGTGGATTTTTATACCTCAGGAACCT |
| | ACAAATACATCTTTGGAACAGGCACCAGGCTGAAGGTTTTAGCAAATATCCAGAACCCTGACCCTGC |
| | CGTGTACCAGCTGAGAGACT |
| 373 | GTCATCCCTCCTCGCTGGTGAATGGAGGCAGTGGTCACAACTCTCCCCAGAGAAGGTGGTGTGAGG |
| | CCATCACGGAAGATGCTGCTGCTTCTGCTGCTTCTGGGGCCAGGCTCCGGGCTTGGTGCTGTCGTC |
| | TCTCAACATCCGAGCAGGGTTATCTGTAAGAGTGGAACCTCTGTGAAGATCGAGTGCCGTTCCCTGG |
| | ACTTTCAGGCCACAACTATGTTTTGGTATCGTCAGTTCCCGAAACAGAGTCTCATGCTGATGGCAACT |
| | TCCAATGAGGGCTCCAAGGCCACATACGAGCAAGGCGTCGAGAAGGACAAGTTTCTCATCAACCAT |
| | GCAAGCCTGACCTTGTCCACTCTGACAGTGACCAGTGCCCATCCTGAAGACAGCAGCTTCTACATCT |
| | GCAGTGCTAGAGATCGGGAGGCGGCCGGCTATGGCTACACCTTCGGTTCGGGGACCAGGTTAACC |
| | GTTGTAGAGGACCTGAACAAGGTGTTCCCACCCGAGGTCGCTGTGTTTGAGCCATCAGA |
| 374 | AAGCACTCTTCTAGCCCAGAGAAGTCTGTTCCAGGACGGGCTCTTTCAGGAGCAGCTAAAGTCAGG |
| | GGCCATGTCCACCATGTGATAGAAAGACAAGATGGTCCTGAAATTCTCCGTGTCCATTCTTTGGATT |
| | CAGTTGGCATGGGTGAGCACCCAGCTGCTGGAGCAGAGCCCTCAGTTTCTAAGCATCCAAGAGGGA |
| | GAAAATCTCACTGTGTACTGCAACTCCTCAAGTGTTTTTTCCAGCTTACAATGGTACAGACAGGAGCC |
| | TGGGGAAGGTCCTGTCCTCCTGGTGACAGTAGTTACGGGTGGAGAAGTGAAGAAGCTGAAGAGACT |
| | AACCTTTCAGTTTGGTGATGCAAGAAAGGACAGTTCTCTCCACATCACTGCAGCCCAGCCTGGTGAT |
| | ACAGGCCTCTACCTCTGTGCAGGGTTAGATGACAAGATCATCTTTGGAAAAGGGACACGACTTCATA |
| | TTCTCCCCAATATCCAGAACCCTGACCCTGCCGTGTACCAGCTGAGAGACT |
| 375 | GGCTTTGTCGGGTGGAGCTGATTGGTTGCAGGAGCAGCAACAGTTCCAGAGCCAAGTCATGACACC |
| | GACCTCCCCAAGGTTTAGTTAAATATATCTTATGGTGAAAATGCCCGGAGCAAGAAGGCAAAGCATC |
| | ATGAAGAGGATATTGGGAGCTCTGCTGGGGCTCTTGAGTGCCCAGGTTTGCTGTGTGAGAGGAATA |
| | CAAGTGGAGCAGAGTCCTCCAGACCTGATTCTCCAGGAGGGAGCCAATTCCACGCTGCGGTGCAAT |
| | TTTTCTGACTCTGTGAACAATTTGCAGTGGTTTCATCAAAACCCTTGGGGACAGCTCATCAACCTGTT |
| | TTACATTCCCTCAGGGACAAAACAGAATGGAAGATTAAGCGCCACGACTGTCGCTACGGAACGCTAC |
| | AGCTTATTGTACATTTCCTCTTCCCAGACCACAGACTCAGGCGTTTATTTCTGTGCTGTGGGGGGTA |
| | GCAACTATCAGTTAATCTGGGGCGCTGGGACCAAGCTAATTATAAAGCCAGATATCCAGAACCCTGA |
| | CCCTGCCGTGTACCAGCTGAGAGACT |
| 376 | ACTAACTGATATCTCTGTTTCGAGTTGCCTTCAACTCGAAACATCCAGCAGAGGATGGGCCTAGAGA |
| | TGGAGTAGGAGATCCAGTCCCCAAGCCCTAGAGATGCATTTGTGGAGGCAATGATGTCACTGTGGG |
| | AACTGCCATGAGAGGACAGGGACGTCCCTCCTTGGGGGCTGTTGCTCACAGTGACCCTGATTGGGC |
| | AAAGCTCCCATCCTTCCCTGACCCTGCCATGGGCACCAGGCTCCTCTGCTGGGCGGCCCTCTGTCT |
| | CCTGGGAGCAGAACTCACAGAAGCTGGAGTTGCCCAGTCTCCCAGATATAAGATTATAGAGAAAAG |
| | GCAGAGTGTGGCTTTTTGGTGCAATCCTATATCTGGCCATGCTACCCTTTACTGGTACCAGCAGATC |
| | CTGGGACAGGGCCCAAAGCTTCTGATTCAGTTTCAGAATAACGGTGTAGTGGATGATTCACAGTTGC |
| | CTAAGGATCGATTTTCTGCAGAGAGGCTCAAAGGAGTAGACTCCACTCTCAAGATCCAACCTGCAAA |
| | GCTTGAGGACTCGGCCGTGTATCTCTGTGCCAGCAGCGACCCCATTAGCGGGAGAGGGGATGAGC |
| | AGTTCTTCGGGCCAGGGACACGGCTCACCGTGCTAGAGGACCTGAAAAACGTGTTCCCACCCGAGG |
| | TCGCTGTGTTTGAGCCATCAGA |
| 377 | GGCTCGTGTGTGTGTGTGTCTGTGTGTGTGTGTGTGTGCTTGAGAGAGAGAGAAGGAGAGAAAGAG |
| | AGAGAATGGGAAGGGCGATAAGAGGAGGGGTTAGTGATATACCAGGGGTTGTGAAAATAACCTCTT |
| | TTTTCTAATTGGGAGGACAGATTCTTTTTACGATTCCTAAAGTGGAAGAAATAAAGTATCTCTGCTATG |
| | TTCATTTCTTTTTGGATTGAAAATTTTAATCCTCAGTGAACCAGGGCAGAAAAGAATGATGATATCCTT |
| | GAGAGTTTTACTGGTGATCCTGTGGCTTCAGTTAAGCTGGGTTTGGAGCCAACGGAAGGAGGTGGA |
| | GCAGGATCCTGGACCCTTCAATGTTCCAGAGGGAGCCACTGTCGCTTTCAACTGTACTTACAGCAAC |
| | AGTGCTTCTCAGTCTTTCTTCTGGTACAGACAGGATTGCAGGAAAGAACCTAAGTTGCTGATGTCCG |
| | TATACTCCAGTGGTAATGAAGATGGAAGGTTTACAGCACAGCTCAATAGAGCCAGCCAGTATATTTC |
| | CCTGCTCATCAGAGACTCCAAGCTCAGTGATTCAGCCACCTACCTCTGTGTGGTGAACAAAGCCACC |
| | TCAGGAACCTACAAATACATCTTTGGAACAGGCACCAGGCTGAAGGTTTTAGCAAATATCCAGAACC |
| | CTGACCCTGCCGTGTACCAGCTGAGAGACT |
| 378 | GTCATCCCTCCTCGCTGGTGAATGGAGGCAGTGGTCACAACTCTCCCCAGAGAAGGTGGTGTGAGG |
| | CCATCACGGAAGATGCTGCTGCTTCTGCTGCTTCTGGGGCCAGGCTCCGGGCTTGGTGCTGTCGTC |
| | TCTCAACATCCGAGCTGGGTTATCTGTAAGAGTGGAACCTCTGTGAAGATCGAGTGCCGTTCCCTGG |
| | ACTTTCAGGCCACAACTATGTTTTGGTATCGTCAGTTCCCGAAACAGAGTCTCATGCTGATGGCAACT |
| | TCCAATGAGGGCTCCAAGGCCACATACGAGCAAGGCGTCGAGAAGGACAAGTTTCTCATCAACCAT |
| | GCAAGCCTGACCTTGTCCACTCTGACAGTGACCAGTGCCCATCCTGAAGACAGCAGCTTCTACATCT |
| | GCAGTGCTAGAGATCGGGAGGCGGCCGGCTATGGCTACACCTTCGGTTCGGGGACCAGGTTAACC |
| | GTTGTAGAGGACCTGAACAAGGTGTTCCCACCCGAGGTCGCTGTGTTTGAGCCATCAGA |
| 379 | GTCATCCCTCCTCGCTGGTGAATGGAGGCAGTGGTCACAACTCTCCCCAGAGAAGGTGGTGTGAGG |
| | CCATCACGGAAGATGCTGCTGCTTCTGCTGCTTCTGGGGCCAGGCTCCGGGCTTGGTGCTGTCGTC |
| | TCTCAACATCCGAGCTGGGTTATCTGTAAGAGTGGAACCTCTGTGAAGATCGAGTGCCGTTCCCTGG |
| | ACTTTCAGGCCACAACTATGTTTTGGTATCGTCAGTTCCCGAAACAGAGTCTCATGCTGATGGCAACT |
| | TCCAATGAGGGCTCCAAGGCCACATACGAGCAAGGCGTCGAGAAGGACAAGTTTCTCATCAACCAT |
| | GCAAGCCTGACCTTGTCCACTCTGACAGTGACCAGTGCCCATCCTGAAGACAGCAGCTTCTACATCT |
| | GCAGTGCTAGATTGGCCCATAGCGGGACCAACACCGGGGAGCTGTTTTTTGGAGAAGGCTCTAGGC |
| | TGACCGTACTGGAGGACCTGAAAAACGTGTTCCCACCCGAGGTCGCTGTGTTTGAGCCATCAGA |
| 380 | AASCEPLASVLRAKLTSRSS |
| 381 | GAGPDPLRLHGHLPVRTSCP |
| 382 | TQRSVLLCKVVGARGVGKSA |
| 383 | KVVGARGVGKSAFLQAFLGR |
| 384 | GQKSPRFRRVSCFLRLGRST |
| 385 | RFRRVSCFLRLGRSTLLELE |
| 386 | ALAFLLLISIAANLSLLLSR |
| 387 | EVQDCLKQLMMSLLQLYRFS |
| 388 | GYSPSLHILAIGTRSGAIKL |
| 389 | VATFPVYTMVAIPIVCKD |
| 390 | PPLYRQRYQFIKNLVDQHEP |
| 391 | PLYRQRYQFIKNLVDQHEPK |
| 392 | VSRPELLRESISAFLVPMPT |
| 393 | TDRALQNKSISAFLVPMPTP |
| 394 | NPLSPYLNVDPRYLVQDT |
| 395 | EPPVDICLSKAISSSLKGFL |
| 396 | GETGMFSLSTIRGHQYATY |
| 397 | MNYVSKRLPFAARLNTPMGP |
| 398 | LGSLGLIFALTLNRHKYPLN |
| 399 | LGLIFALTLNRHKYPLNLYL |
| 400 | APISLSSFFNVSTLEREVTD |
| 401 | LELGAGTGLASIIAATMART |
| 402 | AGTGLASIIAATMARTVYCT |
| 403 | VPREYVRALNATKLERVFAK |
| 404 | LHRDKALLKRLLKGMQKKRP |
| 405 | KALLKRLLKGMQKKRPSDVQ |
| 406 | ITVQTVYVQHLITFLDRPIQ |
| 407 | QTVYVQHLITFLDRPIQMCC |
| 408 | PGLISMFSSSQELGAALAQL |
| 409 | WRVRIALALKGIDYETVPIN |
| 410 | VRIALALKGIDYETVPINLI |
| 411 | DRAEKFNRGIRKLGITPEGQ |
| 412 | EKFNRGIRKLGITPEGQSYL |
| 413 | LTISLLDTSNLELPEAVVFQ |
| 414 | ISLLDTSNLELPEAVVFQDS |
| 415 | AASCEPLASVLRAKPTSRSS |
| 416 | GAGPDPLRLRGHLPVRTSCP |
| 417 | TQRSVLLCKVVGACGVGKSA |
| 418 | KVVGACGVGKSAFLQAFLGR |
| 419 | GQKSPRFRRVTCFLRLGRST |
| 420 | RFRRVTCFLRLGRSTLLELE |
| 421 | ALAFLLLISTAANLSLLLSR |
| 422 | EVQDCLKQLMMSLLRLYRFS |
| 423 | GYSPSLRILAIGTRSGAIKL |
| 424 | VATFPVYTMGAIPIVCKD |
| 425 | PPLYRQRYQFVKNLVDQHEP |
| 426 | PLYRQRYQFVKNLVDQHEPK |
| 427 | VSRPELLREGISAFLVPMPT |
| 428 | TDRALQNKGISAFLVPMPTP |
| 429 | NPLCPYLNVDPRYLVQDT |
| 430 | EPPVDICLSKANSSSLKGFL |
| 431 | GETGMFSLCTIRGHQYATY |
| 432 | MNYVSKSLPFAARLNTPMGP |
| 433 | LGSLGLIFALILNRHKYPLN |
| 434 | LGLIFALILNRHKYPLNLYL |
| 435 | APISLSSFFSVSTLEREVTD |
| 436 | LELGAGTGLTSIIAATMART |
| 437 | AGTGLTSIIAATMARTVYCT |
| 438 | VPREYIRALNATKLERVFAK |
| 439 | LHRDKALLKRLLKGVQKKRP |
| 440 | KALLKRLLKGVQKKRPSDVQ |
| 441 | ITVQTVYVQHPITFLDRPIQ |
| 442 | QTVYVQHPITFLDRPIQMCC |
| 443 | PGLISVFSSSQELGAALAQL |
| 444 | WRVRIALALKGIDYKTVPIN |
| 445 | VRIALALKGIDYKTVPINLI |
| 446 | DRAEKFNRGIRKLGVTPEGQ |
| 447 | EKFNRGIRKLGVTPEGQSYL |
| 448 | ATGYPS |
| 449 | DFQATT |
| 450 | YGATPY |
| 451 | SNHLY |
| 452 | DFQATT |
| 453 | VTNFRS |
| 454 | SGHNS |
| 455 | DSSSTY |
| 456 | MNHEY |
| 457 | TTSDR |
| 458 | MNHEY |
| 459 | MNHEY |
| 460 | SSVSVY |
| 461 | SGHTA |
| 462 | SGHTA |
| 463 | ATGYPS |
| 464 | NTAFDY |
| 465 | DFQATT |
| 466 | DRGSQS |
| 467 | MNHEY |
| 468 | ATGYPS |
| 469 | DFQATT |
| 470 | NIATNDY |
| 471 | SGHRS |
| 472 | TISGTDY |
| 473 | DFQATT |
| 474 | TSGFYG |
| 475 | VSNAYN |
| 476 | SGHNT |
| 477 | VSNAYN |
| 478 | SGHNT |
| 479 | SIFNT |
| 480 | MNHNY |
| 481 | SIFNT |
| 482 | SQVTM |
| 483 | DRGSQS |
| 484 | LGHDT |
| 485 | VGISA |
| 486 | VGISA |
| 487 | MNHEY |
| 488 | TRDTTYY |
| 489 | SGHNT |
| 490 | DRGSQS |
| 491 | KGHDR |
| 492 | TRDTTYY |
| 493 | KGHSH |
| 494 | SIFNT |
| 495 | LNHDA |
| 496 | DSVNN |
| 497 | LGHNT |
| 498 | TISGNEY |
| 499 | MNHEY |
| 500 | SSVSVY |
| 501 | WSHSY |
| 502 | NSASDY |
| 503 | NSASDY |
| 504 | SEHNR |
| 505 | SSVPPY |
| 506 | MNHEY |
| 507 | MNHEY |
| 508 | DSSSTY |
| 509 | MNHEY |
| 510 | TSINN |
| 511 | SNHLY |
| 512 | TSINN |
| 513 | SNHLY |
| 514 | DSASNY |
| 515 | SGHDY |
| 516 | TSINN |
| 517 | SNHLY |
| 518 | NSMFDY |
| 519 | GTSNPN |
| 520 | DSASNY |
| 521 | SGHRS |
| 522 | VSPFSN |
| 523 | SGHRS |
| 524 | TISGTDY |
| 525 | KTLYG |
| 526 | SNHLY |
| 527 | NSASQS |
| 528 | DFQATT |
| 529 | SVFSS |
| 530 | DSVNN |
| 531 | SGHAT |
| 532 | NSASQS |
| 533 | DFQATT |
| 534 | DFQATT |
| 535 | GCCACAGGATACCCTTCC |
| 536 | GACTTTCAGGCCACAACT |
| 537 | TATGGGGCAACACCTTAT |
| 538 | TCTAATCACTTATAC |
| 539 | GACTTTCAGGCCACAACT |
| 540 | GTGACTAACTTTCGAAGC |
| 541 | TCAGGCCACAACTCC |
| 542 | GACAGCTCCTCCACCTAC |
| 543 | ATGAACCATGAGTAT |
| 544 | ACCACTTCAGACAGA |
| 545 | ATGAACCATGAGTAT |
| 546 | ATGAACCATGAGTAT |
| 547 | TCGTCTGTTTCAGTGTAT |
| 548 | TCAGGTCATACTGCC |
| 549 | TCAGGTCATACTGCC |
| 550 | GCCACAGGATACCCTTCC |
| 551 | AACACTGCGTTTGACTAC |
| 552 | GACTTTCAGGCCACAACT |
| 553 | GACCGAGGTTCCCAGTCC |
| 554 | ATGAACCATGAATAC |
| 555 | GCCACAGGATACCCTTCC |
| 556 | GACTTTCAGGCCACAACT |
| 557 | AACATTGCTACAAATGATTAT |
| 558 | TCTGGGCATAGGAGT |
| 559 | ACAATCAGTGGAACTGATTAC |
| 560 | GACTTTCAGGCCACAACT |
| 561 | ACATCTGGGTTTTATGGG |
| 562 | GTGTCCAATGCTTACAAC |
| 563 | TCTGGCCACAATACC |
| 564 | GTGTCCAATGCTTACAAC |
| 565 | TCTGGCCACAATACC |
| 566 | AGCATATTTAACACC |
| 567 | ATGAACCATAACTAC |
| 568 | AGCATATTTAACACC |
| 569 | AGCCAAGTCACCATG |
| 570 | GACCGAGGTTCCCAGTCC |
| 571 | CTGGGCCATGATACT |
| 572 | GTAGGAATAAGTGCC |
| 573 | GTAGGAATAAGTGCC |
| 574 | ATGAACCATGAATAC |
| 575 | ACCCGTGATACTACTTATTAC |
| 576 | TCTGGGCACAACACT |
| 577 | GACCGAGGTTCCCAGTCC |
| 578 | AAGGGTCATGATAGA |
| 579 | ACCCGTGATACTACTTATTAC |
| 580 | AAAGGACACAGTCAT |
| 581 | AGCATATTTAACACC |
| 582 | TTGAACCACGATGCC |
| 583 | GACTCTGTGAACAAT |
| 584 | TTAGGCCACAATACT |
| 585 | ACCATCAGTGGAAATGAGTAT |
| 586 | ATGAACCATGAGTAT |
| 587 | TCGTCTGTTTCAGTGTAT |
| 588 | TGGAGCCACAGCTAT |
| 589 | AACAGCGCCTCAGACTAC |
| 590 | AACAGCGCCTCAGACTAC |
| 591 | TCTGAACACAACCGC |
| 592 | TCGTCTGTTCCACCATAT |
| 593 | ATGAACCATGAATAC |
| 594 | ATGAACCATGAATAC |
| 595 | GACAGCTCCTCCACCTAC |
| 596 | ATGAACCATGAATAC |
| 597 | ACTAGTATAAACAAT |
| 598 | TCTAATCACTTATAC |
| 599 | ACTAGTATAAACAAT |
| 600 | TCTAATCACTTATAC |
| 601 | GACAGTGCCTCAAACTAC |
| 602 | TCAGGACACGACTAC |
| 603 | ACTAGTATAAACAAT |
| 604 | TCTAATCACTTATAC |
| 605 | AACAGCATGTTTGATTAT |
| 606 | GGAACATCAAACCCCAAC |
| 607 | GACAGTGCCTCAAACTAC |
| 608 | TCTGGGCATAGGAGT |
| 609 | GTGAGCCCCTTCAGCAAC |
| 610 | TCTGGGCATAGGAGT |
| 611 | ACAATCAGTGGAACTGATTAC |
| 612 | AAGACGTTATATGGC |
| 613 | TCTAATCACTTATAC |
| 614 | AACAGTGCTTCTCAGTCT |
| 615 | GACTTTCAGGCCACAACT |
| 616 | AGTGTTTTTTCCAGC |
| 617 | GACTCTGTGAACAAT |
| 618 | TCTGGCCATGCTACC |
| 619 | AACAGTGCTTCTCAGTCT |
| 620 | GACTTTCAGGCCACAACT |
| 621 | GACTTTCAGGCCACAACT |
| 622 | ATKADDK |
| 623 | SNEGSKA |
| 624 | YFSGDTLV |
| 625 | FYNNEI |
| 626 | SNEGSKA |
| 627 | LTSSGIE |
| 628 | FNNNVP |
| 629 | IFSNMDM |
| 630 | SMNVEV |
| 631 | LLSNGAV |
| 632 | SMNVEV |
| 633 | SMNVEV |
| 634 | YLSGSTLV |
| 635 | FQGNSA |
| 636 | FQGNSA |
| 637 | ATKADDK |
| 638 | IRPDVSE |
| 639 | SNEGSKA |
| 640 | IYSNGD |
| 641 | SVGAGI |
| 642 | ATKADDK |
| 643 | SNEGSKA |
| 644 | GYKTK |
| 645 | YFSETQ |
| 646 | GLTSN |
| 647 | SNEGSKA |
| 648 | NALDGL |
| 649 | GSKP |
| 650 | YENEEA |
| 651 | GSKP |
| 652 | YENEEA |
| 653 | LYKAGEL |
| 654 | SVGAGI |
| 655 | LYKAGEL |
| 656 | ANQGSEA |
| 657 | IYSNGD |
| 658 | YNNKEL |
| 659 | LSSGK |
| 660 | LSSGK |
| 661 | SVGAGI |
| 662 | RNSFDEQN |
| 663 | YYREEE |
| 664 | IYSNGD |
| 665 | SFDVKD |
| 666 | RNSFDEQN |
| 667 | LQKENI |
| 668 | LYKAGEL |
| 669 | SQIVND |
| 670 | IPSGT |
| 671 | FRNRAP |
| 672 | GLKNN |
| 673 | SMNVEV |
| 674 | YLSGSTLV |
| 675 | SAAADI |
| 676 | IRSNMDK |
| 677 | IRSNMDK |
| 678 | FQNEAQ |
| 679 | YTTGATLV |
| 680 | SVGAGI |
| 681 | SVGAGI |
| 682 | IFSNMDM |
| 683 | SVGAGI |
| 684 | IRSNERE |
| 685 | FYNNEI |
| 686 | IRSNERE |
| 687 | FYNNEI |
| 688 | IRSNVGE |
| 689 | FNNNVP |
| 690 | IRSNERE |
| 691 | FYNNEI |
| 692 | ISSIKDK |
| 693 | SVGIG |
| 694 | IRSNVGE |
| 695 | YFSETQ |
| 696 | MTFSENT |
| 697 | YFSETQ |
| 698 | GLTSN |
| 699 | LQKGGEE |
| 700 | FYNNEI |
| 701 | VYSSG |
| 702 | SNEGSKA |
| 703 | VVTGGEV |
| 704 | IPSGT |
| 705 | FQNNGV |
| 706 | VYSSG |
| 707 | SNEGSKA |
| 708 | SNEGSKA |
| 709 | GCCACGAAGGCTGATGACAAG |
| 710 | TCCAATGAGGGCTCCAAGGCC |
| 711 | TACTTTTCAGGAGACACTCTGGTT |
| 712 | TTTTATAATAATGAAATC |
| 713 | TCCAATGAGGGCTCCAAGGCC |
| 714 | CTAACTTCAAGTGGAATTGAA |
| 715 | TTTAACAACAACGTTCCG |
| 716 | ATTTTTTCAAATATGGACATG |
| 717 | TCAATGAATGTTGAGGTG |
| 718 | TTGCTATCAAATGGAGCAGTG |
| 719 | TCAATGAATGTTGAGGTG |
| 720 | TCAATGAATGTTGAGGTG |
| 721 | TATTTATCAGGATCCACCCTGGTT |
| 722 | TTCCAAGGCAACAGTGCA |
| 723 | TTCCAAGGCAACAGTGCA |
| 724 | GCCACGAAGGCTGATGACAAG |
| 725 | ATACGTCCAGATGTGAGTGAA |
| 726 | TCCAATGAGGGCTCCAAGGCC |
| 727 | ATATACTCCAATGGTGAC |
| 728 | TCAGTTGGTGCTGGTATC |
| 729 | GCCACGAAGGCTGATGACAAG |
| 730 | TCCAATGAGGGCTCCAAGGCC |
| 731 | GGATACAAGACAAAA |
| 732 | TACTTCAGTGAGACACAG |
| 733 | GGTCTTACAAGCAAT |
| 734 | TCCAATGAGGGCTCCAAGGCC |
| 735 | AATGCTCTGGATGGTTTG |
| 736 | GGCTCAAAGCCT |
| 737 | TATGAGAATGAGGAAGCA |
| 738 | GGCTCAAAGCCT |
| 739 | TATGAGAATGAGGAAGCA |
| 740 | TTATATAAGGCTGGTGAATTG |
| 741 | TCAGTTGGTGCTGGTATC |
| 742 | TTATATAAGGCTGGTGAATTG |
| 743 | GCAAATCAGGGCTCTGAGGCC |
| 744 | ATATACTCCAATGGTGAC |
| 745 | TACAATAATAAGGAGCTC |
| 746 | CTGAGCTCAGGGAAG |
| 747 | CTGAGCTCAGGGAAG |
| 748 | TCAGTTGGTGCTGGTATC |
| 749 | CGGAACTCTTTTGATGAGCAAAAT |
| 750 | TATTATAGGGAGGAAGAG |
| 751 | ATATACTCCAATGGTGAC |
| 752 | TCCTTTGATGTCAAAGAT |
| 753 | CGGAACTCTTTTGATGAGCAAAAT |
| 754 | CTCCAGAAAGAAAATATC |
| 755 | TTATATAAGGCTGGTGAATTG |
| 756 | TCACAGATAGTAAATGAC |
| 757 | ATTCCCTCAGGGACA |
| 758 | TTCCGCAACCGGGCTCCT |
| 759 | GGTCTAAAAAACAAT |
| 760 | TCAATGAATGTTGAGGTG |
| 761 | TATTTATCAGGATCCACCCTGGTT |
| 762 | TCAGCAGCTGCTGATATT |
| 763 | ATTCGTTCAAATATGGACAAA |
| 764 | ATTCGTTCAAATATGGACAAA |
| 765 | TTCCAGAATGAAGCTCAA |
| 766 | TACACAACAGGGGCCACCCTGGTT |
| 767 | TCAGTTGGTGCTGGTATC |
| 768 | TCAGTTGGTGCTGGTATC |
| 769 | ATTTTTTCAAATATGGACATG |
| 770 | TCAGTTGGTGCTGGTATC |
| 771 | ATACGTTCAAATGAAAGAGAG |
| 772 | TTTTATAATAATGAAATC |
| 773 | ATACGTTCAAATGAAAGAGAG |
| 774 | TTTTATAATAATGAAATC |
| 775 | ATTCGTTCAAATGTGGGCGAA |
| 776 | TTTAACAACAACGTTCCG |
| 777 | ATACGTTCAAATGAAAGAGAG |
| 778 | TTTTATAATAATGAAATC |
| 779 | ATAAGTTCCATTAAGGATAAA |
| 780 | TCCGTTGGTATTGGC |
| 781 | ATTCGTTCAAATGTGGGCGAA |
| 782 | TACTTCAGTGAGACACAG |
| 783 | ATGACTTTCAGTGAGAACACA |
| 784 | TACTTCAGTGAGACACAG |
| 785 | GGTCTTACAAGCAAT |
| 786 | CTACAGAAAGGTGGGGAAGAG |
| 787 | TTTTATAATAATGAAATC |
| 788 | GTATACTCCAGTGGT |
| 789 | TCCAATGAGGGCTCCAAGGCC |
| 790 | GTAGTTACGGGTGGAGAAGTG |
| 791 | ATTCCCTCAGGGACA |
| 792 | TTTCAGAATAACGGTGTA |
| 793 | GTATACTCCAGTGGT |
| 794 | TCCAATGAGGGCTCCAAGGCC |
| 795 | TCCAATGAGGGCTCCAAGGCC |

1-29. (canceled)

30. A method of selecting an immunotherapeutic agent for treating a disease in an individual, the method comprising
(a) identifying a peptide antigen that is (i) encoded by a germline variation in the individual, (ii) capable of binding to a MHC molecule and (iii) present on the surface of a target cell in the individual and optionally absent on the surface of non-target cells in the individual; and
(b) selecting an immunotherapeutic agent that comprises an antigen binding molecule that binds to the peptide antigen.

31. A method of treating a disease in an individual, the method comprising:
(a) administering to the individual an immunotherapeutic agent that comprises an antigen binding molecule that binds to a peptide antigen that is (i) encoded by a germline variation in the individual, (ii) capable of binding to a MHC molecule, and (iii) present on the surface of a target cell in the individual and optionally absent on the surface of non-target cells in the individual; or
(b) administering a composition comprising a peptide antigen to the individual and thereby inducing an immune response specific for the peptide antigen, wherein the peptide antigen is (i) encoded by a germline variation in the individual, (ii) capable of binding to a MHC molecule, and (iii) present on the surface of a target cell in the individual and optionally absent on the surface of non-target cells in the individual.

32. A composition of matter comprising:
(a) an immunotherapeutic agent comprising an antigen binding molecule that binds to a peptide antigen that is (i) encoded by a germline variation in the individual, (ii) capable of binding to a MHC molecule, and (iii) present on the surface of a target cell in the individual and optionally absent on the surface of non-target cells in the individual;
(b) an antigen binding molecule that binds to a peptide antigen that is (i) encoded by a germline variation in an individual having a disease, (ii) capable of binding to a MHC molecule, and (iii) present on the surface of a target cell in the individual and optionally absent on the surface of non-target cells in the individual; or
(c) a peptide antigen that is (i) encoded by a germline variation in an individual having a disease, (ii) capable of binding to a MHC molecule, and (iii) present on the surface of a target cell in the individual and optionally absent on the surface of non-target cells in the individual.

33. The method of claim 30, wherein:
(I) the MHC molecule is a MHC class I molecule or a MHC Class II molecule;
(II) the peptide antigen binds to an antigen binding domain that is expressed by an immune cell from a donor whose genome does not comprise the germline variation, optionally wherein the donor does not have the disease and/or wherein the germline variation comprises one or more nucleotide substitutions, insertions and/or deletions relative to the donor genome; or
(III) the disease is cancer and the target cell is a cancer cell, optionally wherein the cancer is acute myeloid leukaemia (AML) and the cancer cell is an AML cell.

34. The method of claim 31, wherein:
(I) the MHC molecule is a MHC class I molecule or a MHC Class II molecule;
(II) the peptide antigen binds to an antigen binding domain that is expressed by an immune cell from a donor whose genome does not comprise the germline variation, optionally wherein the donor does not have the disease and/or wherein the germline variation comprises one or more nucleotide substitutions, insertions and/or deletions relative to the donor genome; or
(III) the disease is cancer and the target cell is a cancer cell, optionally wherein the cancer is acute myeloid leukaemia (AML) and the cancer cell is an AML cell.

35. The method of claim 30, wherein:
(I) the antigen binding molecule is a T cell receptor (TCR), a chimeric antigen receptor (CAR), an antibody, antibody fragment or bi-specific T cell engager (BiTE);
(II) the antigen binding molecule is an antibody fragment, wherein the antibody fragment comprises a fragment antigen-binding (Fab) fragment, a F (ab′)2 fragment, a single chain variable fragment (scFv), a scFv-Fc, or a single domain antibody; or
(III) the antibody binding molecule comprises an antigen binding domain that is expressed by an immune cell from a donor whose genome does not comprise the germline variation.

36. The method of claim 31, wherein:
(I) the antigen binding molecule is a T cell receptor (TCR), a chimeric antigen receptor (CAR), an antibody, antibody fragment or bi-specific T cell engager (BiTE);
(II) the antigen binding molecule is an antibody fragment, wherein the antibody fragment comprises a fragment antigen-binding (Fab) fragment, a F (ab′)2 fragment, a single chain variable fragment (scFv), a scFv-Fc, or a single domain antibody; or
(III) the antibody binding molecule comprises an antigen binding domain that is expressed by an immune cell from a donor whose genome does not comprise the germline variation.

37. The method of claim 30, wherein the immunotherapeutic agent further comprises an immune cell that expresses the antigen binding molecule on its surface, optionally wherein:
(I) the immune cell is a T cell;
(II) the immune cell is derived from a donor whose genome does not comprise the germline variation;
(III) the immune cell is derived from a donor whose genome does not comprise the germline variation, wherein the donor does not have the disease;
(IV) the antigen binding molecule is a TCR; or
(V) the antigen binding molecule is a CAR, optionally wherein the immune cell is autologous or allogeneic with respect to the individual.

38. The method of claim 31, wherein the immunotherapeutic agent further comprises an immune cell that expresses the antigen binding molecule on its surface, optionally wherein:
(I) the immune cell is a T cell;
(II) the immune cell is derived from a donor whose genome does not comprise the germline variation;
(III) the immune cell is derived from a donor whose genome does not comprise the germline variation, wherein the donor does not have the disease;
(IV) the antigen binding molecule is a TCR; or
(V) the antigen binding molecule is a CAR, optionally wherein the immune cell is autologous or allogeneic with respect to the individual.

39. The method of claim 31 wherein in step (b):
(I) the immune response is a T cell response, optionally wherein the T cell response comprises a CD4+ T cell response or a CD8+ T cell response; or
(II) the method further comprises administering to the individual immune cells specific for the peptide antigen.

40. The method of claim 30, wherein the peptide antigen comprises or consists of one or more of SEQ ID NOs: 1 to 4 and 380 to 412.

41. The method of claim 31, wherein the peptide antigen comprises or consists of one or more of SEQ ID NOs: 1 to 4 and 380 to 412.

42. The method of claim 30, wherein the antigen binding molecule comprises:
(I) (a) a CDR3 from one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or
(b) a CDR3 from one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292;
(II) (a) CDR1, CDR2 and CDR3 from one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or
(b) CDR1, CDR2 and CDR3 from one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292; or
(III) (a) one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or
(b) one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292.

43. The method of claim 31, wherein the antigen binding molecule comprises:
(I) (a) a CDR3 from one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or
(b) a CDR3 from one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292;
(II) (a) CDR1, CDR2 and CDR3 from one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or
(b) CDR1, CDR2 and CDR3 from one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292; or
(III) (a) one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or
(b) one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292.

44. A polynucleotide encoding the antigen binding molecule or the peptide antigen of claim 32, or a vector comprising the polynucleotide.

45. The composition of matter of claim 32, wherein:
(I) the MHC molecule is a MHC class I molecule or a MHC Class II molecule;
(II) the peptide antigen binds to an antigen binding domain that is expressed by an immune cell from a donor whose genome does not comprise the germline variation, optionally wherein the donor does not have the disease and/or wherein the germline variation comprises one or more nucleotide substitutions, insertions and/or deletions relative to the donor genome; or
(III) the disease is cancer and the target cell is a cancer cell, optionally wherein the cancer is acute myeloid leukaemia (AML) and the cancer cell is an AML cell.

46. The composition of matter of claim 32, wherein:
(I) the antigen binding molecule is a T cell receptor (TCR), a chimeric antigen receptor (CAR), an antibody, antibody fragment or bi-specific T cell engager (BiTE);
(II) the antigen binding molecule is an antibody fragment, wherein the antibody fragment comprises a fragment antigen-binding (Fab) fragment, a F (ab′)2 fragment, a single chain variable fragment (scFv), a scFv-Fc, or a single domain antibody; or
(III) the antibody binding molecule comprises an antigen binding domain that is expressed by an immune cell from a donor whose genome does not comprise the germline variation.

47. The composition of matter of claim 32, wherein the immunotherapeutic agent further comprises an immune cell that expresses the antigen binding molecule on its surface, optionally wherein:
(I) the immune cell is a T cell;
(II) the immune cell is derived from a donor whose genome does not comprise the germline variation;
(III) the immune cell is derived from a donor whose genome does not comprise the germline variation, wherein the donor does not have the disease;
(IV) the antigen binding molecule is a TCR; or
(V) the antigen binding molecule is a CAR, optionally wherein the immune cell is autologous or allogeneic with respect to the individual.

48. The composition of matter of claim 32, wherein the peptide antigen comprises or consists of one or more of SEQ ID NOs: 1 to 4 and 380 to 412.

49. The composition of matter of claim 32, wherein the antigen binding molecule comprises:
(I) (a) a CDR3 from one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or
(b) a CDR3 from one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292;
(II) (a) CDR1, CDR2 and CDR3 from one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or
(b) CDR1, CDR2 and CDR3 from one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292; or
(III) (a) one or more of SEQ ID NOs: 6, 10, 12 16, 20, 206, 208, 211, 213, 215, 218, 221, 222, 224, 226, 228, 230, 232, 233, 235, 237, 239, 241, 243, 244, 246, 248, 250, 252, 254, 256, 258, 260, 261, 263, 266, 268, 270, 272, 274, 276, 278, 280, 282, 283, 285, 287, 288, and 290; and/or
(b) one or more of SEQ ID NOs: 8, 14, 18, 22, 207, 209, 210, 212, 214, 216, 217, 219, 220, 223, 225, 227, 229, 231, 234, 236, 238, 240, 242, 245, 247, 249, 251, 253, 255, 257, 259, 262, 264, 265, 267, 269, 271, 273, 275, 277, 279, 281, 284, 286, 289, 291, and 292.


SEQUENCE LISTING (795 sequences)

SEQ ID NO: 1 - protein, 20 aa, Homo sapiens
LTISLLDTFNLELPEAVVFQ

SEQ ID NO: 2 - protein, 20 aa, Homo sapiens
ISLLDTFNLELPEAVVFQDS

SEQ ID NO: 3 - protein, 16 aa, Homo sapiens
SLLDTFNLELPEAVVF

SEQ ID NO: 4 - protein, 15 aa, Homo sapiens
LDTFNLELPEAVVFQ

SEQ ID NO: 5 - DNA, 763 bp, Homo sapiens
cagaagcctcacacagcccagtaactttgctagtacctcttgagtgcaaggtggagaattaagatctggatttgagacggagcacggaacatttcactcaggggaagagctatgaacatgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaacggccgtatggtggtgctacaaacaagctcatctttggaactggcactctgcttgctgtccagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttca

SEQ ID NO: 6 - protein, 215 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSERPYGGATNKLIFGTGTLLAVQPNIQNPDPAVYQLRDSKSSDKSVCLFTDFDSQTNVSQSKDSDVYITDKTVLDMRSMDFKSNSAVAWSNKSDFACANAF

SEQ ID NO: 7 - DNA, 806 bp, Homo sapiens
aaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaacgcaggacgcaacctgcctacgagcagtacttcgggccgggcaccaggctcacggtcacagaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtgg

SEQ ID NO: 8 - protein, 244 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSERRTQPAYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEW

SEQ ID NO: 9 - DNA, 733 bp, Homo sapiens
aaagcagattctttttatgatttttaaagtagaaatatccattctaggtgcattttttaagggtttaaaatttgaatcctcagtgaaccagggcagagaagaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtccgggggcaggcaggaactgctctgatctttgggaagggaaccaccttatcagtgagttccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttca

SEQ ID NO: 10 - protein, 209 aa, Homo sapiens
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVRGQAGTALIFGKGTTLSVSSNIQNPDPAVYQLRDSKSSDKSVCLFTDFDSQTNVSQSKDSDVYITDKTVLDMRSMDFKSNSAVAWSNKSDFACANAF

SEQ ID NO: 11 - DNA, 748 bp, Homo sapiens
cagaagcctcacacagcccagtaactttgctagtacctcttgagtgcaaggtggagaattaagatctggatttgagacggagcacggaacatttcactcaggggaagagctatgaacatgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctcccggcaggaaacacacctcttgtctttggaaagggcacaagactttctgtgattgcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttca

SEQ ID NO: 12 - protein, 210 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALPAGNTPLVFGKGTRLSVIANIQNPDPAVYQLRDSKSSDKSVCLFTDFDSQTNVSQSKDSDVYITDKTVLDMRSMDFKSNSAVAWSNKSDFACANAF

SEQ ID NO: 13 - DNA, 947 bp, Homo sapiens
ctcagaggaccagtatccctcacagggtgacacctgaccagctctgtcccacctggccatgggctccaggtacctctgatgggaagacctttgtctcttgggaacaagtgaatccttggcacaggcccagtggattctgctgtgcagaacagagagcagtggacctcaggaggcctgcaaggggaggacataggacagtgacatcacagtatgcccctcccaccaggaaaagcaaggctgagaatttagctctttcccaggaggaccaagccctgagcacagacacagtgctgcctgcccctttgtgccatgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttgactgggggaaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgaagatcggaagagc

SEQ ID NO: 14 - protein, 212 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSLTGGNYGYTFGSGTRLTVVEDLNKVFPPEVAVFEPSEAEISHTQKATLVCLATGFFPDHVELSWWVNGKEVHSGVSTDPQPLKEQPALNDSRYCLKIGR

SEQ ID NO: 15 - DNA, 757 bp, Homo sapiens
ccaaaacaagagacttgcctagcccaaccttcctcacgctcgctattctcaagacctgggttccagccactttcctactggccccgaggagaatttccaaagagacgcctgcagtgtttccacagctcagccatgctcctgttgctcataccagtgctggggatgatttttgccctgagagatgccagagcccagtctgtgagccagcataaccaccacgtaattctctctgaagcagcctcactggagttgggatgcaactattcctatggtggaactgttaatctcttctggtatgtccagtaccctggtcaacaccttcagcttctcctcaagtacttttcaggggatccactggttaaaggcatcaagggctttgaggctgaatttataaagagtaaattctcctttaatctgaggaaaccctctgtgcagtggagtgacacagctgagtacttctgtgccgtaggcaccaatgcaggcaaatcaacctttggggatgggactacgctcactgtgaagccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttca

SEQ ID NO: 16 - protein, 208 aa, Homo sapiens
MLLLLIPVLGMIFALRDARAQSVSQHNHHVILSEAASLELGCNYSYGGTVNLFWYVQYPGQHLQLLLKYFSGDPLVKGIKGFEAEFIKSKFSFNLRKPSVQWSDTAEYFCAVGTNAGKSTFGDGTTLTVKPNIQNPDPAVYQLRDSKSSDKSVCLFTDFDSQTNVSQSKDSDVYITDKTVLDMRSMDFKSNSAVAWSNKSDFACANAF

SEQ ID NO: 17 - DNA, 683 bp, Homo sapiens
acctggagcccccagaactggcagacacctgcctgatgctgccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagccgaaagggactagcgggaggcggccccaccggggagctgttttttggagaaggctctaggctgaccgtactggaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacggacccgcagcccctcaaggagcagcccgccctcaatgactccagatactgcctga

SEQ ID NO: 18 - protein, 213 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSRKGLAGGGPTGELFFGEGSRLTVLEDLKNVFPPEVAVFEPSEAEISHTQKATLVCLATGFYPDHVELSWWVNGKEVHSGVSTDPQPLKEQPALNDSRYCL

SEQ ID NO: 19 - DNA, 936 bp, Homo sapiens
aggaaaagttgagggggcttgacagacagaaattctaaactgatgcttatctgtgtgtaaagaaaggattactgattcccaatgaatatatcttcagcaattctaaatttggacaaagtggggaagtgcttcctttgacagagacagctttaagtgaaagcacttgtgaaagggcggggcctgctgaaagaattcagttgagggtgaatttacagagtttcagctggttgggaagactggaagaccacctgggctgtcattgagctctggtgccaggaggaatggacaagatcttaggagcatcatttttagttctgtggcttcaactatgctgggtgagtggccaacagaaggagaaaagtgaccagcagcaggtgaaacaaagtcctcaatctttgatagtccagaaaggagggatttcaattataaactgtgcttatgagaacactgcgtttgactactttccatggtaccaacaattccctgggaaaggccctgcattattgatagccatacgtccagatgtgagtgaaaagaaagaaggaagattcacaatctccttcaataaaagtgccaagcagttctcattgcatatcatggattcccagcctggagactcagccacctacttctgtgcagctgtgacaggaggaggtgctgacggactcacctttggcaaagggactcatctaatcatccagccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttca

SEQ ID NO: 20 - protein, 218 aa, Homo sapiens
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAAVTGGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAF

SEQ ID NO: 21 - DNA, 706 bp, Homo sapiens
agctgtgaggtctggttccccgacgtgctgcagcaagtgcctttgccctgcctgtgggctccctccatggccaactctgctatggacaccagagtactctgctgtgcggtcatctgtcttctgggggcaggtctctcaaatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctccccccagggttacaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacggacccgcagcccctcaaggagcagcccgccctcaatgactccagatactgcctga

SEQ ID NO: 22 - protein, 208 aa, Homo sapiens
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPQGYNEQFFGPGTRLTVLEDLKNVFPPEVAVFEPSEAEISHTQKATLVCLATGFYPDHVELSWWVNGKEVHSGVSTDPQPLKEQPALNDSRYCL

SEQ ID NO: 23 - DNA, 453 bp, Artificial Sequence
  Codon optimised version of SEQ ID NO 5
gcggccgcgccaccatgcttacagcttctctgctgagagccgtgatcgccagcatctgtgtggtgtctagcatggcccagaaagtgacacaggcccagaccgagatcagcgtggtggaaaaagaagatgtgaccctggactgcgtgtacgagacacgggacaccacctactacctgttctggtacaagcagcctcctagcggcgagctggtgttcctgatcagacggaacagcttcgacgagcagaacgagatctccggccggtacagctggaacttccagaagtccaccagcagcttcaacttcaccatcaccgccagccaggtggtggatagcgccgtgtatttttgcgccctgagcgagaggccttacggcggagctacaaacaagctgatcttcggcaccggcacactgctggctgttcaacctaacatccagaaccccgaccccgcgg

SEQ ID NO: 24 - DNA, 530 bp, Artificial Sequence
  Codon optimised version of SEQ ID NO 7
ccatggatacctggctcgtgtgctgggccatcttcagcctgctgaaagccggactgaccgagcctgaagtgacccagacacctagccaccaagtgacacagatgggccaagaagtgatcctgcgctgcgtgcccatcagcaaccacctgtacttctactggtacagacagatcctgggccagaaagtggaattcctggtgtccttctacaacaacgagatcagcgagaagtccgagatcttcgacgaccagttcagcgtggaaagacccgacggcagcaacttcaccctgaagatcagaagcaccaagctcgaggacagcgccatgtacttttgcgccagcagcgagagaagaacccagcctgcctacgagcagtacttcggccctggcacaagactgaccgtgacagaggacctgcggaacgtgaccccccccaaggtgtccctgttcgagcccagcaaggccgagatcgccaacaagcagaaagccacactggtctgtctggctaggggcttcttccccgaccacgtg

SEQ ID NO: 25 - DNA, 435 bp, Artificial Sequence
  Codon optimised version of SEQ ID NO 9
gcggccgcgccaccatgaagtctctgagagtgctgctggtcatcctgtggctgcagctgtcttgggtctggtcccagcagaaagaggtggaacagaacagcggccctctgtctgttcctgaaggcgctatcgccagcctgaactgcacctacagcgatagaggcagccagagcttcttctggtacagacagtacagcggcaagagccccgagctgatcatgagcatctacagcaacggcgacaaagaggacggccggtttacagcccagctgaacaaggccagccagtacgtgtccctgctgatcagagatagccagcctagcgacagcgccacctatctgtgtgccgttagaggccaggctggcacagccctgatctttggcaagggcacaacactgagcgtgtccagcaacatccagaaccccgaccccgcgg

SEQ ID NO: 26 - DNA, 438 bp, Artificial Sequence
  Codon optimised version of SEQ ID NO 11
gcggccgcgccaccatgcttacagcttctctgctgagagccgtgatcgccagcatctgtgtggtgtctagcatggcccagaaagtgacacaggcccagaccgagatcagcgtggtggaaaaagaagatgtgaccctggactgcgtgtacgagacacgggacaccacctactacctgttctggtacaagcagcctcctagcggcgagctggtgttcctgatcagacggaacagcttcgacgagcagaacgagatctccggccggtacagctggaacttccagaagtccaccagcagcttcaacttcaccatcaccgccagccaggtggtggatagcgccgtgtacttttgtgccctgcctgccggaaatacccctctggtgtttggcaagggcaccagactgtctgtgatcgccaacatccagaaccccgaccccgcgg

SEQ ID NO: 27 - DNA, 521 bp, Artificial Sequence
  Codon optimised version of SEQ ID NO 13
ccatgggcagcagactgctgtgttgggtgctgctgtgtctgcttggagccggacctgtgaaagctggcgtgacccagacacctagatacctgatcaagaccagaggccagcaagtgaccctgagctgctctcctatcagcggccacagaagcgtgtcctggtatcagcagacacctggacagggcctgcagttcctgttcgagtacttcagcgagacacagcggaacaagggcaacttccccggcagattttccggcagacagttcagcaacagccgcagcgagatgaacgtgtccacactggaactgggcgacagcgccctgtatctgtgtgcctcttctctgaccggcggcaactacggctacacatttggcagcggcaccagactgacagtggtcgaggacctgcggaacgtgaccccccccaaggtgtccctgttcgagcccagcaaggccgagatcgccaacaagcagaaagccacactggtctgtctggctaggggcttcttccccgaccacgtg

SEQ ID NO: 28 - DNA, 432 bp, Artificial Sequence
  Codon optimised version of SEQ ID NO 15
gcggccgcgccaccatgttgttgctgctgattcctgtgctgggcatgatcttcgccctgagggatgctagagcccagtccgtgtctcagcacaaccaccacgtgatcctgtctgaggccgcctctctggaactgggctgcaattacagctacggcggcaccgtgaacctgttttggtacgtgcagtaccccggccagcatctccagctgctgctgaagtactttagcggcgaccctctggtcaagggcatcaagggattcgaggccgagttcatcaagagcaagttcagcttcaacctgcggaagcccagcgtgcagtggagcgatacagccgagtacttttgtgccgtgggcaccaatgccggcaagagcacatttggcgacggcaccacactgaccgtgaagcctaacatccagaaccccgaccccgcgg

SEQ ID NO: 29 - DNA, 536 bp, Artificial Sequence
  Codon optimised version of SEQ ID NO 17
ccatgggccctcagctgctgggatatgtggtgctgtgtctgcttggagccggacctctggaagcccaagtgacacagaaccccagatacctgatcaccgtgaccggcaagaaactgaccgtgacctgcagccagaacatgaaccacgagtacatgagctggtacagacaggaccctggcctgggcctgagacagatctactacagcatgaacgtggaagtgaccgacaagggcgacgtgcccgagggctacaaggtgtccagaaaagagaagcggaacttcccactgatcctggaaagcccatctcctaaccagaccagcctgtacttctgcgccagcagcagaaaaggactggctggcggaggacctaccggcgagctgttttttggcgagggcagcagactgacagtgctcgaggacctgcggaacgtgaccccccccaaggtgtccctgttcgagcccagcaaggccgagatcgccaacaagcagaaagccacactggtctgtctggctaggggcttcttccccgaccacgtg

SEQ ID NO: 30 - DNA, 462 bp, Artificial Sequence
  Codon optimised version of SEQ ID NO 19
gcggccgcgccaccatggataagattctgggcgccagcttcctggtgctgtggctgcaactttgttgggtgtccggccagcagaaagagaagtccgaccagcagcaagtgaaacagagccctcagagcctgatcgtgcagaaaggcggcatcagcatcatcaactgcgcctacgagaataccgccttcgactacttcccctggtatcagcagttccccggcaagggacctgctctgctgatcgccattagacccgacgtgtccgagaagaaagagggcagattcaccatcagcttcaacaagagcgccaagcagttcagcctgcacatcatggatagccagcctggcgacagcgccacctatttttgtgctgctgttacaggcggcggagccgatggcctgacatttggaaagggcacccacctgatcatccagccttacatccagaaccccgaccccgcgg

SEQ ID NO: 31 - DNA, 521 bp, Artificial Sequence
  Codon optimised version of SEQ ID NO 21
ccatggacaccagagtgctgtgctgcgccgtgatctgtctgcttggagccggactgtctaatgccggcgtgatgcagaaccccagacacctcgttcggagaagaggccaagaggccagactgagatgcagccctatgaagggccacagccatgtgtactggtacagacagctgcccgaagagggcctgaagttcatggtgtacctgcagaaagagaacatcatcgacgagagcggcatgcccaaagagcggttctctgccgagtttcccaaagagggccccagcatcctgagaatccagcaggttgtgcggggagatagcgccgcctacttttgtgctagcagccctcagggctacaacgagcagtttttcggccctggcaccagactgacagtgctcgaggacctgcggaacgtgaccccccccaaggtgtccctgttcgagcccagcaaggccgagatcgccaacaagcagaaagccacactggtctgtctggctaggggcttcttccccgaccacgtg

SEQ ID NO: 32 - protein, 14 aa, Homo sapiens
CALIPTGGGNKLTF

SEQ ID NO: 33 - protein, 17 aa, Homo sapiens
CSARLAHSGTNTGELFF

SEQ ID NO: 34 - protein, 14 aa, Homo sapiens
CAVGAYSSASKIIF

SEQ ID NO: 35 - protein, 15 aa, Homo sapiens
CASRDRGSGANVLTF

SEQ ID NO: 36 - protein, 14 aa, Homo sapiens
CSGGWGPNTGELFF

SEQ ID NO: 37 - protein, 19 aa, Homo sapiens
CAVGHFSSRSSGSARQLTF

SEQ ID NO: 38 - protein, 18 aa, Homo sapiens
CASSLKVGVDSSYNEQFF

SEQ ID NO: 39 - protein, 13 aa, Homo sapiens
CAESPSDGQKLLF

SEQ ID NO: 40 - protein, 15 aa, Homo sapiens
CASSLRQGISPEQFF

SEQ ID NO: 41 - protein, 13 aa, Homo sapiens
CAVEETSGSRLTF

SEQ ID NO: 42 - protein, 15 aa, Homo sapiens
CASSLRQGISPEQFF

SEQ ID NO: 43 - protein, 14 aa, Homo sapiens
CASSERQGITEAFF

SEQ ID NO: 44 - protein, 14 aa, Homo sapiens
CAVSGTGANSKLTF

SEQ ID NO: 45 - protein, 16 aa, Homo sapiens
CASSDPISGRGDEQFF

SEQ ID NO: 46 - protein, 15 aa, Homo sapiens
CASSFGGGAWNEQFF

SEQ ID NO: 47 - protein, 14 aa, Homo sapiens
CALRHKAAGNKLTF

SEQ ID NO: 48 - protein, 15 aa, Homo sapiens
CAALFDGGSQGNLIF

SEQ ID NO: 49 - protein, 11 aa, Homo sapiens
CSASGPEKLFF

SEQ ID NO: 50 - protein, 13 aa, Homo sapiens
CASRGLAGETQYF

SEQ ID NO: 51 - protein, 13 aa, Homo sapiens
CAVIPNSGYALNF

SEQ ID NO: 52 - protein, 14 aa, Homo sapiens
CALRGRNQGGKLIF

SEQ ID NO: 53 - protein, 15 aa, Homo sapiens
CSARLSSGGGYEQFF

SEQ ID NO: 54 - protein, 15 aa, Homo sapiens
CLVVPVYNQGGKLIF

SEQ ID NO: 55 - protein, 16 aa, Homo sapiens
CASSSGTSGMGETQYF

SEQ ID NO: 56 - protein, 14 aa, Homo sapiens
CILRDVGGSEKLVF

SEQ ID NO: 57 - protein, 13 aa, Homo sapiens
CSAKGLENQPQHF

SEQ ID NO: 58 - protein, 12 aa, Homo sapiens
CAVLKKGYALNF

SEQ ID NO: 59 - protein, 12 aa, Homo sapiens
CAVAGGYNKLIF

SEQ ID NO: 60 - protein, 15 aa, Homo sapiens
CASSLEAGDSYEQYF

SEQ ID NO: 61 - protein, 12 aa, Homo sapiens
CAVTGGYNKLIF

SEQ ID NO: 62 - protein, 15 aa, Homo sapiens
CASSLEAGDSYEQYF

SEQ ID NO: 63 - protein, 14 aa, Homo sapiens
CAGHQIQGAQKLVF

SEQ ID NO: 64 - protein, 14 aa, Homo sapiens
CASSYSGMNTEAFF

SEQ ID NO: 65 - protein, 12 aa, Homo sapiens
CAGANNNARLMF

SEQ ID NO: 66 - protein, 15 aa, Homo sapiens
CSVTPGGGVNTEAFF

SEQ ID NO: 67 - protein, 15 aa, Homo sapiens
CAVSAGGTSYGKLTF

SEQ ID NO: 68 - protein, 13 aa, Homo sapiens
CASSQVLRGEQYF

SEQ ID NO: 69 - protein, 14 aa, Homo sapiens
CAVSTGGATNKLIF

SEQ ID NO: 70 - protein, 15 aa, Homo sapiens
CAVQEGETSGSRLTF

SEQ ID NO: 71 - protein, 14 aa, Homo sapiens
CASSPGFNGNTIYF

SEQ ID NO: 72 - protein, 15 aa, Homo sapiens
CALSEATYNTDKLIF

SEQ ID NO: 73 - protein, 14 aa, Homo sapiens
CASSSTGTDYGYTF

SEQ ID NO: 74 - protein, 13 aa, Homo sapiens
CAGRQTSYDKVIF

SEQ ID NO: 75 - protein, 17 aa, Homo sapiens
CATSDVTGQGEMRGYTF

SEQ ID NO: 76 - protein, 14 aa, Homo sapiens
CALSEMNRDDKIIF

SEQ ID NO: 77 - protein, 15 aa, Homo sapiens
CASSPTGVSGNTIYF

SEQ ID NO: 78 - protein, 14 aa, Homo sapiens
CAGQQKTSGSRLTF

SEQ ID NO: 79 - protein, 16 aa, Homo sapiens
CASSSPRDRVGQPQHF

SEQ ID NO: 80 - protein, 14 aa, Homo sapiens
CAARRVDNNNDMRF

SEQ ID NO: 81 - protein, 11 aa, Homo sapiens
CASGLAQPQHF

SEQ ID NO: 82 - protein, 15 aa, Homo sapiens
CIVRVAVTNAGKSTF

SEQ ID NO: 83 - protein, 13 aa, Homo sapiens
CASRYYSADTQYF

SEQ ID NO: 84 - protein, 16 aa, Homo sapiens
CAVSGVLTGGGNKLTF

SEQ ID NO: 85 - protein, 15 aa, Homo sapiens
CASSEGTVSNQPQHF

SEQ ID NO: 86 - protein, 15 aa, Homo sapiens
CAEIGSGAGSYQLTF

SEQ ID NO: 87 - protein, 14 aa, Homo sapiens
CAENQGGSSYKLIF

SEQ ID NO: 88 - protein, 15 aa, Homo sapiens
CASSSQSGVDTEAFF

SEQ ID NO: 89 - protein, 12 aa, Homo sapiens
CAVSPPAQKLVF

SEQ ID NO: 90 - protein, 14 aa, Homo sapiens
CASQETGVGGELFF

SEQ ID NO: 91 - protein, 14 aa, Homo sapiens
CASSTTAVVSPLHF

SEQ ID NO: 92 - protein, 12 aa, Homo sapiens
CAEKKEGFKTIF

SEQ ID NO: 93 - protein, 14 aa, Homo sapiens
CASQETGVGGELFF

SEQ ID NO: 94 - protein, 13 aa, Homo sapiens
CATDEAAGNKLTF

SEQ ID NO: 95 - protein, 15 aa, Homo sapiens
CASKRESLATGELFF

SEQ ID NO: 96 - protein, 15 aa, Homo sapiens
CAAMYSGGGADGLTF

SEQ ID NO: 97 - protein, 14 aa, Homo sapiens
CASSTQGQAYEQYF

SEQ ID NO: 98 - protein, 13 aa, Homo sapiens
CAARGGSNYKLTF

SEQ ID NO: 99 - protein, 11 aa, Homo sapiens
CASSTGGEQYF

SEQ ID NO: 100 - protein, 12 aa, Homo sapiens
CATKGGNNRLAF

SEQ ID NO: 101 - protein, 14 aa, Homo sapiens
CASSAWTGETGYTF

SEQ ID NO: 102 - protein, 16 aa, Homo sapiens
CAATRAGGTSYGKLTF

SEQ ID NO: 103 - protein, 13 aa, Homo sapiens
CAWSVLAGVSQYF

SEQ ID NO: 104 - protein, 12 aa, Homo sapiens
CAASIATDKLIF

SEQ ID NO: 105 - protein, 14 aa, Homo sapiens
CASSWRGQGEGYTF

SEQ ID NO: 106 - protein, 12 aa, Homo sapiens
CVVTGGANNLFF

SEQ ID NO: 107 - protein, 13 aa, Homo sapiens
CASSEAGEWTQYF

SEQ ID NO: 108 - protein, 12 aa, Homo sapiens
CILVEGNEKLTF

SEQ ID NO: 109 - protein, 14 aa, Homo sapiens
CGADVQGSQGNLIF

SEQ ID NO: 110 - protein, 14 aa, Homo sapiens
CASRLGGRTTEAFF

SEQ ID NO: 111 - protein, 15 aa, Homo sapiens
CVVDFYTSGTYKYIF

SEQ ID NO: 112 - protein, 15 aa, Homo sapiens
CSARDREAAGYGYTF

SEQ ID NO: 113 - protein, 10 aa, Homo sapiens
CAGLDDKIIF

SEQ ID NO: 114 - protein, 12 aa, Homo sapiens
CAVAGSNYQLIW

SEQ ID NO: 115 - protein, 16 aa, Homo sapiens
CASSDPISGRGDEQFF

SEQ ID NO: 116 - protein, 15 aa, Homo sapiens
CVVNKATSGTYKYIF

SEQ ID NO: 117 - protein, 15 aa, Homo sapiens
CSARDREAAGYGYTF

SEQ ID NO: 118 - protein, 17 aa, Homo sapiens
CSARLAHSGTNTGELFF

SEQ ID NO: 119 - DNA, 42 bp, Homo sapiens
tgtgctctgattcccacgggaggaggaaacaaactcaccttt

SEQ ID NO: 120 - DNA, 51 bp, Homo sapiens
tgcagtgctagattggcccatagcgggaccaacaccggggagctgtttttt

SEQ ID NO: 121 - DNA, 42 bp, Homo sapiens
tgtgctgtgggtgcgtacagcagtgcttccaagataatcttt

SEQ ID NO: 122 - DNA, 45 bp, Homo sapiens
tgtgccagcagggacaggggatctggggccaacgtcctgactttc

SEQ ID NO: 123 - DNA, 42 bp, Homo sapiens
tgcagtggggggtggggacctaacaccggggagctgtttttt

SEQ ID NO: 124 - DNA, 57 bp, Homo sapiens
tgtgctgtggggcacttctcatctcggtcctctggttctgcaaggcaactgaccttt

SEQ ID NO: 125 - DNA, 54 bp, Homo sapiens
tgtgccagcagtttaaaggtcggtgtagacagctcctacaatgagcagttcttc

SEQ ID NO: 126 - DNA, 39 bp, Homo sapiens
tgtgcagagagcccttcagatggccagaagctgctcttt

SEQ ID NO: 127 - DNA, 45 bp, Homo sapiens
tgtgccagcagtttgagacagggtataagtcctgagcagttcttc

SEQ ID NO: 128 - DNA, 39 bp, Homo sapiens
tgtgccgtggaagaaaccagtggctctaggttgaccttt

SEQ ID NO: 129 - DNA, 45 bp, Homo sapiens; identical to SEQ ID NO: 127

SEQ ID NO: 130 - DNA, 42 bp, Homo sapiens
tgtgccagcagtgagcgacaggggataactgaagctttcttt

SEQ ID NO: 131 - DNA, 42 bp, Homo sapiens
tgtgctgtgagtggtactggagccaatagtaagctgacattt

SEQ ID NO: 132 - DNA, 48 bp, Homo sapiens
tgtgccagcagcgaccccattagcgggagaggggatgagcagttcttc

SEQ ID NO: 133 - DNA, 45 bp, Homo sapiens
tgtgccagcagctttggggggggggcgtggaatgagcagttcttc

SEQ ID NO: 134 - DNA, 42 bp, Homo sapiens
tgtgctctgaggcacaaagctgcaggcaacaagctaactttt

SEQ ID NO: 135 - DNA, 45 bp, Homo sapiens
tgtgcagcgctatttgatggaggaagccaaggaaatctcatcttt

SEQ ID NO: 136 - DNA, 33 bp, Homo sapiens
tgcagtgctagcggccctgaaaaactgtttttt

SEQ ID NO: 137 - DNA, 39 bp, Homo sapiens
tgtgccagcaggggactagcgggagagacccagtacttc

SEQ ID NO: 138 - DNA, 39 bp, Homo sapiens
tgtgccgtgatcccgaattccgggtatgcactcaacttc

SEQ ID NO: 139 - DNA, 42 bp, Homo sapiens
tgtgctctgaggggccggaaccagggaggaaagcttatcttc

SEQ ID NO: 140 - DNA, 45 bp, Homo sapiens
tgcagtgctagactttctagcggggggggctatgagcagttcttc

SEQ ID NO: 141 - DNA, 45 bp, Homo sapiens
tgcctcgtggtccctgtttataaccagggaggaaagcttatcttc

SEQ ID NO: 142 - DNA, 48 bp, Homo sapiens
tgcgccagcagctcggggactagcgggatgggagagacccagtacttc

SEQ ID NO: 143 - DNA, 42 bp, Homo sapiens
tgcatcctgagagacgtgggcggatctgaaaagctggtcttt

SEQ ID NO: 144 - DNA, 39 bp, Homo sapiens
tgcagtgccaaaggcctcgaaaatcagccccagcatttt

SEQ ID NO: 145 - DNA, 36 bp, Homo sapiens
tgcgctgtccttaaaaaggggtatgcactcaacttc

SEQ ID NO: 146 - DNA, 36 bp, Homo sapiens
tgtgctgtggctggtggctacaataagctgattttt

SEQ ID NO: 147 - DNA, 45 bp, Homo sapiens
tgtgccagcagcttagaagcgggggactcctacgagcagtacttc

SEQ ID NO: 148 - DNA, 36 bp, Homo sapiens
tgtgctgtgactggtggctacaataagctgattttt

SEQ ID NO: 149 - DNA, 45 bp, Homo sapiens
tgtgccagcagcttagaagcgggagattcctacgagcagtacttc

SEQ ID NO: 150 - DNA, 42 bp, Homo sapiens
tgtgctgggcaccaaattcagggagcccagaagctggtattt

SEQ ID NO: 151 - DNA, 42 bp, Homo sapiens
tgtgccagcagttactcggggatgaacactgaagctttcttt

SEQ ID NO: 152 - DNA, 36 bp, Homo sapiens
tgtgctggggcgaataacaatgccagactcatgttt

SEQ ID NO: 153 - DNA, 45 bp, Homo sapiens
tgcagcgtcactccggggggcggggtgaacactgaagctttcttt

SEQ ID NO: 154 - DNA, 45 bp, Homo sapiens
tgtgccgtgagcgctggtggtactagctatggaaagctgacattt

SEQ ID NO: 155 - DNA, 39 bp, Homo sapiens
tgtgccagcagccaagttcttaggggtgagcagtacttc

SEQ ID NO: 156 - DNA, 42 bp, Homo sapiens
tgtgctgtcagtactggtggtgctacaaacaagctcatcttt

SEQ ID NO: 157 - DNA, 45 bp, Homo sapiens
tgtgctgttcaggagggagaaaccagtggctctaggttgaccttt

SEQ ID NO: 158 - DNA, 42 bp, Homo sapiens
tgtgccagcagtccagggtttaatggaaacaccatatatttt

SEQ ID NO: 159 - DNA, 45 bp, Homo sapiens
tgtgctctgagtgaggcaacatataacaccgacaagctcatcttt

SEQ ID NO: 160 - DNA, 42 bp, Homo sapiens
tgtgccagcagctccaccgggacggactatggctacaccttc

SEQ ID NO: 161 - DNA, 39 bp, Homo sapiens
tgtgccgggaggcaaacctcctacgacaaggtgatattt

SEQ ID NO: 162 - DNA, 51 bp, Homo sapiens
tgtgccaccagtgatgtgactgggcagggcgagatgcgtggctacaccttc

SEQ ID NO: 163 - DNA, 42 bp, Homo sapiens
tgtgctctgagtgagatgaacagagatgacaagatcatcttt

SEQ ID NO: 164 - DNA, 45 bp, Homo sapiens
tgtgccagctcaccgacaggggtctctggaaacaccatatatttt

SEQ ID NO: 165 - DNA, 42 bp, Homo sapiens
tgtgctgggcagcaaaaaaccagtggctctaggttgaccttt

SEQ ID NO: 166 - DNA, 48 bp, Homo sapiens
tgtgccagtagttccccccgggacagggtcggtcagccccagcatttt

SEQ ID NO: 167 - DNA, 42 bp, Homo sapiens
tgtgctgcccgtcgggtcgacaataacaatgacatgcgcttt

SEQ ID NO: 168 - DNA, 33 bp, Homo sapiens
tgtgctagtggtttagctcagccccagcatttt

SEQ ID NO: 169 - DNA, 45 bp, Homo sapiens
tgcatcgtcagagtcgcggtaaccaatgcaggcaaatcaaccttt

SEQ ID NO: 170 - DNA, 39 bp, Homo sapiens
tgtgccagcagatattatagcgcggatacgcagtatttt

SEQ ID NO: 171 - DNA, 48 bp, Homo sapiens
tgtgctgtgagtggggtactcacgggaggaggaaacaaactcaccttt

SEQ ID NO: 172 - DNA, 45 bp, Homo sapiens
tgcgccagcagtgaggggacagttagcaatcagccccagcatttt

SEQ ID NO: 173 - DNA, 45 bp, Homo sapiens
tgtgcagagatcgggtctggggctgggagttaccaactcactttc

SEQ ID NO: 174 - DNA, 42 bp, Homo sapiens
tgtgcagagaatcagggaggcagcagctataaattgatcttc

SEQ ID NO: 175 - DNA, 45 bp, Homo sapiens
tgtgccagcagctctcagtcgggtgtggacactgaagctttcttt

SEQ ID NO: 176 - DNA, 36 bp, Homo sapiens
tgtgctgtgagtccccccgcgcagaaacttgtattt

SEQ ID NO: 177 - DNA, 42 bp, Homo sapiens
tgtgccagccaagagacaggggttggcggggagctgtttttt

SEQ ID NO: 178 - DNA, 42 bp, Homo sapiens
tgtgccagcagtacgacagccgtggtttcacccctccacttt

SEQ ID NO: 179 - DNA, 36 bp, Homo sapiens
tgtgcagagaagaaggaaggcttcaaaactatcttt

SEQ ID NO: 180 - DNA, 42 bp, Homo sapiens; identical to SEQ ID NO: 177

SEQ ID NO: 181 - DNA, 39 bp, Homo sapiens
tgtgctacggacgaggctgcaggcaacaagctaactttt

SEQ ID NO: 182 - DNA, 45 bp, Homo sapiens
tgtgccagcaaaagggaatcactagccaccggggagctgtttttt

SEQ ID NO: 183 - DNA, 45 bp, Homo sapiens
tgtgctgccatgtattcaggaggaggtgctgacggactcaccttt

SEQ ID NO: 184 - DNA, 42 bp, Homo sapiens
tgtgccagcagtacccagggacaggcctacgagcagtacttc

SEQ ID NO: 185 - DNA, 39 bp, Homo sapiens
tgtgcagcaagaggaggtagcaactataaactgacattt

SEQ ID NO: 186 - DNA, 33 bp, Homo sapiens
tgtgccagcagcacagggggcgagcagtacttc

SEQ ID NO: 187 - DNA, 36 bp, Homo sapiens
tgtgctacaaagggtgggaacaacagactcgctttt

SEQ ID NO: 188 - DNA, 42 bp, Homo sapiens
tgtgccagcagtgcctggacaggggagacgggctacaccttc

SEQ ID NO: 189 - DNA, 48 bp, Homo sapiens
tgtgcagcaacccgcgctggtggtactagctatggaaagctgacattt

SEQ ID NO: 190 - DNA, 39 bp, Homo sapiens
tgtgcctggagtgtactagcaggggtttcccagtacttc

SEQ ID NO: 191 - DNA, 36 bp, Homo sapiens
tgtgcagcaagtatagccaccgacaagctcatcttt

SEQ ID NO: 192 - DNA, 42 bp, Homo sapiens
tgcgccagcagctggaggggacagggggaaggctacaccttc

SEQ ID NO: 193 - DNA, 36 bp, Homo sapiens
tgtgtggtgaccgggggggcaaacaacctcttcttt

SEQ ID NO: 194 - DNA, 39 bp, Homo sapiens
tgcgccagcagtgaggctggggagtggacgcagtatttt

SEQ ID NO: 195 - DNA, 36 bp, Homo sapiens
tgcatcctggtagaaggaaatgagaaattaaccttt

SEQ ID NO: 196 - DNA, 42 bp, Homo sapiens
tgtggagcagacgtacaaggaagccaaggaaatctcatcttt

SEQ ID NO: 197 - DNA, 42 bp, Homo sapiens
tgtgccagcaggctggggggaaggaccactgaagctttcttt

SEQ ID NO: 198 - DNA, 45 bp, Homo sapiens
tgtgtggtggatttttatacctcaggaacctacaaatacatcttt

SEQ ID NO: 199 - DNA, 45 bp, Homo sapiens
tgcagtgctagagatcgggaggcggccggctatggctacaccttc

SEQ ID NO: 200 - DNA, 30 bp, Homo sapiens
tgtgcagggttagatgacaagatcatcttt

SEQ ID NO: 201 - DNA, 36 bp, Homo sapiens
tgtgctgtggcgggtagcaactatcagttaatctgg

SEQ ID NO: 202 - DNA, 48 bp, Homo sapiens; identical to SEQ ID NO: 132

SEQ ID NO: 203 - DNA, 45 bp, Homo sapiens
tgtgtggtgaacaaagccacctcaggaacctacaaatacatcttt

SEQ ID NO: 204 - DNA, 45 bp, Homo sapiens; identical to SEQ ID NO: 199

SEQ ID NO: 205 - DNA, 51 bp, Homo sapiens; identical to SEQ ID NO: 120

SEQ ID NO: 206 - protein, 146 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALIPTGGGNKLTFGTGTQLKVELNIQNPDPAVYQLRD

SEQ ID NO: 207 - protein, 150 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARLAHSGTNTGELFFGEGSRLTVLEDLKNVFPPEVAVFEPS

SEQ ID NO: 208 - protein, 147 aa, Homo sapiens
MLLELIPLLGIHFVLRTARAQSVTQPDIHITVSEGASLELRCNYSYGATPYLFWYVQSPGQGLQLLLKYFSGDTLVQGIKGFEAEFKRSQSSFNLRKPSVHWSDAAEYFCAVGAYSSASKIIFGSGTRLSIRPNIQNPDPAVYQLRD

SEQ ID NO: 209 - protein, 151 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASRDRGSGANVLTFGAGSRLTVLEDLKNVFPPEVAVFEPS

SEQ ID NO: 210 - protein, 147 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSGGWGPNTGELFFGEGSRLTVLEDLKNVFPPEVAVFEPS

SEQ ID NO: 211 - protein, 152 aa, Homo sapiens
MMKCPQALLAIFWLLLSWVSSEDKVVQSPLSLVVHEGDTVTLNCSYEVTNFRSLLWYKQEKKAPTFLFMLTSSGIEKKSGRLSSILDKKELSSILNITATQTGDSAIYLCAVGHFSSRSSGSARQLTFGSGTQLTVLPDIQNPDPAVYQLRD

SEQ ID NO: 212 - protein, 154 aa, Homo sapiens
MDSWTFCCVSLCILVAKHTDAGVIQSPRHEVTEMGQEVTLRCKPISGHNSLFWYRQTMMRGLELLIYFNNNVPIDDSGMPEDRFSAKMPNASFSTLKIQPSEPRDSAVYFCASSLKVGVDSSYNEQFFGPGTRLTVLEDLKNVFPPEVAVFEPS

SEQ ID NO: 213 - protein, 146 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAESPSDGQKLLFARGTMLKVDLNIQNPDPAVYQLRD

SEQ ID NO: 214 - protein, 150 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSLRQGISPEQFFGPGTRLTVLEDLKNVFPPEVAVFEPS

SEQ ID NO: 215 - protein, 143 aa, Homo sapiens
MKKLLAMILWLQLDRLSGELKVEQNPLFLSMQEGKNYTIYCNYSTTSDRLYWYRQDPGKSLESLFVLLSNGAVKQEGRLMASLDTKARLSTLHITAAVHDLSATYFCAVEETSGSRLTFGEGTQLTVNPDIQNPDPAVYQLRD

SEQ ID NO: 216 - protein, 150 aa, Homo sapiens; identical to SEQ ID NO: 214

SEQ ID NO: 217 - protein, 149 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSERQGITEAFFGQGTRLTVVEDLNKVFPPEVAVFEPS

SEQ ID NO: 218 - protein, 147 aa, Homo sapiens
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVSGTGANSKLTFGKGITLSVRPDIQNPDPAVYQLRD

SEQ ID NO: 219 - protein, 152 aa, Homo sapiens
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSDPISGRGDEQFFGPGTRLTVLEDLKNVFPPEVAVFEPS

SEQ ID NO: 220 - protein, 151 aa, Homo sapiens
MGTRLLFWVAFCLLGADHTGAGVSQSPSNKVTEKGKDVELRCDPISGHTALYWYRQSLGQGLEFLIYFQGNSAPDKSGLPSDRFSAERTGGSVSTLTIQRTQQEDSAVYLCASSFGGGAWNEQFFGPGTRLTVLEDLKNVFPPEVAVFEPS

SEQ ID NO: 221 - protein, 146 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALRHKAAGNKLTFGGGTRVLVKPNIQNPDPAVYQLRD

SEQ ID NO: 222 - protein, 156 aa, Homo sapiens
MDKILGASFLVLWLQLCWVSGQQKEKSDQQQVKQSPQSLIVQKGGISIINCAYENTAFDYFPWYQQFPGKGPALLIAIRPDVSEKKEGRFTISFNKSAKQFSLHIMDSQPGDSATYFCAALFDGGSQGNLIFGKGTKLSVKPNIQNPDPAVYQLRD

SEQ ID NO: 223 - protein, 144 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSASGPEKLFFGSGTQLSVLEDLNKVFPPEVAVFEPS

SEQ ID NO: 224 - protein, 146 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVIPNSGYALNFGKGTSLLVTPHIQNPDPAVYQLRD

SEQ ID NO: 225 - protein, 148 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRGLAGETQYFGPGTRLLVLEDLKNVFPPEVAVFEPS

SEQ ID NO: 226 - protein, 146 aa, Homo sapiens
MNYSPGLVSLILLLLGRTRGNSVTQMEGPVTLSEEAFLTINCTYTATGYPSLFWYVQYPGEGLQLLLKATKADDKGSNKGFEATYRKETTSFHLEKGSVQVSDSAVYFCALRGRNQGGKLIFGQGTELSVKPNIQNPDPAVYQLRD

SEQ ID NO: 227 - protein, 148 aa, Homo sapiens
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSARLSSGGGYEQFFGPGTRLTVLEDLKNVFPPEVAVFEPS

SEQ ID NO: 228 - protein, 143 aa, Homo sapiens
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVVPVYNQGGKLIFGQGTELSVKPNIQNPDPAVYQLRD

SEQ ID NO: 229 - protein, 151 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSSGTSGMGETQYFGPGTRLLVLEDLKNVFPPEVAVFEPS

SEQ ID NO: 230 - protein, 142 aa, Homo sapiens
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILRDVGGSEKLVFGKGTKLTVNPYIQNPDPAVYQLRD

SEQ ID NO: 231 - protein, 146 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSAKGLENQPQHFGDGTRLSILEDLNKVFPPEVAVFEPS

SEQ ID NO: 232 - protein, 140 aa, Homo sapiens
MWGAFLLYVSMKMGGTAGQSLEQPSEVTAVEGAIVQINCTYQTSGFYGLSWYQQHDGGAPTFLSYNALDGLEETGRFSSFLSRSDSYGYLLLQELQMKDSASYFCAVLKKGYALNFGKGTSLLVTPHIQNPDPAVYQLRD

SEQ ID NO: 233 - protein, 144 aa, Homo sapiens
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVAGGYNKLIFGAGTRLAVHPYIQNPDPAVYQLRD

SEQ ID NO: 234 - protein, 151 aa, Homo sapiens
MGTRLLCWVAFCLLVEELIEAGVVQSPRYKIIEKKQPVAFWCNPISGHNTLYWYLQNLGQGPELLIRYENEEAVDDSQLPKDRFSAERLKGVDSTLKIQPAELGDSAVYLCASSLEAGDSYEQYFGPGTRLTVTEDLKNVFPPEVAVFEPS

SEQ ID NO: 235 - protein, 144 aa, Homo sapiens
MALQSTLGAVWLGLLLNSLWKVAESKDQVFQPSTVASSEGAVVEIFCNHSVSNAYNFFWYLHFPGCAPRLLVKGSKPSQQGRYNMTYERFSSSLLILQVREADAAVYYCAVTGGYNKLIFGAGTRLAVHPYIQNPDPAVYQLRD

SEQ ID NO: 236 - protein, 151 aa, Homo sapiens; identical to SEQ ID NO: 234

SEQ ID NO: 237 - protein, 144 aa, Homo sapiens
MLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGHQIQGAQKLVFGQGTRLTINPNIQNPDPAVYQLRD

SEQ ID NO: 238 - protein, 149 aa, Homo sapiens
MSISLLCCAAFPLLWAGPVNAGVTQTPKFRILKIGQSMTLQCTQDMNHNYMYWYRQDPGMGLKLIYYSVGAGITDKGEVPNGYNVSRSTTEDFPLRLELAAPSQTSVYFCASSYSGMNTEAFFGQGTRLTVVEDLNKVFPPEVAVFEPS

SEQ ID NO: 239 - protein, 142 aa, Homo sapiens
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGANNNARLMFGDGTQLVVKPNIQNPDPAVYQLRD

SEQ ID NO: 240 - protein, 148 aa, Homo sapiens
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSVTPGGGVNTEAFFGQGTRLTVVEDLNKVFPPEVAVFEPS

SEQ ID NO: 241 - protein, 148 aa, Homo sapiens
MKSLRVLLVILWLQLSWVWSQQKEVEQNSGPLSVPEGAIASLNCTYSDRGSQSFFWYRQYSGKSPELIMSIYSNGDKEDGRFTAQLNKASQYVSLLIRDSQPSDSATYLCAVSAGGTSYGKLTFGQGTILTVHPNIQNPDPAVYQLRD

SEQ ID NO: 242 - protein, 148 aa, Homo sapiens
MGCRLLCCVVFCLLQAGPLDTAVSQTPKYLVTQMGNDKSIKCEQNLGHDTMYWYKQDSKKFLKIMFSYNNKELIINETVPNRFSPKSPDKAHLNLHINSLELGDSAVYFCASSQVLRGEQYFGPGTRLTVTEDLKNVFPPEVAVFEPS

SEQ ID NO: 243 - protein, 146 aa, Homo sapiens
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVSTGGATNKLIFGTGTLLAVQPNIQNPDPAVYQLRD

SEQ ID NO: 244 - protein, 147 aa, Homo sapiens
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVQEGETSGSRLTFGEGTQLTVNPDIQNPDPAVYQLRD

SEQ ID NO: 245 - protein, 149 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPGFNGNTIYFGEGSWLTVVEDLNKVFPPEVAVFEPS

SEQ ID NO: 246 - protein, 150 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEATYNTDKLIFGTGTRLQVFPNIQNPDPAVYQLRD

SEQ ID NO: 247 - protein, 149 aa, Homo sapiens
MGPGLLCWALLCLLGAGSVETGVTQSPTHLIKTRGQQVTLRCSSQSGHNTVSWYQQALGQGPQFIFQYYREEENGRGNFPPRFSGLQFPNYSSELNVNALELDDSALYLCASSSTGTDYGYTFGSGTRLTVVEDLNKVFPPEVAVFEPS

SEQ ID NO: 248 - protein, 146 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAGRQTSYDKVIFGPGTSLSVIPNIQNPDPAVYQLRD

SEQ ID NO: 249 - protein, 152 aa, Homo sapiens
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSDVTGQGEMRGYTFGSGTRLTVVEDLNKVFPPEVAVFEPS

SEQ ID NO: 250 - protein, 149 aa, Homo sapiens
MLTASLLRAVIASICVVSSMAQKVTQAQTEISVVEKEDVTLDCVYETRDTTYYLFWYKQPPSGELVFLIRRNSFDEQNEISGRYSWNFQKSTSSFNFTITASQVVDSAVYFCALSEMNRDDKIIFGKGTRLHILPNIQNPDPAVYQLRD

SEQ ID NO: 251 - protein, 151 aa, Homo sapiens
MDTRVLCCAVICLLGAGLSNAGVMQNPRHLVRRRGQEARLRCSPMKGHSHVYWYRQLPEEGLKFMVYLQKENIIDESGMPKERFSAEFPKEGPSILRIQQVVRGDSAAYFCASSPTGVSGNTIYFGEGSWLTVVEDLNKVFPPEVAVFEPS

SEQ ID NO: 252 - protein, 144 aa, Homo sapiens
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGQQKTSGSRLTFGEGTQLTVNPDIQNPDPAVYQLRD

SEQ ID NO: 253 - protein, 151 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSPRDRVGQPQHFGDGTRLSILEDLNKVFPPEVAVFEPS

SEQ ID NO: 254 - protein, 144 aa, Homo sapiens
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAARRVDNNNDMRFGAGTRLTVKPNIQKPDPAVYQLRD

SEQ ID NO: 255 - protein, 147 aa, Homo sapiens
MATRLLCCVVLCLLGEELIDARVTQTPRHKVTEMGQEVTMRCQPILGHNTVFWYRQTMMQGLELLAYFRNRAPLDDSGMPKDRFSAEMPDATLATLKIQPSEPRDSAVYFCASGLAQPQHFGDGTRLSILEDLNKVFPPEVAVFEPS

SEQ ID NO: 256 - protein, 143 aa, Homo sapiens
MRLVARVTVFLTFGTIIDAKTTQPPSMDCAEGRAANLPCNHSTISGNEYVYWYRQIHSQGPQYIIHGLKNNETNEMASLIITEDRKSSTLILPHATLRDTAVYYCIVRVAVTNAGKSTFGDGTTLTVKPNIQKPDPAVYQLRD

SEQ ID NO: 257 - protein, 148 aa, Homo sapiens
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASRYYSADTQYFGPGTRLTVLEDLKNVFPPEVAVFEPS

SEQ ID NO: 258 - protein, 149 aa, Homo sapiens
MLLLLVPAFQVIFTLGGTRAQSVTQLDSQVPVFEEAPVELRCNYSSSVSVYLFWYVQYPNQGLQLLLKYLSGSTLVKGINGFEAEFNKSQTSFHLRKPSVHISDTAEYFCAVSGVLTGGGNKLTFGTGTQLKVELNIQKPDPAVYQLRD

SEQ ID NO: 259 - protein, 150 aa, Homo sapiens
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSEGTVSNQPQHFGDGTRLSILEDLNKVFPPEVAVFEPS

SEQ ID NO: 260 - protein, 148 aa, Homo sapiens
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAEIGSGAGSYQLTFGKGTKLSVIPNIQNPDPAVYQLRD

SEQ ID NO: 261 - protein, 147 aa, Homo sapiens
MAGIRALFMYLWLQLDWVSRGESVGLHLPTLSVQEGDNSIINCAYSNSASDYFIWYKQESGKGPQFIIDIRSNMDKRQGQRVTVLLNKTVKHLSLQIAATQPGDSAVYFCAENQGGSSYKLIFGSGTRLLVRPDIQNPDPAVYQLRD

SEQ ID NO: 262 - protein, 151 aa, Homo sapiens
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSGVDTEAFFGQGTRLTVVEDLNKVFPPEVAVFEPS

SEQ ID NO: 263 - protein, 145 aa, Homo sapiens
MLLLLVPVLEVIFTLGGTRAQSVTQLGSHVSVSEGALVLLRCNYSSSVPPYLFWYVQYPNQGLQLLLKYTTGATLVKGINGFEAEFKKSETSFHLTKPSAHMSDAAEYFCAVSPPAQKLVFGTGTRLLVSPNIQNPDPAVYQLRD

SEQ ID NO: 264 - protein, 149 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASQETGVGGELFFGEGSRLTVLEDLKNVFPPEVAVFEPS

SEQ ID NO: 265 - protein, 149 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSTTAVVSPLHFGNGTRLTVTEDLNKVFPPEVAVFEPS

SEQ ID NO: 266 - protein, 145 aa, Homo sapiens
MKTFAGFSFLFLWLQLDCMSRGEDVEQSLFLSVREGDSSVINCTYTDSSSTYLYWYKQEPGAGLQLLTYIFSNMDMKQDQRLTVLLNKKDKHLSLRIADTQTGDSAIYFCAEKKEGFKTIFGAGTRLFVKANIQNPDPAVYQLRD

SEQ ID NO: 267 - protein, 149 aa, Homo sapiens; identical to SEQ ID NO: 264

SEQ ID NO: 268 - protein, 145 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDEAAGNKLTFGGGTRVLVKPNIQNPDPAVYQLRD

SEQ ID NO: 269 - protein, 151 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASKRESLATGELFFGEGSRLTVLEDLKNVFPPEVAVFEPS

SEQ ID NO: 270 - protein, 147 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCAAMYSGGGADGLTFGKGTHLIIQPYIQNPDPAVYQLRD

SEQ ID NO: 271 - protein, 150 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSTQGQAYEQYFGPGTRLTVTEDLKNVFPPEVAVFEPS

SEQ ID NO: 272 - protein, 145 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAARGGSNYKLTFGKGTLLTVNPNIQNPDPAVYQLRD

SEQ ID NO: 273 - protein, 147 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSTGGEQYFGPGTRLTVTEDLKNVFPPEVAVFEPS

SEQ ID NO: 274 - protein, 144 aa, Homo sapiens
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATKGGNNRLAFGKGNQVVVIPNIQNPDPAVYQLRD

SEQ ID NO: 275 - protein, 150 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASSAWTGETGYTFGSGTRLTVVEDLNKVFPPEVAVFEPS

SEQ ID NO: 276 - protein, 155 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAATRAGGTSYGKLTFGQGTILTVHPNIQNPDPAVYQLRD

SEQ ID NO: 277 - protein, 146 aa, Homo sapiens
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSVLAGVSQYFGPGTRLLVLEDLKNVFPPEVAVFEPS

SEQ ID NO: 278 - protein, 144 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAASIATDKLIFGTGTRLQVFPNIQNPDPAVYQLRD

SEQ ID NO: 279 - protein, 149 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSWRGQGEGYTFGSGTRLTVVEDLNKVFPPEVAVFEPS

SEQ ID NO: 280 - protein, 146 aa, Homo sapiens
MKKHLTTFLVILWLYFYRGNGKNQVEQSPQSLIILEGKNCTLQCNYTVSPFSNLRWYKQDTGRGPVSLTIMTFSENTKSNGRYTATLDADTKQSSLHITASQLSDSASYICVVTGGANNLFFGTGTRLTVIPYIQNPDPAVYQLRD

SEQ ID NO: 281 - protein, 148 aa, Homo sapiens
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSEAGEWTQYFGPGTRLTVLEDLKNVFPPEVAVFEPS

SEQ ID NO: 282 - protein, 140 aa, Homo sapiens
MKLVTSITVLLSLGIMGDAKTTQPNSMESNEEEPVHLPCNHSTISGTDYIHWYRQLPSQGPEYVIHGLTSNVNNRMASLAIAEDRKSSTLILHRATLRDAAVYYCILVEGNEKLTFGTGTRLTIIPNIQNPDPAVYQLRD

SEQ ID NO: 283 - protein, 146 aa, Homo sapiens
METVLQVLLGILGFQAAWVSSQELEQSPQSLIVQEGKNLTINCTSSKTLYGLYWYKQKYGEGLIFLMMLQKGGEEKSHEKITAKLDEKKQQSSLHITASQPSHAGIYLCGADVQGSQGNLIFGKGTKLSVKPNIQNPDPAVYQLRD

SEQ ID NO: 284 - protein, 150 aa, Homo sapiens
MDTWLVCWAIFSLLKAGLTEPEVTQTPSHQVTQMGQEVILRCVPISNHLYFYWYRQILGQKVEFLVSFYNNEISEKSEIFDDQFSVERPDGSNFTLKIRSTKLEDSAMYFCASRLGGRTTEAFFGQGTRLTVVEDLNKVFPPEVAVFEPS

SEQ ID NO: 285 - protein, 147 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVDFYTSGTYKYIFGTGTRLKVLANIQNPDPAVYQLRD

SEQ ID NO: 286 - protein, 148 aa, Homo sapiens
MLLLLLLLGPGSGLGAVVSQHPSRVICKSGTSVKIECRSLDFQATTMFWYRQFPKQSLMLMATSNEGSKATYEQGVEKDKFLINHASLTLSTLTVTSAHPEDSSFYICSARDREAAGYGYTFGSGTRLTVVEDLNKVFPPEVAVFEPS

SEQ ID NO: 287 - protein, 140 aa, Homo sapiens
MVLKFSVSILWIQLAWVSTQLLEQSPQFLSIQEGENLTVYCNSSSVFSSLQWYRQEPGEGPVLLVTVVTGGEVKKLKRLTFQFGDARKDSSLHITAAQPGDTGLYLCAGLDDKIIFGKGTRLHILPNIQNPDPAVYQLRD

SEQ ID NO: 288 - protein, 142 aa, Homo sapiens
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVAGSNYQLIWGAGTKLIIKPDIQNPDPAVYQLRD

SEQ ID NO: 289 - protein, 152 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSDPISGRGDEQFFGPGTRLTVLEDLKNVFPPEVAVFEPS

SEQ ID NO: 290 - protein, 147 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNKATSGTYKYIFGTGTRLKVLANIQNPDPAVYQLRD

SEQ ID NO: 291 - protein, 148 aa, Homo sapiens
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDREAAGYGYTFGSGTRLTVVEDLNKVFPPEVAVFEPS

SEQ ID NO: 292 - protein, 150 aa, Homo sapiens; identical to SEQ ID NO: 207

SEQ ID NO: 293 - DNA, 495 bp, Homo sapiens
gactgtgatttcttcatgttaaggatcaagaccattatttgggtaacacactaaagatgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgattcccacgggaggaggaaacaaactcacctttgggacaggcactcagctaaaagtggaactcaatatccagaaccctgaccctgccgtgtaccagctgagagact

SEQ ID NO: 294 - DNA, 530 bp, Homo sapiens
gtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagattggcccatagcgggaccaacaccggggagctgttttttggagaaggctctaggctgaccgtactggaggacctgaaaaacgtgttcccacccgaggtcgctgtgtttgagccatcaga

SEQ ID NO: 295 - DNA, 813 bp, Homo sapiens
ggttttgatacatctcagaaattaggagaaacacactaatgagctctctctgctaaactggttctgcacttggcctccagagggcgatgctgcacacactggagcttttgtttctgttgaagatcaatccactgctcagtttcttcttcctgcagctggttgagttctttccagacaaagacaagtgacaagaattagaggtttaaaaagcaaccagattcatctcagcagcttttgtagttttaaataagcaaggagtttctccagcgaaacttcctcacacctcttggtcttggtctcttcagacactttccttcctgttctctggagatcttgcagaaaagagcctgcagtgtttcccttgttcagccatgctcctggagcttatcccactgctggggatacattttgtcctgagaactgccagagcccagtcagtgacccagcctgacatccacatcactgtctctgaaggagcctcactggagttgagatgtaactattcctatggggcaacaccttatctcttctggtatgtccagtcccccggccaaggcctccagctgctcctgaagtacttttcaggagacactctggttcaaggcattaaaggctttgaggctgaatttaagaggagtcaatcttccttcaatctgaggaaaccctctgtgcattggagtgatgctgctgagtacttctgtgctgtgggtgcgtacagcagtgcttccaagataatctttggatcagggaccagactcagcatccggccaaatatccagaaccctgaccctgccgtgtaccagctgagagact

SEQ ID NO: 296 - DNA, 529 bp, Homo sapiens
aaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagggacaggggatctggggccaacgtcctgactttcggggccggcagcaggctgaccgtgctggaggacctgaaaaacgtgttcccacccgaggtcgctgtgtttgagccatcaga

SEQ ID NO: 297 - DNA, 521 bp, Homo sapiens
gtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtggggggtggggacctaacaccggggagctgttttttggagaaggctctaggctgaccgtactggaggacctgaaaaacgtgttcccacccgaggtcgctgtgtttgagccatcaga

SEQ ID NO: 298 - DNA, 652 bp, Homo sapiens
ggttggctgaattgccaaattgctatttccctttttctgagtgtgtgtctgtgtccgtgtgtgtgcatgtgtgtgtgcacgcacgcgcacacaggcagacgctaggggtagagtgtgatggttcaaaaagaaaaggaaacgcttaaagggagtgaatcacgttttgcccaggaaaacacacttgataactgaaggatgatgaagtgtccacaggctttactagctatcttttggcttctactgagctgggtgagcagtgaagacaaggtggtacaaagccctctatctctggttgtccacgagggagacaccgtaactctcaattgcagttatgaagtgactaactttcgaagcctactatggtacaagcaggaaaagaaagctcccacatttctatttatgctaacttcaagtggaattgaaaagaagtcaggaagactaagtagcatattagataagaaagaactttccagcatcctgaacatcacagccacccagaccggagactcggccatctacctctgtgctgtggggcacttctcatctcggtcctctggttctgcaaggcaactgacctttggatctgggacacaattgactgttttacctgatatccagaaccctgaccctgccgtgtaccagctgagagact

SEQ ID NO: 299 - DNA, 516 bp, Homo sapiens
atgctcacagagggcctggtctagaatattccacatctgctctcactctgccatggactcctggaccttctgctgtgtgtccctttgcatcctggtagcgaagcatacagatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttaaaggtcggtgtagacagctcctacaatgagcagttcttcgggccagggacacggctcaccgtgctagaggacctgaaaaacgtgttcccacccgaggtcgctgtgtttgagccatcaga

SEQ ID NO: 300 - DNA, 639 bp, Homo sapiens
ggccacaaggtggcagagcttctctgctcatgtagaatgtacgaaaggatccttttggttgacttctgtgagtaggctgacgacagaagcagatgcctctgtggacagtttaagaaaccacagtgctttggaggaaaggaagagatacttgataatatagctctcttggctggagattgcaggtcccagtggggagaacaatgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagagcccttcagatggccagaagctgctctttgcaaggggaaccatgttaaaggtggatcttaatatccagaaccctgaccctgccgtgtaccagctgagagact

SEQ ID NO: 301 - DNA, 495 bp, Homo sapiens
acctggagcccccagaactggcagacacctgcctgatgctgccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttgagacagggtataagtcctgagcagttcttcgggccagggacacggctcaccgtgctagaggacctgaaaaacgtgttcccacccgaggtcgctgtgtttgagccatcaga

SEQ ID NO: 302 - DNA, 538 bp, Homo sapiens
ggaaatcgtgtttctgtagagaaagaaaaactaccatatttggatagccctggccaactttcaaggctcctaaatctgagttttcagtgaactggacagaaaaaaaaaatgaagaagctactagcaatgattctgtggcttcaactagaccggttaagtggagagctgaaagtggaacaaaaccctctgttcctgagcatgcaggagggaaaaaactataccatctactgcaattattcaaccacttcagacagactgtattggtacaggcaggatcctgggaaaagtctggaatctctgtttgtgttgctatcaaatggagcagtgaagcaggagggacgattaatggcctcacttgataccaaagcccgtctcagcaccctccacatcacagctgccgtgcatgacctctctgccacctacttctgtgccgtggaagaaaccagtggctctaggttgacctttggggaaggaacacagctcacagtgaatcctgatatccagaaccctgaccctgccgtgtaccagctgagagact

SEQ ID NO: 303 - DNA, 495 bp, Homo sapiens; identical to SEQ ID NO: 301

SEQ ID NO: 304 - DNA, 492 bp, Homo sapiens
acctggagcccccagaactggcagacacctgcctgatgctgccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtgagcgacaggggataactgaagctttctttggacaaggcaccagactcacagttgtagaggacctgaacaaggtgttcccacccgaggtcgctgtgtttgagccatcaga

SEQ ID NO: 305 - DNA, 662 bp, Homo sapiens
gtagctcgttgatatctgtgtggatagggagctgtgacgagagcaagaggtcagaacacatccaggctccttaagggaaagcctctttctgtttctgaaacttttcaaagccagggacttgtccaatccaacctcctcacagttcctagctcctgaggctcagcgcccttggcttctgtccgcccagctcaaggtcctgcagcattgccactgctcagccatgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttaaaggcatcaacggttttgaggctgaatttaacaagagtcaaacttccttccacttgaggaaaccctcagtccatataagcgacacggctgagtacttctgtgctgtgagtggtactggagccaatagtaagctgacatttggaaaaggaataactctgagtgttagaccagatatccagaaccctgaccctgccgtgtaccagctgagagact

SEQ ID NO: 306 - DNA, 505 bp, Homo sapiens
acagtgatcctgatctggtaaagctcccatcctgccctgaccctgccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagcagcgaccccattagcgggagaggggatgagcagttcttcgggccagggacacggctcaccgtgctagaggacctgaaaaacgtgttcccacccgaggtcgctgtgtttgagccatcaga

SEQ ID NO: 307 - DNA, 502 bp, Homo sapiens
acagtgatcctgatctggtaaagctcccatcctgccctgaccctgccatgggcaccaggctcctcttctgggtggccttctgtctcctgggggcagatcacacaggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagcagctttggggggggggcgtggaatgagcagttcttcgggccagggacacggctcaccgtgctagaggacctgaaaaacgtgttcccacccgaggtcgctgtgtttgagccatcaga

SEQ ID NO: 308 - DNA, 495 bp, Homo sapiens
gactgtgatttcttcatgttaaggatcaagaccattatttgggtaacacactaaagatgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgaggcacaaagctgcaggcaacaagctaacttttggaggaggaaccagggtgctagttaaaccaaatatccagaaccctgaccctgccgtgtaccagctgagagact

SEQ ID NO: 309 - DNA, 514 bp, Homo sapiens
ctggaagaccacctgggctgtcattgagctctggtgccaggaggaatggacaagatcttaggagcatcatttttagttctgtggcttcaactatgctgggtgagtggccaacagaaggagaaaagtgaccagcagcaggtgaaacaaagtcctcaatctttgatagtccagaaaggagggatttcaattataaactgtgcttatgagaacactgcgtttgactactttccatggtaccaacaattccctgggaaaggccctgcattattgatagccatacgtccagatgtgagtgaaaagaaagaaggaagattcacaatctccttcaataaaagtgccaagcagttctcattgcatatcatggattcccagcctggagactcagccacctacttctgtgcagcgctatttgatggaggaagccaaggaaatctcatctttggaaaaggcactaaactctctgttaaaccaaatatccagaaccctgaccctgccgtgtaccagctgagagact

SEQ ID NO: 310 - DNA, 512 bp, Homo sapiens
gtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagcggccctgaaaaactgttttttggcagtggaacccagctctctgtcttggaggacctgaacaaggtgttcccacccgaggtcgctgtgtttgagccatcaga

SEQ ID NO: 311 - DNA, 544 bp, Homo sapiens
aaagcagattctttttatgatttttaaagtagaaatatccattctaggtgcattttttaagggtttaaaatttgaatcctcagtgaaccagggcagagaagaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgatcccgaattccgggtatgcactcaacttcggcaaaggcacctcgctgttggtcacaccccatatccagaaccctgaccctgccgtgtaccagctgagagact

SEQ ID NO: 312 - DNA, 512 bp, Homo sapiens
gagagtcctgctcccctttcatcaatgcacagatacagaagacccctccgtcatgcagcatctgccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaggggactagcgggagagacccagtacttcgggccaggcacgcggctcctggtgctcgaggacctgaaaaacgtgttcccacccgaggtcgctgtgtttgagccatcaga

SEQ ID NO: 313 - DNA, 495 bp, Homo sapiens
gactgtgatttcttcatgttaaggatcaagaccattatttgggtaacacactaaagatgaactattctccaggcttagtatctctgatactcttactgcttggaagaacccgtggaaattcagtgacccagatggaagggccagtgactctctcagaagaggccttcctgactataaactgcacgtacacagccacaggatacccttcccttttctggtatgtccaatatcctggagaaggtctacagctcctcctgaaagccacgaaggctgatgacaagggaagcaacaaaggttttgaagccacataccgtaaagaaaccacttctttccacttggagaaaggctcagttcaagtgtcagactcagcggtgtacttctgtgctctgaggggccggaaccagggaggaaagcttatcttcggacagggaacggagttatctgtgaaacccaatatccagaaccctgaccctgccgtgtaccagctgagagact

SEQ ID NO: 314 - DNA, 524 bp, Homo sapiens
gtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagactttctagcggggggggctatgagcagttcttcgggccagggacacggctcaccgtgctagaggacctgaaaaacgtgttcccacccgaggtcgctgtgtttgagccatcaga

SEQ ID NO: 315 - DNA, 497 bp, Homo sapiens
ctgggctcattgcagctcagacacagcaaaagagcctagaacctgggtcctagtttgcacctagaatatgaggcaagtggcgagagtgatcgtgttcctgaccctgagtactttgagccttgctaagaccacccagcccatctccatggactcatatgaaggacaagaagtgaacataacctgtagccacaacaacattgctacaaatgattatatcacgtggtaccaacagtttcccagccaaggaccacgatttattattcaaggatacaagacaaaagttacaaacgaagtggcctccctgtttatccctgccgacagaaagtccagcactctgagcctgccccgggtttccctgagcgacactgctgtgtactactgcctcgtggtccctgtttataaccagggaggaaagcttatcttcggacagggaacggagttatctgtgaaacccaatatccagaaccctgaccctgccgtgtaccagctgagagact

SEQ ID NO: 316 - DNA, 761 bp, Homo sapiens
agaggaccagtatccctcacagggtgacacctgaccagctctgtcccacctggccatgggctccaggtacctctgatgggaagacctttgtctcttgggaacaagtgaatccttggcacaggcccagtggattctgctgtgcagaacagagagcagtggacctcaggaggcctgcaaggggaggacataggacagtgacatcacagtatgcccctcccaccaggaaaagcaaggctgagaatttagctctttcccaggaggaccaagccctgagcacagacacagtgctgcctgcccctttgtgccatgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagctcggggactagcgggatgggagagacccagtacttcgggccaggcacgcggctcctggtgctcgaggacctgaaaaacgtgttcccacccgaggtcgctgtgtttgagccatcaga

SEQ ID NO: 317 - DNA, 731 bp, Homo sapiens
ggttcttgttacatttgcgtttgggttcttggtcatgaagtctttgcctaagccagtgtctagaagggtttttctgatggtatcttctagaatttttatggttttaggtgttttattttctttttttgtttatgtggaagctgttctatctttagctgacgatttctggggagtggggaaattgaaacctgcctgatgtgggatgtgctgtggctgctgctttgttgcttgggacctcctctgacctaggatcagacacagagtctgagttctggggcctggaacctcaatgtgcacttgaacaatgaagttggtgacaagcattactgtactcctatctttgggtattatgggtgatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctgagagacgtgggcggatctgaaaagctggtctttggaaagggaacgaaactgacagtaaacccatatatccagaaccctgaccctgccgtgtaccagctgagagact

SEQ ID NO: 318 - DNA, 518 bp, Homo sapiens
gtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgccaaaggcctcgaaaatcagccccagcattttggtgatgggactcgactctccatcctagaggacctgaacaaggtgttcccacccgaggtcgctgtgtttgagccatcaga

SEQ ID NO: 319 - DNA, 487 bp, Homo sapiens
cttctgcagactacagtggctcaggaaccggggatgcagtgccaggctcatggtatcctgcagcagatgtggggagctttccttctctatgtttccatgaagatgggaggcactgcaggacaaagccttgagcagccctctgaagtgacagctgtggaaggagccattgtccagataaactgcacgtaccagacatctgggttttatgggctgtcctggtaccagcaacatgatggcggagcacccacatttctttcttacaatgctctggatggtttggaggagacaggtcgtttttcttcattccttagtcgctctgatagttatggttacctccttctacaggagctccagatgaaagactctgcctcttacttctgcgctgtccttaaaaaggggtatgcactcaacttcggcaaaggcacctcgctgttggtcacaccccatatccagaaccctgaccctgccgtgtaccagctgagagact

SEQ ID NO: 320 - DNA, 528 bp, Homo sapiens
gagattctggctgatgatgtcactgagacaaaggaaaaaatgcaaaacaggtagtcttaaagaagcattctggtgagacggggggcattttggccatggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgtggctggtggctacaataagctgatttttggagcagggaccaggctggctgtacacccatatatccagaaccctgaccctgccgtgtaccagctgagagact

SEQ ID NO: 321 - DNA, 500 bp, Homo sapiens
agtgaccctgatctggcaaagcttccatcctgccctgaccctgccatgggtaccaggctcctctgctgggtggccttctgtctcctggtggaagaactcatagaagctggagtggttcagtctcccagatataagattatagagaaaaaacagcctgtggctttttggtgcaatcctatttctggccacaataccctttactggtacctgcagaacttgggacagggcccggagcttctgattcgatatgagaatgaggaagcagtagacgattcacagttgcctaaggatcgattttctgcagagaggctcaaaggagtagactccactctcaagatccagcctgcagagcttggggactcggccgtgtatctctgtgccagcagcttagaagcgggggactcctacgagcagtacttcgggccgggcaccaggctcacggtcacagaggacctgaaaaacgtgttcccacccgaggtcgctgtgtttgagccatcaga

SEQ ID NO: 322 - DNA, 510 bp, Homo sapiens
gtcactgacacaaaggaaaaaatgcaaaacaggtagtattggagaagcattctggtgtgttggggggcattttggccatggctttgcagagcactctgggggcggtgtggctagggcttctcctcaactctctctggaaggttgcagaaagcaaggaccaagtgtttcagccttccacagtggcatcttcagagggagctgtggtggaaatcttctgtaatcactctgtgtccaatgcttacaacttcttctggtaccttcacttcccgggatgtgcaccaagactccttgttaaaggctcaaagccttctcagcagggacgatacaacatgacctatgaacggttctcttcatcgctgctcatcctccaggtgcgggaggcagatgctgctgtttactactgtgctgtgactggtggctacaataagctgatttttggagcagggaccaggctggctgtacacccatatatccagaaccctgaccctgccgtgtaccagctgagagact

SEQ ID NO: 323 - DNA, 500 bp, Homo sapiens
agtgaccctgatctggcaaagcttccatcctgccctgaccctgccatgggtaccaggctcctctgctgggtggccttctgtctcctggtggaagaactcatagaagctggagtggttcagtctcccagatataagattatagagaaaaaacagcctgtggctttttggtgcaatcctatttctggccacaataccctttactggtacctgcagaacttgggacagggcccggagcttctgattcgatatgagaatgaggaagcagtagacgattcacagttgcctaaggatcgattttctgcagagaggctcaaaggagtagactccactctcaagatccagcctgcagagcttggggactcggccgtgtatctctgtgccagcagcttagaagcgggagattcctacgagcagtacttcgggccgggcaccaggctcacggtcacagaggacctgaaaaacgtgttcccacccgaggtcgctgtgtttgagccatcaga

SEQ ID NO: 324 - DNA, 551 bp, Homo sapiens
ctggaacagagatttaaatttcgtggtagtgtttttagggggactctaagcccaagagagtttttttaaaaaaaaaaaaaaaaaaacccattcaggaaataattctttgctgataaggatgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaacctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctgggcaccaaattcagggagcccagaagctggtatttggccaaggaaccaggctgactatcaacccaaatatccagaaccctgaccctgccgtgtaccagctgagagact

SEQ ID NO: 325 - DNA, 495 bp, Homo sapiens
atcaatgcacagatacagaagacccctccgtcctggagcacctgccatgagcatcagcctcctgtgctgtgcagcctttcctctcctgtgggcaggtccagtgaatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagttactcggggatgaacactgaagctttctttggacaaggcaccagactcacagttgtagaggacctgaacaaggtgttcccacccgaggtcgctgtgtttgagccatcaga

SEQ ID NO: 326 - DNA, 953 bp, Homo sapiens
aagtctctcagctggtacacggcagggtcagggttctggatattgggcttcaccaccagctgagttccatctccaaacatgagtctggcattgttattcgccccagcacagaagtagatgcctacatcactaggtatggatgctgagatattcaggaagctgtcctttctggttataccaaactgagcagtcagtcttccatttgaggtcaattcaccagccttatataaggctatcaagaggacaggaccttccccagggtcctgcttgtaccatagccaggtgttaaatatgcttgaagaagtgcagttcatggagacatcttctccttcctggataaacatagattgaggactctgattcagctgttgaccactgacccatgtcagctgcatccacaagattattaatatgaattcggattgggttggtttttttgtttttagtgtcactctaagcccaatagagtttttttaaaaaaaaaaaaaaaaaaacccattcaggaaataattctttgctgataaggatgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggggcgaataacaatgccagactcatgtttggagatggaactcagctggtggtgaagcccaatatccagaaccctgaccctgccgtgtaccagctgagagact

SEQ ID NO: 327 - DNA, 513 bp, Homo sapiens
gaaggaaagatgaacttgagtttcacttcttagtgccttttctcaggggagaggccatcacttgaagatgctgagtcttctgctccttctcctgggactaggctctgtgttcagtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgtcactccggggggcggggtgaacactgaagctttctttggacaaggcaccagactcacagttgtagaggacctgaacaaggtgttcccacccgaggtcgctgtgtttgagccatcaga

SEQ ID NO: 328 - DNA, 550 bp, Homo sapiens
aaagcagattctttttatgatttttaaagtagaaatatccattccaggtgcattttttaagggtttaaaatttgaatcctcagtgaaccagggcagagaagaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgtgagcgctggtggtactagctatggaaagctgacatttggacaagggaccatcttgactgtccatccaaatatccagaaccctgaccctgccgtgtaccagctgagagact

SEQ ID NO: 329 - DNA, 493 bp, Homo sapiens
agaccagaatcctgccctgggccttgcctggtctgcctcactctgccatgggctgcaggctcctctgctgtgtggtcttctgcctcctccaagcaggtcccttggacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaagttcttaggggtgagcagtacttcgggccgggcaccaggctcacggtcacagaggacctgaaaaacgtgttcccacccgaggtcgctgtgtttgagccatcaga

SEQ ID NO: 330 - DNA, 792 bp, Homo sapiens
ggagagacagggtcttgctctgttgcccaggctagagtggagtaacatgatcatatctcactgtatcctcaaactcctgggctcaagtatttgacctgcataataaatgtctacgcctcatgccactaggtggcaatgtgggtgttatactgaaaagatcacagatggttcacttgggaagtaaaactgtaaatgttcttaagtgtgcatttctgctgcttctgatgggctgaaaatcccctttgatttctaaagtaaatgtagagacgttttaaaaataaaggactcctttgtccaagatatattccgaaatcctccaacagagacctgtgtgagcttctgctgcagtaataatggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgtcagtactggtggtgctacaaacaagctcatctttggaactggcactctgcttgctgtccagccaaatatccagaaccctgaccctgccgtgtaccagctgagagact

SEQ ID NO: 331 - DNA, 775 bp, Homo sapiens
ggttgcccaggctagagtggagtaacatgatcatatctcactgtatcctcaaactcctgggctcaagtatttgacctgcataataaatgtctacgcctcatgccactaggtggcaatgtgggtgttatactgaaaagatcacagatggttcacttgggaagtaaaactgtaaatgttcttaagtgtgcatttctgctgcttctgatgggctgaaaatcccctttgatttctaaagtaaatgtagagacgttttaaaaataaaggactcctttgtccaagatatattccgaaatcctccaacagagacctgtgtgagcttctgctgcagtaataatggtgaagatccggcaatttttgttggctattttgtggcttcagctaagctgtgtaagtgccgccaaaaatgaagtggagcagagtcctcagaacctgactgcccaggaaggagaatttatcacaatcaactgcagttactcggtaggaataagtgccttacactggctgcaacagcatccaggaggaggcattgtttccttgtttatgctgagctcagggaagaagaagcatggaagattaattgccacaataaacatacaggaaaagcacagctccctgcacatcacagcctcccatcccagagactctgccgtctacatctgtgctgttcaggagggagaaaccagtggctctaggttgacctttggggaaggaacacagctcacagtgaatcctgatatccagaaccctgaccctgccgtgtaccagctgagagact

SEQ ID NO: 332 - DNA, 515 bp, Homo sapiens
gagagtcctgctcccctttcatcaatgcacagatacagaagacccctccgtcatgcagcccctgccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagtccagggtttaatggaaacaccatatattttggagagggaagttggctcactgttgtagaggacctgaacaaggtgttcccacccgaggtcgctgtgtttgagccatcaga

SEQ ID NO: 333 - DNA, 568 bp, Homo sapiens
gagaagcctcacacagcccagtaactttgctagtacctcttgagtgcaaggtggagaattaagatctggatttgagacggagcacggaacatttcactcaggggaagagctatgaacatgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgaggcaacatataacaccgacaagctcatctttgggactgggaccagattacaagtctttccaaatatccagaaccctgaccctgccgtgtaccagctgagagact

SEQ ID NO: 334 - DNA, 650 bp, Homo sapiens
ttcagctctgcaggacaggtagagactccaggatcatccactgagcactggacataaggaaggctgcatggggaggacacaggacagtgacatcacaggatacccctcctattaggaaaatcaaggcccagaattcactcggctcttccccaggaggaccaagccctgaatcaggtgcagtgctgcctgccccactgtgccatgggccctgggctcctctgctgggcgctgctttgtctcctgggagcaggctcagtggagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgccagcagctccaccgggacggactatggctacaccttcggttcggggaccaggttaaccgttgtagaggacctgaacaaggtgttcccacccgaggtcgctgtgtttgagccatcaga

SEQ ID NO: 335 - DNA, 544 bp, Homo sapiens
aaagcagattctttttatgatttttaaagtagaaatatccattctaggtgcattttttaagggtttaaaatttgaatcctcagtgaaccagggcagagaagaatgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgggaggcaaacctcctacgacaaggtgatatttgggccagggacaagcttatcagtcattccaaatatccagaaccctgaccctgccgtgtaccagctgagagact

SEQ ID NO: 336 - DNA, 494 bp, Homo sapiens
agagctggaaacacctccatcctgcctcttcatgccatggcctccctgctcttcttctgtggggccttttatctcctgggaacagggtccatggatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatgtgactgggcagggcgagatgcgtggctacaccttcggttcggggaccaggttaaccgttgtagaggacctgaacaaggtgttcccacccgaggtcgctgtgtttgagccatcaga

SEQ ID NO: 337 - DNA, 565 bp, Homo sapiens
gagaagcctcacacagcccagtaactttgctagtacctcttgagtgcaaggtggagaattaagatctggatttgagacggagcacggaacatttcactcaggggaagagctatgaacatgctgactgccagcctgttgagggcagtcatagcctccatctgtgttgtatccagcatggctcagaaggtaactcaagcgcagactgaaatttctgtggtggagaaggaggatgtgaccttggactgtgtgtatgaaacccgtgatactacttattacttattctggtacaagcaaccaccaagtggagaattggttttccttattcgtcggaactcttttgatgagcaaaatgaaataagtggtcggtattcttggaacttccagaaatccaccagttccttcaacttcaccatcacagcctcacaagtcgtggactcagcagtatacttctgtgctctgagtgagatgaacagagatgacaagatcatctttggaaaagggacacgacttcatattctccccaatatccagaaccctgaccctgccgtgtaccagctgagagact

SEQ ID NO: 338 - DNA, 536 bp, Homo sapiens
agctgtgaggtctggttccccgacgtgctgcagcaagtgcctttgccctgcctgtgggctccctccatggccaactctgctatggacaccagagtactctgctgtgcggtcatctgtcttctgggggcaggtctctcaaatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcaccgacaggggtctctggaaacaccatatattttggagagggaagttggctcactgttgtagaggacctgaacaaggtgttcccacccgaggtcgctgtgtttgagccatcaga

SEQ ID NO: 339 - DNA, 552 bp, Homo sapiens
cgggaagagcagagatttaaatttcttggttgtttttagtgggactctaagcccaagagagtttcttgaaaaaaaaaaaaaaaaaaacccattcaggaaataattctttgctgataaggatgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctgggcagcaaaaaaccagtggctctaggttgacctttggggaaggaacacagctcacagtgaatcctgatatccagaaccctgaccctgccgtgtaccagctgagagact

SEQ ID NO: 340 - DNA, 663 bp, Homo sapiens
ggaggtgcgaatgactctgctctctgtcctgtctcctcatctgcaaaattaggaagcctgtcttgattatctccaggaacctcccacctcttcattccagcctctgacaaactctgcacattaggccaggagaagcccccgagccaagtctcttttctcattctcttccaacaagtgcttggagctccaagaaggccccctttgcactatgagcaaccaggtgctctgctgtgtggtcctttgtttcctgggagcaaacaccgtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagttccccccgggacagggtcggtcagccccagcattttggtgatgggactcgactctccatcctagaggacctgaacaaggtgttcccacccgaggtcgctgtgtttgagccatcaga

SEQ ID NO: 341 - DNA, 566 bp, Homo sapiens
ggctttgtcgggtggagctgattggttgcaggagcagcaacagttccagagccaagtcatgacaccgacctccccaaggtttagttaaatatatcttatggtgaaaatgcccggagcaagaaggcaaagcatcatgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgcccgtcgggtcgacaataacaatgacatgcgctttggagcagggaccagactgacagtaaaaccaaatatccagaagcctgaccctgccgtgtaccagctgagagact

SEQ ID NO: 342 - DNA, 507 bp, Homo sapiens
agctcctgtattcgtgcccacaagggcctcatctaggtgaaggctccacctgccccaccctgccatggccaccaggctcctctgctgtgtggttctttgtctcctgggagaagagcttatagatgctagagtcacccagacaccaaggcacaaggtgacagagatgggacaagaagtaacaatgagatgtcagccaattttaggccacaatactgttttctggtacagacagaccatgatgcaaggactggagttgctggcttacttccgcaaccgggctcctctagatgattcggggatgccgaaggatcgattctcagcagagatgcctgatgcaactttagccactctgaagatccagccctcagaacccagggactcagctgtgtatttttgtgctagtggtttagctcagccccagcattttggtgatgggactcgactctccatcctagaggacctgaacaaggtgttcccacccgaggtcgctgtgtttgagccatcaga

SEQ ID NO: 343 - DNA, 639 bp, Homo sapiens
aggaacatgactaaacctggggaggaagacatagaaaagcaactgctctatcattggctgacaagatcctgacaacaacacaatgagagctgcactccttgcaagtctttactagtagctgttgcttctgttctctggaaattctttaaacctagaatcagacacaaaaactgaactctgggtccacaatcctcatttgtccttgaagtatgaggctggtggcaagagtaactgtgtttctgacctttggaactataattgatgctaagaccacccagcccccctccatggattgcgctgaaggaagagctgcaaacctgccttgtaatcactctaccatcagtggaaatgagtatgtgtattggtatcgacagattcactcccaggggccacagtatatcattcatggtctaaaaaacaatgaaaccaatgaaatggcctctctgatcatcacagaagacagaaagtccagcaccttgatcctgccccacgctacgctgagagacactgctgtgtactattgcatcgtcagagtcgcggtaaccaatgcaggcaaatcaacctttggggatgggactacgctcactgtgaagccaaatatccagaagcctgaccctgccgtgtaccagctgagagact

SEQ ID NO: 344 - DNA, 489 bp, Homo sapiens
acctggagcccccagaactggcagacacctgcctgatgctgccatgggcccccagctccttggctatgtggtcctttgccttctaggagcaggccccctggaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagatattatagcgcggatacgcagtattttggcccaggcacccggctgacagtgctcgaggacctgaaaaacgtgttcccacccgaggtcgctgtgtttgagccatcaga

SEQ ID NO: 345 - DNA, 668 bp, Homo sapiens
gtagctcgttgatatctgtgtggatagggagctgtgacgagagcaagaggtcagaacacatccaggctccttaagggaaagcctctttctgtttctgaaacttttcaaagccagggacttgtccaatccaacctcctcacagttcctagctcctgaggctcagcgcccttggcttctgtccgcccagctcaaggtcctgcagcattgccactgctcagccatgctcctgctgctcgtcccagcgttccaggtgatttttaccctgggaggaaccagagcccagtctgtgacccagcttgacagccaagtccctgtctttgaagaagcccctgtggagctgaggtgcaactactcatcgtctgtttcagtgtatctcttctggtatgtgcaataccccaaccaaggactccagcttctcctgaagtatttatcaggatccaccctggttaaaggcatcaacggttttgaggctgaatttaacaagagtcaaacttccttccacttgaggaaaccctcagtccatataagcgacacggctgagtacttctgtgctgtgagtggggtactcacgggaggaggaaacaaactcacctttgggacaggcactcagctaaaagtggaactcaatatccagaagcctgaccctgccgtgtaccagctgagagact

SEQ ID NO: 346 - DNA, 503 bp, Homo sapiens
aatttgcccacagcagggctgggagacacaagatcctgccctggagctgaaatgggcaccaggctcttcttctatgtggccctttgtctgctgtgggcaggacacagggatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtatttctgcgccagcagtgaggggacagttagcaatcagccccagcattttggtgatgggactcgactctccatcctagaggacctgaacaaggtgttcccacccgaggtcgctgtgtttgagccatcaga

SEQ ID NO: 347 - DNA, 515 bp, Homo sapiens
cgatgatggaagtagctcttatggctggagattgcaggtttatgactgatcctatttgggaagaacaatgatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagatcgggtctggggctgggagttaccaactcactttcgggaaggggaccaaactctcggtcataccaaatatccagaaccctgaccctgccgtgtaccagctgagagact

SEQ ID NO: 348 - DNA, 512 bp, Homo sapiens
cgatgatggaagtagctcttatggctggagattgcaggtttatgactgatcctatttgggaagaacaatgatggcaggcattcgagctttatttatgtacttgtggctgcagctggactgggtgagcagaggagagagtgtggggctgcatcttcctaccctgagtgtccaggagggtgacaactctattatcaactgtgcttattcaaacagcgcctcagactacttcatttggtacaagcaagaatctggaaaaggtcctcaattcattatagacattcgttcaaatatggacaaaaggcaaggccaaagagtcaccgttttattgaataagacagtgaaacatctctctctgcaaattgcagctactcaacctggagactcagctgtctacttttgtgcagagaatcagggaggcagcagctataaattgatcttcgggagtgggaccagactgctggtcaggcctgatatccagaaccctgaccctgccgtgtaccagctgagagact

SEQ ID NO: 349 - DNA, 750 bp, Homo sapiens
gtggaaacccacttctgacttatcacttgtcatgaattctatgcttcatggtgttacaccgtttattgtttctgatgagtgacagtaattattttctttcttgctggtacataataaagtggtgcacatcagagttgctgccatcttagacttaactcatcagtatcaggtgatcctgaggctcagtgatgtgactgtgggaactgctctgtggcgacaaggacgtccctcatcctctgctcctggtgacagtgaccctgatctggtaaagctcccatcctgccctgaccctgccatgggcaccagcctcctctgctggatggccctgtgtctcctgggggcagatcacgcagatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagctctcagtcgggtgtggacactgaagctttctttggacaaggcaccagactcacagttgtagaggacctgaacaaggtgttcccacccgaggtcgctgtgtttgagccatcaga

SEQ ID NO: 350 - DNA, 564 bp, Homo sapiens
ttttgaaacccttcaaaggcagagacttgtccagcctaacctgcctgctgctcctagctcctgaggctcagggcccttggcttctgtccgctctgctcagggccctccagcgtggccactgctcagccatgctcctgctgctcgtcccagtgctcgaggtgatttttaccctgggaggaaccagagcccagtcggtgacccagcttggcagccacgtctctgtctctgagggagccctggttctgctgaggtgcaactactcatcgtctgttccaccatatctcttctggtatgtgcaataccccaaccaaggactccagcttctcctgaagtacacaacaggggccaccctggttaaaggcatcaacggttttgaggctgaatttaagaagagtgaaacctccttccacctgacgaaaccctcagcccatatgagcgacgcggctgagtacttctgtgctgtgagtccccccgcgcagaaacttgtatttggaactggcacccgacttctggtcagtccaaatatccagaaccctgaccctgccgtgtaccagctgagagact

SEQ ID NO: 351 - DNA, 515 bp, Homo sapiens
gagagtcctgctcccctttcatcaatgcacagatacagaagacccctccgtcatgcagcatctgccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagccaagagacaggggttggcggggagctgttttttggagaaggctctaggctgaccgtactggaggacctgaaaaacgtgttcccacccgaggtcgctgtgtttgagccatcaga

SEQ ID NO: 352 - DNA, 515 bp, Homo sapiens
gagagtcctgctcccctttcatcaatgcacagatacagaagacccctccgtcatgcagcatctgccatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagtacgacagccgtggtttcacccctccactttgggaatgggaccaggctcactgtgacagaggacctgaacaaggtgttcccacccgaggtcgctgtgtttgagccatcaga

SEQ ID NO: 353 - DNA, 683 bp, Homo sapiens
agaatctcccacagatacaggataattggtttagcgtttggggatctggccacaaggtggcagagcttctctgctcatgtagaatgtacgaaaggatccttttggttgacttctgtgagtaggctgacggcagaagcagatgcctctgtggacagtttaagaaaccacagtgctttggaggaaaggaagagatacttgataatatagctctcttggctggagattgcaggtcccagtggggagaacaatgaagacatttgctggattttcgttcctgtttttgtggctgcagctggactgtatgagtagaggagaggatgtggagcagagtcttttcctgagtgtccgagagggagacagctccgttataaactgcacttacacagacagctcctccacctacttatactggtataagcaagaacctggagcaggtctccagttgctgacgtatattttttcaaatatggacatgaaacaagaccaaagactcactgttctattgaataaaaaggataaacatctgtctctgcgcattgcagacacccagactggggactcagctatctacttctgtgcagagaagaaggaaggcttcaaaactatctttggagcaggaacaagactatttgttaaagcaaatatccagaaccctgaccctgccgtgtaccagctgagagact

SEQ ID NO: 354 - DNA, 515 bp, Homo sapiens; identical to SEQ ID NO: 351

SEQ ID NO: 355 - DNA, 583 bp, Homo sapiens
aggactgagcttgcctgtgactggctagggaggaacctgagactaggggacagaaagactagggattcacccagtaaagagagctcatctgtgactgaggagccttgctccatttcaggtcttctgtgatttcaataaggaagaagaatggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgaggctgcaggcaacaagctaacttttggaggaggaaccagggtgctagttaaaccaaatatccagaaccctgaccctgccgtgtaccagctgagagact

SEQ ID NO: 356 - DNA, 529 bp, Homo sapiens
aaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcaaaagggaatcactagccaccggggagctgttttttggagaaggctctaggctgaccgtactggaggacctgaaaaacgtgttcccacccgaggtcgctgtgtttgagccatcaga

SEQ ID NO: 357 - DNA, 589 bp, Homo sapiens
aggactgagcttgcctgtgactggctagggaggaacctgagactaggggacagaaagactagggattcacccagtaaagagagctcatctgtgactgaggagccttgctccatttcaggtcttctgtgatttcaataaggaagaagaatggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctgccatgtattcaggaggaggtgctgacggactcacctttggcaaagggactcatctaatcatccagccctatatccagaaccctgaccctgccgtgtaccagctgagagact

SEQ ID NO: 358 - DNA, 526 bp, Homo sapiens
aaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtacccagggacaggcctacgagcagtacttcgggccgggcaccaggctcacggtcacagaggacctgaaaaacgtgttcccacccgaggtcgctgtgtttgagccatcaga

SEQ ID NO: 359 - DNA, 459 bp, Homo sapiens
gatcttaattgggaagaacaaggatgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagaggaggtagcaactataaactgacatttggaaaaggaactctcttaaccgtgaatccaaatatccagaaccctgaccctgccgtgtaccagctgagagact

SEQ ID NO: 360 - DNA, 502 bp, Homo sapiens
tatggggatgttcacagagggcctggtctggaatattccacatctgctctcactctgccatgggctcctggaccctctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagcacagggggcgagcagtacttcgggccgggcaccaggctcacggtcacagaggacctgaaaaacgtgttcccacccgaggtcgctgtgtttgagccatcaga

SEQ ID NO: 361 - DNA, 580 bp, Homo sapiens
aggactgagcttgcctgtgactggctagggaggaacctgagactaggggacagaaagactagggattcacccagtaaagagagctcatctgtgactgaggagccttgctccatttcaggtcttctgtgatttcaataaggaagaagaatggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacaaagggtgggaacaacagactcgcttttgggaaggggaaccaagtggtggtcataccaaatatccagaaccctgaccctgccgtgtaccagctgagagact

SEQ ID NO: 362 - DNA, 526 bp, Homo sapiens
aaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgcctggacaggggagacgggctacaccttcggttcggggaccaggttaaccgttgtagaggacctgaacaaggtgttcccacccgaggtcgctgtgtttgagccatcaga

SEQ ID NO: 363 - DNA, 577 bp, Homo sapiens
atatgtaaaatgaagggtctgtggaaggacatgaataaagcacaggaggttgaagtcagatttgcagctttctaggcaggagacaagacaatctgcatcttcacaggagggatggccatgctcctgggggcatcagtgctgattctgtggcttcagccagactgggtaaacagtcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaacccgcgctggtggtactagctatggaaagctgacatttggacaagggaccatcttgactgtccatccaaatatccagaaccctgaccctgccgtgtaccagctgagagact

SEQ ID NO: 364 - DNA, 552 bp, Homo sapiens
ttcctctgctctggcagcagatctcccagagggagcagcctgaccacatcactggcccagaagaggaggcgtctgtcccccagactagctgaaggaaaggctggcttggatgatgctctgctctctccttgcccttctcctgggcactttctttggggtcagatctcagactattcatcaatggccagcgaccctggtgcagcctgtgggcagcccgctctctctggagtgcactgtggagggaacatcaaaccccaacctatactggtaccgacaggctgcaggcaggggcctccagctgctcttctactccgttggtattggccagatcagctctgaggtgccccagaatctctcagcctccagaccccaggaccggcagttcatcctgagttctaagaagctccttctcagtgactctggcttctatctctgtgcctggagtgtactagcaggggtttcccagtacttcgggccaggcacgcggctcctggtgctcgaggacctgaaaaacgtgttcccacccgaggtcgctgtgtttgagccatcaga

SEQ ID NO: 365 - DNA, 456 bp, Homo sapiens
gatcttaattgggaagaacaaggatgacatccattcgagctgtatttatattcctgtggctgcagctggacttggtgaatggagagaatgtggagcagcatccttcaaccctgagtgtccaggagggagacagcgctgttatcaagtgtacttattcagacagtgcctcaaactacttcccttggtataagcaagaacttggaaaaagacctcagcttattatagacattcgttcaaatgtgggcgaaaagaaagaccaacgaattgctgttacattgaacaagacagccaaacatttctccctgcacatcacagagacccaacctgaagactcggctgtctacttctgtgcagcaagtatagccaccgacaagctcatctttgggactgggaccagattacaagtctttccaaatatccagaaccctgaccctgccgtgtaccagctgagagact

SEQ ID NO: 366 - DNA, 755 bp, Homo sapiens
agaggaccagtatccctcacagggtgacacctgaccagctctgtcccacctggccatgggctccaggtacctctgatgggaagacctttgtctcttgggaacaagtgaatccttggcacaggcccagtggattctgctgtgcagaacagagagcagtggacctcaggaggcctgcaaggggaggacataggacagtgacatcacagtatgcccctcccaccaggaaaagcaaggctgagaatttagctctttcccaggaggaccaagccctgagcacagacacagtgctgcctgcccctttgtgccatgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagctggaggggacagggggaaggctacaccttcggttcggggaccaggttaaccgttgtagaggacctgaacaaggtgttcccacccgaggtcgctgtgtttgagccatcaga

SEQ ID NO: 367 - DNA, 475 bp, Homo sapiens
agtcaacttctgggagcagatctctgcagaataaaaatgaaaaagcatctgacgaccttcttggtgattttgtggctttatttttatagggggaatggcaaaaaccaagtggagcagagtcctcagtccctgatcatcctggagggaaagaactgcactcttcaatgcaattatacagtgagccccttcagcaacttaaggtggtataagcaagatactgggagaggtcctgtttccctgacaatcatgactttcagtgagaacacaaagtcgaacggaagatatacagcaactctggatgcagacacaaagcaaagctctctgcacatcacagcctcccagctcagcgattcagcctcctacatctgtgtggtgaccgggggggcaaacaacctcttctttgggactggaacgagactcaccgttattccctatatccagaaccctgaccctgccgtgtaccagctgagagact

SEQ ID NO: 368 - DNA, 755 bp, Homo sapiens
ctcagaggaccagtatccctcacagggtgacacctgaccagctctgtcccacctggccatgggctccaggtacctctgatgggaagacctttgtctcttgggaacaagtgaatccttggcacaggcccagtggattctgctgtgcagaacagagagcagtggacctcaggaggcctgcaaggggaggacataggacagtgacatcacagtatgcccctcccaccaggaaaagcaaggctgagaatttagctctttcccaggaggaccaagccctgagcacagacacagtgctgcctgcccctttgtgccatgggctccaggctgctctgttgggtgctgctttgtctcctgggagcaggcccagtaaaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagtgaggctggggagtggacgcagtattttggcccaggcacccggctgacagtgctcgaggacctgaaaaacgtgttcccacccgaggtcgctgtgtttgagccatcaga

SEQ ID NO: 369 - DNA, 554 bp, Homo sapiens
aggggggaaattgaaacctgcctgatgtgggatgtgctgtggctgctgctttgttgcttgggacctcctctgacctaggatcagacacagagtctgagttctggggcctggaacctcaatgtgcacttgaacaatgaagttggtgacaagcattactgtactcctatctttgggtattatgggtgatgctaagaccacacagccaaattcaatggagagtaacgaagaagagcctgttcacttgccttgtaaccactccacaatcagtggaactgattacatacattggtatcgacagcttccctcccagggtccagagtacgtgattcatggtcttacaagcaatgtgaacaacagaatggcctctctggcaatcgctgaagacagaaagtccagtaccttgatcctgcaccgtgctaccttgagagatgctgctgtgtactactgcatcctggtagaaggaaatgagaaattaacctttgggactggaacaagactcaccatcatacccaatatccagaaccctgaccctgccgtgtaccagctgagagact

SEQ ID NO: 370 - DNA, 557 bp, Homo sapiens
gaatccaggtatggcttctgattggtgcaatctcctgcaccaatgagcaaagtaacttctgctggggaagctcattcagtaaaatctgattgaactgtgttttctaaatagctaagggatggagactgttctgcaagtactcctagggatattggggttccaagcagcctgggtcagtagccaagaactggagcagagtcctcagtccttgatcgtccaagagggaaagaatctcaccataaactgcacgtcatcaaagacgttatatggcttatactggtataagcaaaagtatggtgaaggtcttatcttcttgatgatgctacagaaaggtggggaagagaaaagtcatgaaaagataactgccaagttggatgagaaaaagcagcaaagttccctgcatatcacagcctcccagcccagccatgcaggcatctacctctgtggagcagacgtacaaggaagccaaggaaatctcatctttggaaaaggcactaaactctctgttaaaccaaatatccagaaccctgaccctgccgtgtaccagctgagagact

SEQ ID NO: 371 - DNA, 526 bp, Homo sapiens
aaaatgcccctcctttcctccacaggaccagatgcctgagctaggaaaggcctcattcctgctgtgatcctgccatggatacctggctcgtatgctgggcaatttttagtctcttgaaagcaggactcacagaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcaggctggggggaaggaccactgaagctttctttggacaaggcaccagactcacagttgtagaggacctgaacaaggtgttcccacccgaggtcgctgtgtttgagccatcaga

SEQ ID NO: 372 - DNA, 625 bp, Homo sapiens
gagggcgataagaggagggggtagtgatataccaggggttgtgaaaataacctcttttttctaattggtaggacagattctttttatgattcctaaagtggaagaaataaagtatctctgctatgttcatttctttttggattgaaaattttaatcctcagtgaaccagggcagaaaagaatgatgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtggatttttatacctcaggaacctacaaatacatctttggaacaggcaccaggctgaaggttttagcaaatatccagaaccctgaccctgccgtgtaccagctgagagact

SEQ ID NO: 373 - DNA, 524 bp, Homo sapiens
gtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagatcgggaggcggccggctatggctacaccttcggttcggggaccaggttaaccgttgtagaggacctgaacaaggtgttcccacccgaggtcgctgtgtttgagccatcaga

SEQ ID NO: 374 - DNA, 518 bp, Homo sapiens
aagcactcttctagcccagagaagtctgttccaggacgggctctttcaggagcagctaaagtcaggggccatgtccaccatgtgatagaaagacaagatggtcctgaaattctccgtgtccattctttggattcagttggcatgggtgagcacccagctgctggagcagagccctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcagcccagcctggtgatacaggcctctacctctgtgcagggttagatgacaagatcatctttggaaaagggacacgacttcatattctccccaatatccagaaccctgaccctgccgtgtaccagctgagagact

SEQ ID NO: 375 - DNA, 560 bp, Homo sapiens
ggctttgtcgggtggagctgattggttgcaggagcagcaacagttccagagccaagtcatgacaccgacctccccaaggtttagttaaatatatcttatggtgaaaatgcccggagcaagaaggcaaagcatcatgaagaggatattgggagctctgctggggctcttgagtgcccaggtttgctgtgtgagaggaatacaagtggagcagagtcctccagacctgattctccaggagggagccaattccacgctgcggtgcaatttttctgactctgtgaacaatttgcagtggtttcatcaaaacccttggggacagctcatcaacctgttttacattccctcagggacaaaacagaatggaagattaagcgccacgactgtcgctacggaacgctacagcttattgtacatttcctcttcccagaccacagactcaggcgtttatttctgtgctgtggcgggtagcaactatcagttaatctggggcgctgggaccaagctaattataaagccagatatccagaaccctgaccctgccgtgtaccagctgagagact

SEQ ID NO: 376 - DNA, 685 bp, Homo sapiens
actaactgatatctctgtttcgagttgccttcaactcgaaacatccagcagaggatgggcctagagatggagtaggagatccagtccccaagccctagagatgcatttgtggaggcaatgatgtcactgtgggaactgccatgagaggacagggacgtccctccttgggggctgttgctcacagtgaccctgattgggcaaagctcccatccttccctgaccctgccatgggcaccaggctcctctgctgggcggccctctgtctcctgggagcagaactcacagaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccaacctgcaaagcttgaggactcggccgtgtatctctgtgccagcagcgaccccattagcgggagaggggatgagcagttcttcgggccagggacacggctcaccgtgctagaggacctgaaaaacgtgttcccacccgaggtcgctgtgtttgagccatcaga

SEQ ID NO: 377 - DNA, 701 bp, Homo sapiens
ggctcgtgtgtgtgtgtgtctgtgtgtgtgtgtgtgtgcttgagagagagagaaggagagaaagagagagaatgggaagggcgataagaggaggggttagtgatataccaggggttgtgaaaataacctcttttttctaattgggaggacagattctttttacgattcctaaagtggaagaaataaagtatctctgctatgttcatttctttttggattgaaaattttaatcctcagtgaaccagggcagaaaagaatgatgatatccttgagagttttactggtgatcctgtggcttcagttaagctgggtttggagccaacggaaggaggtggagcaggatcctggacccttcaatgttccagagggagccactgtcgctttcaactgtacttacagcaacagtgcttctcagtctttcttctggtacagacaggattgcaggaaagaacctaagttgctgatgtccgtatactccagtggtaatgaagatggaaggtttacagcacagctcaatagagccagccagtatatttccctgctcatcagagactccaagctcagtgattcagccacctacctctgtgtggtgaacaaagccacctcaggaacctacaaatacatctttggaacaggcaccaggctgaaggttttagcaaatatccagaaccctgaccctgccgtgtaccagctgagagact

SEQ ID NO: 378 - DNA, 524 bp, Homo sapiens
gtcatccctcctcgctggtgaatggaggcagtggtcacaactctccccagagaaggtggtgtgaggccatcacggaagatgctgctgcttctgctgcttctggggccaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagatcgggaggcggccggctatggctacaccttcggttcggggaccaggttaaccgttgtagaggacctgaacaaggtgttcccacccgaggtcgctgtgtttgagccatcaga

SEQ ID NO: 379 - DNA, 530 bp, Homo sapiens; identical to SEQ ID NO: 294

SEQ ID NO: 380 - protein, 20 aa, Homo sapiens
AASCEPLASVLRAKLTSRSS

SEQ ID NO: 381 - protein, 20 aa, Homo sapiens
GAGPDPLRLHGHLPVRTSCP

SEQ ID NO: 382 - protein, 20 aa, Homo sapiens
TQRSVLLCKVVGARGVGKSA

SEQ ID NO: 383 - protein, 20 aa, Homo sapiens
KVVGARGVGKSAFLQAFLGR

SEQ ID NO: 384 - protein, 20 aa, Homo sapiens
GQKSPRFRRVSCFLRLGRST

SEQ ID NO: 385 - protein, 20 aa, Homo sapiens
RFRRVSCFLRLGRSTLLELE

SEQ ID NO: 386 - protein, 20 aa, Homo sapiens
ALAFLLLISIAANLSLLLSR

SEQ ID NO: 387 - protein, 20 aa, Homo sapiens
EVQDCLKQLMMSLLQLYRFS

SEQ ID NO: 388 - protein, 20 aa, Homo sapiens
GYSPSLHILAIGTRSGAIKL

SEQ ID NO: 389 - protein, 18 aa, Homo sapiens
VATFPVYTMVAIPIVCKD

SEQ ID NO: 390 - protein, 20 aa, Homo sapiens
PPLYRQRYQFIKNLVDQHEP

SEQ ID NO: 391 - protein, 20 aa, Homo sapiens
PLYRQRYQFIKNLVDQHEPK

SEQ ID NO: 392 - protein, 20 aa, Homo sapiens
VSRPELLRESISAFLVPMPT

SEQ ID NO: 393 - protein, 20 aa, Homo sapiens
TDRALQNKSISAFLVPMPTP

SEQ ID NO: 394 - protein, 18 aa, Homo sapiens
NPLSPYLNVDPRYLVQDT

SEQ ID NO: 395 - protein, 20 aa, Homo sapiens
EPPVDICLSKAISSSLKGFL

SEQ ID NO: 396 - protein, 19 aa, Homo sapiens
GETGMFSLSTIRGHQYATY

SEQ ID NO: 397 - protein, 20 aa, Homo sapiens
MNYVSKRLPFAARLNTPMGP

SEQ ID NO: 398 - protein, 20 aa, Homo sapiens
LGSLGLIFALTLNRHKYPLN

SEQ ID NO: 399 - protein, 20 aa, Homo sapiens
LGLIFALTLNRHKYPLNLYL

SEQ ID NO: 400 - protein, 20 aa, Homo sapiens
APISLSSFFNVSTLEREVTD

SEQ ID NO: 401 - protein, 20 aa, Homo sapiens
LELGAGTGLASIIAATMART

SEQ ID NO: 402 - protein, 20 aa, Homo sapiens
AGTGLASIIAATMARTVYCT

SEQ ID NO: 403 - protein, 20 aa, Homo sapiens
VPREYVRALNATKLERVFAK

SEQ ID NO: 404 - protein, 20 aa, Homo sapiens
LHRDKALLKRLLKGMQKKRP

SEQ ID NO: 405 - protein, 20 aa, Homo sapiens
KALLKRLLKGMQKKRPSDVQ

SEQ ID NO: 406 - protein, 20 aa, Homo sapiens
ITVQTVYVQHLITFLDRPIQ

SEQ ID NO: 407 - protein, 20 aa, Homo sapiens
QTVYVQHLITFLDRPIQMCC

SEQ ID NO: 408 - protein, 20 aa, Homo sapiens
PGLISMFSSSQELGAALAQL

SEQ ID NO: 409 - protein, 20 aa, Homo sapiens
WRVRIALALKGIDYETVPIN

SEQ ID NO: 410 - protein, 20 aa, Homo sapiens
VRIALALKGIDYETVPINLI

SEQ ID NO: 411 - protein, 20 aa, Homo sapiens
DRAEKFNRGIRKLGITPEGQ

SEQ ID NO: 412 - protein, 20 aa, Homo sapiens
EKFNRGIRKLGITPEGQSYL

SEQ ID NO: 413 - protein, 20 aa, Homo sapiens
LTISLLDTSNLELPEAVVFQ

SEQ ID NO: 414 - protein, 20 aa, Homo sapiens
ISLLDTSNLELPEAVVFQDS

SEQ ID NO: 415 - protein, 20 aa, Homo sapiens
AASCEPLASVLRAKPTSRSS

SEQ ID NO: 416 - protein, 20 aa, Homo sapiens
GAGPDPLRLRGHLPVRTSCP

SEQ ID NO: 417 - protein, 20 aa, Homo sapiens
TQRSVLLCKVVGACGVGKSA

SEQ ID NO: 418 - protein, 20 aa, Homo sapiens
KVVGACGVGKSAFLQAFLGR

SEQ ID NO: 419 - protein, 20 aa, Homo sapiens
GQKSPRFRRVTCFLRLGRST

SEQ ID NO: 420 - protein, 20 aa, Homo sapiens
RFRRVTCFLRLGRSTLLELE

SEQ ID NO: 421 - protein, 20 aa, Homo sapiens
ALAFLLLISTAANLSLLLSR

SEQ ID NO: 422 - protein, 20 aa, Homo sapiens
EVQDCLKQLMMSLLRLYRFS

SEQ ID NO: 423 - protein, 20 aa, Homo sapiens
GYSPSLRILAIGTRSGAIKL

SEQ ID NO: 424 - protein, 18 aa, Homo sapiens
VATFPVYTMGAIPIVCKD

SEQ ID NO: 425 - protein, 20 aa, Homo sapiens
PPLYRQRYQFVKNLVDQHEP

SEQ ID NO: 426 - protein, 20 aa, Homo sapiens
PLYRQRYQFVKNLVDQHEPK

SEQ ID NO: 427 - protein, 20 aa, Homo sapiens
VSRPELLREGISAFLVPMPT

SEQ ID NO: 428 - protein, 20 aa, Homo sapiens
TDRALQNKGISAFLVPMPTP

SEQ ID NO: 429 - protein, 18 aa, Homo sapiens
NPLCPYLNVDPRYLVQDT

SEQ ID NO: 430 - protein, 20 aa, Homo sapiens
EPPVDICLSKANSSSLKGFL

SEQ ID NO: 431 - protein, 19 aa, Homo sapiens
GETGMFSLCTIRGHQYATY

SEQ ID NO: 432 - protein, 20 aa, Homo sapiens
MNYVSKSLPFAARLNTPMGP

SEQ ID NO: 433 - protein, 20 aa, Homo sapiens
LGSLGLIFALILNRHKYPLN

SEQ ID NO: 434 - protein, 20 aa, Homo sapiens
LGLIFALILNRHKYPLNLYL

SEQ ID NO: 435 - protein, 20 aa, Homo sapiens
APISLSSFFSVSTLEREVTD

SEQ ID NO: 436 - protein, 20 aa, Homo sapiens
LELGAGTGLTSIIAATMART

SEQ ID NO: 437 - protein, 20 aa, Homo sapiens
AGTGLTSIIAATMARTVYCT

SEQ ID NO: 438 - protein, 20 aa, Homo sapiens
VPREYIRALNATKLERVFAK

SEQ ID NO: 439 - protein, 20 aa, Homo sapiens
LHRDKALLKRLLKGVQKKRP

SEQ ID NO: 440 - protein, 20 aa, Homo sapiens
KALLKRLLKGVQKKRPSDVQ

SEQ ID NO: 441 - protein, 20 aa, Homo sapiens
ITVQTVYVQHPITFLDRPIQ

SEQ ID NO: 442 - protein, 20 aa, Homo sapiens
QTVYVQHPITFLDRPIQMCC

SEQ ID NO: 443 - protein, 20 aa, Homo sapiens
PGLISVFSSSQELGAALAQL

SEQ ID NO: 444 - protein, 20 aa, Homo sapiens
WRVRIALALKGIDYKTVPIN

SEQ ID NO: 445 - protein, 20 aa, Homo sapiens
VRIALALKGIDYKTVPINLI

SEQ ID NO: 446 - protein, 20 aa, Homo sapiens
DRAEKFNRGIRKLGVTPEGQ

SEQ ID NO: 447 - protein, 20 aa, Homo sapiens
EKFNRGIRKLGVTPEGQSYL

SEQ ID NO: 448 - protein, 6 aa, Homo sapiens
ATGYPS

SEQ ID NO: 449 - protein, 6 aa, Homo sapiens
DFQATT

SEQ ID NO: 450 - protein, 6 aa, Homo sapiens
YGATPY

SEQ ID NO: 451 - protein, 5 aa, Homo sapiens
SNHLY

SEQ ID NO: 452 - protein, 6 aa, Homo sapiens
DFQATT

SEQ ID NO: 453 - protein, 6 aa, Homo sapiens
VTNFRS

SEQ ID NO: 454 - protein, 5 aa, Homo sapiens
SGHNS

SEQ ID NO: 455 - protein, 6 aa, Homo sapiens
DSSSTY

SEQ ID NO: 456 - protein, 5 aa, Homo sapiens
MNHEY

SEQ ID NO: 457 - protein, 5 aa, Homo sapiens
TTSDR

SEQ ID NO: 458 - protein, 5 aa, Homo sapiens
MNHEY

SEQ ID NO: 459 - protein, 5 aa, Homo sapiens
MNHEY

SEQ ID NO: 460 - protein, 6 aa, Homo sapiens
SSVSVY

SEQ ID NO: 461 - protein, 5 aa, Homo sapiens
SGHTA

SEQ ID NO: 462 - protein, 5 aa, Homo sapiens
SGHTA

SEQ ID NO: 463 - protein, 6 aa, Homo sapiens
ATGYPS

SEQ ID NO: 464 - protein, 6 aa, Homo sapiens
NTAFDY

SEQ ID NO: 465 - protein, 6 aa, Homo sapiens
DFQATT

SEQ ID NO: 466 - protein, 6 aa, Homo sapiens
DRGSQS

SEQ ID NO: 467 - protein, 5 aa, Homo sapiens
MNHEY

SEQ ID NO: 468 - protein, 6 aa, Homo sapiens
ATGYPS

SEQ ID NO: 469 - protein, 6 aa, Homo sapiens
DFQATT

SEQ ID NO: 470 - protein, 7 aa, Homo sapiens
NIATNDY

SEQ ID NO: 471 - protein, 5 aa, Homo sapiens
SGHRS

SEQ ID NO: 472 - protein, 7 aa, Homo sapiens
TISGTDY

SEQ ID NO: 473 - protein, 6 aa, Homo sapiens
DFQATT

SEQ ID NO: 474 - protein, 6 aa, Homo sapiens
TSGFYG

SEQ ID NO: 475 - protein, 6 aa, Homo sapiens
VSNAYN

SEQ ID NO: 476 - protein, 5 aa, Homo sapiens
SGHNT

SEQ ID NO: 477 - protein, 6 aa, Homo sapiens
VSNAYN

SEQ ID NO: 478 - protein, 5 aa, Homo sapiens
SGHNT

SEQ ID NO: 479 - protein, 5 aa, Homo sapiens
SIFNT

SEQ ID NO: 480 - protein, 5 aa, Homo sapiens
MNHNY

SEQ ID NO: 481 - protein, 5 aa, Homo sapiens
SIFNT

SEQ ID NO: 482 - protein, 5 aa, Homo sapiens
SQVTM

SEQ ID NO: 483 - protein, 6 aa, Homo sapiens
DRGSQS

SEQ ID NO: 484 - protein, 5 aa, Homo sapiens
LGHDT

SEQ ID NO: 485 - protein, 5 aa, Homo sapiens
VGISA

SEQ ID NO: 486 - protein, 5 aa, Homo sapiens
VGISA

SEQ ID NO: 487 - protein, 5 aa, Homo sapiens
MNHEY

SEQ ID NO: 488 - protein, 7 aa, Homo sapiens
TRDTTYY

SEQ ID NO: 489 - protein, 5 aa, Homo sapiens
SGHNT

SEQ ID NO: 490 - protein, 6 aa, Homo sapiens
DRGSQS

SEQ ID NO: 491 - protein, 5 aa, Homo sapiens
KGHDR

SEQ ID NO: 492 - protein, 7 aa, Homo sapiens
TRDTTYY

SEQ ID NO: 493 - protein, 5 aa, Homo sapiens
KGHSH

SEQ ID NO: 494 - protein, 5 aa, Homo sapiens
SIFNT

SEQ ID NO: 495 - protein, 5 aa, Homo sapiens
LNHDA

SEQ ID NO: 496 - protein, 5 aa, Homo sapiens
DSVNN

SEQ ID NO: 497 - protein, 5 aa, Homo sapiens
LGHNT

SEQ ID NO: 498 - protein, 7 aa, Homo sapiens
TISGNEY

SEQ ID NO: 499 - protein, 5 aa, Homo sapiens
MNHEY

SEQ ID NO: 500 - protein, 6 aa, Homo sapiens
SSVSVY

SEQ ID NO: 501 - protein, 5 aa, Homo sapiens
WSHSY

SEQ ID NO: 502 - protein, 6 aa, Homo sapiens
NSASDY

SEQ ID NO: 503 - protein, 6 aa, Homo sapiens
NSASDY

SEQ ID NO: 504 - protein, 5 aa, Homo sapiens
SEHNR

SEQ ID NO: 505 - protein, 6 aa, Homo sapiens
SSVPPY

SEQ ID NO: 506 - protein, 5 aa, Homo sapiens
MNHEY

SEQ ID NO: 507 - protein, 5 aa, Homo sapiens
MNHEY

SEQ ID NO: 508 - protein, 6 aa, Homo sapiens
DSSSTY

SEQ ID NO: 509 - protein, 5 aa, Homo sapiens
MNHEY

SEQ ID NO: 510 - protein, 5 aa, Homo sapiens
TSINN

SEQ ID NO: 511 - protein, 5 aa, Homo sapiens
SNHLY

SEQ ID NO: 512 - protein, 5 aa, Homo sapiens
TSINN

SEQ ID NO: 513 - protein, 5 aa, Homo sapiens
SNHLY

SEQ ID NO: 514 - protein, 6 aa, Homo sapiens
DSASNY

SEQ ID NO: 515 - protein, 5 aa, Homo sapiens
SGHDY

SEQ ID NO: 516 - protein, 5 aa, Homo sapiens
TSINN

SEQ ID NO: 517 - protein, 5 aa, Homo sapiens
SNHLY

SEQ ID NO: 518 - protein, 6 aa, Homo sapiens
NSMFDY

SEQ ID NO: 519 - protein, 6 aa, Homo sapiens
GTSNPN

SEQ ID NO: 520 - protein, 6 aa, Homo sapiens
DSASNY

SEQ ID NO: 521 - protein, 5 aa, Homo sapiens
SGHRS

SEQ ID NO: 522 - protein, 6 aa, Homo sapiens
VSPFSN

SEQ ID NO: 523 - protein, 5 aa, Homo sapiens
SGHRS

SEQ ID NO: 524 - protein, 7 aa, Homo sapiens
TISGTDY

SEQ ID NO: 525 - protein, 5 aa, Homo sapiens
KTLYG

SEQ ID NO: 526 - protein, 5 aa, Homo sapiens
SNHLY

SEQ ID NO: 527 - protein, 6 aa, Homo sapiens
NSASQS

SEQ ID NO: 528 - protein, 6 aa, Homo sapiens
DFQATT

SEQ ID NO: 529 - protein, 5 aa, Homo sapiens
SVFSS

SEQ ID NO: 530 - protein, 5 aa, Homo sapiens
DSVNN

SEQ ID NO: 531 - protein, 5 aa, Homo sapiens
SGHAT

SEQ ID NO: 532 - protein, 6 aa, Homo sapiens
NSASQS

SEQ ID NO: 533 - protein, 6 aa, Homo sapiens
DFQATT

SEQ ID NO: 534 - protein, 6 aa, Homo sapiens
DFQATT

SEQ ID NO: 535 - DNA, 18 bp, Homo sapiens
gccacaggatacccttcc

SEQ ID NO: 536 - DNA, 18 bp, Homo sapiens
gactttcaggccacaact

SEQ ID NO: 537 - DNA, 18 bp, Homo sapiens
tatggggcaacaccttat

SEQ ID NO: 538 - DNA, 15 bp, Homo sapiens
tctaatcacttatac

SEQ ID NO: 539 - DNA, 18 bp, Homo sapiens
gactttcaggccacaact

SEQ ID NO: 540 - DNA, 18 bp, Homo sapiens
gtgactaactttcgaagc

SEQ ID NO: 541 - DNA, 15 bp, Homo sapiens
tcaggccacaactcc

SEQ ID NO: 542 - DNA, 18 bp, Homo sapiens
gacagctcctccacctac

SEQ ID NO: 543 - DNA, 15 bp, Homo sapiens
atgaaccatgagtat

SEQ ID NO: 544 - DNA, 15 bp, Homo sapiens
accacttcagacaga

SEQ ID NO: 545 - DNA, 15 bp, Homo sapiens
atgaaccatgagtat

SEQ ID NO: 546 - DNA, 15 bp, Homo sapiens
atgaaccatgagtat

SEQ ID NO: 547 - DNA, 18 bp, Homo sapiens
tcgtctgtttcagtgtat

SEQ ID NO: 548 - DNA, 15 bp, Homo sapiens
tcaggtcatactgcc

SEQ ID NO: 549 - DNA, 15 bp, Homo sapiens
tcaggtcatactgcc

SEQ ID NO: 550 - DNA, 18 bp, Homo sapiens
gccacaggatacccttcc

SEQ ID NO: 551 - DNA, 18 bp, Homo sapiens
aacactgcgtttgactac

SEQ ID NO: 552 - DNA, 18 bp, Homo sapiens
gactttcaggccacaact

SEQ ID NO: 553 - DNA, 18 bp, Homo sapiens
gaccgaggttcccagtcc

SEQ ID NO: 554 - DNA, 15 bp, Homo sapiens
atgaaccatgaatac

SEQ ID NO: 555 - DNA, 18 bp, Homo sapiens
gccacaggatacccttcc

SEQ ID NO: 556 - DNA, 18 bp, Homo sapiens
gactttcaggccacaact

SEQ ID NO: 557 - DNA, 21 bp, Homo sapiens
aacattgctacaaatgattat

SEQ ID NO: 558 - DNA, 15 bp, Homo sapiens
tctgggcataggagt

SEQ ID NO: 559 - DNA, 21 bp, Homo sapiens
acaatcagtggaactgattac

SEQ ID NO: 560 - DNA, 18 bp, Homo sapiens
gactttcaggccacaact

SEQ ID NO: 561 - DNA, 18 bp, Homo sapiens
acatctgggttttatggg

SEQ ID NO: 562 - DNA, 18 bp, Homo sapiens
gtgtccaatgcttacaac

SEQ ID NO: 563 - DNA, 15 bp, Homo sapiens
tctggccacaatacc

SEQ ID NO: 564 - DNA, 18 bp, Homo sapiens
gtgtccaatgcttacaac

SEQ ID NO: 565 - DNA, 15 bp, Homo sapiens
tctggccacaatacc

SEQ ID NO: 566 - DNA, 15 bp, Homo sapiens
agcatatttaacacc

SEQ ID NO: 567 - DNA, 15 bp, Homo sapiens
atgaaccataactac

SEQ ID NO: 568 - DNA, 15 bp, Homo sapiens
agcatatttaacacc

SEQ ID NO: 569 - DNA, 15 bp, Homo sapiens
agccaagtcaccatg

SEQ ID NO: 570 - DNA, 18 bp, Homo sapiens
gaccgaggttcccagtcc

SEQ ID NO: 571 - DNA, 15 bp, Homo sapiens
ctgggccatgatact

SEQ ID NO: 572 - DNA, 15 bp, Homo sapiens
gtaggaataagtgcc

SEQ ID NO: 573 - DNA, 15 bp, Homo sapiens
gtaggaataagtgcc

SEQ ID NO: 574 - DNA, 15 bp, Homo sapiens
atgaaccatgaatac

SEQ ID NO: 575 - DNA, 21 bp, Homo sapiens
acccgtgatactacttattac

SEQ ID NO: 576 - DNA, 15 bp, Homo sapiens
tctgggcacaacact

SEQ ID NO: 577 - DNA, 18 bp, Homo sapiens
gaccgaggttcccagtcc

SEQ ID NO: 578 - DNA, 15 bp, Homo sapiens
aagggtcatgataga

SEQ ID NO: 579 - DNA, 21 bp, Homo sapiens
acccgtgatactacttattac

SEQ ID NO: 580 - DNA, 15 bp, Homo sapiens
aaaggacacagtcat

SEQ ID NO: 581 - DNA, 15 bp, Homo sapiens
agcatatttaacacc

SEQ ID NO: 582 - DNA, 15 bp, Homo sapiens
ttgaaccacgatgcc

SEQ ID NO: 583 - DNA, 15 bp, Homo sapiens
gactctgtgaacaat

SEQ ID NO: 584 - DNA, 15 bp, Homo sapiens
ttaggccacaatact

SEQ ID NO: 585 - DNA, 21 bp, Homo sapiens
accatcagtggaaatgagtat

SEQ ID NO: 586 - DNA, 15 bp, Homo sapiens
atgaaccatgagtat

SEQ ID NO: 587 - DNA, 18 bp, Homo sapiens
tcgtctgtttcagtgtat

SEQ ID NO: 588 - DNA, 15 bp, Homo sapiens
tggagccacagctat

SEQ ID NO: 589 - DNA, 18 bp, Homo sapiens
aacagcgcctcagactac

SEQ ID NO: 590 - DNA, 18 bp, Homo sapiens
aacagcgcctcagactac

SEQ ID NO: 591 - DNA, 15 bp, Homo sapiens
tctgaacacaaccgc

SEQ ID NO: 592 - DNA, 18 bp, Homo sapiens
tcgtctgttccaccatat

SEQ ID NO: 593 - DNA, 15 bp, Homo sapiens
atgaaccatgaatac

SEQ ID NO: 594 - DNA, 15 bp, Homo sapiens
atgaaccatgaatac

SEQ ID NO: 595 - DNA, 18 bp, Homo sapiens
gacagctcctccacctac

SEQ ID NO: 596 - DNA, 15 bp, Homo sapiens
atgaaccatgaatac

SEQ ID NO: 597 - DNA, 15 bp, Homo sapiens
actagtataaacaat

SEQ ID NO: 598 - DNA, 15 bp, Homo sapiens
tctaatcacttatac

SEQ ID NO: 599 - DNA, 15 bp, Homo sapiens
actagtataaacaat

SEQ ID NO: 600 - DNA, 15 bp, Homo sapiens
tctaatcacttatac

SEQ ID NO: 601 - DNA, 18 bp, Homo sapiens
gacagtgcctcaaactac

SEQ ID NO: 602 - DNA, 15 bp, Homo sapiens
tcaggacacgactac

SEQ ID NO: 603 - DNA, 15 bp, Homo sapiens
actagtataaacaat

SEQ ID NO: 604 - DNA, 15 bp, Homo sapiens
tctaatcacttatac

SEQ ID NO: 605 - DNA, 18 bp, Homo sapiens
aacagcatgtttgattat

SEQ ID NO: 606 - DNA, 18 bp, Homo sapiens
ggaacatcaaaccccaac

SEQ ID NO: 607 - DNA, 18 bp, Homo sapiens
gacagtgcctcaaactac

SEQ ID NO: 608 - DNA, 15 bp, Homo sapiens
tctgggcataggagt

SEQ ID NO: 609 - DNA, 18 bp, Homo sapiens
gtgagccccttcagcaac

SEQ ID NO: 610 - DNA, 15 bp, Homo sapiens
tctgggcataggagt

SEQ ID NO: 611 - DNA, 21 bp, Homo sapiens
acaatcagtggaactgattac

SEQ ID NO: 612 - DNA, 15 bp, Homo sapiens
aagacgttatatggc

SEQ ID NO: 613 - DNA, 15 bp, Homo sapiens
tctaatcacttatac

SEQ ID NO: 614 - DNA, 18 bp, Homo sapiens
aacagtgcttctcagtct

SEQ ID NO: 615 - DNA, 18 bp, Homo sapiens
gactttcaggccacaact

SEQ ID NO: 616 - DNA, 15 bp, Homo sapiens
agtgttttttccagc

SEQ ID NO: 617 - DNA, 15 bp, Homo sapiens
gactctgtgaacaat

SEQ ID NO: 618 - DNA, 15 bp, Homo sapiens
tctggccatgctacc

SEQ ID NO: 619 - DNA, 18 bp, Homo sapiens
aacagtgcttctcagtct

SEQ ID NO: 620 - DNA, 18 bp, Homo sapiens
gactttcaggccacaact

SEQ ID NO: 621 - DNA, 18 bp, Homo sapiens
gactttcaggccacaact

SEQ ID NO: 622 - protein, 7 aa, Homo sapiens
ATKADDK

SEQ ID NO: 623 - protein, 7 aa, Homo sapiens
SNEGSKA

SEQ ID NO: 624 - protein, 8 aa, Homo sapiens
YFSGDTLV

SEQ ID NO: 625 - protein, 6 aa, Homo sapiens
FYNNEI

SEQ ID NO: 626 - protein, 7 aa, Homo sapiens
SNEGSKA

SEQ ID NO: 627 - protein, 7 aa, Homo sapiens
LTSSGIE

SEQ ID NO: 628 - protein, 6 aa, Homo sapiens
FNNNVP

SEQ ID NO: 629 - protein, 7 aa, Homo sapiens
IFSNMDM

SEQ ID NO: 630 - protein, 6 aa, Homo sapiens
SMNVEV

SEQ ID NO: 631 - protein, 7 aa, Homo sapiens
LLSNGAV

SEQ ID NO: 632 - protein, 6 aa, Homo sapiens
SMNVEV

SEQ ID NO: 633 - protein, 6 aa, Homo sapiens
SMNVEV

SEQ ID NO: 634 - protein, 8 aa, Homo sapiens
YLSGSTLV

SEQ ID NO: 635 - protein, 6 aa, Homo sapiens
FQGNSA

SEQ ID NO: 636 - protein, 6 aa, Homo sapiens
FQGNSA

SEQ ID NO: 637 - protein, 7 aa, Homo sapiens
ATKADDK

SEQ ID NO: 638 - protein, 7 aa, Homo sapiens
IRPDVSE

SEQ ID NO: 639 - protein, 7 aa, Homo sapiens
SNEGSKA

SEQ ID NO: 640 - protein, 6 aa, Homo sapiens
IYSNGD

SEQ ID NO: 641 - protein, 6 aa, Homo sapiens
SVGAGI

SEQ ID NO: 642 - protein, 7 aa, Homo sapiens
ATKADDK

SEQ ID NO: 643 - protein, 7 aa, Homo sapiens
SNEGSKA

SEQ ID NO: 644 - protein, 5 aa, Homo sapiens
GYKTK

SEQ ID NO: 645 - protein, 6 aa, Homo sapiens
YFSETQ

SEQ ID NO: 646 - protein, 5 aa, Homo sapiens
GLTSN

SEQ ID NO: 647 - protein, 7 aa, Homo sapiens
SNEGSKA

SEQ ID NO: 648 - protein, 6 aa, Homo sapiens
NALDGL

SEQ ID NO: 649 - protein, 4 aa, Homo sapiens
GSKP

SEQ ID NO: 650 - protein, 6 aa, Homo sapiens
YENEEA

SEQ ID NO: 651 - protein, 4 aa, Homo sapiens
GSKP

SEQ ID NO: 652 - protein, 6 aa, Homo sapiens
YENEEA

SEQ ID NO: 653 - protein, 7 aa, Homo sapiens
LYKAGEL

SEQ ID NO: 654 - protein, 6 aa, Homo sapiens
SVGAGI

SEQ ID NO: 655 - protein, 7 aa, Homo sapiens
LYKAGEL

SEQ ID NO: 656 - protein, 7 aa, Homo sapiens
ANQGSEA

SEQ ID NO: 657 - protein, 6 aa, Homo sapiens
IYSNGD

SEQ ID NO: 658 - protein, 6 aa, Homo sapiens
YNNKEL

SEQ ID NO: 659 - protein, 5 aa, Homo sapiens
LSSGK

SEQ ID NO: 660 - protein, 5 aa, Homo sapiens
LSSGK

SEQ ID NO: 661 - protein, 6 aa, Homo sapiens
SVGAGI

SEQ ID NO: 662 - protein, 8 aa, Homo sapiens
RNSFDEQN

SEQ ID NO: 663 - protein, 6 aa, Homo sapiens
YYREEE

SEQ ID NO: 664 - protein, 6 aa, Homo sapiens
IYSNGD

SEQ ID NO: 665 - protein, 6 aa, Homo sapiens
SFDVKD

SEQ ID NO: 666 - protein, 8 aa, Homo sapiens
RNSFDEQN

SEQ ID NO: 667 - protein, 6 aa, Homo sapiens
LQKENI

SEQ ID NO: 668 - protein, 7 aa, Homo sapiens
LYKAGEL

SEQ ID NO: 669 - protein, 6 aa, Homo sapiens
SQIVND

SEQ ID NO: 670 - protein, 5 aa, Homo sapiens
IPSGT

SEQ ID NO: 671 - protein, 6 aa, Homo sapiens
FRNRAP

SEQ ID NO: 672 - protein, 5 aa, Homo sapiens
GLKNN

SEQ ID NO: 673 - protein, 6 aa, Homo sapiens
SMNVEV

SEQ ID NO: 674 - protein, 8 aa, Homo sapiens
YLSGSTLV

SEQ ID NO: 675 - protein, 6 aa, Homo sapiens
SAAADI

SEQ ID NO: 676 - protein, 7 aa, Homo sapiens
IRSNMDK

SEQ ID NO: 677 - protein, 7 aa, Homo sapiens
IRSNMDK

SEQ ID NO: 678 - protein, 6 aa, Homo sapiens
FQNEAQ

SEQ ID NO: 679 - protein, 8 aa, Homo sapiens
YTTGATLV

SEQ ID NO: 680 - protein, 6 aa, Homo sapiens
SVGAGI

SEQ ID NO: 681 - protein, 6 aa, Homo sapiens
SVGAGI

SEQ ID NO: 682 - protein, 7 aa, Homo sapiens
IFSNMDM

SEQ ID NO: 683 - protein, 6 aa, Homo sapiens
SVGAGI

SEQ ID NO: 684 - protein, 7 aa, Homo sapiens
IRSNERE

SEQ ID NO: 685 - protein, 6 aa, Homo sapiens
FYNNEI

SEQ ID NO: 686 - protein, 7 aa, Homo sapiens
IRSNERE

SEQ ID NO: 687 - protein, 6 aa, Homo sapiens
FYNNEI

SEQ ID NO: 688 - protein, 7 aa, Homo sapiens
IRSNVGE

SEQ ID NO: 689 - protein, 6 aa, Homo sapiens
FNNNVP

SEQ ID NO: 690 - protein, 7 aa, Homo sapiens
IRSNERE

SEQ ID NO: 691 - protein, 6 aa, Homo sapiens
FYNNEI

SEQ ID NO: 692 - protein, 7 aa, Homo sapiens
ISSIKDK

SEQ ID NO: 693 - protein, 5 aa, Homo sapiens
SVGIG

SEQ ID NO: 694 - protein, 7 aa, Homo sapiens
IRSNVGE

SEQ ID NO: 695 - protein, 6 aa, Homo sapiens
YFSETQ

SEQ ID NO: 696 - protein, 7 aa, Homo sapiens
MTFSENT

SEQ ID NO: 697 - protein, 6 aa, Homo sapiens
YFSETQ

SEQ ID NO: 698 - protein, 5 aa, Homo sapiens
GLTSN

SEQ ID NO: 699 - protein, 7 aa, Homo sapiens
LQKGGEE

SEQ ID NO: 700 - protein, 6 aa, Homo sapiens
FYNNEI

SEQ ID NO: 701 - protein, 5 aa, Homo sapiens
VYSSG

SEQ ID NO: 702 - protein, 7 aa, Homo sapiens
SNEGSKA

SEQ ID NO: 703 - protein, 7 aa, Homo sapiens
VVTGGEV

SEQ ID NO: 704 - protein, 5 aa, Homo sapiens
IPSGT

SEQ ID NO: 705 - protein, 6 aa, Homo sapiens
FQNNGV

SEQ ID NO: 706 - protein, 5 aa, Homo sapiens
VYSSG

SEQ ID NO: 707 - protein, 7 aa, Homo sapiens
SNEGSKA

SEQ ID NO: 708 - protein, 7 aa, Homo sapiens
SNEGSKA

SEQ ID NO: 709 - DNA, 21 bp, Homo sapiens
gccacgaaggctgatgacaag

SEQ ID NO: 710 - DNA, 21 bp, Homo sapiens
tccaatgagggctccaaggcc

SEQ ID NO: 711 - DNA, 24 bp, Homo sapiens
tacttttcaggagacactctggtt

SEQ ID NO: 712 - DNA, 18 bp, Homo sapiens
ttttataataatgaaatc

SEQ ID NO: 713 - DNA, 21 bp, Homo sapiens
tccaatgagggctccaaggcc

SEQ ID NO: 714 - DNA, 21 bp, Homo sapiens
ctaacttcaagtggaattgaa

SEQ ID NO: 715 - DNA, 18 bp, Homo sapiens
tttaacaacaacgttccg

SEQ ID NO: 716 - DNA, 21 bp, Homo sapiens
attttttcaaatatggacatg

SEQ ID NO: 717 - DNA, 18 bp, Homo sapiens
tcaatgaatgttgaggtg

SEQ ID NO: 718 - DNA, 21 bp, Homo sapiens
ttgctatcaaatggagcagtg

SEQ ID NO: 719 - DNA, 18 bp, Homo sapiens
tcaatgaatgttgaggtg

SEQ ID NO: 720 - DNA, 18 bp, Homo sapiens
tcaatgaatgttgaggtg

SEQ ID NO: 721 - DNA, 24 bp, Homo sapiens
tatttatcaggatccaccctggtt

SEQ ID NO: 722 - DNA, 18 bp, Homo sapiens
ttccaaggcaacagtgca

SEQ ID NO: 723 - DNA, 18 bp, Homo sapiens
ttccaaggcaacagtgca

SEQ ID NO: 724 - DNA, 21 bp, Homo sapiens
gccacgaaggctgatgacaag

SEQ ID NO: 725 - DNA, 21 bp, Homo sapiens
atacgtccagatgtgagtgaa

SEQ ID NO: 726 - DNA, 21 bp, Homo sapiens
tccaatgagggctccaaggcc

SEQ ID NO: 727 - DNA, 18 bp, Homo sapiens
atatactccaatggtgac

SEQ ID NO: 728 - DNA, 18 bp, Homo sapiens
tcagttggtgctggtatc

SEQ ID NO: 729 - DNA, 21 bp, Homo sapiens
gccacgaaggctgatgacaag

SEQ ID NO: 730 - DNA, 21 bp, Homo sapiens
tccaatgagggctccaaggcc

SEQ ID NO: 731 - DNA, 15 bp, Homo sapiens
ggatacaagacaaaa

SEQ ID NO: 732 - DNA, 18 bp, Homo sapiens
tacttcagtgagacacag

SEQ ID NO: 733 - DNA, 15 bp, Homo sapiens
ggtcttacaagcaat

SEQ ID NO: 734 - DNA, 21 bp, Homo sapiens
tccaatgagggctccaaggcc

SEQ ID NO: 735 - DNA, 18 bp, Homo sapiens
aatgctctggatggtttg

SEQ ID NO: 736 - DNA, 12 bp, Homo sapiens
ggctcaaagcct

SEQ ID NO: 737 - DNA, 18 bp, Homo sapiens
tatgagaatgaggaagca

SEQ ID NO: 738 - DNA, 12 bp, Homo sapiens
ggctcaaagcct

SEQ ID NO: 739 - DNA, 18 bp, Homo sapiens
tatgagaatgaggaagca

SEQ ID NO: 740 - DNA, 21 bp, Homo sapiens
ttatataaggctggtgaattg

SEQ ID NO: 741 - DNA, 18 bp, Homo sapiens
tcagttggtgctggtatc

SEQ ID NO: 742 - DNA, 21 bp, Homo sapiens
ttatataaggctggtgaattg

SEQ ID NO: 743 - DNA, 21 bp, Homo sapiens
gcaaatcagggctctgaggcc

SEQ ID NO: 744 - DNA, 18 bp, Homo sapiens
atatactccaatggtgac

SEQ ID NO: 745 - DNA, 18 bp, Homo sapiens
tacaataataaggagctc

SEQ ID NO: 746 - DNA, 15 bp, Homo sapiens
ctgagctcagggaag

SEQ ID NO: 747 - DNA, 15 bp, Homo sapiens
ctgagctcagggaag

SEQ ID NO: 748 - DNA, 18 bp, Homo sapiens
tcagttggtgctggtatc

SEQ ID NO: 749 - DNA, 24 bp, Homo sapiens
cggaactcttttgatgagcaaaat

SEQ ID NO: 750 - DNA, 18 bp, Homo sapiens
tattatagggaggaagag

SEQ ID NO: 751 - DNA, 18 bp, Homo sapiens
atatactccaatggtgac

SEQ ID NO: 752 - DNA, 18 bp, Homo sapiens
tcctttgatgtcaaagat

SEQ ID NO: 753 - DNA, 24 bp, Homo sapiens
cggaactcttttgatgagcaaaat

SEQ ID NO: 754 - DNA, 18 bp, Homo sapiens
ctccagaaagaaaatatc

SEQ ID NO: 755 - DNA, 21 bp, Homo sapiens
ttatataaggctggtgaattg

SEQ ID NO: 756 - DNA, 18 bp, Homo sapiens
tcacagatagtaaatgac

SEQ ID NO: 757 - DNA, 15 bp, Homo sapiens
attccctcagggaca

SEQ ID NO: 758 - DNA, 18 bp, Homo sapiens
ttccgcaaccgggctcct

SEQ ID NO: 759 - DNA, 15 bp, Homo sapiens
ggtctaaaaaacaat

SEQ ID NO: 760 - DNA, 18 bp, Homo sapiens
tcaatgaatgttgaggtg

SEQ ID NO: 761 - DNA, 24 bp, Homo sapiens
tatttatcaggatccaccctggtt

SEQ ID NO: 762 - DNA, 18 bp, Homo sapiens
tcagcagctgctgatatt

SEQ ID NO: 763 - DNA, 21 bp, Homo sapiens
attcgttcaaatatggacaaa

SEQ ID NO: 764 - DNA, 21 bp, Homo sapiens
attcgttcaaatatggacaaa

SEQ ID NO: 765 - DNA, 18 bp, Homo sapiens
ttccagaatgaagctcaa

SEQ ID NO: 766 - DNA, 24 bp, Homo sapiens
tacacaacaggggccaccctggtt

SEQ ID NO: 767 - DNA, 18 bp, Homo sapiens
tcagttggtgctggtatc

SEQ ID NO: 768 - DNA, 18 bp, Homo sapiens
tcagttggtgctggtatc

SEQ ID NO: 769 - DNA, 21 bp, Homo sapiens
attttttcaaatatggacatg

SEQ ID NO: 770 - DNA, 18 bp, Homo sapiens
tcagttggtgctggtatc

SEQ ID NO: 771 - DNA, 21 bp, Homo sapiens
atacgttcaaatgaaagagag

SEQ ID NO: 772 - DNA, 18 bp, Homo sapiens
ttttataataatgaaatc

SEQ ID NO: 773 - DNA, 21 bp, Homo sapiens
atacgttcaaatgaaagagag

SEQ ID NO: 774 - DNA, 18 bp, Homo sapiens
ttttataataatgaaatc

SEQ ID NO: 775 - DNA, 21 bp, Homo sapiens
attcgttcaaatgtgggcgaa

SEQ ID NO: 776 - DNA, 18 bp, Homo sapiens
tttaacaacaacgttccg

SEQ ID NO: 777 - DNA, 21 bp, Homo sapiens
atacgttcaaatgaaagagag

SEQ ID NO: 778 - DNA, 18 bp, Homo sapiens
ttttataataatgaaatc

SEQ ID NO: 779 - DNA, 21 bp, Homo sapiens
ataagttccattaaggataaa

SEQ ID NO: 780 - DNA, 15 bp, Homo sapiens
tccgttggtattggc

SEQ ID NO: 781 - DNA, 21 bp, Homo sapiens
attcgttcaaatgtgggcgaa

SEQ ID NO: 782 - DNA, 18 bp, Homo sapiens
tacttcagtgagacacag

SEQ ID NO: 783 - DNA, 21 bp, Homo sapiens
atgactttcagtgagaacaca

SEQ ID NO: 784 - DNA, 18 bp, Homo sapiens
tacttcagtgagacacag

SEQ ID NO: 785 - DNA, 15 bp, Homo sapiens
ggtcttacaagcaat

SEQ ID NO: 786 - DNA, 21 bp, Homo sapiens
ctacagaaaggtggggaagag

SEQ ID NO: 787 - DNA, 18 bp, Homo sapiens
ttttataataatgaaatc

SEQ ID NO: 788 - DNA, 15 bp, Homo sapiens
gtatactccagtggt

SEQ ID NO: 789 - DNA, 21 bp, Homo sapiens
tccaatgagggctccaaggcc

SEQ ID NO: 790 - DNA, 21 bp, Homo sapiens
gtagttacgggtggagaagtg

SEQ ID NO: 791 - DNA, 15 bp, Homo sapiens
attccctcagggaca

SEQ ID NO: 792 - DNA, 18 bp, Homo sapiens
tttcagaataacggtgta

SEQ ID NO: 793 - DNA, 15 bp, Homo sapiens
gtatactccagtggt

SEQ ID NO: 794 - DNA, 21 bp, Homo sapiens
tccaatgagggctccaaggcc

SEQ ID NO: 795 - DNA, 21 bp, Homo sapiens
tccaatgagggctccaaggcc